(19) United States
(12) Patent Application Publication
(10) Pub. No.: US 2024/0059773 A1
(43) Pub. Date: Feb. 22, 2024

(54) SIALIDASE-PD-1-ANTIBODY FUSION PROTEINS AND METHODS OF USE THEREOF

(71) Applicant: Palleon Pharmaceuticals Inc., Waltham, MA (US)

(72) Inventors: Li Peng, Lexington, MA (US); Lihui Xu, Chestnut Hill, MA (US); Autumn Turner, Waltham, MA (US)

(21) Appl. No.: 18/260,378

(22) PCT Filed: Jan. 6, 2022
(86) PCT No.: PCT/US2022/011487

§ 371 Date: Jul. 5, 2023

Related U.S. Application Data
(60) Provisional application No. 63/134,415, filed on Jan. 6, 2021.

(51) Int. Cl. C07K 16/28; C12N 9/24; A61P 35/00

(52) U.S. Cl. CPC C07K 16/2818; C12N 9/2402; C12Y 302/01018; A61P 35/00; C07K 2319/33; C07K 2317/55; C07K 2317/76; C07K 2317/92; A61K 2039/505

(57) ABSTRACT

The invention relates generally to recombinant sialidase and anti-PD-1 immunoglobulin antigen-binding domain fusion proteins. The invention also provides antibody conjugates including a sialidase and an anti-PD-1 antibody or a portion thereof. The invention further relates to methods of using the sialidase fusion proteins or antibody conjugates for treating cancer.

### CROSS-REFERENCE TO RELATED APPLICATIONS

This application claims the benefit of and priority to U.S. Provisional Patent Application No. 63/134,415, filed Jan. 6, 2021, the disclosure of which is incorporated herein by reference in its entirety for all purposes.

### FIELD OF THE INVENTION

The invention relates generally to recombinant sialidase fusion proteins and antibody conjugates, and their use in the treatment of cancer.

### BACKGROUND

A growing body of evidence supports roles for glycans, and sialoglycans in particular, at various pathophysiological steps of tumor progression. Glycans regulate tumor proliferation, invasion, hematogenous metastasis and angiogenesis (Fuster et al. (2005) NAT. REV. CANCER 5(7): 526-42). The sialylation of cell surface glycoconjugates is frequently altered in cancers, resulting in the expression of sialylated tumor-associated carbohydrate antigens. The expression of sialylated glycans by tumor cells is often associated with increased aggressiveness and metastatic potential of a tumor (Julien S., Delannoy P. (2015) Sialic Acid and Cancer. In: Taniguchi N., Endo T., Hart G., Seeberger P., Wong CH. (eds) Glycoscience: Biology and Medicine. Springer, Tokyo. https://doi.org/10.1007/978-4-431-54841-6_193).

It has recently become apparent that Siglecs (sialic acid-binding immunoglobulin-like lectins), a family of sialic acid binding lectins, play a role in cancer immune suppression by binding to hypersialylated cells (e.g., hypersialylated cancer cells) and mediating the suppression of signals from activating NK cell receptors, thereby inhibiting NK cell-mediated killing of tumor cells (Jandus et al. (2014) J. CLIN. INVEST. 124: 1810-1820; Läubli et al. (2014) PROC. NATL. ACAD. SCI. USA 111: 14211-14216; Hudak et cd. (2014) NAT. CHEM. BIOL. 10: 69-75). Likewise, enzymatic removal of sialic acids by treatment with sialidase can enhance NK cell-mediated killing of tumor cells (Jandus, supra; Hudak, supra; Xiao et al. (2016) PROC. NATL. ACAD. SCI. USA 113(37): 10304-9).

PD-1 is a receptor present on the surface of T-cells that serves as an immune system checkpoint that inhibits or otherwise modulates T-cell activity at the appropriate time to prevent an overactive immune response. Cancer cells, however, can take advantage of this checkpoint by expressing ligands, for example, PD-L1, that interact with PD-1 on the surface of T-cells to reduce T-cell activity. Many anti-PD-1 antibodies have been developed for use in immuno-oncology therapies. For example, nivolumab has been approved in the United States for use in the treatment of, for example, certain melanomas, non-small cell lung cancers (NSCLC), small cell lung cancers (SCLC), mesotheliomas, renal cell carcinomas (RCC), Hodgkin lymphomas, squamous cell carcinomas of the head and neck, urothelial carcinomas, colorectal cancers, hepatocellular carcinomas, and esophageal squamous cell carcinomas; and pembrolizumab has been approved in the United States for use in the treatment of, for example, certain melanomas, non-small cell lung cancers (NSCLC), small cell lung cancers (SCLC), head and neck squamous cell cancers, Hodgkin lymphomas, primary mediastinal large B-cell lymphomas, urothelial carcinomas, gastric cancers, esophageal cancers, cervical cancers, hepatocellular carcinomas, Merkel cell carcinomas, renal cell carcinomas (RCC), endometrial carcinomas, cutaneous squamous cell carcinomas, and triple negative breast cancers.

Cancer immunotherapy with immune checkpoint inhibitors, including antibodies blocking the PD-1/PD-L1 pathway, has improved the outcome of many cancer patients. However, despite advances that have been made to date, many patients do not respond to currently available immune checkpoint inhibitors. Accordingly, there is still a need for effective interventions that overcome the immune suppressive tumor microenvironment and for treating cancers associated with hypersialylated cells.

### SUMMARY OF THE INVENTION

The invention is based, in part, upon the discovery that it is possible to produce fusion proteins containing a sialidase enzyme and an anti-PD-1 immunoglobulin or a portion thereof, e.g., an antigen-binding domain and/or an immunoglobulin Fc domain, and/or antibody conjugates including a sialidase enzyme and an anti-PD-1 antibody or a portion thereof, e.g., an antigen-binding domain and/or an immunoglobulin Fc domain. The sialidase enzyme portion of the fusion protein and/or antibody conjugate may comprise at least one mutation relative to a wild-type sialidase. The mutations, or combination of mutations, can improve the expression, activity or both the expression and activity of the sialidase to improve its use in cancer diagnosis and/or treatment. The fusion proteins and/or antibody conjugates have suitable substrate specificities and activities to be useful in removing sialic acid and/or sialic acid containing molecules from the surface of cells, e.g., PD-1-expressing cells, and/or removing sialic acid and/or sialic acid containing molecules from the tumor microenvironment, and/or reducing the concentration of sialic acid and/or sialic acid containing molecules in the tumor microenvironment.

Accordingly, in one aspect, the invention provides a fusion protein comprising (or consisting essentially of): (a) sialidase enzyme; and (b) an anti-PD-1 immunoglobulin antigen-binding domain.

In certain embodiments, the sialidase is a human sialidase, e.g., a recombinant mutant human sialidase. In certain embodiments, the sialidase comprises: (a) a substitution or deletion of a methionine residue at a position corresponding to position 1 of wild-type human Neu2 (M1); (b) a substitution of a valine residue at a position corresponding to position 6 of wild-type human Neu2 (V6); (c) a substitution of a lysine residue at a position corresponding to position 9 of wild-type human Neu2 (K9); (d) a substitution of an alanine residue at a position corresponding to position 42 of wild-type human Neu2 (A42); (e) a substitution of a proline residue at a position corresponding to position 62 of wild-type human Neu2 (P62); (f) a substitution of an alanine residue at a position corresponding to position 93 of wild-type human Neu2 (A93); (g) a substitution of a glutamine residue at a position corresponding to position 126 of wild-type human Neu2 (Q126); (h) a substitution of an isoleucine residue at a position corresponding to position 187 of wild-type human Neu2 (I187); (i) a substitution of an alanine residue at a position corresponding to position 242 of wild-type human Neu2 (A242); (j) a substitution of a glutamine residue at a position corresponding to position 270 of wild-type human Neu2 (Q270); (k) a substitution of a serine residue at a position corresponding to position 301 of wild-type human Neu2 (S301); (l) a substitution of a tryptophan residue at a position corresponding to position 302 of wild-type human Neu2 (W302); (m) a substitution of a cysteine residue at a position corresponding to position 332 of wild-type human Neu2 (C332); (n) a substitution of a valine residue at a position corresponding to position 363 of wild-type human Neu2 (V363); or (o) a substitution of a leucine residue at a position corresponding to position 365 of wild-type human Neu2 (L365); or a combination of any of the foregoing substitutions.

In certain embodiments, in the sialidase: (a) the methionine residue at a position corresponding to position 1 of wild-type human Neu2 is deleted (ΔM1), is substituted by alanine (M1A), or is substituted by aspartic acid (M1D); (b) the valine residue at a position corresponding to position 6 of wild-type human Neu2 is substituted by tyrosine (V6Y); (c) the lysine residue at a position corresponding to position 9 of wild-type human Neu2 is substituted by aspartic acid (K9D); (d) the alanine residue at a position corresponding to position 42 of wild-type human Neu2 is substituted by arginine (A42R) or aspartic acid (A42D); (e) the proline residue at a position corresponding to position 62 of wild-type human Neu2 is substituted by asparagine (P62N), aspartic acid (P62D), histidine (P62H), glutamic acid (P62E), glycine (P62G), serine (P62S), or threonine (P62T); (f) the alanine residue at a position corresponding to position 93 of wild-type human Neu2 is substituted by glutamic acid (A93E) or lysine (A93K); (g) the glutamine residue at a position corresponding to position 126 of wild-type human Neu2 is substituted by leucine (Q126L), glutamic acid (Q126E), phenylalanine (Q126F), histidine (Q126H), isoleucine (Q126I), or tyrosine (Q126Y); (h) the isoleucine residue at a position corresponding to position 187 of wild-type human Neu2 is substituted by lysine (I187K); (i) the alanine residue at a position corresponding to position 242 of wild-type human Neu2 is substituted by cysteine (A242C), phenylalanine (A242F), glycine (A242G), histidine (A242H), isoleucine (A242I), lysine (A242K), leucine (A242L), methionine (A242M), asparagine (A242N), glutamine (A242Q), arginine (A242R), serine (A242S), valine (A242V), tryptophan (A242W), or tyrosine (A242Y); (j) the glutamine residue at a position corresponding to position 270 of wild-type human Neu2 is substituted by alanine (Q270A), histidine (Q270H), phenylalanine (Q270F), proline (Q270P), serine (Q270S), or threonine (Q270T); (k) the serine residue at a position corresponding to position 301 of wild-type human Neu2 is substituted by alanine (S301A), aspartic acid (S301D), glutamic acid (S301E), phenylalanine (S301F), histidine (S301H), lysine (S301K), leucine (S301L), methionine (S301M), asparagine (S301N), proline (S301P), glutamine (S301Q), arginine (S301R), threonine (S301T), valine (S301V), tryptophan (S301W), or tyrosine (S301Y); (l) the tryptophan residue at a position corresponding to position 302 of wild-type human Neu2 is substituted by alanine (W302A), aspartic acid (W302D), phenylalanine (W302F), glycine (W302G), histidine (W302H), isoleucine (W3021), lysine (W302K), leucine (W302L), methionine (W302M), asparagine (W302N), proline (W302P), glutamine (W302Q), arginine (W302R), serine (W302S), threonine (W302T), valine (W302V), or tyrosine (W302Y); (m) the cysteine residue at a position corresponding to position 332 of wild-type human Neu2 is substituted by alanine (C332A); (n) the valine residue at a position corresponding to position 363 of wild-type human Neu2 is substituted by arginine (V363R); or (o) the leucine residue at a position corresponding to position 365 of wild-type human Neu2 is substituted by glutamine (L365Q), histidine (L365H), isoleucine (L365I), lysine (L365K) or serine (L365S); or the sialidase comprises a combination of any of the foregoing substitutions. For example, the sialidase may comprise a modification selected from ΔM1, M1A, M1D, V6Y, K9D, A42R, P62G, P62N, P62S, P62T, A93E, Q126Y, I187K, A242F, A242W, A242Y, Q270A, Q270T, S301A, S301R, W302K, W302R, C332A, V363R, and L365I, or a combination of any of the foregoing modifications.

In certain embodiments, the sialidase comprises: (a) the M1D, V6Y, P62G, A93E, I187K, and C332A substitutions; (b) the M1D, V6Y, K9D, A93E, I187K, C332A, V363R, and L365I substitutions; (c) the M1D, V6Y, P62N, I187K, and C332A substitutions; (d) the M1D, V6Y, I187K, Q270A, S301R, W302K, and C332A substitutions; (e) the M1D, V6Y, P62S, I187K, Q270A, S301R, W302K, and C332A substitutions; (f) the M1D, V6Y, P62T, I187K, Q270A, S301R, W302K, and C332A substitutions; (g) the M1D, V6Y, P62N, I187K, Q270A, S301R, W302K, and C332A substitutions; (h) the M1D, V6Y, P62G, A93E, I187K, S301A, W302R, and C332A substitutions; (i) the M1D, V6Y, P62G, A93E, Q126Y, I187K, Q270T, and C332A substitutions; (j) the M1D, V6Y, P62G, A93E, Q126Y, I187K, and C332A substitutions; (k) the M1D, V6Y, P62G, A93E, Q126Y, I187K, A242F, Q270T, and C332A substitutions; or (l) the M1D, V6Y, A42R, P62G, A93E, Q126Y, I187K, A242F, Q270T, and C332A mutations.

In certain embodiments, the sialidase is selected from Neu1, Neu2, Neu3, and Neu4, e.g., the sialidase is Neu2.

In certain embodiments, the sialidase has a different substrate specificity than the corresponding wild-type sialidase. For example, in certain embodiments the sialidase can cleave α2,3, α2,6, and/or α2,8 linkages. In certain embodiments the sialidase can cleave α2,3 and α2,8 linkages.

In certain embodiments, the sialidase comprises any one of SEQ ID NOs: 48-63, 94, 97, 100, or 126, or an amino acid sequence that has at least 85%, 90%, 95%, 96%, 97%, 98%, or 99% sequence identity to any one of SEQ ID NOs: 48-63, 94, 97, 100, or 126.

In certain embodiments, the sialidase comprises mutation or combination of mutations set forth in any one of Tables 1-9.

In certain embodiments, the fusion protein further comprises an immunoglobulin Fc domain. In certain embodiments, the immunoglobulin Fc domain is derived from a human IgG1, IgG2, IgG3, IgG4, IgA1, IgA2, IgD, IgE, or IgM Fc domain, e.g., the immunoglobulin Fc domain is derived from a human IgG1, IgG2, IgG3, or IgG4 Fc domain, e.g., the immunoglobulin Fc domain is derived from a human IgG1 Fc domain.

In certain embodiments, the anti-PD-1 immunoglobulin antigen-binding domain is associated (for example, covalently or non-covalently associated) with a second anti-PD-1 immunoglobulin antigen-binding domain to produce an anti-PD-1 antigen-binding site.

In certain embodiments, the anti-PD-1 immunoglobulin antigen-binding domain is derived from an antibody selected from nivolumab, pembrolizumab, cemiplimab, spartalizumab (PDR001), TX-4014, camrelizumab (SHR1210), sintilimab (IBI308), tislelizumab (BGB-A317), toripalimab (JS 001), dostarlimab (TSR-042, WBP-285), INCMGA00012 (MGA012), AMP-514, and pidilizumab, e.g., the anti-PD-1 immunoglobulin antigen-binding domain is derived from nivolumab or pembrolizumab.

In certain embodiments, the sialidase and the immunoglobulin Fc domain and/or the anti-PD-1 immunoglobulin antigen-binding domain are linked by a peptide bond or an amino acid linker.

In certain embodiments, the fusion protein comprises any one of SEQ ID NOs: 67-73, 78, 81-87, 95, 96, 98, 99, 101, 102, 105, 106, 108, 111, 112, 115, 122, 123, 125, 127, 128, 130, 132, 134, or 145.

In another aspect, the invention provides an antibody conjugate comprising any of the foregoing fusion proteins. In certain embodiments, the antibody conjugate comprises a single sialidase. In other embodiments, the antibody conjugate comprises two sialidases, which can be the same or different. In certain embodiments the antibody conjugate comprises two identical sialidases. In certain embodiments, the antibody conjugate comprises a single anti-PD-1 antigen-binding site. In other embodiments, the antibody conjugate comprises two anti-PD-1 antigen-binding sites, which can be the same or different. In certain embodiments, the antibody conjugate comprises two identical anti-PD-1 antigen-binding sites.

In certain embodiments, the antibody conjugate has a molecular weight from about 135 kDa to about 165 kDa, or the antibody conjugate has a molecular weight from about 215 kDa to about 245 kDa.

In certain embodiments, the antibody conjugate comprises: (a) a first polypeptide comprising an immunoglobulin light chain; (b) a second polypeptide comprising an immunoglobulin heavy chain; and (c) a third polypeptide comprising an immunoglobulin Fc domain and a sialidase; wherein the first and second polypeptides are covalently linked together and the second and third polypeptides are covalently linked together, and wherein the first polypeptide and the second polypeptide together define an anti-PD-1 antigen-binding site. The third polypeptide may, for example, comprise the sialidase and the immunoglobulin Fc domain in an N- to C-terminal orientation. The first polypeptide may, for example, comprise SEQ ID NO: 77, the second polypeptide may, for example, comprise SEQ ID NO: 105, and/or the third polypeptide may, for example, comprise any one of SEQ ID NOs: 67-73, 78, 81-87, 95, 96, 98, 99, 101, 102, 106, 108, 111, 112, 115, 122, 123, 125, 127, or 128.

In certain embodiments, the antibody conjugate comprises: (a) a first polypeptide comprising a first immunoglobulin light chain; (b) a second polypeptide comprising a first immunoglobulin heavy chain and a first sialidase; (c) a third polypeptide comprising a second immunoglobulin heavy chain and a second sialidase; and (d) a fourth polypeptide comprising a second immunoglobulin light chain; wherein the first and second polypeptides are covalently linked together, the third and fourth polypeptides are covalently linked together, and the second and third polypeptides are covalently linked together, and wherein the first polypeptide and the second polypeptide together define a first anti-PD-1 antigen-binding site, and the third polypeptide and the fourth polypeptide together define a second anti-PD-1 antigen-binding site. The second and third polypeptides may, for example, comprise the first and second immunoglobulin heavy chain and the first and second sialidase, respectively, in an N- to C-terminal orientation. The first and fourth polypeptide may, for example, comprise SEQ ID NO: 77. The second and third polypeptide may, for example, comprise SEQ ID NO: 145.

In certain embodiments, the antibody conjugate comprises: (a) a first polypeptide comprising a first sialidase, a first immunoglobulin Fc domain, and a first single chain variable fragment (scFv); and (b) a second polypeptide comprising a second sialidase, a second immunoglobulin Fc domain, and an optional second single chain variable fragment (scFv); wherein the first and second polypeptides are covalently linked together, and wherein the first scFv defines a first anti-PD-1 antigen-binding site, and the second scFv, when present, defines a second anti-PD-1 antigen-binding site. The first polypeptide may, for example comprise the first sialidase, the first immunoglobulin Fc domain, and the first scFv in an N- to C-terminal orientation. The second polypeptide may, for example, comprise the second sialidase, the second immunoglobulin Fc domain, and the optional second scFv in an N- to C-terminal orientation.

In certain embodiments, the antibody conjugate comprises: (a) a first polypeptide comprising an immunoglobulin light chain; (b) a second polypeptide comprising an immunoglobulin heavy chain and a single chain variable fragment (scFv); and (c) a third polypeptide comprising an immunoglobulin Fc domain and a sialidase, wherein the first and second polypeptides are covalently linked together and the second and third polypeptides are covalently linked together, and wherein the immunoglobulin light chain and immunoglobulin heavy chain together define a first anti-PD-1 antigen-binding site and the scFv defines a second anti-PD-1 antigen-binding site. The second polypeptide may, for example comprise the immunoglobulin heavy chain and the scFv in an N- to C-terminal orientation. The third polypeptide may, for example, comprise the sialidase and the immunoglobulin Fc domain in an N- to C-terminal orientation.

In another aspect, the invention provides an isolated nucleic acid comprising a nucleotide sequence encoding any of the foregoing fusion proteins or at least a portion of any of the foregoing antibody conjugates. In another aspect, the invention provides an expression vector comprising any of the foregoing nucleic acids. In another aspect, the invention provides a host cell comprising any of the foregoing expression vectors.

In another aspect, the invention provides a pharmaceutical composition comprising any of the foregoing fusion proteins or any of the foregoing antibody conjugates.

In another aspect, the invention provides a method of treating cancer in a subject in need thereof. The method comprises administering to the subject an effective amount of any of the foregoing fusion proteins, any of the foregoing antibody conjugates, or any of the foregoing pharmaceutical compositions.

In certain embodiments, the cancer is selected from melanoma, bladder cancer, breast cancer, cervical cancer, esophageal cancer, gastric cancer, kidney cancer, lung cancer (e.g., NSCLC), ovarian cancer, metastatic Merkel cell carcinoma (MCC), metastatic urothelial carcinoma (UC), pancreatic cancer, colon cancer, stomach cancer, AML, and multiple myeloma. For example, in certain embodiments, the cancer is NSCLC or melanoma.

These and other aspects and features of the invention are described in the following detailed description and claims.

### DESCRIPTION OF THE DRAWINGS

The invention can be more completely understood with reference to the following drawings.

FIG. 1 depicts an SDS-PAGE gel showing recombinant human Neu1, Neu2, Neu3, and Salmonella typhimurium (St-sialidase) under non-reducing and reducing conditions. Monomer and dimer species are indicated.

FIG. 2 is a bar graph showing the enzymatic activity of recombinant human Neu1, Neu2, and Neu3.

FIG. 3 is a line graph showing enzymatic activity as a function of substrate concentration for recombinant human Neu2 and Neu3 at the indicated pH.

FIGS. 4A-4I depict schematic representations of certain antibody conjugate constructs containing a sialidase enzyme, e.g., a human sialidase enzyme, and an anti-PD-1 antigen binding site. For each antibody conjugate construct that contains more than one (e.g., two) sialidase, each sialidase may be the same or different. For each antibody conjugate construct that contains more than one (e.g., two) anti-PD-1 antigen binding site, each anti-PD-1 antigen binding site may be the same or different. For each antibody conjugate construct that contains an Fc domain, it is understood that the Fc domain can be a wild type Fc domain or can be an engineered Fc domain. For example, the Fc domain may be engineered to contain either a “knob” mutation, e.g., T366Y, or a “hole” mutation, e.g., Y407T, or both, to promote heterodimerization, or the Fc domain may be engineered to contain one or more modifications, e.g., point mutations, to provide any other modified Fc domain functionality.

FIG. 5 depicts schematic representations of certain antibody conjugate constructs containing a sialidase enzyme, e.g., a human sialidase enzyme, and an antigen binding site. For each antibody conjugate construct that contains more than one (e.g., two) antigen binding site, each antigen binding site may be the same or different. For each antibody conjugate construct that contains an Fc domain, it is understood that the Fc domain can be a wild type Fc domain or can be an engineered Fc domain. For example, the Fc domain may be engineered to contain either a “knob” mutation, e.g., T366Y, or a “hole” mutation, e.g., Y407T, or both, to promote heterodimerization, or the Fc domain may be engineered to contain one or more modifications, e.g., point mutations, to provide any other modified Fc domain functionality.

FIGS. 6A-6E are schematic representations of exemplary fusion protein conjugates referred to as a Raptor antibody sialidase conjugate (FIG. 6A), a Janus antibody sialidase conjugate (FIG. 6B), a Lobster antibody sialidase conjugate (FIG. 6C), a Bunk antibody sialidase conjugate (FIG. 6D), and a Lobster-Fab antibody sialidase conjugate (FIG. 6E).

FIG. 7A depicts a size exclusion chromatography (SEC) trace of ASC #3. FIG. 7B depicts a size exclusion chromatography (SEC) trace of ASC #6. Peaks (triangles) and the corresponding elution time are indicated.

FIG. 8 depicts blocking of the PD-L1 and PD-1 interaction by the indicated ASCs, as measured by fold induction of a PD-1/PD-L1 linked NFAT driven luciferase reporter. Pembrolizumab was included as a control. ASC #3-1, 3-2 and 3-3 refer to ASC #3 from three different preparations.

FIG. 9 depicts blocking of the interaction between human PD-L1-Fc and human PD-1 by the indicated ASCs, as measured by ELISA. Pembrolizumab was included as a control.

FIG. 10 depicts in vivo efficacy of anti-PD-1 antibody sialidase conjugate ASC #3 in a MC38 mouse syngeneic subcutaneous tumor model. Pembrolizumab and isotype antibody were included as controls. Mean tumor volume ±SEM over 18 days for the indicated treatments is depicted in FIG. 10A. Triangles indicate drug administration. Individual tumor volumes on day 18 are depicted in FIG. 10B. * adjusted p<0.05; *** adjusted p<0.001; ns non-significant, as determined by one-way ANOVA.

FIG. 11 depicts in vivo efficacy of anti-PD-1 antibody sialidase conjugate ASC #3 in a CT26 mouse syngeneic subcutaneous tumor model (female BALB/c-hPD-1/hPD-L1 mice bearing CT26-hPD-L1 tumors). Pembrolizumab and isotype antibody were included as controls. Mean tumor volume ±SEM over 18 days for the indicated treatment is depicted in FIG. 11A. Triangles indicate drug administration. Individual tumor volumes on day 18 are depicted in FIG. 11B. **** adjusted p<0.001 as determined by one-way ANOVA (which applies to both pembrolizumab and ASC #3 relative to isotype).

### DETAILED DESCRIPTION

The invention is based, in part, upon the discovery that it is possible to produce fusion proteins containing a sialidase enzyme and an anti-PD-1 immunoglobulin or a portion thereof, e.g., an antigen-binding domain and/or an immunoglobulin Fc domain, and/or antibody conjugates including a sialidase enzyme and an anti-PD-1 antibody or a portion thereof, e.g., an antigen-binding domain and/or an immunoglobulin Fc domain. The sialidase enzyme portion of the fusion protein and/or antibody conjugate may comprise at least one mutation relative to a wild-type sialidase. The mutations, or combination of mutations, can improve the expression, activity or both the expression and activity of the sialidase to improve its use in cancer diagnosis and/or treatment. The fusion proteins and/or antibody conjugates have suitable substrate specificities and activities to be useful in removing sialic acid and/or sialic acid containing molecules from the surface of cells, e.g., PD-1-expressing cells, and/or removing sialic acid and/or sialic acid containing molecules from the tumor microenvironment, and/or reducing the concentration of sialic acid and/or sialic acid containing molecules in the tumor microenvironment.

The invention further relates to pharmaceutical compositions and methods of using fusion proteins and/or antibody conjugates to treat cancer.

### I. Sialidase Anti-PD-1 Fusion Proteins

To promote the selective removal of sialic acids on cells, e.g., PD-1 expressing cells, and/or in the tumor microenvironment, it may be helpful to target a sialidase as described herein to such a cell or to such a tumor microenvironment. Additionally, in order to promote the removal of sialic acid by a sialidase in a subject, it may be helpful to extend the plasma half-life of the sialidase in the subject. These can be achieved by including the sialidase in a fusion protein and/or antibody conjugate (e.g., a chemically conjugated conjugate).

Accordingly, the invention further provides fusion proteins comprising a sialidase enzyme, or a functional fragment thereof, and a portion or fragment of an anti-PD-1 antibody, such as an immunoglobulin Fc domain (also referred to herein as an Fc domain), or an immunoglobulin antigen-binding domain (also referred to herein as an antigen-binding domain). In certain embodiments, the sialidase and anti-PD-1 antibody or portion thereof (e.g., immunoglobulin Fc domain or antigen-binding domain) are linked by a peptide bond or an amino acid linker.

As used herein, unless otherwise indicated, the term “fusion protein” is understood to refer to a single polypeptide chain comprising amino acid sequences based upon two or more separate proteins or polypeptide chains, where the two amino acid sequences may be fused together directly or via an intervening linker sequence, e.g., via an intervening amino acid linker. A nucleotide sequence encoding such a fusion protein can, for example, be created using conventional recombinant DNA technologies.

In certain embodiments, a fusion protein comprises a tag, such as a Strep tag (e.g., a Strep II tag), a His tag (e.g., a 10× His tag), a myc tag, or a FLAG tag. The tag can be located on the C-terminus or the N-terminus of the fusion protein.

a. Sialidase Portion

As used herein, the term “sialidase” refers to any enzyme, or a functional fragment thereof, that cleaves a terminal sialic acid residue from a substrate, for example, a glycoprotein or a glycolipid. The term sialidase includes variants having one or more amino acid substitutions, deletions, or insertions relative to a wild-type sialidase sequence, and/or fusion proteins or conjugates including a sialidase. Sialidases are also called neuraminidases, and, unless indicated otherwise, the two terms are used interchangeably herein. As used herein, the term “functional fragment” of a sialidase refers to fragment of a full-length sialidase that retains, for example, at least 10%, at least 20%, at least 30%, at least 40%, at least 50%, at least 60%, at least 70%, at least 80%, at least 90%, at least 95%, or 100% of the enzymatic activity of the corresponding full-length, naturally occurring sialidase. Sialidase enzymatic activity may be assayed by any method known in the art, including, for example, by measuring the release of sialic acid from the fluorogenic substrate 4-methylumbelliferyl-N-acetylneuraminic acid (4MU-NeuAc). In certain embodiments, the functional fragment comprises at least 100, 150, 200, 250, 300, 310, 320, 330, 340, 350, 360, or 370 consecutive amino acids present in a full-length, naturally occurring sialidase.

In certain embodiments, a sialidase portion of a sialidase-anti-PD-1 fusion protein is derived from a eukaryotic sialidase, e.g., a mammalian sialidase, e.g., a human or mouse sialidase.

Four sialidases are encoded in the human genome: Neu1, Neu2, Neu3 and Neu4. Human Neu1 is a lysosomal neuraminidase enzyme which functions in a complex with beta-galactosidase and cathepsin A. The amino acid sequence of human Neu1 is depicted in SEQ ID NO: 7, and a nucleotide sequence encoding human Neu1 is depicted in SEQ ID NO: 23.

Human Neu2 is a cytosolic sialidase enzyme. The amino acid sequence of human Neu2 is depicted in SEQ ID NO: 1, and a nucleotide sequence encoding human Neu2 is depicted in SEQ ID NO: 24. Unless stated otherwise, as used herein, wild-type human Neu2 refers to human Neu2 having the amino acid sequence of SEQ ID NO: 1.

Human Neu3 is a plasma membrane sialidase with an activity specific for gangliosides. Human Neu3 has two isoforms: isoform 1 and isoform 2. The amino acid sequence of human Neu3, isoform 1 is depicted in SEQ ID NO: 8, and a nucleotide sequence encoding human Neu3, isoform 1 is depicted in SEQ ID NO: 25. The amino acid sequence of human Neu3, isoform 2 is depicted in SEQ ID NO: 9, and a nucleotide sequence encoding human Neu3, isoform 2 is depicted in SEQ ID NO: 34.

Human Neu4 has two isoforms: isoform 1 is a peripheral membrane protein and isoform 2 localizes to the lysosome lumen. The amino acid sequence of human Neu4, isoform 1 is depicted in SEQ ID NO: 10, and a nucleotide sequence encoding human Neu4, isoform 1 is depicted in SEQ ID NO: 26. The amino acid sequence of human Neu4, isoform 2 is depicted in SEQ ID NO: 11, and a nucleotide sequence encoding human Neu4, isoform 2 is depicted in SEQ ID NO: 35.

Four sialidases have also been found in the mouse genome and are referred to as Neu1, Neu2, Neu3 and Neu4. The amino acid sequence of mouse Neu1 is depicted in SEQ ID NO: 38, and a nucleotide sequence encoding mouse Neu1 is depicted in SEQ ID NO: 42. The amino acid sequence of mouse Neu2 is depicted in SEQ ID NO: 39 and a nucleotide sequence encoding mouse Neu2 is depicted in SEQ ID NO: 43. The amino acid sequence of mouse Neu3 is depicted in SEQ ID NO: 40, and a nucleotide sequence encoding mouse Neu3 is depicted in SEQ ID NO: 44. The amino acid sequence of mouse Neu4 is depicted in SEQ ID NO: 41, and a nucleotide sequence encoding mouse Neu4 is depicted in SEQ ID NO: 45.

In certain embodiments, a sialidase portion of a sialidase-anti-PD-1 fusion protein is derived from a prokaryotic sialidase. Exemplary prokaryotic sialidases include sialidases from Salmonella typhimurium and Vibrio cholera. The amino acid sequence of Salmonella typhimurium sialidase (St-sialidase) is depicted in SEQ ID NO: 30, and a nucleotide sequence encoding Salmonella typhimurium sialidase is depicted in SEQ ID NO: 6. The amino acid sequence of Vibrio cholera sialidase is depicted in SEQ ID NO: 36, and a nucleotide sequence encoding Vibrio cholera sialidase is depicted in SEQ ID NO: 37.

In certain embodiments, the sialidase portion of a sialidase-anti-PD-1 fusion protein is a mutant sialidase, e.g., a recombinant mutant human sialidase. In certain embodiments, the recombinant mutant human sialidase has about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or more than 100% of the enzymatic activity of a corresponding (or template) wild-type human sialidase.

In certain embodiments, the recombinant mutant human sialidase has the same substrate specificity as the corresponding wild-type human sialidase. In other embodiments, the recombinant mutant human sialidase has a different substrate specificity than the corresponding wild-type human sialidase. For example, in certain embodiments the recombinant mutant human sialidase can cleave α2,3, α2,6, and/or α2,8 linkages. In certain embodiments the sialidase can cleave α2,3 and α2,8 linkages.

In certain embodiments, the expression yield of the recombinant mutant human sialidase in mammalian cells, e.g., HEK293 cells, CHO cells, murine myeloma cells (NS0, Sp2/0), or human fibrosarcoma cells (HT-1080), e.g., HEK293 cells, is greater than about 10%, about 20%, about 50%, about 75%, about 100%, about 150%, about 200%, about 250%, about 300%, about 400%, about 500%, about 600%, about 700%, about 800%, about 900%, or about 1,000% of the expression yield of the corresponding wild-type human sialidase.

In certain embodiments, the recombinant mutant human sialidase has about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or more than 100% of the enzymatic activity of a corresponding wild-type human sialidase, and the expression yield of the recombinant mutant human sialidase in mammalian cells, e.g., HEK293 cells, is greater than about 10%, about 20%, about 50%, about 75%, about 100%, about 150%, about 200%, about 250%, about 300%, about 400%, about 500%, about 600%, about 700%, about 800%, about 900%, or about 1,000% of the expression yield of a corresponding wild-type human sialidase.

In certain embodiments, the amino acid sequence of the recombinant mutant human sialidase has at least 50%, 55%, 60%, 65%, 70%, 75%, 80%, 85%, 90%, 95%, 96%, 97%, 98%, or 99% sequence identity to the amino acid sequence of a corresponding wild-type human sialidase.

1. Substitution of Cysteine Residues

In certain embodiments, the recombinant mutant human sialidase comprises a substitution of at least one cysteine (cys, C) residue. It has been discovered that certain cysteine residues in sialidases may inhibit expression of functional protein as a result of protein aggregation. Accordingly, in certain embodiments, the recombinant mutant human sialidase contains at least one mutation to remove a free cysteine (e.g., for Neu1 (SEQ ID NO: 7), a mutation of, for example, one or more of C111, C117, C171, C183, C218, C240, C242, and C252; for Neu2 (SEQ ID NO: 1), a mutation of, for example, one or more of C125, C196, C219, C272, C332, and C352; for Neu3 (SEQ ID NO: 8), a mutation of, for example, one or more of C7, C90, C99, C106, C127, C136, C189, C194, C226, C242, C250, C273, C279, C295, C356, C365, C368, C384, C383, C394, and C415; and for Neu4 (SEQ ID NO: 10), a mutation of, for example, one or more of C88, C125, C126, C186, C191, C211, C223, C239, C276, C437, C453, C480, and C481). Free cysteines can be substituted with any amino acid. In certain embodiments, the free cysteine is substituted with serine (ser, S), isoleucine (iso, I), valine (val, V), phenylalanine (phe, F), leucine (leu, L), or alanine (ala, A). Exemplary cysteine substitutions in Neu2 include C125A, C1251, C125S, C125V, C196A, C196L, C196V, C272S, C272V, C332A, C332S, C332V, C352L, and C352V.

In certain embodiments, the recombinant mutant human sialidase comprises two or more cysteine substitutions. Exemplary double or triple cysteine substitutions in Neu2 include: C125S and C332S; C272V and C332A; C272V and C332S; C332A and C352L; C125S and C196L; C196L and C352L; C196L and C332A; C332A and C352L; and C196L, C332A and C352L.

In certain embodiments, the recombinant mutant human sialidase is a Neu2 sialidase and comprises the substitutions C322A and C352L (SEQ ID NO: 5).

In certain embodiments, the sialidase contains an amino acid substitution at 2, 3, 4, 5, or 6 cysteines typically present in a human sialidase, e.g., Neu2 or Neu3.

In certain embodiments, the recombinant mutant human sialidase comprises a substitution or combination of substitutions corresponding to a substitution or combination of substitutions listed in TABLE 1 (amino acid positions corresponding to wild-type human Neu2 (SEQ ID NO: 1)).

TABLE 1
Substitution(s)
C125A
C125I
C125S
C125V
C196A
C196L
C196V
C272S
C272V
C332A
C332S
C332V
C352L
C352V
C125S + C332S
C272V + C332A
C272V + C332S
C332A + C352L
C125S + C196L
C196L + C352L
C196L + C332A
C196L + C332A + C352L

2. Substitutions of Residues to Increase pI and/or Decrease Hydrophobicity

The isoelectric point (pI) of a protein is the pH at which the net charge is zero. The pI also generally indicates the pH at which the protein is least soluble, which may affect the ability to express and purify the protein. Generally, a protein has good solubility if its pI is greater than 2 units above the pH of the solution. Human Neu2 has a predicted pI of 7.5. Thus, human Neu2 is least soluble around neutral pH, which is undesirable because expression and physiological systems are at neutral pH. In contrast, the sialidase from Salmonella typhimurium (St-sialidase), which exhibits good solubility and recombinant expression, has a pI of 9.6. Accordingly, to increase expression of human Neu2 or the other human sialidases, a recombinant mutant human sialidase may be designed to contain one or more amino acid substitution(s) wherein the substitution(s) increase(s) the pI of the sialidase relative to a sialidase without the substitution. Additionally, decreasing the number of hydrophobic amino acids on the surface of a sialidase may improve expression of sialidase by, for example, reducing aggregation. Accordingly, to increase expression of human Neu2 or the other human sialidases, a recombinant mutant human sialidase may be designed to contain one or more amino acid substitution(s) wherein the substitution(s) decrease(s) the hydrophobicity of a surface of the sialidase relative to a sialidase without the substitution(s).

Accordingly, in certain embodiments, the recombinant mutant human sialidase comprises at least one amino acid substitution, wherein the substitution increases the isoelectric point (pI) of the sialidase and/or decreases the hydrophobicity of the sialidase relative to a sialidase without the substitution. This may be achieved by introducing one or more charged amino acids, for example, positively or negatively charged amino acids, into the recombinant sialidase. In certain embodiments, the amino acid substitution is to a charged amino acid, for example, a positively charged amino acid such as lysine (lys, K), histidine (his, H), or arginine (arg, R), or a negatively charged amino acid such as aspartic acid (asp, D) or glutamic acid (glu, E). In certain embodiments, the amino acid substitution is to a lysine residue. In certain embodiments, the substitution increases the pI of the sialidase to about 7.75, about 8, about 8.25, about 8.5, about 8.75, about 9, about 9.25, about 9.5, or about 9.75.

In certain embodiments, the amino acid substitution occurs at a surface exposed D or E amino acid, in a helix or loop, or in a position that has a K or R in the corresponding position of St-sialidase. In certain embodiments, the amino acid substitution occurs at an amino acid that is remote from the catalytic site or otherwise not involved in catalysis, an amino acid that is not conserved with the other human Neu proteins or with St-Sialidase or Clostridium NanH, or an amino acid that is not located in a domain important for function (e.g., an Asp-box or beta strand).

Exemplary amino acid substitutions in Neu2 that increase the isoelectric point (pI) of the sialidase and/or decrease the hydrophobicity of the sialidase relative to a sialidase without the substitution include A2E, A2K, D215K, V325E, V325K, E257K, and E319K. In certain embodiments, the recombinant mutant human sialidase comprises two or more amino acid substitutions, including, for example, A2K and V325E, A2K and V325K, E257K and V325K, A2K and E257K, and E257K and A2K and V325K.

In certain embodiments, the recombinant mutant human sialidase comprises a substitution or combination of substitutions corresponding to a substitution or combination of substitutions listed in TABLE 2 (amino acid positions corresponding to wild-type human Neu2 (SEQ ID NO: 1)).

TABLE 2
Substitution(s)
A2K
E72K
D215K
E257K
V325K
A2K + E257K
A2K + V325E
A2K + V325K
E257K + V325K

3. Addition of N-Terminal Peptides and N- or C-Terminal Substitutions

It has been discovered that the addition of a peptide sequence of two or more amino acids to the N-terminus of a human sialidase can improve expression and/or activity of the sialidase. In certain embodiments, the peptide is at least 2 amino acids in length, for example, from 2 to 20, from 2 to 10, from 2 to 5, or 2, 3, 4, 5, 6, 7, 8, 9, 10, 11, 12, 13, 14, 15, 16, 17, 18, 19, or 20 amino acids in length. In certain embodiments, the peptide may form, or have a propensity to form, an α-helix.

In mice, a Neu2 isoform (type B) found in thymus contains six amino acids not present in the canonical isoform of Neu2 found in skeletal muscle. In certain embodiments herein, the N-terminal six amino acids of the mouse thymus Neu2 isoform, MEDLRP (SEQ ID NO: 4), or variations thereof, can be added onto a human Neu, e.g., human Neu2. In certain embodiments, the recombinant mutant human sialidase comprises a peptide at least two amino acid residues in length covalently associated with an N-terminal amino acid of the sialidase. In certain embodiments the recombinant mutant human sialidase comprises the peptide MEDLRP (SEQ ID NO: 4) or EDLRP (SEQ ID NO: 3) covalently associated with an N-terminal amino acid of the sialidase. In certain embodiments, the sialidase may further comprise a cleavage site, e.g., a proteolytic cleavage site, located between the peptide, e.g., MEDLRP (SEQ ID NO: 4) or EDLRP (SEQ ID NO: 3), and the remainder of the sialidase. In certain embodiments, the peptide, e.g., MEDLRP (SEQ ID NO: 4) or EDLRP (SEQ ID NO: 3), may be post-translationally cleaved from the remainder of the sialidase.

Alternatively to, or in combination with, the N-terminal addition, 1-5 amino acids of the 12 amino acid N-terminal region of the recombinant mutant human sialidase may be removed, e.g., the N-terminal methionine can be removed. In certain embodiments, if the recombinant mutant human sialidase is Neu2, the N-terminal methionine can be removed, the first five amino acids (MASLP; SEQ ID NO: 12) can be removed, or the second through fourth amino acids (ASLP; SEQ ID NO: 13) can be removed.

In certain embodiments, 1-5 amino acids of the 12 amino acid N-terminal region of the recombinant mutant human sialidase are substituted with MEDLRP (SEQ ID NO: 4), EDLRP (SEQ ID NO: 3), or TVEKSVVF (SEQ ID NO: 14). For example, in certain embodiments, if the recombinant mutant human sialidase is Neu2, the amino acids MASLP (SEQ ID NO: 12), ASLP (SEQ ID NO: 13) or M are substituted with MEDLRP (SEQ ID NO: 4), EDLRP (SEQ ID NO: 3) or TVEKSVVF (SEQ ID NO: 14).

Human sialidases have a β-propeller structure, characterized by 6 blade-shaped β-sheets arranged toroidally around a central axis. Generally, hydrophobic interactions between the blades of a β-propeller, including between the N- and C-terminal blades, enhance stability. Accordingly, in order to increase expression of human Neu2 or the other human sialidases, a recombinant mutant human sialidase can be designed comprising an amino acid substitution that increases hydrophobic interactions and/or hydrogen bonding between the N- and C-terminal β-propeller blades of the sialidase.

Accordingly, in certain embodiments, the recombinant mutant human sialidase comprises a substitution of at least one wild-type amino acid residue, wherein the substitution increases hydrophobic interactions and/or hydrogen bonding between the N- and C-termini of the sialidase relative to a sialidase without the substitution. In certain embodiments, the wild-type amino acid is substituted with asparagine (asn, N), lysine (lys, K), tyrosine (tyr, Y), phenylalanine (phe, F), or tryptophan (trp, W). Exemplary substitutions in Neu2 that increase hydrophobic interactions and/or hydrogen bonding between the N- and C-termini include L4N, L4K, V6Y, L7N, L4N and L7N, L4N and V6Y and L7N, V12N, V12Y, V12L, V6Y, V6F, or V6W. In certain embodiments, the sialidase comprises the V6Y substitution.

In certain embodiments, the recombinant mutant human sialidase comprises a combination of the above substitutions. For example, a recombinant mutant human Neu2 sialidase can comprise the additional amino acids MEDLRP (SEQ ID NO: 4), EDLRP (SEQ ID NO: 3), or TVEKSVVF (SEQ ID NO: 14) at the N-terminus and, in combination, can comprise at least one L4N, L4K, V6Y, L7N, L4N and L7N, L4N and V6Y and L7N, V12N, V12Y, V12L, V6Y, V6F, or V6W substitution. In certain embodiments, the amino acids MASLP (SEQ ID NO: 12), ASLP (SEQ ID NO: 13) or M of a recombinant mutant human Neu2 sialidase are replaced with MEDLRP (SEQ ID NO: 4), EDLRP (SEQ ID NO: 3) or TVEKSVVF (SEQ ID NO: 14) and the recombinant mutant human Neu2 sialidase also comprises at least one L4N, L4K, V6Y, L7N, L4N and L7N, L4N and V6Y and L7N, V12N, V12Y, V12L, V6Y, V6F, or V6W substitution.

In certain embodiments, the recombinant mutant human sialidase comprises a mutation or combination of mutations corresponding to a mutation or combination of mutations listed in TABLE 3 (amino acid positions corresponding to wild-type human Neu2 (SEQ ID NO: 1)).

TABLE 3
Mutation(s)
Substitute M at the N-terminus with EDLRP (SEQ ID NO: 3)
Substitute M at the N-terminus with MEDLRP (SEQ ID NO: 4)
Insert MEDLRP (SEQ ID NO: 4) at the N-terminus
Substitute MASLP (SEQ ID NO: 12) at the N-terminus with
MEDLRP (SEQ ID NO: 4)
L4N
V6Y
L7N
V6F
V6W

Additionally, in certain embodiments, the sialidase comprises a substitution or deletion of an N-terminal methionine at the N-terminus of the sialidase. For example, in certain embodiments, the sialidase comprises a substitution of a methionine residue at a position corresponding to position 1 of wild-type human Neu2 (SEQ ID NO: 1), e.g., the methionine at a position corresponding to position 1 of wild-type human Neu2 is substituted by alanine (M1A) or aspartic acid (M1D). In other embodiments, the sialidase comprises a deletion of a methionine residue at a position corresponding to position 1 (ΔM1) of wild-type human Neu2 (SEQ ID NO: 1).

In certain embodiments, the recombinant mutant human sialidase comprises a substitution or combination of substitutions corresponding to a substitution or combination of substitutions listed in TABLE 4 (amino acid positions corresponding to wild-type human Neu2 (SEQ ID NO: 1)).

TABLE 4
Mutation(s)
Deletion of M1, V6Y, I187K
MIR, V6Y, I187K
MIH, V6Y, I187K
MIK, V6Y, I187K
MID, V6Y, I187K
MIT, V6Y, I187K
MIN, V6Y, I187K
MIQ, V6Y, I187K
MIG, V6Y, I187K
MIA, V6Y, I187K
MIV, V6Y, I187K
MIL, V6Y, I187K
MIF, V6Y, I187K
MIY, V6Y, I187K

4. Substitutions of Residues to Decrease Proteolytic Cleavage

It has been discovered that certain sialidases (e.g., human Neu2) are susceptible to cleavage by a protease (e.g., trypsin). As a result, proteolytic cleavage of the sialidase may occur during recombinant protein production, harvesting, purification, or formulation, during administration to a subject, or after administration to a subject. Accordingly, in certain embodiments, the recombinant mutant human sialidase comprises a substitution of at least one wild-type amino acid residue, wherein the substitution decreases cleavage of the sialidase by a protease (e.g., trypsin) relative to a sialidase without the substitution.

In certain embodiments, incubation of the recombinant mutant human sialidase with a protease (e.g., trypsin) results in from about 1% to about 50%, from about 1% to about 40%, from about 1%, to about 30%, from about 1% to about 20%, from about 1% to about 10%, from about 1% to about 5%, from about 5% to about 50%, from about 5% to about 40%, from about 5% to about 30%, from about 5% to about 20%, from about 5% to about 10%, from about 10% to about 50%, from about 10% to about 40%, from about 10% to about 30%, from about 10% to about 20%, from about 20% to about 50%, from about 20% to about 40%, from about 20% to about 30%, from about 30% to about 50%, from about 30% to about 40%, or from about 40% to about 50% of the proteolytic cleavage of a corresponding wild-type sialidase when incubated with the protease under the same conditions. In certain embodiments, incubation of the recombinant mutant human sialidase with a protease (e.g., trypsin) results in less than 50%, less than 40%, less than 30%, less than 10%, less than 5%, less than 3%, less than 1%, or less than 0.5% of the proteolytic cleavage of a corresponding wild-type sialidase when incubated with the protease under the same conditions. Proteolytic cleavage can be assayed by any method known in the art, including for example, by SDS-PAGE as described in Example 4 herein.

Exemplary substitutions that increase resistance to proteolytic cleavage include: (i) a substitution of an alanine residue at a position corresponding to position 242 of wild-type human Neu2 (SEQ ID NO: 1), e.g., a substitution by cysteine (A242C), phenylalanine (A242F), glycine (A242G), histidine (A242H), isoleucine (A242I), lysine (A242K), leucine (A242L), methionine (A242M), asparagine (A242N), glutamine (A242Q), arginine (A242R), serine (A242S), valine (A242V), tryptophan (A242W), or tyrosine (A242Y); (ii) a substitution of an arginine residue at a position corresponding to position 243 of wild-type human Neu2 (SEQ ID NO: 1), e.g., a substitution by glutamic acid (R243E), histidine (R243H), asparagine (R243N), glutamine (R243Q), or lysine (R243K); (iii) a substitution of a valine residue at a position corresponding to position 244 of wild-type human Neu2 (SEQ ID NO: 1), e.g., a substitution by isoleucine (V244I), lysine (V244K), or proline (V244P); or (iv) a combination of any of the foregoing. In certain embodiments, the recombinant mutant human sialidase comprises a substitution selected from A242C, A242F, A242Y, and A242W. In certain embodiments, the recombinant mutant human sialidase comprises a substitution or a combination of substitutions corresponding to a substitution or combination of substitutions listed in TABLE 5 (amino acid positions corresponding to wild-type human Neu2 (SEQ ID NO: 1)).

TABLE 5

| Wild Type | |
|---|---|
| Human Neu2 | |
| (SEQ ID NO: 1) | Exemplary Substitution(s) |
| Amino Acid | at Specified Position(s) |
| A242 | C, F, G, H, I, K, L, M, N, P, Q, R, S, V, W, Y |
| R243 | E, H, N, Q, K |
| V244 | I, K, P |

Additional exemplary substitutions that increase resistance to proteolytic cleavage (and/or increase expression yield and/or enzymatic activity) include: (i) a substitution of a leucine residue at a position corresponding to position 240 of wild-type human Neu2 (SEQ ID NO: 1), e.g., a substitution by aspartic acid (L240D), asparagine (L240N), or tyrosine (L240Y); (ii) a substitution of an alanine residue at a position corresponding to position 213 of wild-type human Neu2 (SEQ ID NO: 1), e.g., a substitution by cysteine (A213C), asparagine (A213N), serine (A213S), or threonine (A213T); (iii) a substitution of an arginine residue at a position corresponding to position 241 of wild-type human Neu2 (SEQ ID NO: 1), e.g., a substitution by alanine (R241A), aspartic acid (R241D), leucine (R241L), glutamine (R241Q). or tyrosine (R241Y); (iv) a substitution of a serine residue at a position corresponding to position 258 of wild-type human Neu2 (SEQ ID NO: 1), e.g., a substitution by cysteine (S258C); (v) a substitution of a leucine residue at a position corresponding to position 260 of wild-type human Neu2 (SEQ ID NO: 1), e.g., a substitution by aspartic acid (L260D), phenylalanine (L260F), glutamine (L260Q), or threonine (L260T); (vi) a substitution of a valine residue at a position corresponding to position 265 of wild-type human Neu2 (SEQ ID NO: 1), e.g., a substitution by phenylalanine (V265F); or (vii) a combination of any of the foregoing. It is contemplated that, in certain embodiments, a substitution or a combination of substitutions at these positions may improve hydrophobic and/or aromatic interaction between secondary structure elements in the sialidase (e.g., between an α-helix and the nearest (3-sheet) thereby stabilizing the structure and improving resistance to proteolytic cleavage.

In certain embodiments, the recombinant mutant sialidase comprises a mutation at position L240. In certain embodiments, the recombinant mutant sialidase comprises a combination of mutations at positions (i) A213 and A242, (ii) A213, A242, and S258, (iii) L240 and L260, (iv) R241 and A242, (v) A242 and L260, (vi) A242 and V265, or (vii) L240 and A242. In certain embodiments, the recombinant mutant human sialidase comprises a combination of substitutions selected from (i) A213C, A242F, and S258C, (ii) A213C and A242F, (iii) A213T and A242F, (iv) R241Y and A242F, and (v) L240Y and A242F. In certain embodiments, the recombinant mutant human sialidase comprises a substitution or combination of substitutions corresponding to a substitution or combination of substitutions listed in TABLE 6 (amino acid positions corresponding to wild-type human Neu2 (SEQ ID NO: 1)).

TABLE 6
Substitution(s)
A242C, V244P
A242R, V244R
A242R, V244H
A242Y, V244P
A242T, V244P
A242N, V244P
A213C, A242F
A213S, A242F
A213T, A242F
A213N, A242F
A213C, A242F, S258C
A242F, L260F
A242F, V265F
L240Y
L240Y, L260F
L240D, L260T
L240N, L260T
L240N, L260D
L240N, L260Q
L240Y, A242F
R241A, A242F
R241Y, A242F

5. Other Substitutions

In certain embodiments, the recombinant mutant human sialidase comprises at least one of the following substitutions: I187K, A328E, K370N, or H210N. In certain embodiments, a recombinant mutant human Neu2 comprises the substitution of the amino acids GDYDAPTHQVQW (SEQ ID NO: 15) with the amino acids SMDQGSTW (SEQ ID NO: 16) or STDGGKTW (SEQ ID NO: 17). In certain embodiments, a recombinant mutant human Neu2 comprises the substitution of the amino acids PRPPAPEA (SEQ ID NO: 18) with the amino acids QTPLEAAC (SEQ ID NO: 19). In certain embodiments, a recombinant mutant human Neu2 comprises the substitution of the amino acids NPRPPAPEA (SEQ ID NO: 20) with the amino acids SQNDGES (SEQ ID NO: 21).

In certain embodiments, the recombinant mutant human sialidase comprises at least one substitution at a position corresponding to V212, A213, Q214, D215, T216, L217, E218, C219, Q220, V221, A222, E223, V224, E225, or T225.

In certain embodiments, the recombinant mutant human sialidase comprises an amino acid substitution at a position identified in TABLE 7 (amino acid positions corresponding to wild-type human Neu2 (SEQ ID NO: 1). In certain embodiments, the sialidase comprises an amino acid substitution identified in TABLE 7. In certain embodiments, the sialidase comprises a combination of any amino acid substitutions identified in TABLE 7.

TABLE 7

| Wild Type | |
|---|---|
| Human Neu2 | |
| (SEQ ID NO: 1) | Exemplary Substitution(s) |
| Amino Acid | at Specified Position(s) |
| M1 | D |
| L4 | S, T, Y, L, F, A, P, V, I, N, D, or H |
| P5 | G |
| V6 | Y |
| L7 | F, Y, S, I, T, or N |
| K9 | D |
| V12 | L, A, P, V, N, D, or H |
| F13 | S, N, R, K, T, G, D, E, or A |
| I22 | S, N, R, K, T, G, D, E, A, Y, L, F, P, V, I, or H |
| A24 | S, N, R, K, T, G, D, E, A, Y, L, F, P, V, I, or H |
| L34 | S, T, Y, L, F, A, P, V, I, N, D, or H |
| A36 | S, T, Y, L, F, A, P, V, I, N, D, or H |
| A42 | R or D |
| K44 | R or E |
| K45 | A, E, or R |
| L54 | M |
| P62 | H, G, N, T, S, F, I, D, or E |
| H64 | F, Y, S, I, T, or N |
| Q69 | H |
| R78 | K |
| D80 | P |
| P89 | S, T, Y, L, F, A, P, V, I, N, D, H, or M |
| A93 | E or K |
| G107 | D |
| Q108 | H |
| Q112 | R or K |
| C125 | Y, F, or L |
| Q126 | E, F, H, I, L, or Y |
| A150 | V |
| T156 | R, N, D, C, G, H, I, L, F, S, Y, V, A, P, or T |
| F157 | R, N, D, C, G, H, I, L, F, S, Y, V, A, or P |
| A158 | R, N, D, C, G, H, I, L, F, S, Y, V, A, P, or T |
| V159 | R, N, D, C, G, H, I, L, F, S, Y, V, A, or P |
| G160 | R, N, D, C, G, H, I, L, F, S, Y, V, A, P, or T |
| P161 | R, N, D, C, G, H, I, L, F, S, Y, V, A, or P |
| G162 | R, N, D, C, G, H, I, L, F, S, Y, V, A, P, or T |
| H163 | R, N, D, C, G, H, I, L, F, S, Y, V, A, or P |
| C164 | R, N, D, C, G, H, I, L, F, S, Y, V, A, P, or T |
| L165 | R, N, D, C, G, H, I, L, F, S, Y, V, A, or P |
| R170 | P |
| A171 | G |
| V176 | R, N, D, C, G, H, I, L, F, S, Y, V, P, or A |
| P177 | S, T, Y, L, F, A, P, V, I, N, D, or H |
| A178 | S, T, Y, L, F, A, P, V, I, N, D, or H |
| L184 | S, N, R, K, T, G, D, E, A, F, H, I, L, P, V, or Y |
| H185 | S, N, R, K, T, G, D, E, or A |
| P186 | S, N, R, K, T, G, D, E, A, F, H, I, L, P, V, or Y |
| I187 | S, N, R, K, T, G, D, E, or A |
| Q188 | P, S, N, R, K, T, G, D, E, or A |
| R189 | P |
| P190 | F, M, A, D, G, H, N, P, R, S, or T |
| I191 | M, A, D, F, H, I, L, N, P, S, T, V, Y, E, G, K, or R |
| A194 | S, T, Y, L, F, A, P, V, I, N, D, or H |
| A213 | C, N, S, or T |
| L217 | R, N, D, C, G, H, I, L, F, S, Y, or V |
| C219 | R, N, D, C, G, H, I, L, F, S, Y, or V |
| A222 | D |
| E225 | P |
| H239 | P |
| L240 | D, N, or Y |
| R241 | A, D, L, Q, or Y |
| A242 | C, F, G, H, I, K, L, M, N, Q, R, S, V, W, or Y |
| V244 | I or P |
| T249 | A |
| D251 | G |
| E257 | P |
| S258 | C |
| L260 | D, F, Q, or T |
| V265 | F |
| Q270 | S, T, A, H, P, or F |
| G271 | S, N, R, K, T, G, D, E, or A |
| C272 | S, N, R, K, T, G, D, E, A, C, H, Y, F, H, L, P, or V |
| W292 | R |
| S301 | A, D, E, F, G, H, I, K, L, M, N, P, Q, T, V, W, Y, |
| | C, or R |
| W302 | A, D, E, F, G, H, I, L, M, N, P, Q, R, S, T, V, Y, or |
| | K |
| E319 | D |
| V325 | F, Y, S, I, T, N, A, D, H, L, P, or V |
| L326 | F, Y, S, I, T, N, A, D, H, L, P, or V |
| L327 | F, Y, S, I, T, N, A, D, H, L, P, or V |
| C332 | A, D, G, H, N, P, R, S, or T |
| Y359 | A or S |
| V363 | R, S, T, Y, L, F, A, P, V, I, N, D, or H |
| L365 | K, Q, F, Y, S, I, T, N, A, D, H, L, P, or V |

For example, in certain embodiments, the recombinant mutant human sialidase comprises: (a) a substitution of a proline residue at a position corresponding to position 5 of wild-type human Neu2 (P5); (b) a substitution of a lysine residue at a position corresponding to position 9 of wild-type human Neu2 (K9); (c) a substitution of an alanine residue at a position corresponding to position 42 of wild-type human Neu2 (A42); (d) a substitution of a lysine residue at a position corresponding to position 44 of wild-type human Neu2 (K44); (e) a substitution of a lysine residue at a position corresponding to position 45 of wild-type human Neu2 (K45); (f) a substitution of a leucine residue at a position corresponding to position 54 of wild-type human Neu2 (L54); (g) a substitution of a proline residue at a position corresponding to position 62 of wild-type human Neu2 (P62); (h) a substitution of a glutamine residue at a position corresponding to position 69 of wild-type human Neu2 (Q69); (i) a substitution of an arginine residue at a position corresponding to position 78 of wild-type human Neu2 (R78); (j) a substitution of an aspartic acid residue at a position corresponding to position 80 of wild-type human Neu2 (D80); (k) a substitution of an alanine residue at a position corresponding to position 93 of wild-type human Neu2 (A93); (l) a substitution of a glycine residue at a position corresponding to position 107 of wild-type human Neu2 (G107); (m) a substitution of a glutamine residue at a position corresponding to position 108 of wild-type human Neu2 (Q108); (n) a substitution of a glutamine residue at a position corresponding to position 112 of wild-type human Neu2 (Q112); (o) a substitution of a cysteine residue at a position corresponding to position 125 of wild-type human Neu2 (C125); (p) a substitution of a glutamine residue at a position corresponding to position 126 of wild-type human Neu2 (Q126); (q) a substitution of an alanine residue at a position corresponding to position 150 of wild-type human Neu2 (A150); (r) a substitution of a cysteine residue at a position corresponding to position 164 of wild-type human Neu2 (C164); (s) a substitution of an arginine residue at a position corresponding to position 170 of wild-type human Neu2 (R170); (t) a substitution of an alanine residue at a position corresponding to position 171 of wild-type human Neu2 (A171); (u) a substitution of a glutamine residue at a position corresponding to position 188 of wild-type human Neu2 (Q188); (v) a substitution of an arginine residue at a position corresponding to position 189 of wild-type human Neu2 (R189); (w) a substitution of an alanine residue at a position corresponding to position 213 of wild-type human Neu2 (A213); (x) a substitution of a leucine residue at a position corresponding to position 217 of wild-type human Neu2 (L217); (y) a substitution of a glutamic acid residue at a position corresponding to position 225 of wild-type human Neu2 (E225); (z) a substitution of a histidine residue at a position corresponding to position 239 of wild-type human Neu2 (H239); (aa) a substitution of a leucine residue at a position corresponding to position 240 of wild-type human Neu2 (L240); (bb) a substitution of an arginine residue at a position corresponding to position 241 of wild-type human Neu2 (R241); (cc) a substitution of an alanine residue at a position corresponding to position 242 of wild-type human Neu2 (A242); (dd) a substitution of a valine residue at a position corresponding to position 244 of wild-type human Neu2 (V244); (ee) a substitution of a threonine residue at a position corresponding to position 249 of wild-type human Neu2 (T249); (ff) a substitution of an aspartic acid residue at a position corresponding to position 251 of wild-type human Neu2 (D251); (gg) a substitution of a glutamic acid residue at a position corresponding to position 257 of wild-type human Neu2 (E257); (hh) a substitution of a serine residue at a position corresponding to position 258 of wild-type human Neu2 (S258); (ii) a substitution of a leucine residue at a position corresponding to position 260 of wild-type human Neu2 (L260); (jj) a substitution of a valine residue at a position corresponding to position 265 of wild-type human Neu2 (V265); (kk) a substitution of a glutamine residue at a position corresponding to position 270 of wild-type human Neu2 (Q270); (ll) a substitution of a tryptophan residue at a position corresponding to position 292 of wild-type human Neu2 (W292); (mm) a substitution of a serine residue at a position corresponding to position 301 of wild-type human Neu2 (S301); (nn) a substitution of a tryptophan residue at a position corresponding to position 302 of wild-type human Neu2 (W302); (oo) a substitution of a valine residue at a position corresponding to position 363 of wild-type human Neu2 (V363); or (pp) a substitution of a leucine residue at a position corresponding to position 365 of wild-type human Neu2 (L365); or a combination of any of the foregoing substitutions. For example, the sialidase may comprise a substitution of K9, A42, P62, A93, Q216, A242, Q270, S301, W302, V363, or L365, or a combination of any of the foregoing substitutions.

In certain embodiments, in the sialidase: (a) the proline residue at a position corresponding to position 5 of wild-type human Neu2 is substituted by histidine (P5H); (b) the lysine residue at a position corresponding to position 9 of wild-type human Neu2 is substituted by aspartic acid (K9D); (c) the alanine residue at a position corresponding to position 42 of wild-type human Neu2 is substituted by arginine (A42R) or aspartic acid (A42D); (d) the lysine residue at a position corresponding to position 44 of wild-type human Neu2 is substituted by arginine (K44R) or glutamic acid (K44E); (e) the lysine residue at a position corresponding to position 45 of wild-type human Neu2 is substituted by alanine (K45A), arginine (K45R), or glutamic acid (K45E); (f) the leucine residue at a position corresponding to position 54 of wild-type human Neu2 is substituted by methionine (L54M); (g) the proline residue at a position corresponding to position 62 of wild-type human Neu2 is substituted by asparagine (P62N), aspartic acid (P62D), histidine (P62H), glutamic acid (P62E), glycine (P62G), serine (P62S), or threonine (P62T); (h) the glutamine residue at a position corresponding to position 69 of wild-type human Neu2 is substituted by histidine (Q69H); (i) the arginine residue at a position corresponding to position 78 of wild-type human Neu2 is substituted by lysine (R78K); (j) the aspartic acid residue at a position corresponding to position 80 of wild-type human Neu2 is substituted by proline (D80P); (k) the alanine residue at a position corresponding to position 93 of wild-type human Neu2 is substituted by glutamic acid (A93E) or lysine (A93K); (l) the glycine residue at a position corresponding to position 107 of wild-type human Neu2 is substituted by aspartic acid (G107D); (m) the glutamine residue at a position corresponding to position 108 of wild-type human Neu2 is substituted by histidine (Q108H); (n) the glutamine residue at a position corresponding to position 112 of wild-type human Neu2 is substituted by arginine (Q112R) or lysine (Q112K); (o) the cysteine residue at a position corresponding to position 125 of wild-type human Neu2 is substituted by leucine (C125L); (p) the glutamine residue at a position corresponding to position 126 of wild-type human Neu2 is substituted by leucine (Q126L), glutamic acid (Q126E), phenylalanine (Q126F), histidine (Q126H), isoleucine (Q126I), or tyrosine (Q126Y); (q) the alanine residue at a position corresponding to position 150 of wild-type human Neu2 is substituted by valine (A150V); (r) the cysteine residue at a position corresponding to position 164 of wild-type human Neu2 is substituted by glycine (C164G); (s) the arginine residue at a position corresponding to position 170 of wild-type human Neu2 is substituted by proline (R170P); (t) the alanine residue at a position corresponding to position 171 of wild-type human Neu2 is substituted by glycine (A171G); (u) the glutamine residue at a position corresponding to position 188 of wild-type human Neu2 is substituted by proline (Q188P); (v) the arginine residue at a position corresponding to position 189 of wild-type human Neu2 is substituted by proline (R189P); (w) the alanine residue at a position corresponding to position 213 of wild-type human Neu2 is substituted by cysteine (A213C), asparagine (A213N), serine (A213S), or threonine (A213T); (x) the leucine residue at a position corresponding to position 217 of wild-type human Neu2 is substituted by alanine (L217A) or valine (L217V); (y) the threonine residue at a position corresponding to position 249 of wild-type human Neu2 is substituted by alanine (T249A); (z) the aspartic acid residue at a position corresponding to position 251 of wild-type human Neu2 is substituted by glycine (D251G); (aa) the glutamic acid residue at a position corresponding to position 225 of wild-type human Neu2 is substituted by proline (E225P); (bb) the histidine residue at a position corresponding to position 239 of wild-type human Neu2 is substituted by proline (H239P); (cc) the leucine residue at a position corresponding to position 240 of wild-type human Neu2 is substituted by aspartic acid (L240D), asparagine (L240N), or tyrosine (L240Y); (dd) the arginine residue at a position corresponding to position 241 of wild-type human Neu2 is substituted by alanine (R241A), aspartic acid (R241D), leucine (R241L), glutamine (R241Q). or tyrosine (R241Y); (ee) the alanine residue at a position corresponding to position 242 of wild-type human Neu2 is substituted by cysteine (A242C), phenylalanine (A242F), glycine (A242G), histidine (A242H), isoleucine (A242I), lysine (A242K), leucine (A242L), methionine (A242M), asparagine (A242N), glutamine (A242Q), arginine (A242R), serine (A242S), valine (A242V), tryptophan (A242W), or tyrosine (A242Y); (ff) the valine residue at a position corresponding to position 244 of wild-type human Neu2 is substituted by isoleucine (V244I), lysine (V244K), or proline (V244P); (gg) the glutamic acid residue at a position corresponding to position 257 of wild-type human Neu2 is substituted by proline (E257P); (hh) the serine residue at a position corresponding to position 258 is substituted by cysteine (S258C); (ii) the leucine residue at a position corresponding to position 260 of wild-type human Neu2 is substituted by aspartic acid (L260D), phenylalanine (L260F), glutamine (L260Q), or threonine (L260T); (jj) the valine residue at a position corresponding to position 265 of wild-type human Neu2 is substituted by phenylalanine (V265F); (kk) the glutamine residue at a position corresponding to position 270 of wild-type human Neu2 is substituted by alanine (Q270A), histidine (Q270H), phenylalanine (Q270F), proline (Q270P), serine (Q270S), or threonine (Q270T); (ll) the tryptophan residue at a position corresponding to position 292 of wild-type human Neu2 is substituted by arginine (W292R); (mm) the serine residue at a position corresponding to position 301 of wild-type human Neu2 is substituted by alanine (S301A), aspartic acid (S301D), glutamic acid (S301E), phenylalanine (S301F), glycine (S301G), histidine (S301H), isoleucine (S301I), lysine (S301K), leucine (S301L), methionine (S301M), asparagine (S301N), proline (S301P), glutamine (S301Q), arginine (S301R), threonine (S301T), valine (S301V), tryptophan (S301W), or tyrosine (S301Y); (nn) the tryptophan residue at a position corresponding to position 302 of wild-type human Neu2 is substituted by alanine (W302A), aspartic acid (W302D), glutamic acid (W302E), phenylalanine (W302F), glycine (W302G), histidine (W302H), isoleucine (W3021), lysine (W302K), leucine (W302L), methionine (W302M), asparagine (W302N), proline (W302P), glutamine (W302Q), arginine (W302R), serine (W302S), threonine (W302T), valine (W302V), or tyrosine (W302Y); (oo) the valine residue at a position corresponding to position 363 of wild-type human Neu2 is substituted by arginine (V363R); or (pp) the leucine residue at a position corresponding to position 365 of wild-type human Neu2 is substituted by glutamine (L365Q), histidine (L365H), isoleucine (L365I), lysine (L365K) or serine (L365S); or the sialidase comprises a combination of any of the foregoing substitutions. For example, the sialidase may comprise a substitution selected from K9D, A42R, P62G, P62N, P62S, P62T, D80P, A93E, Q126H, Q126Y, R189P, H239P, A242T, Q270A, Q270S, Q270T, S301A, S301R, W302K, W302R, V363R, and L365I, or a combination of any of the foregoing substitutions.

In certain embodiments, the recombinant mutant human sialidase comprises a deletion of a leucine residue at a position corresponding to position 184 of wild-type human Neu2 (ΔL184), a deletion of a histidine residue at a position corresponding to position 185 of wild-type human Neu2 (ΔH185), a deletion of a proline residue at a position corresponding to position 186 of wild-type human Neu2 (ΔP186), a deletion of an isoleucine residue at a position corresponding to position 187 of wild-type human Neu2 (ΔI187), and a deletion of a glutamine residue at a position corresponding to position 184 of wild-type human Neu2 (ΔQ188), or a combination of any of the foregoing deletions.

In certain embodiments, the recombinant mutant human sialidase comprises an insertion between a threonine residue at a position corresponding to position 216 of wild-type human Neu2 and a leucine residue at a position corresponding to position 217 of wild-type human Neu2, for example, an insertion of an amino acid selected from S, T, Y, L, F, A, P, V, I, N, D, and H.

Additional exemplary sialidase mutations, and combinations of sialidase mutations, are described in International (PCT) Patent Application Publication No. WO 2019/136167, including in the Detailed Description in the section entitled “I. Recombinant Human Sialidases,” and in the Examples in Examples 1, 2, 3, 4, 5, and 6, and International (PCT) Patent Application Publication No. WO 2021/003469, including in the Detailed Description in the section entitled “I. Recombinant Human Sialidases,” and in the Examples in Examples 2, 3, 4, and 5, and in International (PCT) Patent Application No. PCT/US2021/040240, filed Jul. 2, 2021, including in the Detailed Description in the section entitled “I. Recombinant Human Sialidases,” and in the Examples in Examples 2, 3, 4, and 5.

6. Combinations of Substitutions

In certain embodiments, the recombinant mutant human sialidase comprises a combination of any of the mutations contemplated herein. For example, the recombinant mutant sialidase enzyme may comprise a combination of 2, 3, 4, 5, 6, 7, 8, 9, 10, 11, 12, 13, 14, 15 or more of the mutations contemplated herein. It is contemplated that the recombinant mutant sialidase enzyme may comprise 1-15, 1-10, 1-7, 1-6, 1-5, 1-4, 1-3, 1-2, 2-15, 2-10, 2-7, 2-6, 2-5, 2-4, 2-3, 3-15, 3-10, 3-7, 3-6, 3-5, or 3-4 of the mutations contemplated herein.

For example, the recombinant mutant sialidase enzyme may comprise a M1 deletion (ΔM1), M1A substitution, M1D substitution, V6Y substitution, K9D substitution, P62G substitution, P62N substitution, P62S substitution, P62T substitution, A93E substitution, I187K substitution, Q270A substitution, S301R substitution, W302K substitution, C332A substitution, V363R substitution, L365I substitution, or a combination of any of the foregoing.

In certain embodiments, the recombinant mutant sialidase enzyme comprises a M1 deletion (ΔM1), M1A substitution, M1D substitution, V6Y substitution, I187K substitution, C332A substitution, or a combination of any of the foregoing. For example, the recombinant mutant sialidase enzyme may comprise a combination of mutations selected from: M1A and V6Y; M1A and I187K; M1A and C332A; M1D and V6Y; M1D and I187K; M1D and C332A; ΔM1 and V6Y; ΔM1 and I187K; ΔM1 and C332A; V6Y and I187K; V6Y and C332A; I187K and C332A; M1A, V6Y, and I187K; M1A, V6Y, and C332A; M1A, I187K, and C332A; M1D, V6Y, and I187K; M1D, V6Y, and C332A; M1D, I187K, and C332A; ΔM1, V6Y, and I187K; ΔM1, V6Y, and C332A; ΔM1, I187K, and C332A; V6Y, I187K, and C332A; M1A, V6Y, I187K, and C332A; M1D, V6Y, I187K, and C332A; and ΔM1, V6Y, I187K, and C332A.

In certain embodiments, the recombinant mutant sialidase enzyme comprises (i) an amino acid substitution identified in TABLE 7, or a combination of any amino acid substitutions identified in TABLE 7, and (ii) an M1 deletion (ΔM1), M1A substitution, M1D substitution, V6Y substitution, I187K substitution, C332A substitution, or a combination of any of the foregoing. For example, the recombinant mutant sialidase enzyme may comprise (i) an amino acid substitution identified in TABLE 7, or a combination of any amino acid substitutions identified in TABLE 7, and (ii) a combination of mutations selected from: M1A and V6Y; M1A and I187K; M1A and C332A; M1D and V6Y; M1D and I187K; M1D and C332A; ΔM1 and V6Y; ΔM1 and I187K; ΔM1 and C332A; V6Y and I187K; V6Y and C332A; I187K and C332A; M1A, V6Y, and I187K; M1A, V6Y, and C332A; M1A, I187K, and C332A; M1D, V6Y, and I187K; M1D, V6Y, and C332A; M1D, I187K, and C332A; ΔM1, V6Y, and I187K; ΔM1, V6Y, and C332A; ΔM1, I187K, and C332A; V6Y, I187K, and C332A; M1A, V6Y, I187K, and C332A; M1D, V6Y, I187K, and C332A; and ΔM1, V6Y, I187K, and C332A.

In certain embodiments, the recombinant mutant sialidase enzyme comprises: (a) the M1D, V6Y, P62G, A93E, I187K, and C332A substitutions; (b) the M1D, V6Y, K9D, A93E, I187K, C332A, V363R, and L365I substitutions; (c) the M1D, V6Y, P62N, I187K, and C332A substitutions; (d) the M1D, V6Y, I187K, Q270A, S301R, W302K, and C332A substitutions; (e) the M1D, V6Y, P62S, I187K, Q270A, S301R, W302K, and C332A substitutions; (f) the M1D, V6Y, P62T, I187K, Q270A, S301R, W302K, and C332A substitutions; (g) the M1D, V6Y, P62N, I187K, Q270A, S301R, W302K, and C332A substitutions; (h) the M1D, V6Y, P62G, A93E, I187K, S301A, W302R, and C332A substitutions; (i) the M1D, V6Y, P62G, A93E, Q126Y, I187K, Q270T, and C332A substitutions; (j) the M1D, V6Y, P62G, A93E, Q126Y, I187K, and C332A substitutions; (k) the M1D, V6Y, P62G, A93E, Q126Y, I187K, A242F, Q270T, and C332A substitutions; or (l) the M1D, V6Y, A42R, P62G, A93E, Q126Y, I187K, A242F, Q270T, and C332A mutations.

In certain embodiments, the recombinant mutant human sialidase comprises a substitution of a serine residue at a position corresponding to position 301 of wild-type human Neu2 (S301) in combination with a substitution of a tryptophan residue at a position corresponding to position 302 of wild-type human Neu2 (W302). For example, the recombinant mutant human sialidase may comprise a combination of substitutions corresponding to a combination of substitutions listed in a row of TABLE 8 (amino acid positions corresponding to wild-type human Neu2 (SEQ ID NO: 1)). For example, the recombinant mutant human sialidase may comprise: the S301K and W302R substitutions; the S301K and W302K substitutions; or the S301A and W302 S substitutions.

TABLE 8
Substitutions
S301A, W302R
S301A, W302S
S301A, W302T
S301K, W302S
S301N, W302S
S301T, W302S
S301T, W302T
S301T, W302R
S301A, W302A
S301K, W302R
S301K, W302T
S301N, W302T
S301K, W302K
S301P, W302R
S301P, W302S
S301P, W302T

In certain embodiments, the recombinant mutant human sialidase comprises a combination of substitutions corresponding to a combination of substitutions listed in a row of TABLE 9 (amino acid positions corresponding to wild-type human Neu2 (SEQ ID NO: 1)).

TABLE 9
Substitutions
M1D, V6Y, P62G, I187K, C332A
M1D, V6Y, K9D, I187K, C332A, V363R, L365I
M1D, V6Y, P62G, A93E, I187K, C332A
M1D, V6Y, K9D, I187K, C332A, V363R, L365K
M1D, V6Y, K9D, I187K, C332A, V363R, L365S
M1D, V6Y, K9D, I187K, C332A, V363R, L365Q
M1D, V6Y, K9D, I187K, C332A, V363R, L365H
M1D, V6Y, A93K, I187K, C332A
M1D, V6Y, A93E, I187K, C332A
V6Y, I187K, W292R
V6Y, G107D, I187K
V6Y, C125L
C125L, I187K
V6Y, C125L, I187K
M1D, V6Y, K45A, I187K, C332A
M1D, V6Y, Q270A, I187K, C332A
M1D, V6Y, K44R, K45R, I187K, C332A
M1D, V6Y, Q112R, I187K, C332A
M1D, V6Y, Q270F, I187K, C332A
M1D, V6Y, I187K, S301R, W302K, C332A
M1D, V6Y, K44E, K45E, I187K, C332A
M1D, V6Y, I187K, L217V, C332A
M1D, V6Y, I187K, L217A, C332A
M1D, V6Y, K44E, K45E, I187K, S301R, W302K, C332A
M1D, V6Y, Q112R, I187K, S301R, W302K, C332A
M1D, V6Y, I187K, Q270A, S301R, W302K, C332A
M1D, V6Y, K44E, K45E, Q112R, I187K, C332A
M1D, V6Y, K44E, K45E, I187K, Q270A, C332A
M1D, V6Y, K45A, I187K, Q270A, C332A
M1D, V6Y, I187K, Q270H, C332A
M1D, V6Y, I187K, Q270P, C332A
M1D, V6Y, Q112K, I187K, C332A
M1D, V6Y, P62S, I187K, Q270A, S301R, W302K, C332A
M1D, V6Y, P62T, I187K, Q270A, S301R, W302K, C332A
M1D, V6Y, P62N, I187K, Q270A, S301R, W302K, C332A
V6Y, P62H, I187K
V6Y, Q108H, I187K
M1D, V6Y, P62H, I187K, C332A
M1D, V6Y, P62G, I187K, C332A
V6Y, P62G, I187K
M1D, V6Y, P62H, I187K
M1D, V6Y, Q108H, I187K
M1D, V6Y, P62N, I187K, C332A
M1D, V6Y, P62D, I187K, C332A
M1D, V6Y, P62E, I187K, C332A
V6Y, C164G, I187K, T249A
V6Y, C164G, I187K
V6Y, Q126L, I187K D251G
V6Y, L54M, Q69H, R78K, A171G, I187K
V6Y, P62T, I187K
V6Y, A150V, I187K
P5H, V6Y, P62S, I187K
V6Y, C164G, I187K
Q126Y, Q170T
Q126Y, A242F, Q270T
M1D, V6Y, P62G, A93E, Q126E, I187K, C332A
M1D, V6Y, P62G, A93E, Q126I, I187K, C332A
M1D, V6Y, P62G, A93E, Q126L, I187K, C332A
M1D, V6Y, P62G, A93E, Q126Y, I187K, C332A
M1D, V6Y, P62G, A93E, Q126F, I187K, C332A
M1D, V6Y, P62G, A93E, Q126H, I187K, C332A
M1D, V6Y, P62G, A93E, I187K, Q270S, C332A
M1D, V6Y, P62G, A93E, I187K, Q270T, C332A
M1D, V6Y, P62G, A93E, Q126Y, I187K, Q270T, C332A
M1D, V6Y, P62G, A93E, Q126Y, I187K, A242F, Q270T, C332A
M1D, V6Y, P62G, D80P, A93E, I187K, C332A
M1D, V6Y, P62G, A93E, R170P, I187K, C332A
M1D, V6Y, P62G, A93E, I187K, Q188P, C332A
M1D, V6Y, P62G, A93E, I187K, R189P, C332A
M1D, V6Y, P62G, A93E, I187K, E225P, C332A
M1D, V6Y, P62G, A93E, I187K, H239P, C332A
M1D, V6Y, P62G, A93E, I187K, E257P, C332A
M1D, V6Y, P62G, A93E, I187K, S301A, C332A
M1D, V6Y, P62G, A93E, I187K, S301D, C332A
M1D, V6Y, P62G, A93E, I187K, S301E, C332A
M1D, V6Y, P62G, A93E, I187K, S301F, C332A
M1D, V6Y, P62G, A93E, I187K, S301H, C332A
M1D, V6Y, P62G, A93E, I187K, S301K, C332A
M1D, V6Y, P62G, A93E, I187K, S301L, C332A
M1D, V6Y, P62G, A93E, I187K, S301M, C332A
M1D, V6Y, P62G, A93E, I187K, S301N, C332A
M1D, V6Y, P62G, A93E, I187K, S301P, C332A
M1D, V6Y, P62G, A93E, I187K, S301Q, C332A
M1D, V6Y, P62G, A93E, I187K, S301R, C332A
M1D, V6Y, P62G, A93E, I187K, S301T, C332A
M1D, V6Y, P62G, A93E, I187K, S301V, C332A
M1D, V6Y, P62G, A93E, I187K, S301W, C332A
M1D, V6Y, P62G, A93E, I187K, S301Y, C332A
M1D, V6Y, P62G, A93E, I187K, W302A, C332A
M1D, V6Y, P62G, A93E, I187K, W302D, C332A
M1D, V6Y, P62G, A93E, I187K, W302F, C332A
M1D, V6Y, P62G, A93E, I187K, W302G, C332A
M1D, V6Y, P62G, A93E, I187K, W302H, C332A
M1D, V6Y, P62G, A93E, I187K, W302I, C332A
M1D, V6Y, P62G, A93E, I187K, W302L, C332A
M1D, V6Y, P62G, A93E, I187K, W302M, C332A
M1D, V6Y, P62G, A93E, I187K, W302N, C332A
M1D, V6Y, P62G, A93E, I187K, W302P, C332A
M1D, V6Y, P62G, A93E, I187K, W302Q, C332A
M1D, V6Y, P62G, A93E, I187K, W302R, C332A
M1D, V6Y, P62G, A93E, I187K, W302S, C332A
M1D, V6Y, P62G, A93E, I187K, W302T, C332A
M1D, V6Y, P62G, A93E, I187K, W302V, C332A
M1D, V6Y, P62G, A93E, I187K, W302Y, C332A
M1D, V6Y, P62G, A93E, I187K, S301A, W302A, C332A
M1D, V6Y, P62G, A93E, I187K, S301A, W302R, C332A
M1D, V6Y, P62G, A93E, I187K, S301A, W302S, C332A
M1D, V6Y, P62G, A93E, I187K, S301A, W302T, C332A
M1D, V6Y, P62G, A93E, I187K, S301K, W302S, C332A
M1D, V6Y, P62G, A93E, I187K, S301K, W302R, C332A
M1D, V6Y, P62G, A93E, I187K, S301K, W302T, C332A
M1D, V6Y, P62G, A93E, I187K, S301N, W302S, C332A
M1D, V6Y, P62G, A93E, I187K, S301N, W302T, C332A
M1D, V6Y, P62G, A93E, I187K, S301T, W302R, C332A
Q126Y, Q270T
Q126Y, A242F, Q270T
M1D, V6Y, A42R, P62G, A93E, Q126Y, I187K, A242F, Q270T, C332A

In certain embodiments, the recombinant mutant human sialidase comprises the amino acid sequence of any one of SEQ ID NOs: 48-63, 94, 97, 100, or 126, or an amino acid sequence that has at least 85%, 90%, 95%, 96%, 97%, 98%, or 99% sequence identity to any one of SEQ ID NOs: 48-63, 94, 97, 100, or 126.

In certain embodiments, the recombinant mutant human sialidase comprises the amino acid sequence of

(SEQ ID NO: 64)
X₁X₂SX₃X₄X₅LQXESVFQSGAHAYRIPALLYLPGQQSLLAFAEQRX₇SX₈
X₉DEHAELIVX₁₀RRGDYDAX₁₁THQVQWX₁₂AQEVVAQAX₁₃LX₁₄GHRSMN
PCPLYDX₁₅QTGTLFLFFIAIPX₁₆X₁₇VTEX₁₈QQLQTRANVTRLX₁₉X₂₀V
TSTDHGRTWSSPRDLTDAAIGPX₂₁YREWSTFAVGPGHX₂₂LQLHDX₂₃X₂₄
RSLVVPAYAYRKLHPX₂₅X₂₆X₂₇PIPSAFX₂₈FLSHDHGRTWARGHFVX₂₉
QDTX₃₀ECQVAEVX₃₁TGEQRVVTLNARSX₃₂X₃₃X₃₄X₃₅RX₃₆QAQSX₃₇N
X₃₈GLDFQX₃₉X₄₀QX₄₁VKKLX₄₂EPPPX₄₃GX₄₄QGSVISFPSPRSGPGSPA
QX₄₅LLYTHPTHX₄₆X₄₇QRADLGAYLNPRPPAPEAWSEPX₄₈LLAKGSX₄₉
AYSDLQSMGTGPDGSPLFGX₅₀LYEANDYEEIX₅₁FX₅₂MFTLKQAFPAEY
LPQ,

wherein X₁ is Ala, Arg, Asn, Asp, Gln, Glu, Gly, His, Leu, Lys, Met, Phe, Thr, Val, or not present, X₂ is Ala or Lys, X₃ is Asn or Leu, X₄ is Pro or His, X₅ is Phe, Trp, Tyr or Val, X₆ is Lys or Asp, X₇ is Ala or Arg, X₈ is Lys, Arg, or Glu, X₉ is Lys, Ala, Arg, or Glu, X₁₀ is Leu or Met, X₁₁ is Pro, Asn, Asp, His, Glu, Gly, Ser or Thr, X₁₂ is Gln or His, X₁₃ is Arg or Lys, X₁₄ is Asp or Pro, X₁₅ is Ala, Glu or Lys, X₁₆ is Gly or Asp, X₁₇ is Gln or His, X₁₈ is Gln, Arg, or Lys, X₁₉ is Ala, Cys, Ile, Ser, Val, or Leu, X₂₀ is Gln, Leu, Glu, Phe, His, Ile, Leu, or Tyr, X₂₁ is Ala or Val, X₂₂ is Cys or Gly, X₂₃ is Arg or Pro, X₂₄ is Ala or Gly, X₂₅ is Arg, Ile, or Lys, X₂₆ is Gln or Pro, X₂₇ is Arg or Pro, X₂₈ is Ala, Cys, Leu, or Val, X₂₉ is Ala, Cys, Asn, Ser, or Thr, X₃₀ is Leu, Ala, or Val, X₃₁ is Glu or Pro, X₃₂ is His or Pro, X₃₃ is Leu, Asp, Asn, or Tyr, X₃₄ is Arg, Ala, Asp, Leu, Gln, or Tyr, X₃₅ is Ala, Cys, Phe, Gly, His, Ile, Lys, Leu, Met, Asn, Gln, Arg, Ser, Val, Trp, or Tyr, X₃₆ is Val, Ile, or Lys, X₃₇ is Thr or Ala, X₃₈ is Asp or Gly, X₃₉ is Glu, Lys, or Pro, X₄₀ is Ser or Cys, X₄₁ is Leu, Asp, Phe, Gln, or Thr, X₄₂ is Val or Phe, X₄₃ is Gln, Ala, His, Phe, Pro, Ser, or Thr, X₄₄ is Cys or Val, X₄₅ is Trp or Arg, X₄₆ is Ser, Arg, Ala, Asp, Glu, Phe, Gly, His, Ile, Lys, Leu, Met, Asn, Pro, Gln, Thr, Val, Trp, or Tyr, X₄₇ is Trp, Lys, Ala, Asp, Glu, Phe, Gly, His, Ile, Lys, Leu, Met, Asn, Pro, Gln, Arg, Ser, Thr, Val, or Tyr, X₄₈ is Lys or Val, X₄₉ is Ala, Cys, Ser, or Val, X₅₀ is Cys, Leu, or Val, X₅₁ is Val or Arg, and X₅₂ is Leu, Gln, His, Ile, Lys, or Ser, and the sialidase comprises at least one mutation relative to wild-type human Neu2 (SEQ ID NO: 1).

In certain embodiments, the recombinant mutant human sialidase comprises the amino acid sequence of

(SEQ ID NO: 65)
X₁ASLPX₂LQX₃ESVFQSGAHAYRIPALLYLPGQQSLLAFAEQRX₄SKKDE
HAELIVLRRGDYDAX₅THQVQWQAQEVVAQARLDGHRSMNPCPLYDX₆QT
GTLFLFFIAIPGQVTEQQQLQTRANVTRLCX₇VTSTDHGRTWSSPRDLT
DAAIGPAYREWSTFAVGPGHCLQLHDRARSLVVPAYAYRKLHPX₈QRPI
PSAFCFLSHDHGRTWARGHFVAQDTLECQVAEVETGEQRVVTLNARSHL
RX₉RVQAQSTNDGLDFQESQLVKKLVEPPPX₁₀GCQGSVISFPSPRSGPG
SPAQWLLYTHPTHX₁₁X₁₂QRADLGAYLNPRPPAPEAWSEPVLLAKGSX₁₃
AYSDLQSMGTGPDGSPLFGCLYEANDYEEIX₁₄FX₁₅MFTLKQAFPAEYL
PQ,

wherein X₁ is Ala, Arg, Asn, Asp, Gln, Glu, Gly, His, Leu, Lys, Met, Phe, Thr, Val, or not present, X₂ is Phe, Trp, Tyr or Val, X₃ is Lys or Asp, X₄ is Arg or Ala, X₅ is Pro, Asn, Asp, His, Glu, Gly, Ser or Thr, X₆ is Ala, Glu, or Lys, X₇ is Gln, Leu, Glu, Phe, His, Ile, Leu, or Tyr, X₈ is Arg, Ile, or Lys, X₉ is Ala, Cys, Phe, Gly, His, Ile, Lys, Leu, Met, Asn, Gln, Arg, Ser, Val, Trp, or Tyr, X₁₀ is Gln, Ala, His, Phe, Pro, Ser, or Thr, X₁₁ is Ser, Arg, Ala, Asp, Glu, Phe, Gly, His, Ile, Lys, Leu, Met, Asn, Pro, Gln, Thr, Val, Trp, or Tyr, X₁₂ is Trp, Lys, Ala, Asp, Glu, Phe, Gly, His, Ile, Lys, Leu, Met, Asn, Pro, Gln, Arg, Ser, Thr, Val, or Tyr, X₁₃ is Ala, Cys, Ser, or Val, X₁₄ is Val or Arg, and X₁₅ is Leu, Gln, His, Ile, Lys, or Ser, and the sialidase comprises at least one mutation relative to wild-type human Neu2 (SEQ ID NO: 1). In certain embodiments, X₁ is Ala, Asp, Met, or not present, X₂ is Tyr or Val, X₃ is Lys or Asp, X₄ is Arg or Ala, X₅ is Pro, Asn, Gly, Ser or Thr, X₆ is Ala or Glu, X₇ is Gln or Tyr, X₈ is Ile or Lys, X₉ is Ala or Thr, X₁₀ is Gln, Ala, or Thr, X₁₁ is Ser, Arg, or Ala, X₁₂ is Trp, Lys, or Arg, X₁₃ is Ala or Cys, X₁₄ is Val or Arg, and X₁₅ is Leu or Ile.

In certain embodiments, the recombinant mutant human sialidase comprises a conservative substitution relative to a recombinant mutant human sialidase sequence disclosed herein. As used herein, the term “conservative substitution” refers to a substitution with a structurally similar amino acid. For example, conservative substitutions may include those within the following groups: Ser and Cys; Leu, Ile, and Val; Glu and Asp; Lys and Arg; Phe, Tyr, and Trp; and Gln, Asn, Glu, Asp, and His. Conservative substitutions may also be defined by the BLAST (Basic Local Alignment Search Tool) algorithm, the BLOSUM substitution matrix (e.g., BLOSUM 62 matrix), or the PAM substitution:p matrix (e.g., the PAM 250 matrix).

Sequence identity may be determined in various ways that are within the skill of a person skilled in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL. EVOL. 36:290-300; Altschul et al., (1997) NUCLEIC ACIDS RES. 25:3389-3402, incorporated by reference herein) are tailored for sequence similarity searching. For a discussion of basic issues in searching sequence databases see Altschul et al., (1994) NATURE GENETICS 6:119-129, which is fully incorporated by reference herein. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919, fully incorporated by reference herein). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent blastp parameter settings may be Q=9; R=2; wink=1; and gapw=32. Searches may also be conducted using the NCBI (National Center for Biotechnology Information) BLAST Advanced Option parameter (e.g.: −G, Cost to open gap [Integer]: default=5 for nucleotides/11 for proteins; −E, Cost to extend gap [Integer]: default=2 for nucleotides/1 for proteins; −q, Penalty for nucleotide mismatch [Integer]: default=−3; −r, reward for nucleotide match [Integer]: default=1; −e, expect value [Real]: default=10; −W, wordsize [Integer]: default=11 for nucleotides/28 for megablast/3 for proteins; −y, Dropoff (X) for blast extensions in bits: default=20 for blastn/7 for others; −X, X dropoff value for gapped alignment (in bits): default=15 for all programs, not applicable to blastn; and −Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty). The equivalent settings in Bestfit protein comparisons are GAP=8 and LEN=2.

b. Antibody Portion

As used herein, unless otherwise indicated, the term “antibody” is understood to mean an intact antibody (e.g., an intact monoclonal antibody) or a fragment thereof, such as an antigen-binding fragment of an antibody (e.g., an antigen-binding fragment of a monoclonal antibody) or a Fc fragment of an antibody (e.g., an Fc fragment of a monoclonal antibody), including an intact antibody, antigen-binding fragment, or Fc fragment that has been modified, engineered, or chemically conjugated. Examples of antigen-binding fragments include Fab, Fab′, (Fab′)₂, Fv, single chain antibodies (e.g., scFv), minibodies, and diabodies. Examples of antibodies that have been modified or engineered include chimeric antibodies, humanized antibodies, and multispecific antibodies (e.g., bispecific antibodies). An example of a chemically conjugated antibody is an antibody conjugated to a toxin moiety.

In certain embodiments, the fusion protein comprises an immunoglobulin Fc domain. As used herein, unless otherwise indicated, the term “immunoglobulin Fc domain” refers to a fragment of an immunoglobulin heavy chain constant region which, either alone or in combination with a second immunoglobulin Fc domain, is capable of binding to an Fc receptor. An immunoglobulin Fc domain may include, e.g., immunoglobulin CH2 and CH3 domains. An immunoglobulin Fc domain may include, e.g., immunoglobulin CH2 and CH3 domains and an immunoglobulin hinge region. Boundaries between immunoglobulin hinge regions, CH2, and CH3 domains are well known in the art, and can be found, e.g., in the PROSITE database (available on the world wide web at prosite.expasy.org).

In certain embodiments, the immunoglobulin Fc domain is derived from a human IgG1, IgG2, IgG3, IgG4, IgA1, IgA2, IgD, IgE, and IgM Fc domain. A single amino acid substitution (S228P according to Kabat numbering; designated IgG4Pro) may be introduced to abolish the heterogeneity observed in recombinant IgG4 antibody. See Angal, S. et al. (1993) MOL. IMMUNOL. 30:105-108.

In certain embodiments, the immunoglobulin Fc domain is derived from a human IgG1 isotype or another isotype that elicits antibody-dependent cell-mediated cytotoxicity (ADCC) and/or complement mediated cytotoxicity (CDC). In certain embodiments, the immunoglobulin Fc domain is derived from a human IgG1 isotype (e.g., SEQ ID NO: 31, SEQ ID NO: 5, or SEQ ID NO: 139).

In certain embodiments, the immunoglobulin Fc domain is derived from a human IgG4 isotype or another isotype that elicits little or no antibody-dependent cell-mediated cytotoxicity (ADCC) and/or complement mediated cytotoxicity (CDC). In certain embodiments, the immunoglobulin Fc domain is derived from a human IgG4 isotype.

In certain embodiments, the immunoglobulin Fc domain comprises either a “knob” mutation, e.g., T366Y, or a “hole” mutation, e.g., Y407T, for heterodimerization with a second polypeptide (residue numbers according to EU numbering, Kabat, E. A., et al. (1991) SEQUENCES OF PROTEINS OF IMMUNOLOGICAL INTEREST, FIFTH EDITION, U.S. Department of Health and Human Services, NIH Publication No. 91-3242). For example, in certain embodiments, the immunoglobulin Fc domain is derived from a human IgG1 Fc domain and comprises a Y407T mutation (e.g., the fusion protein comprises SEQ ID NO: 32, SEQ ID NO: 92, SEQ ID NO: 141, or SEQ ID NO: 143). In certain embodiments, the immunoglobulin Fc domain is derived from a human IgG1 Fc domain and comprises a T366Y mutation (e.g., the fusion protein comprises SEQ ID NO: 33, SEQ ID NO: 93, SEQ ID NO: 142, or SEQ ID NO: 144).

In certain embodiments, the immunoglobulin Fc domain is modified to prevent to glycosylation of the Fc domain. For example, in certain embodiments, the immunoglobulin Fc domain is derived from a human IgG1 Fc domain and comprises a mutation at position N297, for example, an N297A or N297G mutation (residue numbers according to EU numbering, Kabat, E. A., et al., supra). For example, in certain embodiments, the fusion protein comprises SEQ ID NO: 140, SEQ ID NO: 143, or SEQ ID NO: 144.

In certain embodiments, the fusion protein comprises an immunoglobulin antigen-binding domain. The inclusion of such a domain may improve targeting of a fusion protein to a sialylated cell, e.g., a PD-1 expressing cell, and/or to the tumor microenvironment. As used herein, unless otherwise indicated, the term “immunoglobulin antigen-binding domain” refers to a polypeptide that, alone or in combination with another immunoglobulin antigen-binding domain, defines an antigen-binding site. Exemplary immunoglobulin antigen-binding domains include, for example, immunoglobulin heavy chain variable region and an immunoglobulin light chain variable region, where the variable regions together define an antigen binding site, e.g., an anti-PD-1 antigen binding site.

In certain embodiments, the immunoglobulin antigen-binding domain is derived from an anti-PD-1 antibody. Exemplary anti-PD-1 antibodies are described, for example, in U.S. Pat. Nos. 8,952,136, 8,779,105, 8,008,449, 8,741,295, 9,205,148, 9,181,342, 9,102,728, 9,102,727, 8,952,136, 8,927,697, 8,900,587, 8,735,553, and 7,488,802. Exemplary anti-PD-1 antibodies include, for example, nivolumab (Opdivo®, Bristol-Myers Squibb Co.), pembrolizumab (Keytruda®, Merck Sharp & Dohme Corp.), spartalizumab (PDR001, Novartis Pharmaceuticals), pidilizumab (CT-011, Cure Tech), cemiplimab, TX-4014, camrelizumab (SHR1210), sintilimab (IBI308), tislelizumab (BGB-A317), toripalimab (JS 001), dostarlimab (TSR-042, WBP-285), INCMGA00012 (MGA012), and AMP-514.

In certain embodiments, the immunoglobulin antigen-binding domain is derived from pembrolizumab. The pembrolizumab heavy chain amino acid sequence is depicted in SEQ ID NO: 136, and the pembrolizumab light chain amino acid sequence is depicted in SEQ ID NO: 77. A heavy chain variable region derived from pembrolizumab is depicted in SEQ ID NO: 137, and a light chain variable region derived from pembrolizumab is depicted in SEQ ID NO: 138.

c. Linker

In certain embodiments, the sialidase portion of the fusion protein can be linked or fused directly to the anti-PD-1 antibody portion (e.g., immunoglobulin Fc domain and/or immunoglobulin antigen-binding domain) of the fusion protein. In other embodiments, the sialidase portion can be covalently bound to the anti-PD-1 antibody portion by a linker.

The linker may couple, with one or more natural amino acids, the sialidase, or functional fragment thereof, and the antibody portions or fragments, where the amino acid (for example, a cysteine amino acid) may be introduced by site-directed mutagenesis. The linker may include one or more unnatural amino acids. It is contemplated that, in certain circumstances, a linker containing for example, one or more sulfhydryl reactive groups (e.g., a maleimide) may covalently link a cysteine in the sialidase portion or the antibody portion that is a naturally occurring cysteine residue or is the product of site-specific mutagenesis.

The linker may be a cleavable linker or a non-cleavable linker. Optionally or in addition, the linker may be a flexible linker or an inflexible linker.

The linker should be a length sufficiently long to allow the sialidase and the antibody portions to be linked without steric hindrance from one another and sufficiently short to retain the intended activity of the fusion protein. The linker preferably is sufficiently hydrophilic to avoid or minimize instability of the fusion protein. The linker preferably is sufficiently hydrophilic to avoid or minimize insolubility of the fusion protein. The linker should be sufficiently stable in vivo (e.g., it is not cleaved by serum, enzymes, etc.) to permit the fusion protein to be operative in vivo.

The linker may be from about 1 angstroms (Å) to about 150 Å in length, or from about 1 Å to about 120 Å in length, or from about 5 Å to about 110 Å in length, or from about 10 Å to about 100 Å in length. The linker may be greater than about 2, 3, 4, 5, 6, 7, 8, 9, 10, 11, 12, 13, 14, 15, 16, 17, 18, 19, 20, 25, 27, 30 or greater angstroms in length and/or less than about 110, 100, 90, 85, 80, 75, 70, 65, 60, 55, 50, 45, 43, 42, 41, 40, 39, 38, 37, 36, 35, 34, 33, 32, 31, or fewer A in length. Furthermore, the linker may be about 5, 10, 15, 20, 25, 30, 35, 40, 45, 50, 55, 60, 65, 70, 75, 80, 85, 90, 95, 100, 110, and 120 Å in length.

In certain embodiments, the linker comprises a polypeptide linker that connects or fuses the sialidase portion of the fusion protein to the anti-PD-1 antibody portion (e.g., immunoglobulin Fc domain and/or immunoglobulin antigen-binding domain) of the fusion protein. For example, it is contemplated that a gene encoding a sialidase portion linked directly or indirectly (for example, via an amino acid containing linker) to an antibody portion can be created and expressed using conventional recombinant DNA technologies. For example, the amino terminus of a sialidase portion can be linked to the carboxy terminus of either the light or the heavy chain of an antibody portion. For example, for a Fab fragment, the amino terminus or carboxy terminus of the sialidase can be linked to the first constant domain of the heavy antibody chain (CH1). When a linker is employed, the linker may comprise hydrophilic amino acid residues, such as Gln, Ser, Gly, Glu, Pro, His and Arg. In certain embodiments, the linker is a peptide containing 1-25 amino acid residues, 1-20 amino acid residues, 2-15 amino acid residues, 3-10 amino acid residues, 3-7 amino acid residues, 4-25 amino acid residues, 4-20 amino acid residues, 4-15 amino acid residues, 4-10 amino acid residues, 5-25 amino acid residues, 5-20 amino acid residues, 5-15 amino acid residues, or 5-10 amino acid residues. Exemplary linkers include glycine and serine-rich linkers, e.g., (GlyGlyPro)ₙ, or (GlyGlyGlyGlySer)ₙ, where n is 1-5. In certain embodiments, the linker comprises, consists, or consists essentially of GGGGS (SEQ ID NO: 121). In certain embodiments, the linker comprises, consists, or consists essentially of GGGGSGGGGS (SEQ ID NO: 90). In certain embodiments, the linker comprises, consists, or consists essentially of EPKSS (SEQ ID NO: 91). Additional exemplary linker sequences are disclosed, e.g., in George et al. (2003) PROTEIN ENGINEERING 15:871-879, and U.S. Pat. Nos. 5,482,858 and 5,525,491.

In certain embodiments, the fusion protein comprises the amino acid sequence of any one of SEQ ID NOs: 67-73, 78, 81-87, 95, 96, 98, 99, 101, 102, 105, 106, 108, 111, 112, 115, 122, 123, 125, 127, 128, 130, 132, 134, or 145, or an amino acid sequence that has at least 85%, 90%, 95%, 96%, 97%, 98%, or 99% sequence identity to any one of SEQ ID NOs: 67-73, 78, 81-87, 95, 96, 98, 99, 101, 102, 105, 106, 108, 111, 112, 115, 122, 123, 125, 127, 128, 130, 132, 134, or 145.

d. Antibody Conjugates

The invention further provides antibody conjugates containing one or more of the fusion proteins disclosed herein. As used herein, unless otherwise indicated, the term “antibody conjugate” is understood to refer to an antibody, or a functional fragment thereof, that comprises antigen-binding activity (e.g., anti-PD-1 antigen-binding activity) and/or Fc receptor-binding activity, conjugated (e.g., covalently coupled) to an additional functional moiety. In certain embodiments, the antibody or functional antibody fragment is conjugated to a sialidase enzyme, e.g., a recombinant mutant human sialidase enzyme disclosed herein. In certain embodiments, an antibody conjugate comprises a single polypeptide chain. In certain embodiments, an antibody conjugate comprises two, three, four, or more polypeptide chains that are covalently or non-covalently associated together to produce a multimeric complex, e.g., a dimeric, trimeric or tetrameric complex. For example, an antibody conjugate may comprise a first polypeptide (fusion protein) comprising a recombinant mutant human sialidase enzyme and an immunoglobulin heavy chain, and a second polypeptide comprising an immunoglobulin light chain, where, for example, the immunoglobulin heavy and light chains together define a single antigen-binding site, e.g., an anti-PD-1 antigen-binding site.

In certain embodiments, the antibody conjugate can include a single sialidase. In other embodiments, the antibody conjugate can include more than one (e.g., two) sialidases. If more than one sialidase is included, the sialidases can be the same or different. In certain embodiments, the antibody conjugate can include a single anti-PD-1 antigen-binding site. In other embodiments, the antibody conjugate can include more than one (e.g., two) anti-PD-1 antigen-binding sites. If two antigen-binding sites are used, they can be the same or different. In certain embodiments, the antibody conjugate comprises an immunoglobulin Fc fragment.

In certain embodiments, the antibody conjugate comprises one or two immunoglobulin heavy chains, or a functional fragment thereof. In certain embodiments, the antibody conjugate comprises one or two immunoglobulin light chains, or a functional fragment thereof. In certain embodiments, the antibody conjugate comprises a sialidase fused to the N- or C-terminus of an immunoglobulin heavy chain or an immunoglobulin light chain.

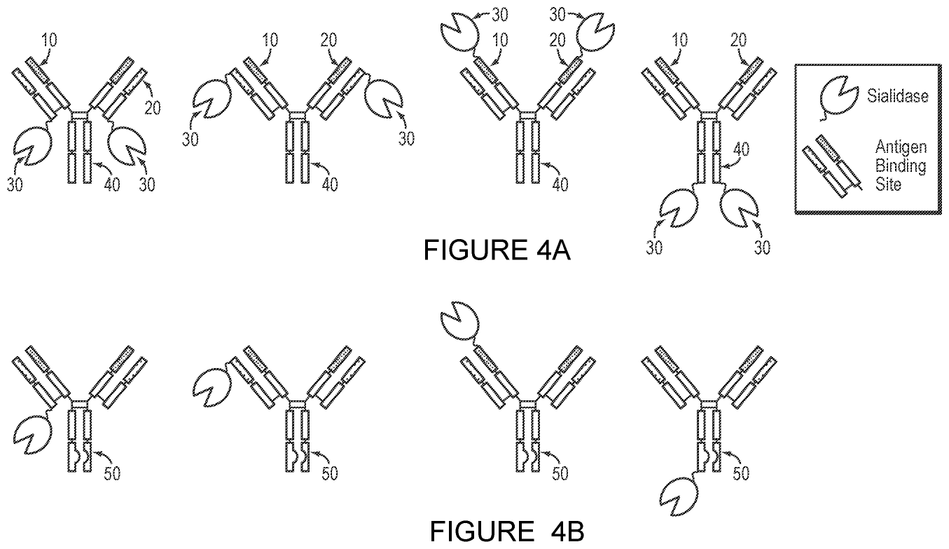

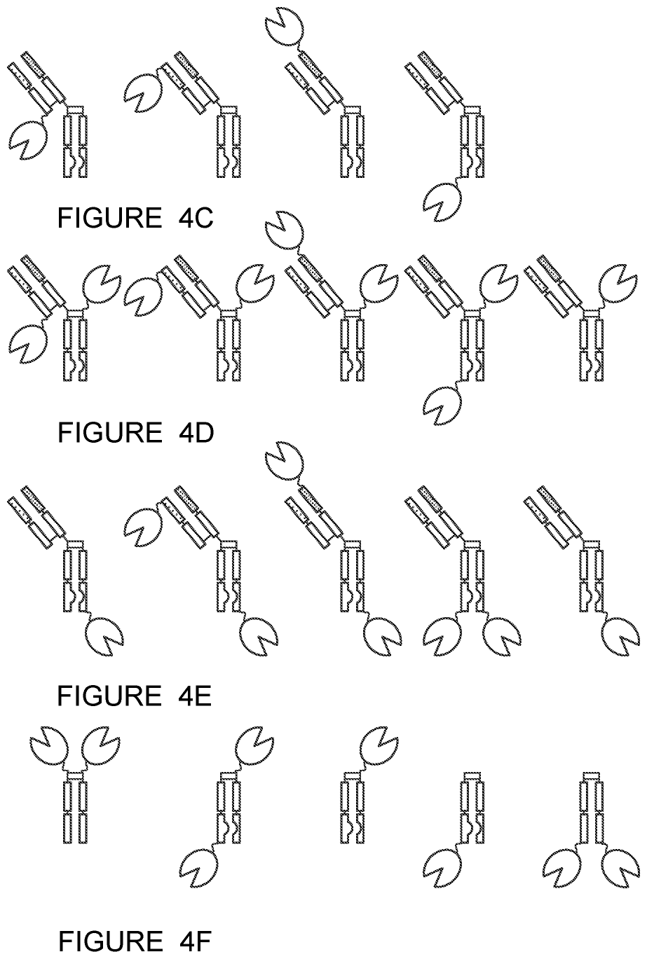

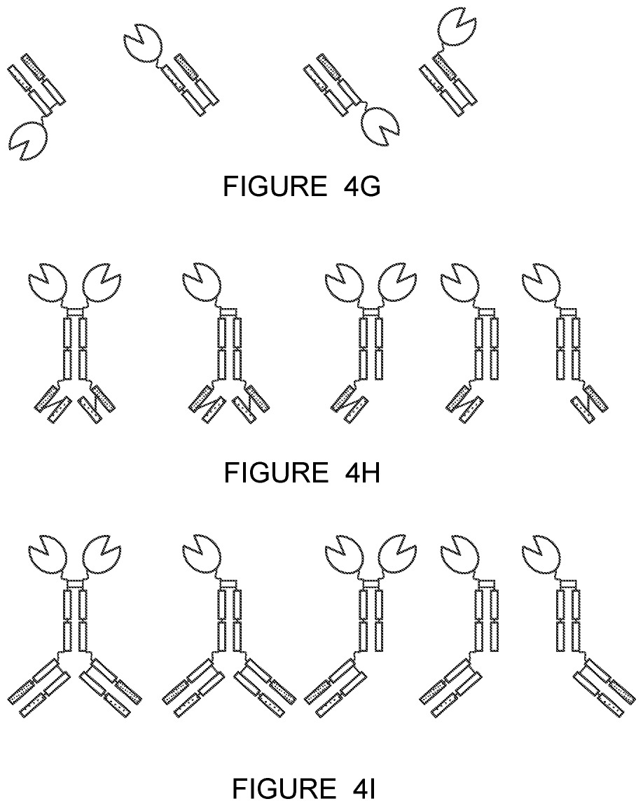

FIG. 4 depicts exemplary antibody conjugate constructs containing one or more sialidase enzymes. For example, in FIG. 4A, a first anti-PD-1 antigen-binding site (e.g., defined by a V_{H} and V_{L} domains) is depicted as 10, a second anti-PD-1 antigen-binding site is depicted as 20, a sialidase is depicted as 30, and a Fc is depicted as 40. In each of the constructs depicted in FIGS. 4A-4I it is understood that the Fc may optionally be modified in some manner, e.g. using Knobs-into-Holes type technology, e.g., as depicted by 50 in FIG. 4B. Throughout FIG. 4 similar structures are depicted by similar schematic representations.

FIG. 4A depicts antibody conjugate constructs comprising a first polypeptide comprising a first immunoglobulin light chain; a second polypeptide comprising a first immunoglobulin heavy chain; a third polypeptide comprising a second immunoglobulin heavy chain; and a fourth polypeptide comprising a second immunoglobulin light chain. The first and second polypeptides can be covalently linked together, the third and fourth polypeptides can be covalently linked together, and the second and third polypeptides can be covalently linked together. The covalent linkages can be disulfide bonds. In certain embodiments, the first polypeptide and the second polypeptide together define a first anti-PD-1 antigen-binding site as depicted as 10, and the third polypeptide and the fourth polypeptide together define a second anti-PD-1 antigen-binding site as depicted as 20. A sialidase enzyme as depicted as 30 can be conjugated to the N- or C-terminus of the first and second immunoglobulin light chain or the first and second immunoglobulin heavy chain.

FIG. 4B depicts antibody conjugate constructs comprising a first polypeptide comprising a first immunoglobulin light chain; a second polypeptide comprising a first immunoglobulin heavy chain; a third polypeptide comprising a second immunoglobulin heavy chain; and a fourth polypeptide comprising a second immunoglobulin light chain. The first and second polypeptides can be covalently linked together, the third and fourth polypeptides can be covalently linked together, and the second and third polypeptides can be covalently linked together. The covalent linkages can be disulfide bonds. In certain embodiments, the first polypeptide and the second polypeptide together define a first anti-PD-1 antigen-binding site, and the third polypeptide and the fourth polypeptide together define a second anti-PD-1 antigen-binding site. A sialidase enzyme can be conjugated to the N- or C-terminus of the first immunoglobulin light chain or the first immunoglobulin heavy chain.

FIG. 4C depicts antibody conjugate constructs comprising a first polypeptide comprising an immunoglobulin light chain; a second polypeptide comprising an immunoglobulin heavy chain; and a third polypeptide comprising an immunoglobulin Fc domain. The first and second polypeptides can be covalently linked together and the second and third polypeptides can be covalently linked together. The covalent linkages can be disulfide bonds. In certain embodiments, the first polypeptide and the second polypeptide together define an anti-PD-1 antigen-binding site. A sialidase enzyme can be conjugated to the N- or C-terminus of the first immunoglobulin light chain or the first immunoglobulin heavy chain.

FIG. 4D depicts antibody conjugate constructs comprising a first polypeptide comprising an immunoglobulin light chain; a second polypeptide comprising an immunoglobulin heavy chain; and a third polypeptide comprising an immunoglobulin Fc domain and a first sialidase enzyme. The first and second polypeptides can be covalently linked together and the second and third polypeptides can be covalently linked together. The covalent linkages can be disulfide bonds. The third polypeptide comprises the sialidase and the immunoglobulin Fc domain in an N- to C-terminal orientation. In certain embodiments, the first polypeptide and the second polypeptide together define an anti-PD-1 antigen-binding site. An optional second sialidase enzyme can be conjugated to the N- or C-terminus of the first immunoglobulin light chain or the first immunoglobulin heavy chain.

FIG. 4E depicts antibody conjugate constructs comprising a first polypeptide comprising an immunoglobulin light chain; a second polypeptide comprising an immunoglobulin heavy chain; and a third polypeptide comprising an immunoglobulin Fc domain and a first sialidase enzyme. The first and second polypeptides can be covalently linked together and the second and third polypeptides can be covalently linked together. The covalent linkages can be disulfide bonds. The third polypeptide comprises the immunoglobulin Fc domain and the sialidase in an N- to C-terminal orientation. In certain embodiments, the first polypeptide and the second polypeptide together define an anti-PD-1 antigen-binding site. An optional second sialidase enzyme can be conjugated to the N- or C-terminus of the first immunoglobulin light chain or the first immunoglobulin heavy chain.

FIG. 4F depicts antibody conjugate constructs comprising a first polypeptide comprising a first immunoglobulin Fc domain, and a second polypeptide comprising a second immunoglobulin Fc domain. The first and second polypeptides can be covalently linked together. The covalent linkages can be disulfide bonds. A sialidase enzyme can be conjugated to the N- or C-terminus of the first immunoglobulin Fc domain or to the N- or C-terminus of the second immunoglobulin Fc domain. An optional second sialidase enzyme can be conjugated to the N- or C-terminus of the first immunoglobulin Fc domain or to the N- or C-terminus of the second immunoglobulin Fc domain.

FIG. 4G depicts antibody conjugate constructs comprising a first polypeptide comprising an immunoglobulin light chain; and a second polypeptide comprising an immunoglobulin heavy chain variable region. The first and second polypeptides can be covalently linked together. The covalent linkages can be disulfide bonds. In certain embodiments, the first polypeptide and the second polypeptide together define an anti-PD-1 antigen-binding site. The sialidase enzyme can be conjugated to the N- or C-terminus of the immunoglobulin light chain or the immunoglobulin heavy chain variable region.

FIG. 4H depicts antibody conjugate constructs comprising a first polypeptide comprising a first immunoglobulin Fc domain, and a second polypeptide comprising a second immunoglobulin Fc domain. The first and second polypeptides can be covalently linked together. The covalent linkages can be disulfide bonds. A sialidase enzyme can be conjugated to the N-terminus of the first immunoglobulin Fc domain or the second immunoglobulin Fc domain. An optional second sialidase enzyme can be conjugated to the N-terminus of the second immunoglobulin Fc domain or the first immunoglobulin Fc domain, respectively. A single chain variable fragment (scFv) can be conjugated to the C-terminus of the first immunoglobulin Fc domain or the second immunoglobulin Fc domain. An optional second single chain variable fragment (scFv) can be conjugated to the C-terminus of the first immunoglobulin Fc domain or the second immunoglobulin Fc domain, respectively.

FIG. 4I depicts antibody conjugate constructs similar to those depicted in FIG. 4H except that each scFv is replaced with an immunoglobulin antigen binding fragment, e.g., a Fab. For example, FIG. 4I depicts antibody conjugate constructs comprising a first polypeptide comprising a first immunoglobulin Fc domain, and a second polypeptide comprising a second immunoglobulin Fc domain. The first and second polypeptides can be covalently linked together. The covalent linkages can be disulfide bonds. A sialidase enzyme can be conjugated to the N-terminus of the first immunoglobulin Fc domain or the second immunoglobulin Fc domain. An optional second sialidase enzyme can be conjugated to the N-terminus of the second immunoglobulin Fc domain or the first immunoglobulin Fc domain, respectively. An antibody fragment (Fab) can be conjugated or fused to the C-terminus of the first immunoglobulin Fc domain or the second immunoglobulin Fc domain. An optional second antibody fragment (Fab) can be conjugated or fused to the C-terminus of the second immunoglobulin Fc domain or the first immunoglobulin Fc domain, respectively. In the case of a fusion, the C terminus of the Fc domain is linked (either by a bond or an amino acid linker) to a first polypeptide chain defining an anti-PD-1 immunoglobulin antigen binding fragment. In the case of antibodies that have an antigen binding site defined by a single variable region, then this may be sufficient to impart binding affinity to a target antigen, e.g., PD-1. In other instances, e.g., in the case of a human antibody, the first polypeptide chain defining an immunoglobulin antigen binding fragment can be conjugated (e.g., covalently conjugated, e.g., via a disulfide bond) to a second polypeptide chain defining an immunoglobulin antigen binding fragment, there the two antigen binding fragments together define an antigen binding site for binding the target antigen, e.g., PD-1.

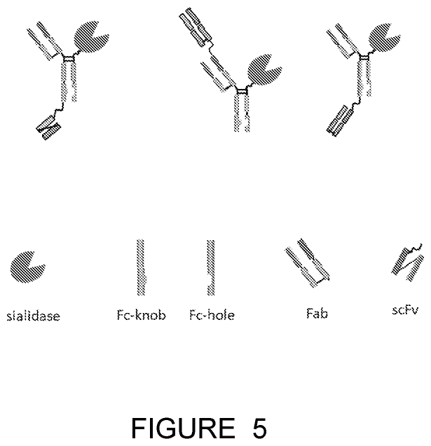

FIG. 5 depicts additional antibody conjugate constructs. For example, FIG. 5 depicts an antibody conjugate construct comprising a first polypeptide comprising an immunoglobulin light chain; a second polypeptide comprising an immunoglobulin heavy chain and an scFv; and a third polypeptide comprising an immunoglobulin Fc domain and a first sialidase enzyme. The first and second polypeptides can be covalently linked together and the second and third polypeptides can be covalently linked together. The covalent linkages can be disulfide bonds. The second polypeptide comprises the heavy chain and the scFv in an N- to C-terminal orientation. The third polypeptide comprises the sialidase and the immunoglobulin Fc domain in an N- to C-terminal orientation. In certain embodiments, the first polypeptide and the second polypeptide together define a first antigen-binding site. In certain embodiments, the scFv defines a second antigen-binding site. FIG. 5 depicts an additional antibody construct comprising a first polypeptide comprising an immunoglobulin light chain; a second polypeptide comprising an immunoglobulin heavy chain; and a third polypeptide comprising an immunoglobulin Fc domain and a first sialidase enzyme, wherein a Fab fragment is conjugated to the N-terminus of the immunoglobulin heavy chain. The first and second polypeptides can be covalently linked together and the second and third polypeptides can be covalently linked together. The covalent linkages can be disulfide bonds. The third polypeptide comprises the sialidase and the immunoglobulin Fc domain in an N- to C-terminal orientation. In certain embodiments, the first polypeptide and the second polypeptide together define a first antigen-binding site. In certain embodiments, the Fab fragment defines a second antigen-binding site. In each of the constructs depicted in FIG. 5 it is understood that an scFv, when present, may be replaced with a Fab fragment, or a Fab fragment, when present, may be replaced with an scFv. In each of the constructs depicted in FIG. 5, it is understood that the Fc may optionally be modified in some manner.

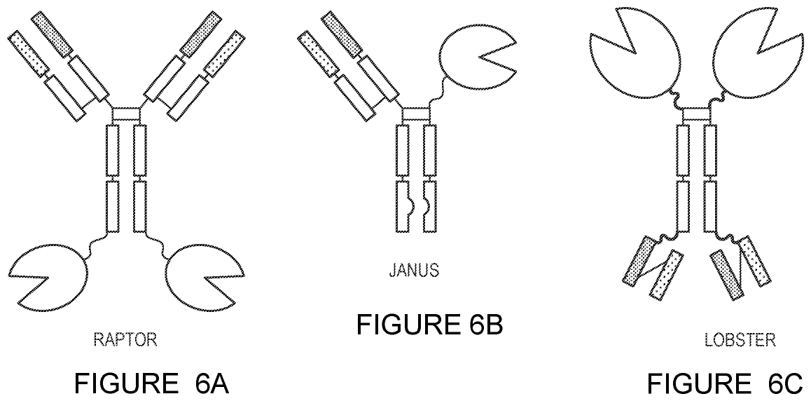

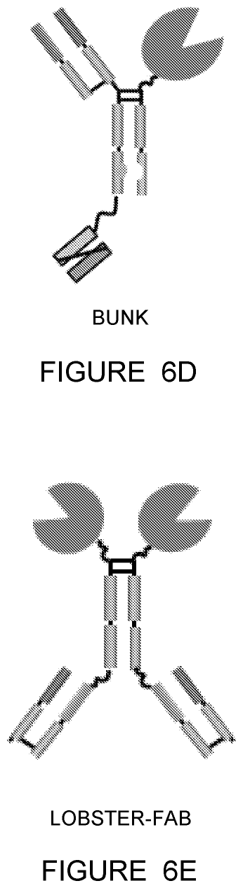

In certain embodiments, the antibody conjugate comprises a first polypeptide comprising a first immunoglobulin light chain; a second polypeptide comprising a first immunoglobulin heavy chain and a first sialidase; a third polypeptide comprising a second immunoglobulin heavy chain and a second sialidase; and a fourth polypeptide comprising a second immunoglobulin light chain. An example of this embodiment is shown in FIG. 6A. The first and second polypeptides can be covalently linked together, the third and fourth polypeptides can be covalently linked together, and the second and third polypeptides can be covalently linked together. The covalent linkages can be disulfide bonds. In certain embodiments, the first polypeptide and the second polypeptide together define a first anti-PD-1 antigen-binding site, and the third polypeptide and the fourth polypeptide together define a second anti-PD-1 antigen-binding site. In certain embodiments, the second and third polypeptides comprise the first and second immunoglobulin heavy chain and the first and second sialidase, respectively, in an N- to C-terminal orientation. In certain embodiments, the second and third polypeptides comprise the first and second sialidase and the first and second immunoglobulin heavy chain, respectively, in an N- to C-terminal orientation.

In certain embodiments, the first and fourth polypeptides comprise the amino acid sequence of SEQ ID NO: 77, or an amino acid sequence that has at least 85%, 90%, 95%, 96%, 97%, 98%, or 99% sequence identity to SEQ ID NO: 77. In certain embodiments, the second and third polypeptides comprise the amino acid sequence of SEQ ID NO: 145, or an amino acid sequence that has at least 85%, 90%, 95%, 96%, 97%, 98%, or 99% sequence identity to SEQ ID NO: 145.

In certain embodiments, the antibody conjugate comprises a first polypeptide comprising an immunoglobulin light chain; a second polypeptide comprising an immunoglobulin heavy chain; and a third polypeptide comprising an immunoglobulin Fc domain and a sialidase. An example of this embodiment is shown in FIG. 6B. The first and second polypeptides can be covalently linked together and the second and third polypeptides can be covalently linked together. The covalent linkages can be disulfide bonds. In certain embodiments, the first polypeptide and the second polypeptide together define an anti-PD-1 antigen-binding site. In certain embodiments, the third polypeptide comprises the sialidase and the immunoglobulin Fc domain in an N- to C-terminal orientation, or the immunoglobulin Fc domain and the sialidase in an N- to C-terminal orientation.

In certain embodiments, the first polypeptide comprises the amino acid sequence of SEQ ID NO: 77, or an amino acid sequence that has at least 85%, 90%, 95%, 96%, 97%, 98%, or 99% sequence identity to SEQ ID NO: 77. In certain embodiments, the second polypeptide comprises the amino acid sequence of SEQ ID NO: 105, or an amino acid sequence that has at least 85%, 90%, 95%, 96%, 97%, 98%, or 99% sequence identity to SEQ ID NO: 105. In certain embodiments, the third polypeptide comprises the amino acid sequence of any one of SEQ ID NOs: 67-73, 78, 81-87, 95, 96, 98, 99, 101, 102, 106, 108, 111, 112, 115, 122, 123, 125, 127, or 128, or an amino acid sequence that has at least 85%, 90%, 95%, 96%, 97%, 98%, or 99% sequence identity to any one of SEQ ID NOs: 67-73, 78, 81-87, 95, 96, 98, 99, 101, 102, 106, 108, 111, 112, 115, 122, 123, 125, 127, or 128.

In certain embodiments, the third polypeptide comprises the amino acid sequence of

(SEQ ID NO: 66)
X₁X₂SX₃X₄X₅LQX₆ESVFQSGAHAYRIPALLYLPGQQSLLAFAEQRX₇SX₈
X₉DEHAELIVX₁₀RRGDYDAX₁₁THQVQWX₁₂AQEVVAQAX₁₃LX₁₄GHRSMN
PCPLYDX₁₅QTGTLFLFFIAIPX₁₆X₁₇VTEX₁₈QQLQTRANVTRLX₁₉X₂₀V
TSTDHGRTWSSPRDLTDAAIGPX₂₁YREWSTFAVGPGHX₂₂LQLHDX₂₃X₂₄
RSLVVPAYAYRKLHPX₂₅X₂₆X₂₇PIPSAFX₂₈FLSHDHGRTWARGHFVX₂₉
QDTX₃₀ECQVAEVX₃₁TGEQRVVTLNARSX₃₂X₃₃X₃₄X₃₅RX₃₆QAQSX₃₇NX₃₈
GLDFQX₃₉X₄₀QX₄₁VKKLX₄₂EPPPX₄₃GX₄₄QGSVISFPSPRSGPGSPAQ
X₄₅LLYTHPTHX₄₆X₄₇QRADLGAYLNPRPPAPEAWSEPX₄₈LLAKGSX₄₉AY
SDLQSMGTGPDGSPLFGX₅₀LYEANDYEEIX₅₁FX₅₂MFTLKQAFPAEYLP
QX₅₃DKTHTCPPCPAPELLGGPSVFLFPPKPKDTLMISRTPEVTCVVVDV
SHEDPEVKENWYVDGVEVHNAKTKPREEQYNSTYRVVSVLTVLHQDWL
NGKEYKCKVSNKALPAPIEKTISKAKGQPREPQVYTLPPSREEMTKNQV
SLTCLVKGFYPSDIAVEWESNGQPENNYKTTPPVLDSDGSFFLTSKLTV
DKSRWQQGNVFSCSVMHEALHNHYTQKSLSLSPGK,

wherein X₁ is Ala, Arg, Asn, Asp, Gln, Glu, Gly, His, Leu, Lys, Met, Phe, Thr, Val, or not present, X₂ is Ala or Lys, X₃ is Asn or Leu, X₄ is Pro or His, X₅ is Phe, Trp, Tyr or Val, X₆ is Lys or Asp, X₇ is Ala or Arg, X₈ is Lys, Arg, or Glu, X₉ is Lys, Ala, Arg, or Glu, X₁₀ is Leu or Met, X₁₁ is Pro, Asn, Asp, His, Glu, Gly, Ser or Thr, X₁₂ is Gln or His, X₁₃ is Arg or Lys, X₁₄ is Asp or Pro, X₁₅ is Ala, Glu or Lys, X₁₆ is Gly or Asp, X₁₇ is Gln or His, X₁₈ is Gln, Arg, or Lys, X₁₉ is Ala, Cys, Ile, Ser, Val, or Leu, X₂₀ is Gln, Leu, Glu, Phe, His, Ile, Leu, or Tyr, X₂₁ is Ala or Val, X₂₂ is Cys or Gly, X₂₃ is Arg or Pro, X₂₄ is Ala or Gly, X₂₅ is Arg, Ile, or Lys, X₂₆ is Gln or Pro, X₂₇ is Arg or Pro, X₂₈ is Ala, Cys, Leu, or Val, X₂₉ is Ala, Cys, Asn, Ser, or Thr, X₃₀ is Leu, Ala, or Val, X₃₁ is Glu or Pro, X₃₂ is His or Pro, X₃₃ is Leu, Asp, Asn, or Tyr, X₃₄ is Arg, Ala, Asp, Leu, Gln, or Tyr, X₃₅ is Ala, Cys, Phe, Gly, His, Ile, Lys, Leu, Met, Asn, Gln, Arg, Ser, Val, Trp, or Tyr, X₃₆ is Val, Ile, or Lys, X₃₇ is Thr or Ala, X₃₈ is Asp or Gly, X₃₉ is Glu, Lys, or Pro, X₄₀ is Ser or Cys, X₄₁ is Leu, Asp, Phe, Gln, or Thr, X₄₂ is Val or Phe, X₄₃ is Gln, Ala, His, Phe, Pro, Ser, or Thr, X₄₄ is Cys or Val, X₄₅ is Trp or Arg, X₄₆ is Ser, Arg, Ala, Asp, Glu, Phe, Gly, His, Ile, Lys, Leu, Met, Asn, Pro, Gln, Thr, Val, Trp, or Tyr, X₄₇ is Trp, Lys, Ala, Asp, Glu, Phe, Gly, His, Ile, Lys, Leu, Met, Asn, Pro, Gln, Arg, Ser, Thr, Val, or Tyr, X₄₈ is Lys or Val, X₄₉ is Ala, Cys, Ser, or Val, X₅₀ is Cys, Leu, or Val, X₅₁ is Val or Arg, X₅₂ is Leu, Gln, His, Ile, Lys, or Ser, and X₅₃ is GGGGS (SEQ ID NO: 121), GGGGSGGGGS (SEQ ID NO: 90), or EPKSS (SEQ ID NO: 91), and the sialidase comprises at least one mutation relative to wild-type human Neu2 (SEQ ID NO: 1).

In certain embodiments, the third polypeptide comprises the amino acid sequence of

(SEQ ID NO: 76)
X₁ASLPX₂LQX₃ESVFQSGAHAYRIPALLYLPGQQSLLAFAEQRX₄SKKDE
HAELIVLRRGDYDAX₅THQVQWQAQEVVAQARLDGHRSMNPCPLYDX₆QT
GTLFLFFIAIPGQVTEQQQLQTRANVTRLCX₇VTSTDHGRTWSSPRDLTD
AAIGPAYREWSTFAVGPGHCLQLHDRARSLVVPAYAYRKLHPX₈QRPIPS
AFCFLSHDHGRTWARGHFVAQDTLECQVAEVETGEQRVVTLNARSHLR
X₉RVQAQSTNDGLDFQESQLVKKLVEPPPX₁₀GCQGSVISFPSPRSGPGS
PAQWLLYTHPTHX₁₁X₁₂QRADLGAYLNPRPPAPEAWSEPVLLAKGSX₁₃A
YSDLQSMGTGPDGSPLFGCLYEANDYEEIX₁₄FX₁₅MFTLKQAFPAEYLPQ
X₁₆DKTHTCPPCPAPELLGGPSVELFPPKPKDTLMISRTPEVTCVVVDVS
HEDPEVKFNWYVDGVEVHNAKTKPREEQYNSTYRVVSVLTVLHQDWLNG
KEYKCKVSNKALPAPIEKTISKAKGQPREPQVYTLPPSREEMTKNQVSL
TCLVKGFYPSDIAVEWESNGQPENNYKTTPPVLDSDGSFFLTSKLTVDK
SRWQQGNVFSCSVMHEALHNHYTQKSLSLSPGK,

wherein X₁ is Ala, Arg, Asn, Asp, Gln, Glu, Gly, His, Leu, Lys, Met, Phe, Thr, Val, or not present, X₂ is Phe, Trp, Tyr or Val, X₃ is Lys or Asp, X₄ is Arg or Ala, X₅ is Pro, Asn, Asp, His, Glu, Gly, Ser or Thr, X₆ is Ala, Glu, or Lys, X₇ is Gln, Leu, Glu, Phe, His, Ile, Leu, or Tyr, X₈ is Arg, Ile, or Lys, X₉ is Ala, Cys, Phe, Gly, His, Ile, Lys, Leu, Met, Asn, Gln, Arg, Ser, Val, Trp, or Tyr, X₁₀ is Gln, Ala, His, Phe, Pro, Ser, or Thr, X₁₁ is Ser, Arg, Ala, Asp, Glu, Phe, Gly, His, Ile, Lys, Leu, Met, Asn, Pro, Gln, Thr, Val, Trp, or Tyr, X₁₂ is Trp, Lys, Ala, Asp, Glu, Phe, Gly, His, Ile, Lys, Leu, Met, Asn, Pro, Gln, Arg, Ser, Thr, Val, or Tyr, X₁₃ is Ala, Cys, Ser, or Val, X₁₄ is Val or Arg, X₁₅ is Leu, Gln, His, Ile, Lys, or Ser, and X₁₆ is GGGGS (SEQ ID NO: 121), GGGGSGGGGS (SEQ ID NO: 90), or EPKSS (SEQ ID NO: 91), and the sialidase comprises at least one mutation relative to wild-type human Neu2 (SEQ ID NO: 1). In certain embodiments, X₁ is Ala, Asp, Met, or not present, X₂ is Tyr or Val, X₃ is Lys or Asp, X₄ is Arg or Ala, X₅ is Pro, Asn, Gly, Ser or Thr, X₆ is Ala or Glu, X₇ is Gln or Tyr, X₈ is Ile or Lys, X₉ is Ala or Thr, X₁₀ is Gln, Ala, or Thr, X₁₁ is Ser, Arg, or Ala, X₁₂ is Trp, Lys, or Arg, X₁₃ is Ala or Cys, X₁₄ is Val or Arg, and X₁₅ is Leu or Ile.

In certain embodiments, the first polypeptide comprises SEQ ID NO: 77, the second polypeptide comprises SEQ ID NO: 105, and the third polypeptide comprises SEQ ID NO: 111. In certain embodiments, the first polypeptide comprises SEQ ID NO: 77, the second polypeptide comprises SEQ ID NO: 105, and the third polypeptide comprises SEQ ID NO: 115. In certain embodiments, the first polypeptide comprises SEQ ID NO: 77, the second polypeptide comprises SEQ ID NO: 105, and the third polypeptide comprises SEQ ID NO: 125.

In certain embodiments, the antibody conjugate comprises a first polypeptide comprising a first sialidase, a first immunoglobulin Fc domain, and a first single chain variable fragment (scFv) (it is also understood that the scFv may be replaced by a first polypeptide chain of an immunoglobulin antigen binding fragment, e.g., Fab fragment); and a second polypeptide comprising a second sialidase, a second immunoglobulin Fc domain, and a second single chain variable fragment (scFv) (it is also understood that the scFv may be replaced by a second polypeptide chain of an immunoglobulin antigen binding fragment, e.g., Fab fragment). An example of this embodiment is shown in FIG. 6C (in the construct depicted in FIG. 6C it is understood that an scFv, when present, may be replaced with a Fab fragment, or a Fab fragment, when present, may be replaced with an scFv). The first and second polypeptides can be covalently linked together. The covalent linkages can be disulfide bonds. In certain embodiments, the first scFv defines a first anti-PD-1 antigen-binding site, and the second scFv defines a second anti-PD-1 antigen-binding site. In certain embodiments, the first polypeptide comprises the first sialidase, the first immunoglobulin Fc domain, and the first scFv in an N- to C-terminal orientation. In certain embodiments, the first polypeptide comprises the first scFv, the first immunoglobulin Fc domain, and the first sialidase in an N- to C-terminal orientation. In certain embodiments, the second polypeptide comprises the second sialidase, the second immunoglobulin Fc domain, and the second scFv in an N- to C-terminal orientation. In certain embodiments, the second polypeptide comprises the second scFv, the second immunoglobulin Fc domain, and the second sialidase in an N- to C-terminal orientation.

In certain embodiments, the antibody conjugate comprises: a first polypeptide comprising an immunoglobulin light chain; a second polypeptide comprising an immunoglobulin heavy chain and a single chain variable fragment (scFv) (it is also understood that the scFv may be replaced by a first polypeptide chain of an immunoglobulin antigen binding fragment, e.g., Fab fragment); and a third polypeptide comprising an immunoglobulin Fc domain and a sialidase. An example of this embodiment is shown in FIG. 6D. The first and second polypeptides can be covalently linked together and the second and third polypeptides can be covalently linked together. The covalent linkages can be disulfide bonds. In certain embodiments, the first polypeptide and the second polypeptide together define a first anti-PD-1 antigen-binding site (i.e., the immunoglobulin light chain and immunoglobulin heavy chain together define a first anti-PD-1 antigen-binding site). In certain embodiments, the scFv defines a second anti-PD-1 antigen-binding site. In certain embodiments, the second polypeptide comprises the immunoglobulin heavy chain and the scFv in an N- to C-terminal orientation, or the scFv and the immunoglobulin heavy chain in an N- to C-terminal orientation. In certain embodiments, the third polypeptide comprises the sialidase and the immunoglobulin Fc domain in an N- to C-terminal orientation, or the sialidase and the immunoglobulin Fc domain in an N- to C-terminal orientation.

In certain embodiments, the antibody conjugate comprises a first polypeptide comprising a first immunoglobulin light chain; a second polypeptide comprising a first sialidase, a first immunoglobulin Fc domain, and a first immunoglobulin heavy chain variable region; a third polypeptide comprising a second sialidase, a second immunoglobulin Fc domain, and a second immunoglobulin heavy chain variable region; and a fourth polypeptide comprising a second immunoglobulin light chain. It is also understood that an immunoglobulin light chain may be replaced by an immunoglobulin heavy chain variable region and an immunoglobulin heavy chain variable region may be replaced by an immunoglobulin light chain (e.g., the antibody conjugate may comprise a first polypeptide comprising a first immunoglobulin heavy chain variable region; a second polypeptide comprising a first sialidase, a first immunoglobulin Fc domain, and a first immunoglobulin light chain; a third polypeptide comprising a second sialidase, a second immunoglobulin Fc domain, and a second immunoglobulin light chain; and a fourth polypeptide comprising a second immunoglobulin heavy chain variable region). An example of this embodiment is shown in FIG. 6E. The second and third polypeptides can be covalently linked together. The covalent linkages can be disulfide bonds. In certain embodiments, the first and second polypeptides defines a first anti-PD-1 antigen-binding site, and the third and fourth polypeptides defines a second anti-PD-1 antigen-binding site. In certain embodiments, the second polypeptide comprises the first sialidase, the first immunoglobulin Fc domain, and the first immunoglobulin heavy chain variable region in an N- to C-terminal orientation. In certain embodiments, the third polypeptide comprises the second sialidase, the second immunoglobulin Fc domain, and the second immunoglobulin heavy chain variable region in an N- to C-terminal orientation.

In certain embodiments, the antibody conjugate has a molecular weight from about 135 kDa to about 165 kDa, e.g., about 140 kDa. In other embodiments, the antibody conjugate has a molecular weight from about 215 kDa to about 245 kDa, e.g., about 230 kDa.

In certain embodiments, the antibody conjugate comprises two polypeptides that each comprise an immunoglobulin Fc domain, and the first polypeptide has either a “knob” mutation, e.g., T366Y, or a “hole” mutation, e.g., Y407T, for heterodimerization with the second polypeptide, and the second polypeptide has either a respective “knob” mutation, e.g., T366Y, or a “hole” mutation, e.g., Y407T, for heterodimerization with the first polypeptide (residue numbers according to EU numbering, Kabat, E. A., et al. (1991) supra). For example, in certain embodiments, the antibody comprises two polypeptides that each comprise an immunoglobulin Fc domain derived from human IgG1 Fc domain, and the first polypeptide comprises a Y407T mutation (e.g., the first polypeptide comprises SEQ ID NO: 32 or SEQ ID NO: 92), and the second polypeptide comprises a T366Y mutation (e.g., the second polypeptide comprises SEQ ID NO: 33 or SEQ ID NO: 93).

As used herein, the term “multispecific antibody” is understood to mean an antibody that specifically binds to at least two different antigens, i.e., an antibody that comprises at least two antigen-binding sites that bind to at least two different antigens. As used herein, the term “bispecific antibody” is understood to mean an antibody that specifically binds to two different antigens, i.e., an antibody that comprises two antigen-binding sites each of which bind to separate and distinct antigens. In other words, a first binding site binds a first antigen and a second binding site binds a second, different antigen. A multispecific or bispecific antibody may, for example, be a human or humanized antibody, and/or be a full length antibody or an antibody fragment (e.g., a F(ab′)₂ bispecific antibody).

The present invention encompasses antibody conjugates comprising antibody fragments, which may be generated by traditional means, such as enzymatic digestion, or by recombinant techniques. For a review of certain antibody fragments, see Hudson et al. (2003) supra.

In certain embodiments, the antibody conjugate or fusion protein can be covalently or non-covalently associated with a biological modifier, wherein the biological modifier can be used to enhance the solubility of the antibody, increase binding specificity, decrease immunogenicity or toxicity or modify the pharmacokinetic profile of the antibody. For example, the biological modifier can be used to increase the molecular weight of the antibody to increase its circulating half-life.

It is contemplated that the antibody conjugate or fusion protein may be covalently bound to one or more (for example, 2, 3, 4, 5, 6, 8, 9, 10 or more) biological modifiers that may comprise linear or branched polymers. Exemplary biological modifiers may include, for example, a variety of polymers, such as those described in U.S. Pat. No. 7,842,789. Particularly useful are polyalkylene ethers such as polyethylene glycol (PEG) and derivatives thereof (for example, alkoxy polyethylene glycol, for example, methoxypolyethylene glycol, ethoxypolyethylene glycol and the like); block copolymers of polyoxyethylene and polyoxypropylene (Pluronics); polymethacrylates; carbomers; and branched or unbranched polysaccharides which comprise the saccharide monomers such as D-mannose, D- and L-galactose, fucose, fructose, D-xylose, L-arabinose, and D-glucuronic acid.

In other embodiments, the biological modifier can be a hydrophilic polyvinyl polymer such as polyvinyl alcohol and polyvinylpyrrolidone (PVP)-type polymers. The biological modifier can be a functionalized polyvinylpyrrolidone, for example, carboxy or amine functionalized on one (or both) ends of the polymer (as available from PolymerSource). Alternatively, the biological modifier can include Poly N-(2-hydroxypropyl)methacrylamide (HPMA), or functionalized HPMA (amine, carboxy, etc.), Poly(N-isopropylacrylamide) or functionalized poly(N-isopropylacrylamide). Alternatively, the biological modifier can include Poly N-(2-hydroxypropyl)methacrylamide (HPMA), or functionalized HPMA (amine, carboxy, etc.), Poly(N-isopropylacrylamide) or functionalized poly(N-isopropylacrylamide). The modifier prior to conjugation need not be, but preferably is, water soluble, but the final conjugate should be water soluble.

In general, the biological modifier may have a molecular weight from about 2 kDa to about 5 kDa, from about 2 kDa to about 10 kDa, from about 2 kDa to about 20 kDa, from about 2 kDa to about 30 kDa, from about 2 kDa to about 40 kDa, from about 2 kDa to about 50 kDa, from about 2 kDa to about 60 kDa, from about 2 kDa to about 70 kDa, from about 2 kDa to about 80 kDa, from about 2 kDa to about 90 kDa, from about 2 kDa to about 100 kDa, from about 2 kDa to about 150 kDa, from about 5 kDa to about 10 kDa, from about 5 kDa to about 20 kDa, from about 5 kDa to about 30 kDa, from about 5 kDa to about 40 kDa, from about 5 kDa to about 50 kDa, from about 5 kDa to about 60 kDa, from about 5 kDa to about 70 kDa, from about 5 kDa to about 80 kDa, from about 5 kDa to about 90 kDa, from about 5 kDa to about 100 kDa, from about 5 kDa to about 150 kDa, from about 10 kDa to about 20 kDa, from about 10 kDa to about 30 kDa, from about 10 kDa to about 40 kDa, from about 10 kDa to about 50 kDa, from about 10 kDa to about 60 kDa, from about 10 kDa to about 70 kDa, from about 10 kDa to about 80 kDa, from about 10 kDa to about 90 kDa, from about 10 kDa to about 100 kDa, from about 10 kDa to about 150 kDa, from about 20 kDa to about 30 kDa, from about 20 kDa to about 40 kDa, from about 20 kDa to about 50 kDa, from about 20 kDa to about 60 kDa, from about 20 kDa to about 70 kDa, from about 20 kDa to about 80 kDa, from about 20 kDa to about 90 kDa, from about 20 kDa to about 100 kDa, from about 20 kDa to about 150 kDa, from about 30 kDa to about 40 kDa, from about 30 kDa to about 50 kDa, from about 30 kDa to about 60 kDa, from about 30 kDa to about 70 kDa, from about 30 kDa to about 80 kDa, from about 30 kDa to about 90 kDa, from about 30 kDa to about 100 kDa, from about 30 kDa to about 150 kDa, from about 40 kDa to about 50 kDa, from about 40 kDa to about 60 kDa, from about 40 kDa to about 70 kDa, from about 40 kDa to about 80 kDa, from about 40 kDa to about 90 kDa, from about 40 kDa to about 100 kDa, from about 40 kDa to about 150 kDa, from about 50 kDa to about 60 kDa, from about 50 kDa to about 70 kDa, from about 50 kDa to about 80 kDa, from about 50 kDa to about 90 kDa, from about 50 kDa to about 100 kDa, from about 50 kDa to about 150 kDa, from about 60 kDa to about 70 kDa, from about 60 kDa to about 80 kDa, from about 60 kDa to about 90 kDa, from about 60 kDa to about 100 kDa, from about 60 kDa to about 150 kDa, from about 70 kDa to about 80 kDa, from about 70 kDa to about 90 kDa, from about 70 kDa to about 100 kDa, from about 70 kDa to about 150 kDa, from about 80 kDa to about 90 kDa, from about 80 kDa to about 100 kDa, from about 80 kDa to about 150 kDa, from about 90 kDa to about 100 kDa, from about 90 kDa to about 150 kDa, or from about 100 kDa to about 150 kDa.

It is contemplated that the antibody conjugate or fusion protein is attached to about 10 or fewer polymer molecules (e.g., 9, 8, 7, 6, 5, 4, 3, 2, or 1), each polymer molecule having a molecular weight of at least about 20,000 D, or at least about 30,000 D, or at least about 40,000 D.

Although a variety of polymers can be used as biological modifiers, it is contemplated that the antibody conjugates or fusion proteins described herein may be attached to polyethylene glycol (PEG) polymers. In one embodiment, the antibody conjugate or fusion protein described herein is covalently attached to at least one PEG having an actual MW of at least about 20,000 D. In another embodiment, the antibody conjugate or fusion protein described herein is covalently attached to at least one PEG having an actual MW of at least about 30,000 D. In another embodiment, the antibody conjugate or fusion protein described herein is covalently attached to at least one PEG having an actual MW of at least about 40,000 D. In certain embodiments, the PEG is methoxyPEG (5000)-succinimidylpropionate (mPEG-SPA), methoxyPEG (5000)-succinimidylsuccinate (mPEG-SS). Such PEGS are commercially available from Nektar Therapeutics or SunBiowest.

Attachment sites on an antibody conjugate or fusion protein for a biological modifier include the N-terminal amino group and epsilon amino groups found on lysine residues, as well as other amino, imino, carboxyl, sulfhydryl, hydroxyl or other hydrophilic groups. The polymer may be covalently bonded directly to the antibody conjugate or fusion protein with or without the known use of a multifunctional (ordinarily bifunctional) crosslinking agent using chemistries and used in the art. For example, sulfhydryl groups can be derivatized by coupling to maleimido-substituted PEG (e.g. alkoxy-PEG amine plus sulfosuccinimidyl 4-(N-maleimidomethyl)cyclohexane-1-carboxylate), or PEG-maleimide commercially available from Shearwater Polymers, Inc., Huntsville, Ala).

### II. Methods of Making a Fusion Protein or Antibody Conjugate

Methods for producing fusion proteins, e.g., those disclosed herein, antibodies, or antibody conjugates, e.g., those disclosed herein, are known in the art. For example, DNA molecules encoding light chain variable regions and/or heavy chain variable regions can be synthesized chemically or by recombinant DNA methodologies. For example, the sequences of the antibodies can be cloned from hybridomas by conventional hybridization techniques or polymerase chain reaction (PCR) techniques, using the appropriate synthetic nucleic acid primers. The resulting DNA molecules encoding the variable regions of interest can be ligated to other appropriate nucleotide sequences, including, for example, constant region coding sequences, and expression control sequences, to produce conventional gene expression constructs (i.e., expression vectors) encoding the desired antibodies. Production of defined gene constructs is within routine skill in the art.

Nucleic acids encoding desired fusion proteins, and/or antibody conjugates can be incorporated (ligated) into expression vectors, which can be introduced into host cells through conventional transfection or transformation techniques. Exemplary host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells that do not otherwise produce IgG protein. Transformed host cells can be grown under conditions that permit the host cells to express the genes that encode the immunoglobulin light and/or heavy chain variable regions.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed protein may be secreted. The expressed protein may accumulate in refractile or inclusion bodies, which can be harvested after disruption of the cells by French press or sonication. The refractile bodies then are solubilized, and the protein may be refolded and/or cleaved by methods known in the art.

If the engineered gene is to be expressed in eukaryotic host cells, e.g., CHO cells, it is first inserted into an expression vector containing a suitable eukaryotic promoter, a secretion signal, a poly A sequence, and a stop codon. Optionally, the vector or gene construct may contain enhancers and introns. In embodiments involving fusion proteins comprising an antibody or portion thereof, the expression vector optionally contains sequences encoding all or part of a constant region, enabling an entire, or a part of, a heavy or light chain to be expressed. The gene construct can be introduced into eukaryotic host cells using conventional techniques.

The host cells express a fusion protein and/or antibody conjugate comprising a sialidase and V_{L} or V_{H} fragments, V_{L}-V_{H} heterodimers, V_{H}-V_{L} or V_{L}-V_{H} single chain polypeptides, complete heavy or light immunoglobulin chains, or portions thereof, each of which may be attached to a moiety having another function (e.g., cytotoxicity). In some embodiments involving fusion proteins and/or antibody conjugates, a host cell is transfected with a single vector expressing a polypeptide expressing a sialidase and an entire, or part of, a heavy chain (e.g., a heavy chain variable region) or a sialidase and a light chain (e.g., a light chain variable region), or a polypeptide expressing an entire, or part of, a heavy chain (e.g., a heavy chain variable region) or a light chain (e.g., a light chain variable region). In some embodiments, a host cell is transfected with a single vector encoding (a) a polypeptide comprising a heavy chain variable region and a polypeptide comprising a light chain variable region, or (b) an entire immunoglobulin heavy chain and an entire immunoglobulin light chain, wherein in (a) or in (b), the polypeptide may also comprise a sialidase. In some embodiments, a host cell is co-transfected with more than one expression vector (e.g., one expression vector expressing a polypeptide comprising an entire, or part of, a heavy chain or heavy chain variable region, optionally comprising a sialidase fused thereto, and another expression vector expressing a polypeptide comprising an entire, or part of, a light chain or light chain variable region, optionally comprising a sialidase fused thereto).

A polypeptide comprising a fusion protein, e.g., a fusion protein comprising an immunoglobulin heavy chain variable region or light chain variable region, can be produced by growing (culturing) a host cell transfected with an expression vector encoding such a variable region, under conditions that permit expression of the polypeptide. Following expression, the polypeptide can be harvested and purified or isolated using techniques known in the art, e.g., affinity tags such as glutathione-S-transferase (GST) or histidine tags.

In embodiments in which a fusion protein and/or antibody conjugate is produced, a sialidase fused to a monoclonal antibody, Fc domain, or an antigen-binding domain of the antibody, can be produced by growing (culturing) a host cell transfected with: (a) an expression vector that encodes a complete or partial immunoglobulin heavy chain, and a separate expression vector that encodes a complete or partial immunoglobulin light chain; or (b) a single expression vector that encodes both chains (e.g., complete or partial heavy and light chains), under conditions that permit expression of both chains. The sialidase will be fused to one or more of the chains. The intact fusion protein and/or antibody conjugate can be harvested and purified or isolated using techniques known in the art, e.g., Protein A, Protein G, affinity tags such as glutathione-S-transferase (GST) or histidine tags. It is within ordinary skill in the art to express the heavy chain and the light chain from a single expression vector or from two separate expression vectors.

In certain embodiments, in order to express a protein, e.g., a fusion protein, as a secreted protein, a native N-terminal signal sequence of the protein is replaced, e.g., with MDMRVPAQLLGLLLLWLPGARC (SEQ ID NO: 28). In certain embodiments, to express a protein, e.g., a fusion protein, as a secreted protein, an N-terminal signal sequence, e.g., MDMRVPAQLLGLLLLWLPGARC (SEQ ID NO: 28), is added. Additional exemplary N-terminal signal sequences include signal sequences from interleukin-2, CD-5, IgG kappa light chain, trypsinogen, serum albumin, and prolactin. In certain embodiments, in order to express a protein, e.g., a fusion protein, as a secreted protein, a C terminal lysosomal signal motif, e.g., YGTL (SEQ ID NO: 29) is removed.

Methods for reducing or eliminating the antigenicity of antibodies and antibody fragments are known in the art. When the antibodies are to be administered to a human, the antibodies preferably are “humanized” to reduce or eliminate antigenicity in humans. Preferably, each humanized antibody has the same or substantially the same affinity for the antigen as the non-humanized mouse antibody from which it was derived.

In one humanization approach, chimeric proteins are created in which mouse immunoglobulin constant regions are replaced with human immunoglobulin constant regions. See, e.g., Morrison et al., 1984, PROC. NAT. ACAD. SCI. 81:6851-6855, Neuberger et al., 1984, NATURE 312:604-608; U.S. Pat. No. 6,893,625 (Robinson); U.S. Pat. No. 5,500,362 (Robinson); and U.S. Pat. No. 4,816,567 (Cabilly).

In an approach known as CDR grafting, the CDRs of the light and heavy chain variable regions are grafted into frameworks from another species. For example, murine CDRs can be grafted into human FRs. In some embodiments, the CDRs of the light and heavy chain variable regions of an antibody are grafted into human FRs or consensus human FRs. To create consensus human FRs, FRs from several human heavy chain or light chain amino acid sequences are aligned to identify a consensus amino acid sequence. CDR grafting is described in U.S. Pat. No. 7,022,500 (Queen); U.S. Pat. No. 6,982,321 (Winter); U.S. Pat. No. 6,180,370 (Queen); U.S. Pat. No. 6,054,297 (Carter); U.S. Pat. No. 5,693,762 (Queen); U.S. Pat. No. 5,859,205 (Adair); U.S. Pat. No. 5,693,761 (Queen); U.S. Pat. No. 5,565,332 (Hoogenboom); U.S. Pat. No. 5,585,089 (Queen); U.S. Pat. No. 5,530,101 (Queen); Jones et al. (1986) NATURE 321: 522-525; Riechmann et al. (1988) NATURE 332: 323-327; Verhoeyen et al. (1988) SCIENCE 239: 1534-1536; and Winter (1998) FEBS LETT 430: 92-94.

In an approach called “SUPERHUMANIZATION™,” human CDR sequences are chosen from human germline genes, based on the structural similarity of the human CDRs to those of the mouse antibody to be humanized. See, e.g., U.S. Pat. No. 6,881,557 (Foote); and Tan et al., 2002, J. IMMUNOL. 169:1119-1125.

Other methods to reduce immunogenicity include “reshaping,” “hyperchimerization,” and “veneering/resurfacing.” See, e.g., Vaswami et al., 1998, ANNALS OF ALLERGY, ASTHMA, & IMMUNOL. 81:105; Roguska et al., 1996, PROT. ENGINEER 9:895-904; and U.S. Pat. No. 6,072,035 (Hardman). In the veneering/resurfacing approach, the surface accessible amino acid residues in the murine antibody are replaced by amino acid residues more frequently found at the same positions in a human antibody. This type of antibody resurfacing is described, e.g., in U.S. Pat. No. 5,639,641 (Pedersen).

Another approach for converting a mouse antibody into a form suitable for medical use in humans is known as ACTIVMAB™ technology (Vaccinex, Inc., Rochester, NY), which involves a vaccinia virus-based vector to express antibodies in mammalian cells. High levels of combinatorial diversity of IgG heavy and light chains can be produced. See, e.g., U.S. Pat. No. 6,706,477 (Zauderer); U.S. Pat. No. 6,800,442 (Zauderer); and U.S. Pat. No. 6,872,518 (Zauderer). Another approach for converting a mouse antibody into a form suitable for use in humans is technology practiced commercially by KaloBios Pharmaceuticals, Inc. (Palo Alto, CA). This technology involves the use of a proprietary human “acceptor” library to produce an “epitope focused” library for antibody selection. Another approach for modifying a mouse antibody into a form suitable for medical use in humans is HUMAN ENGINEERING™ technology, which is practiced commercially by XOMA (US) LLC. See, e.g., International (PCT) Publication No. WO 93/11794 and U.S. Pat. No. 5,766,886 (Studnicka); U.S. Pat. No. 5,770,196 (Studnicka); U.S. Pat. No. 5,821,123 (Studnicka); and 5,869,619 (Studnicka).

Any suitable approach, including any of the above approaches, can be used to reduce or eliminate human immunogenicity of an antibody.

In addition, it is possible to create fully human antibodies in mice. Fully human mAbs lacking any non-human sequences can be prepared from human immunoglobulin transgenic mice by techniques referenced in, e.g., Lonberg et al., NATURE 368:856-859, 1994; Fishwild et al., NATURE BIOTECHNOLOGY 14:845-851, 1996; and Mendez et al., NATURE GENETICS 15:146-156, 1997. Fully human monoclonal antibodies can also be prepared and optimized from phage display libraries by techniques referenced in, e.g., Knappik et al., J. MOL. BIOL. 296:57-86, 2000; and Krebs et al., J. IMMUNOL. METH. 254:67-84 2001).

The present invention encompasses fusion proteins comprising antibody fragments, which may be generated by traditional means, such as enzymatic digestion, or by recombinant techniques. For a review of certain antibody fragments, see Hudson et al. (2003) NAT. MED. 9:129-134.

Various techniques have been developed for the production of antibody fragments. Traditionally, these fragments were derived via proteolytic digestion of intact antibodies (see, e.g., Morimoto et al. (1992) JOURNAL OF BIOCHEMICAL AND BIOPHYSICAL METHODS 24:107-117; and Brennan et al. (1985) SCIENCE 229:81). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries. Alternatively, Fab′-SH fragments can be directly recovered from E. coli and chemically coupled to form F(ab′)₂ fragments (Carter et al. (1992) BIO/TECHNOLOGY 10:163-167). According to another approach, F(ab′)₂ fragments can be isolated directly from recombinant host cell culture. Fab and F(ab′)₂ fragments with increased in vivo half-life comprising salvage receptor binding epitope residues are described in U.S. Pat. No. 5,869,046. Other techniques for the production of antibody fragments will be apparent to the skilled practitioner. In certain embodiments, an antibody is a single chain Fv fragment (scFv). See U.S. Pat. Nos. 5,571,894 and 5,587,458.

Methods for making bispecific antibodies are known in the art. See Milstein and Cuello (1983) NATURE 305:537, International (PCT) Publication No. WO93/08829, and Traunecker et al. (1991) EMBO J., 10:3655. For further details of generating bispecific antibodies see, for example, Suresh et al. (1986) METHODS ENZYMOL. 121:210. Bispecific antibodies include cross-linked or “heteroconjugate” or “heterodimer” antibodies. For example, one of the antibodies in the heterodimer can be coupled to avidin, the other to biotin. Heterodimer antibodies may be made using any convenient cross-linking method. Suitable cross-linking agents are well known in the art, and are disclosed in U.S. Pat. No. 4,676,980, along with a number of cross-linking techniques.

Examples of heterodimeric or asymmetric IgG-like molecules include but are not limited to those obtained with the following technologies or using the following formats: Triomab/Quadroma, Knobs-into-Holes, CrossMabs, electrostatically-matched antibodies, LUZ-Y, Strand Exchange Engineered Domain body, Biclonic and DuoBody.

Advantages of using antibody fragments (e.g., F(ab) and F(ab′)₂ fragments) include the elimination of non-specific binding between Fc portions of antibodies and Fc receptors on cells (such as macrophages, dendritic cells, neutrophils, NK cells and B cells). In addition, they may be able to penetrate tissues more efficiently due to their smaller size.

Heterodimeric antibodies, or asymmetric antibodies, allow for greater flexibility and new formats for attaching a variety of drugs to the antibody arms. One of the general formats for creating a heterodimeric antibody is the “knobs-into-holes” format. This format is specific to the heavy chain part of the constant region in antibodies. The “knobs” part is engineered by replacing a small amino acid with a larger one, which fits into a “hole”, which is engineered by replacing a large amino acid with a smaller one. What connects the “knobs” to the “holes” are the disulfide bonds between each chain. The “knobs-into-holes” shape facilitates antibody dependent cell mediated cytotoxicity. Single chain variable fragments (scFv) are connected to the variable domain of the heavy and light chain via a short linker peptide. The linker is rich in glycine, which gives it more flexibility, and serine/threonine, which gives it specificity. Two different scFv fragments can be connected together, via a hinge region, to the constant domain of the heavy chain or the constant domain of the light chain. This gives the antibody bispecificity, allowing for the binding specificities of two different antigens. The “knobs-into-holes” format enhances heterodimer formation but doesn't suppress homodimer formation.

Several approaches to support heterodimerization have been described, for example in International (PCT) Publication Nos. WO96/27011, WO98/050431, WO2007/110205, WO2007/147901, WO2009/089004, WO2010/129304, WO2011/90754, WO2011/143545, WO2012/058768, WO2013/157954, and WO2013/096291, and European Patent Publication No. EP1870459. Typically, in the approaches known in the art, the CH3 domain of the first heavy chain and the CH3 domain of the second heavy chain are both engineered in a complementary manner so that the heavy chain comprising one engineered CH3 domain can no longer homodimerize with another heavy chain of the same structure (e.g. a CM-engineered first heavy chain can no longer homodimerize with another CM-engineered first heavy chain; and a CH3-engineered second heavy chain can no longer homodimerize with another CM-engineered second heavy chain). Thereby the heavy chain comprising one engineered CH3 domain is forced to heterodimerize with another heavy chain comprising the CH3 domain, which is engineered in a complementary manner. As a result, the CH3 domain of the first heavy chain and the CH3 domain of the second heavy chain are engineered in a complementary manner by amino acid substitutions, such that the first heavy chain and the second heavy chain are forced to heterodimerize, whereas the first heavy chain and the second heavy chain can no longer homodimerize (e.g., for steric reasons).

### III. Pharmaceutical Compositions

For therapeutic use, a fusion protein and/or antibody conjugate preferably is combined with a pharmaceutically acceptable carrier. The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein refers to buffers, carriers, and excipients suitable for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Martin, Remington's Pharmaceutical Sciences, 15th Ed., Mack Publ. Co., Easton, PA [1975]. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

In certain embodiments, a pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants (see, Remington's Pharmaceutical Sciences, 18th ed. (Mack Publishing Company, 1990).

In certain embodiments, a pharmaceutical composition may contain nanoparticles, e.g., polymeric nanoparticles, liposomes, or micelles (See Anselmo et al. (2016) BIOENG. TRANSL. MED. 1: 10-29).

In certain embodiments, a pharmaceutical composition may contain a sustained- or controlled-delivery formulation. Techniques for formulating sustained- or controlled-delivery means, such as liposome carriers, bio-erodible microparticles or porous beads and depot injections, are also known to those skilled in the art. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, poly (2-hydroxyethyl-inethacrylate), ethylene vinyl acetate, or poly-D(—)-3-hydroxybutyric acid. Sustained release compositions may also include liposomes that can be prepared by any of several methods known in the art.

Pharmaceutical compositions containing a sialidase fusion protein or an antibody conjugate disclosed herein can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, intrathecal and rectal administration. In certain embodiments, a sialidase fusion protein or an antibody conjugate disclosed herein is administered by IV infusion. In certain embodiments, a sialidase fusion protein or an antibody conjugate disclosed herein is administered by intratumoral injection. Useful formulations can be prepared by methods known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyethylene glycol), and suitable mixtures thereof.

In certain embodiments, a pharmaceutical composition may contain a stabilizing agent. In certain embodiments, the stabilizing agent is a cation, such as a divalent cation. In certain embodiments, the cation is calcium or magnesium. The cation can be in the form of a salt, such as calcium chloride (CaCl₂)) or magnesium chloride (MgCl₂).

In certain embodiments, the stabilizing agent is present in an amount from about 0.05 mM to about 5 mM. For example, the stabilizing agent may be present in an amount of from about 0.05 mM to about 4 mM, from about 0.05 mM to about 3 mM, from about 0.05 mM to about 2 mM, from about 0.05 mM to about 1 mM, from about 0.05 mM to about 0.5 mM, from about 0.5 mM to about 4 mM, from about 0.5 mM to about 3 mM, from about 0.5 mM to about 2 mM, from about 0.5 mM to about 1 mM, from about 1 mM to about 4 mM, from about 1 mM to about 3 mM, of from about 1 mM to about 2 mM.

Pharmaceutical formulations preferably are sterile. Sterilization can be accomplished by any suitable method, e.g., filtration through sterile filtration membranes. Where the composition is lyophilized, filter sterilization can be conducted prior to or following lyophilization and reconstitution.

The compositions described herein may be administered locally or systemically. Administration will generally be parenteral administration. In a preferred embodiment, the pharmaceutical composition is administered subcutaneously and in an even more preferred embodiment intravenously. Preparations for parenteral administration include sterile aqueous or non-aqueous solutions, suspensions, and emulsions.

Generally, a therapeutically effective amount of active component, for example, a fusion protein and/or antibody conjugate, is in the range of 0.1 mg/kg to 100 mg/kg, e.g., 1 mg/kg to 100 mg/kg, 1 mg/kg to 10 mg/kg. The amount administered will depend on variables such as the type and extent of disease or indication to be treated, the overall health of the patient, the in vivo potency of the antibody, the pharmaceutical formulation, and the route of administration. The initial dosage can be increased beyond the upper level in order to rapidly achieve the desired blood-level or tissue-level. Alternatively, the initial dosage can be smaller than the optimum, and the daily dosage may be progressively increased during the course of treatment. Human dosage can be optimized, e.g., in a conventional Phase I dose escalation study designed to run from 0.5 mg/kg to 20 mg/kg. Dosing frequency can vary, depending on factors such as route of administration, dosage amount, serum half-life of the fusion protein and/or antibody conjugate, and the disease being treated. Exemplary dosing frequencies are once per day, once per week and once every two weeks. A preferred route of administration is parenteral, e.g., intravenous infusion. In certain embodiments, a fusion protein and/or antibody conjugate is lyophilized, and then reconstituted in buffered saline, at the time of administration.

### IV. Therapeutic Uses

The compositions and methods disclosed herein can be used to treat various forms of cancer in a subject or inhibit cancer growth in a subject. The invention provides a method of treating a cancer in a subject. The method comprises administering to the subject an effective amount of a sialidase anti-PD-1 fusion protein and/or antibody conjugate, e.g., a fusion protein or antibody conjugate disclosed herein, either alone or in a combination with another therapeutic agent to treat the cancer in the subject. The term “effective amount” as used herein refers to the amount of an active agent (e.g., fusion protein according to the present invention) sufficient to effect beneficial or desired results. An effective amount can be administered in one or more administrations, applications or dosages and is not intended to be limited to a particular formulation or administration route.

As used herein, “treat”, “treating” and “treatment” mean the treatment of a disease in a subject, e.g., in a human. This includes: (a) inhibiting the disease, i.e., arresting its development; and (b) relieving the disease, i.e., causing regression of the disease state. As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably includes humans.

Examples of cancers include solid tumors, soft tissue tumors, hematopoietic tumors and metastatic lesions. Examples of hematopoietic tumors include, leukemia, acute leukemia, acute lymphoblastic leukemia (ALL), B-cell, T-cell or FAB ALL, acute myeloid leukemia (AML), chronic myelocytic leukemia (CML), chronic lymphocytic leukemia (CLL), e.g., transformed CLL, diffuse large B-cell lymphomas (DLBCL), follicular lymphoma, hairy cell leukemia, myelodyplastic syndrome (MDS), a lymphoma, Hodgkin's disease, a malignant lymphoma, non-Hodgkin's lymphoma, Burkitt's lymphoma, multiple myeloma, or Richter's Syndrome (Richter's Transformation). Examples of solid tumors include malignancies, e.g., sarcomas, adenocarcinomas, and carcinomas, of the various organ systems, such as those affecting head and neck (including pharynx), thyroid, lung (small cell or non-small cell lung carcinoma (NSCLC)), breast, lymphoid, gastrointestinal (e.g., oral, esophageal, stomach, liver, pancreas, small intestine, colon and rectum, anal canal), genitals and genitourinary tract (e.g., renal, urothelial, bladder, ovarian, uterine, cervical, endometrial, prostate, testicular), CNS (e.g., neural or glial cells, e.g., neuroblastoma or glioma), or skin (e.g., melanoma and metastatic Merkel cell carcinoma (MCC)).

In certain embodiments the cancer is an epithelial cancer, e.g., an epithelial cancer that upregulates the expression of sialylated glycans. Exemplary epithelial cancers include, but are not limited to, endometrial cancer, colon cancer, ovarian cancer, cervical cancer, vulvar cancer, uterine cancer or fallopian tube cancer, breast cancer, prostate cancer, lung cancer, pancreatic cancer, urinary cancer, bladder cancer, head and neck cancer, oral cancer and liver cancer. Epithelial cancers also include carcinomas, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, baso squamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

In certain embodiments the cancer is selected from lung bronchioloalveolar carcinoma (BAC), bladder cancer, a female genital tract malignancy (e.g., uterine serous carcinoma, endometrial carcinoma, vulvar squamous cell carcinoma, and uterine sarcoma), an ovarian surface epithelial carcinoma (e.g., clear cell carcinoma of the ovary, epithelial ovarian cancer, fallopian tube cancer, and primary peritoneal cancer), breast carcinoma, non-small cell lung cancer (NSCLC), a male genital tract malignancy (e.g., testicular cancer), retroperitoneal or peritoneal carcinoma, gastroesophageal adenocarcinoma, esophagogastric junction carcinoma, liver hepatocellular carcinoma, esophageal and esophagogastric junction carcinoma, cervical cancer, cholangiocarcinoma, pancreatic adenocarcinoma, extrahepatic bile duct adenocarcinoma, a small intestinal malignancy, gastric adenocarcinoma, cancer of unknown primary (CUP), colorectal adenocarcinoma, esophageal carcinoma, prostatic adenocarcinoma, kidney cancer, head and neck squamous carcinoma, thymic carcinoma, non-melanoma skin cancer, thyroid carcinoma (e.g., papillary carcinoma), a head and neck cancer, anal carcinoma, non-epithelial ovarian cancer (non-EOC), metastatic urothelial carcinoma (UC), uveal melanoma, malignant pleural mesothelioma, small cell lung cancer (SCLC), a central nervous system cancer, a neuroendocrine tumor, and a soft tissue tumor.

In certain embodiments, the cancer is melanoma, non-small cell lung cancer, colon cancer, breast cancer, bladder cancer, or kidney cancer.

In certain embodiments, the cancer is an adenocarcinoma. In certain embodiments, the cancer is a metastatic cancer. In certain embodiments, the cancer is a refractory cancer.

In certain embodiments, the cancer is resistant to or non-responsive to treatment with an antibody, e.g., pembrolizumab.

In certain embodiments, the cancer is associated with or otherwise mediated by PD-1. In certain embodiments, the cancer is a PD-L1-expressing cancer, e.g., the cancer comprises cells that express PD-L1.

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In certain embodiments, a method or composition described herein, is administered in combination with one or more additional therapies, e.g., surgery, radiation therapy, or administration of another therapeutic preparation. In certain embodiments, the additional therapy may include chemotherapy, e.g., a cytotoxic agent. In certain embodiments the additional therapy may include a targeted therapy, e.g. a tyrosine kinase inhibitor, a proteasome inhibitor, or a protease inhibitor. In certain embodiments, the additional therapy may include an anti-inflammatory, anti-angiogenic, anti-fibrotic, or anti-proliferative compound, e.g., a steroid, a biologic immunomodulator, a monoclonal antibody, an antibody fragment, an aptamer, an siRNA, an antisense molecule, a fusion protein, a cytokine, a cytokine receptor, a bronchodilator, a statin, an anti-inflammatory agent (e.g. methotrexate), or an NSAID. In certain embodiments, the additional therapy may include a combination of therapeutics of different classes.

In certain embodiments, a method or composition described herein is administered in combination with a second checkpoint inhibitor. The checkpoint inhibitor may, for example, be selected from a second PD-1 antagonist, a PD-L1 antagonist, CTLA-4 antagonist, adenosine A2A receptor antagonist, B7-H3 antagonist, B7-H4 antagonist, BTLA antagonist, KIR antagonist, LAG3 antagonist, TIM-3 antagonist, VISTA antagonist or TIGIT antagonist.

In certain embodiments, the checkpoint inhibitor is a second PD-1 inhibitor or a PD-L1 inhibitor. PD-1 is a receptor present on the surface of T-cells that serves as an immune system checkpoint that inhibits or otherwise modulates T-cell activity at the appropriate time to prevent an overactive immune response. Cancer cells, however, can take advantage of this checkpoint by expressing ligands, for example, PD-L1, that interact with PD-1 on the surface of T-cells to shut down or modulate T-cell activity. Exemplary PD-1/PD-L1 based immune checkpoint inhibitors include antibody based therapeutics. Exemplary treatment methods that employ PD-1/PD-L1 based immune checkpoint inhibition are described in U.S. Pat. Nos. 8,728,474 and 9,073,994, and EP Patent No. 1537878B1, and, for example, include the use of anti-PD-1 antibodies. Exemplary anti-PD-1 antibodies are described, for example, in U.S. Pat. Nos. 8,952,136, 8,779,105, 8,008,449, 8,741,295, 9,205,148, 9,181,342, 9,102,728, 9,102,727, 8,952,136, 8,927,697, 8,900,587, 8,735,553, and 7,488,802. Exemplary anti-PD-1 antibodies include, for example, nivolumab (Opdivo®, Bristol-Myers Squibb Co.), pembrolizumab (Keytruda®, Merck Sharp & Dohme Corp.), PDR001 (Novartis Pharmaceuticals), and pidilizumab (CT-011, Cure Tech). Exemplary anti-PD-L1 antibodies are described, for example, in U.S. Pat. Nos. 9,273,135, 7,943,743, 9,175,082, 8,741,295, 8,552,154, and 8,217,149. Exemplary anti-PD-L1 antibodies include, atezolizumab (Tecentriq®, Genentech), durvalumab (AstraZeneca), MEDI4736, avelumab, and BMS 936559 (Bristol Myers Squibb Co.).

In certain embodiments, a method or composition described herein is administered in combination with a CTLA-4 inhibitor. In the CTLA-4 pathway, the interaction of CTLA-4 on a T-cell with its ligands (e.g., CD80, also known as B7-1, and CD86) on the surface of an antigen presenting cells (rather than cancer cells) leads to T-cell inhibition. Exemplary CTLA-4 based immune checkpoint inhibition methods are described in U.S. Pat. Nos. 5,811,097, 5,855,887, 6,051,227. Exemplary anti-CTLA-4 antibodies are described in U.S. Pat. Nos. 6,984,720, 6,682,736, 7,311,910; 7,307,064, 7,109,003, 7,132,281, 6,207,156, 7,807,797, 7,824,679, 8,143,379, 8,263,073, 8,318,916, 8,017,114, 8,784,815, and 8,883,984, International (PCT) Publication Nos. WO98/42752, WO00/37504, and WO01/14424, and European Patent No. EP 1212422 B1. Exemplary CTLA-4 antibodies include ipilimumab or tremelimumab.

In certain embodiments, a method or composition described herein is administered in combination with a CTLA-4 inhibitor, e.g., a CTLA-4 inhibitor disclosed herein.

In certain embodiments, a method or composition described herein is administered in combination with an IDO inhibitor. Exemplary IDO inhibitors include 1-methyl-D-tryptophan (known as indoximod), epacadostat (INCB24360), navoximod (GDC-0919), and BMS-986205.

Exemplary cytotoxic agents that can be administered in combination with a method or composition described herein include, for example, antimicrotubule agents, topoisomerase inhibitors, antimetabolites, protein synthesis and degradation inhibitors, mitotic inhibitors, alkylating agents, platinating agents, inhibitors of nucleic acid synthesis, histone deacetylase inhibitors (HDAC inhibitors, e.g., vorinostat (SAHA, MK0683), entinostat (MS-275), panobinostat (LBH589), trichostatin A (TSA), mocetinostat (MGCD0103), belinostat (PXD101), romidepsin (FK228, depsipeptide)), DNA methyltransferase inhibitors, nitrogen mustards, nitrosoureas, ethylenimines, alkyl sulfonates, triazenes, folate analogs, nucleoside analogs, ribonucleotide reductase inhibitors, vinca alkaloids, taxanes, epothilones, intercalating agents, agents capable of interfering with a signal transduction pathway, agents that promote apoptosis and radiation, or antibody molecule conjugates that bind surface proteins to deliver a toxic agent. In one embodiment, the cytotoxic agent that can be administered with a method or composition described herein is a platinum-based agent (such as cisplatin), cyclophosphamide, dacarbazine, methotrexate, fluorouracil, gemcitabine, capecitabine, hydroxyurea, topotecan, irinotecan, azacytidine, vorinostat, ixabepilone, bortezomib, taxanes (e.g., paclitaxel or docetaxel), cytochalasin B, gramicidin D, ethidium bromide, emetine, mitomycin, etoposide, tenoposide, vincristine, vinblastine, vinorelbine, colchicin, anthracyclines (e.g., doxorubicin or epirubicin) daunorubicin, dihydroxy anthracin dione, mitoxantrone, mithramycin, actinomycin D, adriamycin, 1-dehydrotestosterone, glucocorticoids, procaine, tetracaine, lidocaine, propranolol, puromycin, ricin, or maytansinoids.

The invention also provides a method of increasing the expression of HLA-DR, CD86, CD83, IFNγ, IL-1b, IL-6, TNFα, IL-17A, IL-2, or IL-6 in a cell, tissue, or subject. The method comprises contacting the cell, tissue, or subject with an effective amount of a fusion protein and/or antibody conjugate, e.g., a fusion protein or antibody conjugate disclosed herein. In certain embodiments, the cell is selected from a dendritic cell and a peripheral blood mononuclear cell (PBMC).

In certain embodiments, expression of HLA-DR, CD86, CD83, IFNγ, IL-1b, IL-6, TNFα, IL-17A, IL-2, or IL-6 in the cell, tissue, or subject is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical cell or tissue that has not been contacted with the fusion protein or antibody conjugate. Gene expression may be measured by any suitable method known in the art, for example, by ELISA, or by Luminex multiplex assays.

The invention also provides a method of promoting infiltration of immune cells into a tumor in a subject in need thereof. The method comprises administering to the subject an effective amount of a fusion protein and/or antibody conjugate, e.g., a fusion protein or antibody conjugate disclosed herein. In certain embodiments, the immune cells are T-cells, e.g., CD4+ and/or CD8+ T-cells, e.g., CD69+CD8+ and/or GzmB+CD8⁺ T-cells. In certain embodiments, the immune cells are natural killer (NK) cells.

In certain embodiments, the infiltration of immune cells into the tumor in the subject is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical tumor and/or subject that has not been administered the fusion protein or antibody conjugate. Infiltration of immune cells into a tumor may be measured by any suitable method known in the art, for example, antibody staining.

The invention also provides a method of increasing the number of circulating natural killer (NK) cells in a subject in need thereof. The method comprises administering to the subject an effective amount of a fusion protein and/or antibody conjugate, e.g., a fusion protein or antibody conjugate disclosed herein, so as to increase the number of circulating NK cells relative to prior to administration of the fusion protein, antibody conjugate or pharmaceutical composition.

In certain embodiments, the number of circulating NK cells in the subject is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical subject that has not been administered the fusion protein or antibody conjugate. Circulating NK cells in a subject may be measured by any suitable method known in the art, for example, antibody staining.

The invention also provides a method of increasing the number of T-cells in the draining lymph node in a subject in need thereof. The method comprises administering to the subject an effective amount of a fusion protein, antibody conjugate and/or pharmaceutical composition, e.g., a fusion protein, antibody conjugate and/or pharmaceutical composition disclosed herein, so as to increase the number of T-cells in the draining lymph node relative to prior to administration of the fusion protein, antibody conjugate or pharmaceutical composition. In certain embodiments, the immune cells are T-cells, e.g., CD4+ and/or CD8+ T-cells.

In certain embodiments, the number of T-cells in the draining lymph node in the subject is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical subject that has not been administered the fusion protein, antibody conjugate, or pharmaceutical composition. T-cells in the draining lymph node in a subject may be measured by any suitable method known in the art, for example, antibody staining.

The invention also provides a method of increasing expression of Cd3, Cd4, Cd8, Cd274, Ctla4, Icos, Pdcd1, Lag3, Il6, Il1b, Il2, Ifng, Ifna1, Mx1, Gzmb, Cxcl9, Cxcl12, and/or Ccl5 in a cell, tissue, or subject. The method comprises contacting the cell, tissue, or subject with an effective amount of a fusion protein, antibody conjugate, and/or pharmaceutical composition, e.g., a fusion protein, antibody conjugate, and/or pharmaceutical composition disclosed herein, so as to increase the expression of Cd3, Cd4, Cd8, Cd274, Ctla4, Icos, Pdcd1, Lag3, 116, Il1b, Il2, Ifng, Ifna1, Mx1, Gzmb, Cxcl9, Cxcl12, and/or Cc15 relative to the cell, tissue or subject prior to contact with the fusion protein, antibody conjugate or pharmaceutical composition.

In certain embodiments, expression of Cd3, Cd4, Cd8, Cd274, Ctla4, Icos, Pdcd1, Lag3, 116, Il1b, Il2, Ifng, Ifna1, Mx1, Gzmb, Cxcl9, Cxcl12, and/or Cc15 in the cell, tissue, or subject is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical cell, tissue, or subject that has not been contacted with the fusion protein, antibody conjugate, or pharmaceutical composition. Gene expression may be measured by any suitable method known in the art, for example, by ELISA, Luminex multiplex assays, or Nanostring technology.

The invention also provides a method of removing sialic acid from a cell or tissue. The method comprises contacting the cell or tissue with an effective amount of a fusion protein and/or antibody conjugate, e.g., a fusion protein or antibody conjugate disclosed herein. The invention also provides a method of removing sialic acid from a cell in a subject, the method comprising administering to the subject an effective amount of a pharmaceutical composition comprising a fusion protein and/or antibody conjugate, e.g., a fusion protein or antibody conjugate disclosed herein, thereby to remove sialic acid from the cell.

In certain embodiments, the cell is tumor cell, dendritic cell (DC) or monocyte. In certain embodiments, the cell is a monocyte, and the method results in increased expression of an MHC-II molecule (e.g., HLA-DR) on the monocyte. In certain embodiments, expression of an MHC-II molecule in the cell or tissue is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical cell or tissue that has not been contacted with the fusion protein and/or antibody conjugate. Gene expression may be measured by any suitable method known in the art, for example, by ELISA, by Luminex multiplex assays, or by flow cytometry.

The invention also provides a method of enhancing phagocytosis of a tumor cell. The method comprises contacting the tumor cell with a fusion protein and/or antibody conjugate, e.g., a fusion protein or antibody conjugate disclosed herein, in an amount effective to remove sialic acid from the tumor cell, thereby enhancing phagocytosis of the tumor cell. In certain embodiments, the disclosure relates to a method of increasing phagocytosis of a tumor cell in a subject, the method comprising administering to the subject an effective amount of a pharmaceutical composition a fusion protein and/or antibody conjugate, e.g., a fusion protein or antibody conjugate disclosed herein, in an amount effective to remove sialic acid from the tumor cell, thereby to increase phagocytosis of the tumor cell.

In certain embodiments, phagocytosis is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical tumor cell or population of tumor cells that has not or have not been contacted with the fusion protein and/or antibody conjugate. Phagocytosis may be measured by any suitable method known in the art.

The invention also provides a method of activating a dendritic cell (DC). The method comprises contacting the DC with a tumor cell that has been treated with a fusion protein and/or antibody conjugate, e.g., a fusion protein or antibody conjugate disclosed herein. In certain embodiments, the disclosure relates to a method of activating a dendritic cell (DC) or a population of DCs in a subject, the method comprising administering to the subject an amount of a pharmaceutical composition comprising a fusion protein and/or antibody conjugate, e.g., a fusion protein or antibody conjugate disclosed herein, effective to remove sialic acid from a tumor cell in the subject, thereby to activate the DC or the population of DCs in the subject.

In certain embodiments, activation of the DC or a population of DCs is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical DC or population of DCs that has not or have not been contacted with a tumor cell that has been treated with the fusion protein and/or antibody conjugate. Activation may be measured by any suitable method known in the art.

The invention also provides a method of reducing Siglec-15 binding activity, thereby to increase anti-tumor activity in a tumor microenvironment, the method comprising contacting a T cell with a fusion protein and/or antibody conjugate, e.g., a fusion protein or antibody conjugate disclosed herein. In certain embodiments, the disclosure relates to a method of reducing Siglec-15 binding activity, thereby to increase anti-tumor activity in a tumor microenvironment of a patient, the method comprising administering to the subject an effective amount of a pharmaceutical composition comprising a fusion protein and/or antibody conjugate, e.g., a fusion protein or antibody conjugate disclosed herein, thereby to increase anti-tumor activity (e.g., T cell activity) in the subject.

In certain embodiments, Siglec-15 binding activity is reduced by at least about 10%, at least about 20%, at least about 50%, at least about 75%, or about 100%, relative to Siglec-15 that has not or have not been contacted with the fusion protein and/or antibody conjugate. Binding may be measured by any suitable method known in the art.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following Examples are merely illustrative and are not intended to limit the scope or content of the invention in any way.

### Example 1

This example describes the construction of recombinant human sialidases (Neu1, Neu2, and Neu3).

The human sialidases Neu1, Neu2, Neu3 (isoform 1), and Neu4 (isoform 1) were expressed as secreted proteins with a 10×His tag. To express Neu1 as a secreted protein, the native N terminal signal peptide (MTGERPSTALPDRRWGPRILGFWGGCRVWVFAAIFLLLSLAASWSKA; SEQ ID NO: 27) was replaced by MDMRVPAQLLGLLLLWLPGARC (SEQ ID NO: 28), and the C terminal lysosomal signal motif (YGTL; SEQ ID NO: 29) was removed. To express Neu2, Neu3, and Neu4 as secreted proteins, the N terminal signal peptide MDMRVPAQLLGLLLLWLPGARC (SEQ ID NO: 28) was added to each.

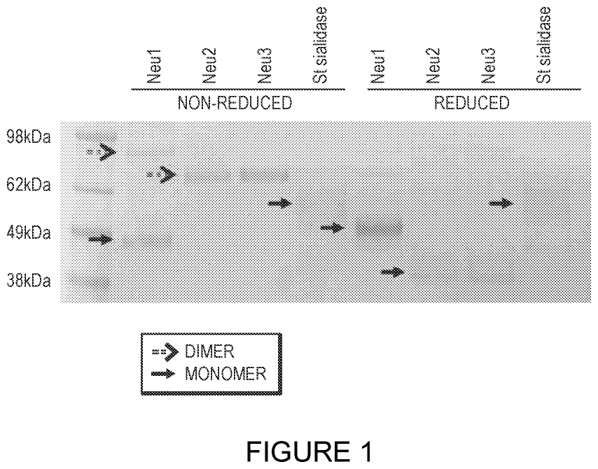

Sialidases were expressed in a 200 mL transfection of HEK293F human cells in 24-well plates using the pCEP4 mammalian expression vector with an N-terminal 6×His tag. Sialidases were purified using Ni-NTA columns, quantified with a UV-Vis spectrophotometer (NanoDrop), and examined by SDS-PAGE as shown in FIG. 1. Neu1 expressed well, with a yield of ˜3 μg/ml, and was present primarily in a monomeric form. Neu2 and Neu3 expression each gave yields of ˜0.15 μg/mL and each were present primarily in a dimeric form. Neu4 had no detectable expression yield as measured by NanoDrop. Bacterial sialidase from Salmonella typhimurium (St-sialidase; SEQ ID NO: 30), which was used as a positive control for expression, gave a comparable yield to Neu1, and was present primarily in a monomeric form.

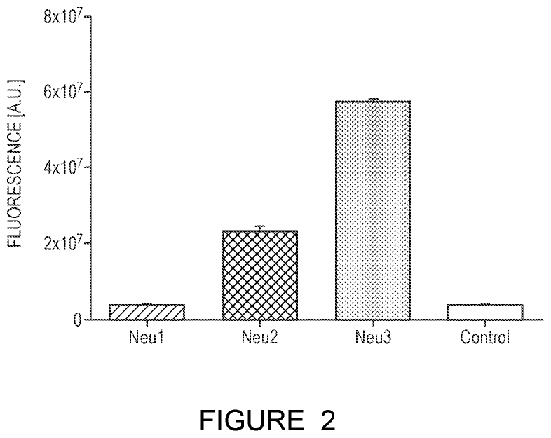

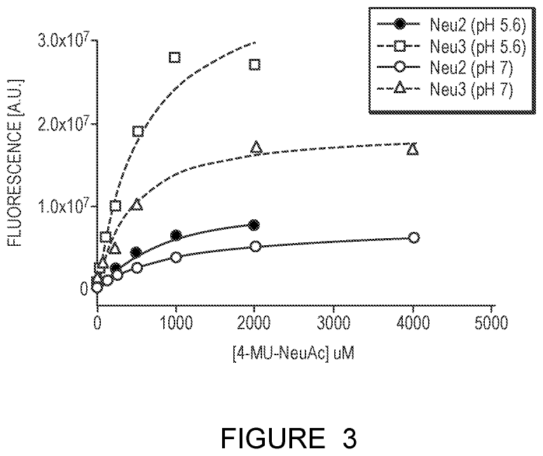

The activity of the recombinantly expressed sialidases was assayed by measuring the release of sialic acid from the fluorogenic substrate 4-methylumbelliferyl-N-acetylneuraminic acid (4MU-NeuAc). As shown in FIG. 2, Neu1 has no detectable activity above a no-enzyme control, which is consistent with previous reports indicating that Neu1 is inactive unless it is in complex with beta-galactosidase and protective protein/cathepsin A (PPCA). Neu2 and Neu3 were active. An enzyme kinetics assay was performed with Neu2 and Neu3. A fixed concentration of enzyme at 1 nM was incubated with fluorogenic substrate 4MU-NeuAc at concentrations ranging from 4000 μM to 7.8 μM. Assays were conducted at both acidic (pH 5.6) and neutral (pH 7) conditions. As shown in FIG. 3, both Neu2 and Neu3 were active at acidic and neutral conditions and showed enzyme kinetics that were comparable to those previously reported.

Most of the recombinantly expressed sialidases ran as aggregates or dimers on a non-reducing SDS-PAGE gel. Subsequent treatment with the reducing agent dithiothreitol (DTT) resulted in a monomeric form of the enzyme that ran at 42 kDa on a reducing SDS-PAGE gel (FIG. 1).

### Example 2

This example describes the construction of anti-PD-1 antibody sialidase conjugates (ASCs).

An exemplary configuration of an anti-PD-1 antibody ASC is referred to as “Janus,” and contains one antibody arm (with one heavy chain and one light chain), and one sialidase-Fc fusion with a sialidase fused at the N-terminus of one arm of the Fc. Each Fc domain polypeptide in the Janus ASC contains either the “knob” (T366Y) or “hole” (Y407T) mutation for heterodimerization (residue numbers according to EU numbering, Kabat, E. A., et al. (1991) supra) (see, e.g., FIG. 6B).

A first Janus ASC is constructed using Neu2 with M1D, V6Y, P62G, A93E, I187K, and C332A mutations and pembrolizumab, an anti-PD-1 antibody. This ASC (referred to as ASC #1, and including a first polypeptide chain with amino acid sequence SEQ ID NO: 77, encoded by nucleotide sequence SEQ ID NO: 104, a second polypeptide chain with amino acid sequence SEQ ID NO: 105, encoded by nucleotide sequence SEQ ID NO: 110, and a third polypeptide chain with amino acid sequence SEQ ID NO: 111, encoded by nucleotide sequence SEQ ID NO: 114) is expressed and characterized for purity using SDS-PAGE and enzymatic activity using 4MU-NeuAc as described below.

ASC #1 is expressed in a 1000 mL transfection of Expi293 human cells using the pCEP4 mammalian expression vector. The ASC is purified using protein A followed by Ceramic Hydroxyapatite chromatography, quantified with a UV-Vis spectrophotometer (NanoDrop), and examined by SDS-PAGE.

The activity of ASC #1 is assayed by measuring the release of sialic acid from the fluorogenic substrate 4-methylumbelliferyl-N-acetylneuraminic acid (4MU-NeuAc). Specifically, an enzyme kinetics assay is performed by incubating a fixed concentration of enzyme at 1 nM with fluorogenic substrate 4MU-NeuAc at concentrations ranging from 4000 μM to 7.8 μM. Active enzyme causes the release of sialic acid which generates fluorescence. Assays are conducted at pH 5.6.

A second Janus ASC is constructed using Neu2 with M1D, V6Y, P62G, A93E, Q126Y, I187K, A242F, Q270T, and C332A mutations and pembrolizumab. This ASC (referred to as ASC #2, and including a first polypeptide chain with amino acid sequence SEQ ID NO: 77, encoded by nucleotide sequence SEQ ID NO: 104, a second polypeptide chain with amino acid sequence SEQ ID NO: 105, encoded by nucleotide sequence SEQ ID NO: 110, and a third polypeptide chain with amino acid sequence SEQ ID NO: 115, encoded by nucleotide sequence SEQ ID NO: 124) is expressed and characterized for purity using SDS-PAGE and enzymatic activity using 4MU-NeuAc as described above.

An additional exemplary configuration of an ASC, referred to as “Lobster-Fab,” contains two Fc domain polypeptides each with a sialidase fused at the N-terminus of the Fc and a Fab fused at the C-terminus of the Fc (FIG. 6E).

A first Lobster-Fab ASC is constructed using Neu2 with M1D, V6Y, A42R, P62G, A93E, Q126Y, I187K, A242F, Q270T, and C332A mutations and pembrolizumab. This ASC is referred to as ASC #4, and includes first and fourth polypeptide chains with an amino acid sequence of SEQ ID NO: 77, encoded by nucleotide sequence SEQ ID NO: 104, and second and third polypeptide chains with an amino acid sequence of SEQ ID NO: 130, encoded by nucleotide sequence SEQ ID NO: 131. A second Lobster-Fab ASC is constructed using Neu2 with M1D, V6Y, A42R, P62G, A93E, Q126Y, I187K, A242F, Q270T, and C332A mutations and pembrolizumab. This ASC is referred to as ASC #5, and includes first and fourth polypeptide chains with an amino acid sequence of SEQ ID NO: 132, encoded by nucleotide sequence SEQ ID NO: 133, and second and third polypeptide chains with an amino acid sequence of SEQ ID NO: 134, encoded by nucleotide sequence SEQ ID NO: 135. ASC Nos. 4 and 5 are expressed and characterized for purity using SDS-PAGE and enzymatic activity using 4MU-NeuAc as described above.

An additional exemplary configuration of an ASC, referred to as “Raptor,” includes an antibody (with two heavy chains and two light chains) with a sialidase fused at the C-terminus of each heavy chain of the antibody (see, e.g., FIG. 6A).

### Example 3

This example describes the construction and characterization of anti-PD-1 antibody sialidase conjugates (ASCs).

A Janus ASC was constructed using Neu2 with M1D, V6Y, A42R, P62G, A93E, Q126Y, I187K, A242F, Q270T, and C332A mutations, the variable region of pembrolizumab, and a human IgG1 Fc domain including an N297A mutation. This ASC, referred to as ASC #3, includes a first polypeptide chain with amino acid sequence SEQ ID NO: 77, encoded by nucleotide sequence SEQ ID NO: 104, a second polypeptide chain with amino acid sequence SEQ ID NO: 105, encoded by nucleotide sequence SEQ ID NO: 110, and a third polypeptide chain with amino acid sequence SEQ ID NO: 125, encoded by nucleotide sequence SEQ ID NO: 129. A Raptor ASC was constructed using Neu2 with M1D, V6Y, P62G, A93E, Q126Y, I187K, A242F, Q270T, and C332A mutations, the variable region of pembrolizumab, and a human IgG1 Fc domain including an N297A mutation. This ASC, referred to as ASC #6, includes a first polypeptide chain with amino acid sequence SEQ ID NO: 77, encoded by nucleotide sequence SEQ ID NO: 104, and a second polypeptide chain with amino acid sequence SEQ ID NO: 145, encoded by nucleotide sequence SEQ ID NO: 146.

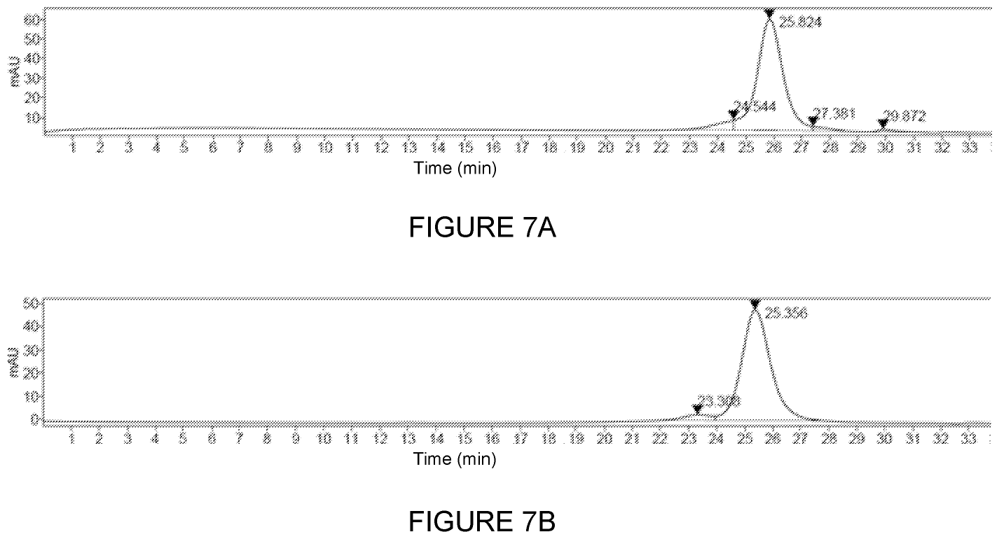

ASC #3 and ASC #6 were expressed in a 1,000 mL transfection of Expi293 human cells using the pCEP4 mammalian expression vector. The ASCs were purified using protein A followed by Ceramic Hydroxyapatite chromatography, quantified with a UV-Vis spectrophotometer (NanoDrop), and examined by SDS-PAGE and size exclusion chromatography (SEC). FIG. 7 depicts SEC traces for ASC #3 and ASC #6, demonstrating 91.6 and 95.6% monomeric form, respectively, and no detectable endotoxin.

The activity of ASC #3 and ASC #6 was assayed by measuring the release of sialic acid from the fluorogenic substrate 4-methylumbelliferyl-N-acetylneuraminic acid (4MU-NeuAc). An enzyme kinetic assay was performed by incubating a fixed concentration of enzyme at 1 nM with fluorogenic substrate 4MU-NeuAc at concentrations ranging from 4,000 μM to 7.8 μM. Active enzyme causes the release of sialic acid which generates fluorescence. Assays were conducted at pH 5.6. ASC #3 had an A_{1/2} (the concentration of ASC needed to convert half of the substrate) of 2.54 μg/mL and ASC #6 had an A_{1/2} of 86.84 μg/mL.

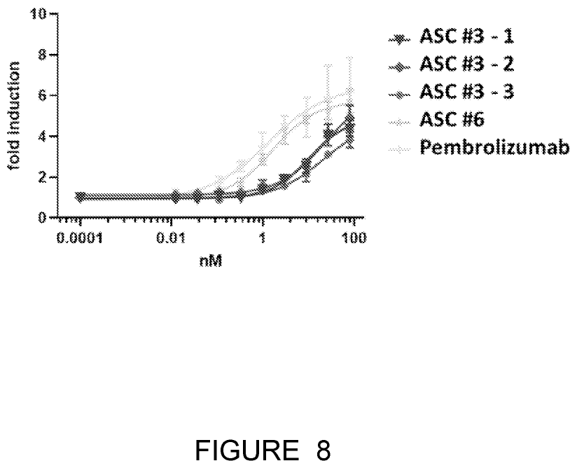

ASC #3 and #6 were tested for their ability to block the interaction between PD-1 and PD-L1. ASC #3 and #6 were incubated with (i) engineered CHO-K1 cells expressing human PD-L1 and TCR activating protein and (ii) Jurkat T cells expressing human PD-1, TCR and a luciferase reporter driven by an NFAT response element. Absent intervention, PD-L1 interacting with PD-1 inhibits TCR-mediated luminescence, while blockade of the PD-L1/PD-1 interaction results in a luminescent signal. A luciferase substrate was added after 6 hours of incubation and luminescence was measured. Relative light units (RLU) were calculated by subtracting background (substrate and media only) from assay wells. Fold induction was calculated by dividing the RLU of induced cells minus background by the RLU of a no antibody control minus background (Fold induction=RLU (induced−background)/RLU (no antibody control−background)). Pembrolizumab was used as a positive control, and produced a fold induction of 4 to 5 over a range of 0.1 to 10 μg/mL in the assay, with an EC₅₀ of 0.92 nM. As shown in FIG. 8, ASC #3 and ASC #6 caused a dose dependent increase in luminescence, indicating that the ASCs are capable of disrupting the PD-L1/PD-1 interaction. EC₅₀ for ASC #3 and ASC #6 were 7.23 nM and 0.53 nM, respectively, comparable to the EC₅₀ of 0.92 nM for pembrolizumab. Two other preparations of ASC #3 displayed similar EC₅₀ values. It is contemplated that the difference in EC₅₀ values resulted from the different ASC formats (e.g., ASC #3 has only a single PD-1 binding antibody arm whereas ASC #6 has two PD-1 binding antibody arms).

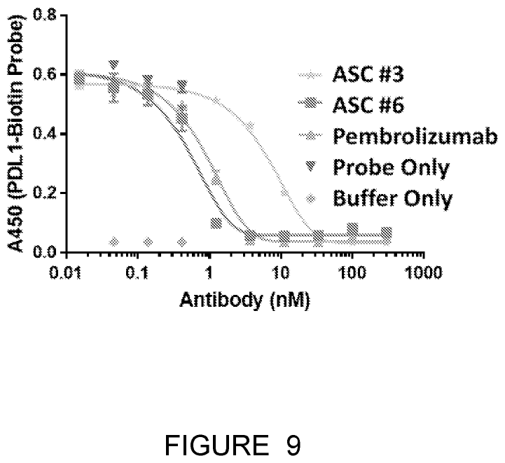

ASC #3 and ASC #6 were also tested for their ability to block a biotinylated human PD-L1 Fc fusion (hPD-L1-Fc) from binding to human PD-1 (hPD-1). ASCs as well as pembrolizumab were 3× titrated and mixed with hPD-L1-Fc at a final concentration of 1 μg/mL. The mixture of antibody and hPD-L1-Fc was loaded on to hPD-1 coated ELISA wells for binding. ASCs or antibodies that bind to the hPD-L1 binding epitope on hPD-1 compete for binding and result in a reduction of hPD-L1-Fc binding signal. The residual binding of hPD-L1-Fc to hPD-1 was detected with HRP conjugated streptavidin. The plate was developed with TMB and Stop buffer and the absorbance at 450 nm was read using a SpectraMax plate reader. A450 absorbance curves and IC_{50S} were generated using GraphPad Prism software. As depicted in FIG. 9, ASC #3 demonstrated single digit nanomolar blocking of hPD-L1 binding to hPD-1 and ASC #6 demonstrated sub-nanomolar blocking. These results are consistent with those from the cell-based assay described above.

Binding of purified ASC #3 and ASC #6 to recombinant human (hPD-1) was measured by ForteBio octet. ASCs were captured on an AHC (Anti-human IgG-Capture) biosensor. hPD-1 analytes were titrated from 100 nM in a 2× series dilution. The signal was subtracted with buffer reference and aligned to baseline. KD, Kon and Koff values were generated using a 1:1 fitting model. The KD for ASC #3 and ASC #6 was determined to be 10.3 nM and 10.6 nM, respectively.

A summary of some of the properties of ASC #3 and ASC #6 is set forth in TABLE 10.

TABLE 10

| | Yield | A_{1/2} | Kd | EC₅₀ | Monomer | |
|---|---|---|---|---|---|---|
| | (mg/L) | (μg/mL) | (nM) | (nM) | % | EU/μg |
| ASC # 3 | 10.2 | 2.54 | 10.3 | 7.23 | 91.6 | <0.0002 |
| ASC # 6 | 16.5 | 86.84 | 10.6 | 0.53 | 95.6 | <0.0002 |

### Example 4

This Example describes the in vivo administration of anti-PD-1 antibody sialidase conjugates (ASCs) containing human sialidases.

A MC38 mouse tumor line engineered to express human PD-L1 was grown as a syngeneic subcutaneous tumor in a C57BL6 transgenic mouse engineered to express human PD-L1 and human PD-1 and in which mouse PD-L1 and mouse PD-1 have been disrupted (Biocytogen Inc.). Mice, 6-8 weeks of age, were inoculated subcutaneously in the right lower flank region with tumor cells for tumor development. Mice were randomly allocated to three groups of eight animals each when tumors reached 90-136 mm³, with a group mean of 109 mm³.

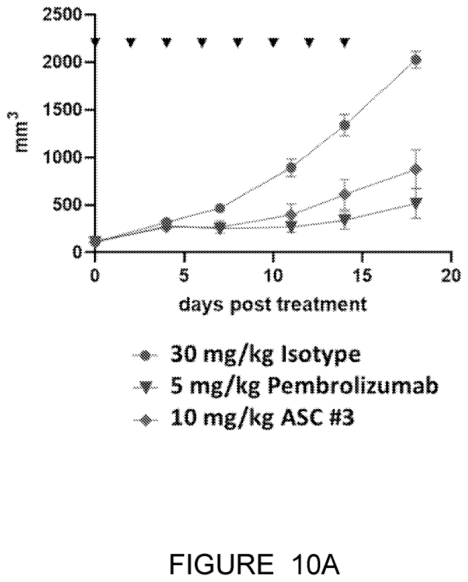

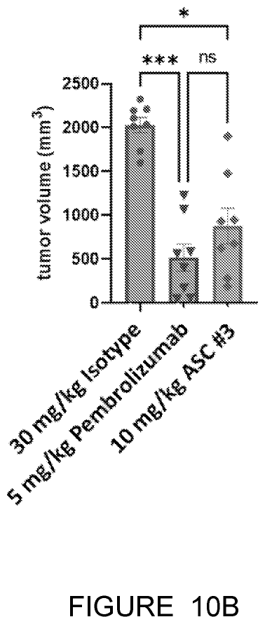

Mice were treated via intraperitoneal injection of ASC #3 (as described in Example 3 above; 10 mg/kg), pembrolizumab (5 mg/kg), or isotype control (30 mg/kg), and tumor volume (mm³) was recorded. Individual tumor volumes are depicted in FIG. 10A and mean tumor volumes on day 18 are depicted in FIG. 10B. As shown in FIG. 10B, there was a significant reduction in tumor growth upon administration of ASC #3, comparable to the response of the positive control pembrolizumab.

A CT26 mouse tumor line engineered to express human PD-L1 was grown as a syngeneic subcutaneous tumor in a transgenic BALB/c mouse engineered to express human PD-L1 and human PD-1 and in which mouse PD-L1 and mouse PD-1 have been disrupted (Gempharmatech Inc.). Mice, 6-8 weeks of age, were inoculated subcutaneously in the right lower flank region with tumor cells for tumor development. Mice were randomly allocated to three groups of six animals each when tumors reached 90-120 mm³, with a group mean of 104.06-104.36 mm³.

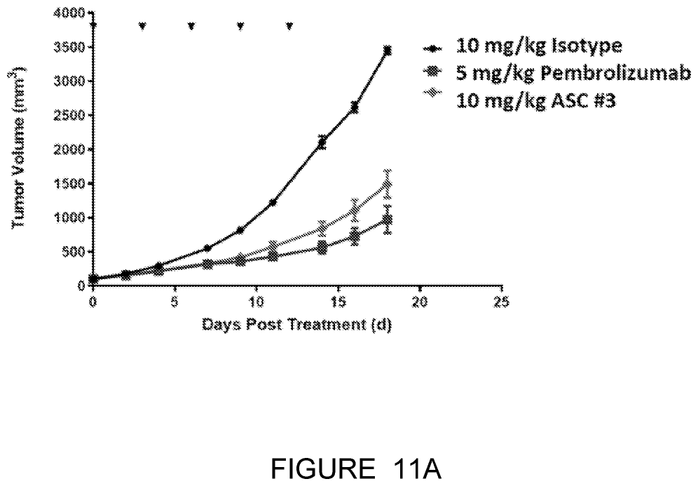

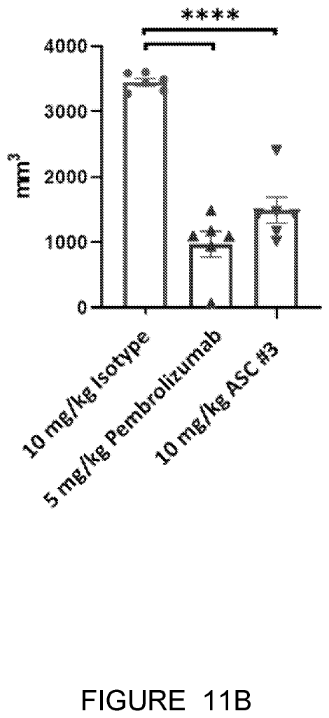

Mice were treated via intraperitoneal injection of ASC #3 (as described in Example 3 above; 10 mg/kg), pembrolizumab (5 mg/kg), or isotype control (10 mg/kg), and tumor volume (mm³) was recorded. Individual tumor volumes are depicted in FIG. 11A and mean tumor volumes on day 18 are depicted in FIG. 11B. As shown in FIG. 11B, there was a significant reduction in tumor growth upon administration of ASC #3, comparable to the response of the positive control pembrolizumab.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

SEQUENCE LISTING
SEQ ID NO: 1:
MASLPVLQKESVFQSGAHAYRIPALLYLPGQQSLLAFAEQRASKKDEHAELIVLRRGDYDAPTH
QVQWQAQEVVAQARLDGHRSMNPCPLYDAQTGTLFLFFIAIPGQVTEQQQLQTRANVTRLCQVT
STDHGRTWSSPRDLTDAAIGPAYREWSTFAVGPGHCLQLHDRARSLVVPAYAYRKLHPIQRPIP
SAFCFLSHDHGRTWARGHFVAQDTLECQVAEVETGEQRVVTLNARSHLRARVQAQSINDGLDFQ
ESQLVKKLVEPPPQGCQGSVISFPSPRSGPGSPAQWLLYTHPTHSWQRADLGAYLNPRPPAPEA
WSEPVLLAKGSCAYSDLQSMGTGPDGSPLFGCLYEANDYEEIVELMFTLKQAFPAEYLPQ
SEQ ID NO: 2:
MEDLRPMATCPVLQKETLERTGVHAYRIPALLYLKKQKTLLAFAEKRASKTDEHAELIVLRRGS
YNEATNRVKWQPEEVVTQAQLEGHRSMNPCPLYDKQTKTLFLFFIAVPGRVSEHHQLHTKVNVT
RLCCVSSTDHGRTWSPIQDLTETTIGSTHQEWATFAVGPGHCLQLRNPAGSLLVPAYAYRKLHP
AQKPTPFAFCFISLDHGHTWKLGNFVAENSLECQVAEVGTGAQRMVYLNARSELGARVQAQSPN
DGLDFQDNRVVSKLVEPPHGCHGSVVAFHNPISKPHALDTWLLYTHPTDSRNRTNLGVYLNQMP
LDPTAWSEPTLLAMGICAYSDLQNMGQGPDGSPQFGCLYESGNYEEIIFLIFTLKQAFPTVEDA
Q
SEQ ID NO: 3:
EDLRP
SEQ ID NO: 4:
MEDLRP
SEQ ID NO: 5:
DKTHTCPPCPAPELLGGPSVELFPPKPKDTLMISRTPEVTCVVVDVSHEDPEVKENWYVDGVEV
HNAKTKPREEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYSKLTV
DKSRWQQGNVFSCSVMHEALHNHYTQKSLSLSPGK
SEQ ID NO: 6:
ACAGTGGAAAAGTCCGTGGTGTTCAAGGCCGAGGGCGAGCACTTCACCGACCAGAAAGGCAATA
CCATCGTCGGCTCTGGCAGCGGCGGCACCACCAAGTACTTTAGAATCCCCGCCATGTGCACCAC
CAGCAAGGGCACCATTGTGGTGTTCGCCGACGCCAGACACAACACCGCCAGCGATCAGAGCTTC
ATCGATACCGCTGCCGCCAGATCTACCGATGGCGGCAAGACCTGGAACAAGAAGATCGCCATCT
ACAACGACCGCGTGAACAGCAAGCTGAGCAGAGTGATGGACCCTACCTGCATCGTGGCCAACAT
CCAGGGCAGAGAAACCATCCTGGTCATGGTCGGAAAGTGGAACAACAACGATAAGACCTGGGGC
GCCTACAGAGACAAGGCCCCTGATACCGATTGGGACCTCGTGCTGTACAAGAGCACCGATGACG
GCGTGACCTTCAGCAAGGTGGAAACAAACATCCACGACATCGTGACCAAGAACGGCACCATCTC
TGCCATGCTCGGCGGCGTTGGATCTGGCCTGCAACTGAATGATGGCAAGCTGGTGTTCCCCGTG
CAGATGGTCCGAACAAAGAATATCACCACCGTGCTGAATACCAGCTTCATCTACAGCACCGACG
GCATCACATGGTCCCTGCCTAGCGGCTACTGTGAAGGCTTTGGCAGCGAGAACAACATCATCGA
GTTCAACGCCAGCCTGGTCAACAACATCCGGAACAGCGGCCTGCGGAGAAGCTTCGAGACAAAG
GACTTCGGAAAGACGTGGACCGAGTTTCCTCCAATGGACAAGAAGGTGGACAACCGGAACCACG
GCGTGCAGGGCAGCACAATCACAATCCCTAGCGGCAACAAACTGGTGGCCGCTCACTCTAGCGC
CCAGAACAAGAACAACGACTACACCAGAAGCGACATCAGCCTGTACGCCCACAACCTGTACAGC
GGCGAAGTGAAGCTGATCGACGACTTCTACCCCAAAGTGGGCAATGCCAGCGGAGCCGGCTACA
GCTGTCTGAGCTACCGGAAAAATGTGGACAAAGAAACCCTGTACGTGGTGTACGAGGCCAACGG
CAGCATCGAGTTTCAGGACCTGAGCAGACATCTGCCCGTGATCAAGAGCTACAAC
SEQ ID NO: 7:
ENDFGLVQPLVTMEQLLWVSGRQIGSVDTFRIPLITATPRGTLLAFAEARKMSSSDEGAKFIAL
RRSMDQGSTWSPTAFIVNDGDVPDGLNLGAVVSDVETGVVELFYSLCAHKAGCQVASTMLVWSK
DDGVSWSTPRNLSLDIGTEVFAPGPGSGIQKQREPRKGRLIVCGHGTLERDGVFCLLSDDHGAS
WRYGSGVSGIPYGQPKQENDENPDECQPYELPDGSVVINARNQNNYHCHCRIVLRSYDACDTLR
PRDVTFDPELVDPVVAAGAVVTSSGIVEFSNPAHPEFRVNLTLRWSFSNGTSWRKETVQLWPGP
SGYSSLATLEGSMDGEEQAPQLYVLYEKGRNHYTESISVAKISV
SEQ ID NO: 8:
MEEVTTCSENSPLFRQEDDRGITYRIPALLYIPPTHTFLAFAEKRSTRRDEDALHLVLRRGLRI
GQLVQWGPLKPLMEATLPGHRTMNPCPVWEQKSGCVFLFFICVRGHVTERQQIVSGRNAARLCF
IYSQDAGCSWSEVRDLTEEVIGSELKHWATFAVGPGHGIQLQSGRLVIPAYTYYIPSWFFCFQL
PCKTRPHSLMIYSDDLGVTWHHGRLIRPMVTVECEVAEVTGRAGHPVLYCSARTPNRCRAEALS
TDHGEGFQRLALSRQLCEPPHGCQGSVVSFRPLEIPHRCQDSSSKDAPTIQQSSPGSSLRLEEE
AGTPSESWLLYSHPTSRKQRVDLGIYLNQTPLEAACWSRPWILHCGPCGYSDLAALEEEGLFGC
LFECGTKQECEQIAFRLFTHREILSHLQGDCTSPGRNPSQFKSN
SEQ ID NO: 9:
MRPADLPPRPMEESPASSSAPTETEEPGSSAEVMEEVTTCSENSPLERQEDDRGITYRIPALLY
IPPTHTFLAFAEKRSTRRDEDALHLVLRRGLRIGQLVQWGPLKPLMEATLPGHRTMNPCPVWEQ
KSGCVFLFFICVRGHVTERQQIVSGRNAARLCFIYSQDAGCSWSEVRDLTEEVIGSELKHWATF
AVGPGHGIQLQSGRLVIPAYTYYIPSWFFCFQLPCKTRPHSLMIYSDDLGVTWHHGRLIRPMVT
VECEVAEVTGRAGHPVLYCSARTPNRCRAEALSTDHGEGFQRLALSRQLCEPPHGCQGSVVSER
PLEIPHRCQDSSSKDAPTIQQSSPGSSLRLEEEAGTPSESWLLYSHPTSRKQRVDLGIYLNQTP
LEAACWSRPWILHCGPCGYSDLAALEEEGLFGCLFECGTKQECEQIAFRLFTHREILSHLQGDC
TSPGRNPSQFKSN
SEQ ID NO: 10:
MGVPRTPSRTVLFERERTGLTYRVPSLLPVPPGPTLLAFVEQRLSPDDSHAHRLVLRRGTLAGG
SVRWGALHVLGTAALAEHRSMNPCPVHDAGTGTVELFFIAVLGHTPEAVQIATGRNAARLCCVA
SRDAGLSWGSARDLTEEAIGGAVQDWATFAVGPGHGVQLPSGRLLVPAYTYRVDRRECFGKICR
TSPHSFAFYSDDHGRTWRCGGLVPNLRSGECQLAAVDGGQAGSFLYCNARSPLGSRVQALSTDE
GTSFLPAERVASLPETAWGCQGSIVGFPAPAPNRPRDDSWSVGPGSPLQPPLLGPGVHEPPEEA
AVDPRGGQVPGGPFSRLQPRGDGPRQPGPRPGVSGDVGSWTLALPMPFAAPPQSPTWLLYSHPV
GRRARLHMGIRLSQSPLDPRSWTEPWVIYEGPSGYSDLASIGPAPEGGLVFACLYESGARTSYD
EISFCTESLREVLENVPASPKPPNLGDKPRGCCWPS
SEQ ID NO: 11:
MMSSAAFPRWLSMGVPRTPSRTVLFERERTGLTYRVPSLLPVPPGPTLLAFVEQRLSPDDSHAH
RLVLRRGTLAGGSVRWGALHVLGTAALAEHRSMNPCPVHDAGTGTVFLFFIAVLGHTPEAVQIA
TGRNAARLCCVASRDAGLSWGSARDLTEEAIGGAVQDWATFAVGPGHGVQLPSGRLLVPAYTYR
VDRRECFGKICRTSPHSFAFYSDDHGRTWRCGGLVPNLRSGECQLAAVDGGQAGSFLYCNARSP
LGSRVQALSTDEGTSFLPAERVASLPETAWGCQGSIVGFPAPAPNRPRDDSWSVGPGSPLQPPL
LGPGVHEPPEEAAVDPRGGQVPGGPFSRLQPRGDGPRQPGPRPGVSGDVGSWTLALPMPFAAPP
QSPTWLLYSHPVGRRARLHMGIRLSQSPLDPRSWTEPWVIYEGPSGYSDLASIGPAPEGGLVFA
CLYESGARTSYDEISFCTFSLREVLENVPASPKPPNLGDKPRGCCWPS
SEQ ID NO: 12:
MASLP
SEQ ID NO: 13:
ASLP
SEQ ID NO: 14:
TVEKSVVE
SEQ ID NO: 15:
GDYDAPTHQVQW
SEQ ID NO: 16:
SMDQGSTW
SEQ ID NO: 17:
STDGGKTW
SEQ ID NO: 18:
PRPPAPEA
SEQ ID NO: 19:
QTPLEAAC
SEQ ID NO: 20:
NPRPPAPEA
SEQ ID NO: 21:
SQNDGES
SEQ ID NO: 22:
LSHSLST
SEQ ID NO: 23:
GAGAACGACTTTGGACTGGTGCAGCCTCTGGTCACCATGGAACAGCTGCTGTGGGTTTCCGGCA
GACAGATCGGCAGCGTGGACACCTTCAGAATCCCTCTGATCACCGCCACACCTAGAGGCACCCT
GCTGGCCTTTGCCGAGGCCAGAAAGATGAGCAGCTCTGACGAGGGCGCCAAGTTTATTGCCCTG
AGGCGGTCTATGGACCAGGGCTCTACATGGTCCCCTACCGCCTTCATCGTGAACGATGGCGACG
TGCCCGATGGCCTGAATCTGGGAGCTGTGGTGTCCGATGTGGAAACCGGCGTGGTGTTCCTGTT
CTACAGCCTGTGTGCCCACAAGGCCGGTTGTCAGGTGGCCAGCACAATGCTCGTGTGGTCCAAG
GACGACGGCGTGTCCTGGTCTACCCCTAGAAACCTGAGCCTGGACATCGGCACCGAAGTGTTTG
CTCCAGGACCTGGCTCTGGCATCCAGAAGCAGAGAGAGCCCAGAAAGGGCAGACTGATCGTGTG
TGGCCACGGCACCCTTGAGAGAGATGGCGTTTTCTGCCTGCTGAGCGACGATCATGGCGCCTCT
TGGAGATACGGCAGCGGAGTGTCTGGAATCCCTTACGGCCAGCCTAAGCAAGAGAACGATTTCA
ACCCCGACGAGTGCCAGCCTTACGAGCTGCCTGATGGCAGCGTCGTGATCAACGCCCGGAACCA
GAACAACTACCACTGCCACTGCCGGATCGTGCTGAGAAGCTACGACGCCTGCGATACCCTGCGG
CCTAGAGATGTGACCTTCGATCCTGAGCTGGTGGACCCTGTTGTTGCCGCTGGTGCCGTCGTGA
CATCTAGCGGCATCGTGTTCTTCAGCAACCCTGCTCACCCCGAGTTCAGAGTGAATCTGACCCT
GCGGTGGTCCTTCAGCAATGGCACAAGCTGGCGGAAAGAAACCGTGCAGCTTTGGCCTGGACCT
AGCGGCTACTCTTCTCTGGCTACACTGGAAGGCAGCATGGACGGCGAAGAACAGGCCCCTCAGC
TGTACGTGCTGTACGAGAAGGGCAGAAACCACTACACCGAGAGCATCAGCGTGGCCAAGATCAG
CGTT
SEQ ID NO: 24:
ATGGCCAGCCTGCCTGTGCTGCAGAAAGAAAGCGTGTTCCAGTCTGGCGCCCACGCCTACAGAA
TTCCCGCTCTGCTGTATCTGCCAGGCCAGCAGTCTCTGCTGGCTTTCGCTGAACAGCGGGCCAG
CAAGAAGGATGAGCACGCCGAACTGATCGTGCTGCGGAGAGGCGATTACGACGCCCCTACACAT
CAGGTGCAGTGGCAGGCTCAAGAGGTGGTGGCTCAGGCTAGACTGGACGGCCACAGATCTATGA
ACCCCTGTCCTCTGTACGATGCCCAGACCGGCACACTGTTTCTGTTCTTTATCGCTATCCCCGG
CCAAGTGACCGAGCAGCAGCAGCTGCAGACAAGAGCCAACGTGACCAGACTGTGTCAAGTGACC
TCCACCGACCACGGCAGAACCTGGTCTAGCCCTAGAGATCTGACCGACGCCGCCATCGGACCTG
CCTATAGAGAGTGGTCCACCTTCGCCGTTGGACCTGGACACTGTCTCCAGCTGCACGACAGGGC
TAGATCTCTGGTGGTGCCTGCCTACGCCTATAGAAAGCTGCACCCCATCCAGCGGCCTATTCCT
AGCGCCTTCTGCTTTCTGAGCCACGATCACGGCAGGACATGGGCCAGAGGACATTTCGTGGCCC
AGGACACACTGGAATGCCAGGTGGCCGAAGTGGAAACCGGCGAGCAGAGAGTCGTGACCCTGAA
CGCCAGATCTCACCTGAGAGCCAGAGTGCAGGCCCAGAGCACAAACGACGGCCTGGATTTCCAA
GAGAGCCAGCTGGTCAAGAAACTGGTGGAACCTCCTCCACAGGGCTGTCAGGGAAGCGTGATCA
GCTTTCCATCTCCTAGAAGCGGCCCTGGCTCTCCTGCTCAGTGGCTGCTGTATACACACCCCAC
ACACAGCTGGCAGAGAGCCGATCTGGGCGCCTACCTGAATCCTAGACCTCCTGCTCCTGAGGCT
TGGAGCGAACCTGTTCTGCTGGCCAAGGGCAGCTGTGCCTACAGCGATCTGCAGTCTATGGGCA
CAGGCCCTGATGGCAGCCCTCTGTTTGGCTGTCTGTACGAGGCCAACGACTACGAAGAGATCGT
GTTCCTGATGTTCACCCTGAAGCAGGCCTTTCCAGCCGAGTACCTGCCTCAA
SEQ ID NO: 25:
ATGGAGGAAGTGACCACCTGTAGCTTCAACAGCCCTCTGTTCCGGCAAGAGGACGACCGGGGCA
TCACCTACAGAATCCCTGCTCTGCTGTACATCCCTCCTACACACACCTTTCTGGCCTTCGCCGA
GAAGCGGAGCACCAGACGAGATGAAGATGCCCTGCACCTGGTGCTGAGAAGAGGCCTGAGAATC
GGACAGCTGGTGCAGTGGGGACCTCTGAAGCCTCTGATGGAAGCCACACTGCCCGGCCACAGAA
CCATGAATCCTTGTCCTGTGTGGGAGCAGAAAAGCGGCTGCGTGTTCCTGTTCTTCATCTGCGT
GCGGGGCCACGTGACCGAGAGACAGCAAATCGTGTCCGGCAGAAACGCCGCCAGACTGTGCTTC
ATCTACAGCCAGGATGCCGGCTGCTCTTGGAGCGAAGTTCGGGATCTGACCGAAGAAGTGATCG
GCAGCGAGCTGAAGCACTGGGCCACATTTGCTGTTGGCCCTGGCCACGGAATCCAGCTGCAATC
TGGCAGACTGGTCATCCCCGCCTACACCTACTATATCCCCAGCTGGTTCTTCTGCTTCCAACTG
CCTTGCAAGACCCGGCCTCACAGCCTGATGATCTACAGCGACGATCTGGGCGTGACATGGCACC
ACGGCAGACTGATCAGACCCATGGTCACCGTGGAATGCGAGGTGGCCGAAGTGACAGGCAGAGC
TGGACACCCTGTGCTGTACTGCTCTGCCAGAACACCCAACCGGTGTAGAGCCGAGGCTCTGTCT
ACAGATCACGGCGAGGGCTTTCAGAGACTGGCCCTCTCTAGACAGCTGTGCGAACCTCCTCATG
GCTGTCAGGGCAGCGTGGTGTCCTTCAGACCTCTGGAAATCCCTCACCGGTGCCAGGACAGCAG
CTCTAAGGATGCCCCTACCATCCAGCAGTCTAGCCCTGGCAGCAGCCTGAGACTGGAAGAGGAA
GCCGGAACACCTAGCGAGAGCTGGCTGCTGTACTCTCACCCCACCAGCAGAAAGCAGAGAGTGG
ACCTGGGCATCTACCTGAATCAGACCCCTCTGGAAGCCGCCTGTTGGAGCAGACCTTGGATTCT
GCACTGTGGCCCTTGCGGCTACTCTGATCTGGCCGCTCTGGAAGAAGAGGGCCTGTTCGGCTGC
CTGTTTGAGTGCGGCACAAAGCAAGAGTGCGAGCAGATCGCCTTCCGGCTGTTCACCCACAGAG
AGATCCTGAGCCATCTGCAGGGCGACTGCACAAGCCCAGGCAGAAATCCCAGCCAGTTCAAGAG
CAAC
SEQ ID NO: 26:
ATGGGCGTGCCCAGAACACCCAGCAGAACCGTGCTGTTCGAGAGAGAGAGGACCGGCCTGACCT
ACAGAGTGCCTTCTCTGCTGCCTGTGCCTCCTGGACCTACACTGCTGGCCTTCGTGGAACAGAG
ACTGAGCCCCGATGATTCTCACGCCCACAGACTGGTGCTGAGAAGAGGAACACTGGCTGGCGGC
TCTGTTAGATGGGGAGCACTGCATGTGCTGGGCACAGCTGCTCTTGCCGAGCACAGATCCATGA
ATCCCTGTCCTGTGCACGACGCCGGAACCGGCACAGTGTTTCTGTTCTTTATCGCCGTGCTGGG
CCACACACCTGAGGCCGTTCAAATTGCCACCGGCAGAAATGCCGCCAGACTGTGTTGTGTGGCC
TCCAGAGATGCCGGCCTGTCTTGGGGATCTGCCAGAGATCTGACCGAGGAAGCCATTGGCGGAG
CCGTTCAGGATTGGGCCACATTTGCTGTTGGACCTGGACACGGCGTGCAGCTGCCAAGTGGTAG
ACTGCTGGTGCCTGCCTACACATACAGAGTGGATCGGAGAGAGTGCTTCGGAAAGATCTGCCGG
ACAAGCCCTCACAGCTTCGCCTTCTACTCCGACGATCACGGCCGGACTTGGAGATGTGGTGGCC
TGGTGCCTAATCTGAGAAGCGGCGAATGTCAACTGGCCGCCGTTGATGGTGGACAGGCTGGCAG
CTTCCTGTACTGCAACGCCAGATCTCCTCTGGGCTCTAGAGTGCAGGCCCTGTCTACCGATGAG
GGCACCAGTTTTCTGCCCGCCGAAAGAGTTGCCTCTCTGCCTGAAACAGCCTGGGGCTGTCAGG
GCTCTATCGTGGGATTTCCTGCTCCTGCTCCAAACAGACCCCGGGACGATTCTTGGAGTGTCGG
CCCTGGATCTCCACTGCAGCCTCCATTGCTTGGACCAGGCGTTCACGAGCCACCTGAAGAGGCT
GCCGTTGATCCTAGAGGCGGACAAGTTCCTGGCGGCCCTTTTAGCAGACTGCAGCCAAGAGGCG
ACGGCCCTAGACAACCTGGACCAAGACCTGGCGTCAGCGGAGATGTTGGCTCTTGGACACTGGC
CCTGCCTATGCCTTTTGCCGCTCCTCCTCAGTCTCCTACCTGGCTGCTGTACTCTCACCCTGTT
GGCAGACGGGCCAGACTGCACATGGGCATCAGACTGTCTCAGAGCCCTCTGGACCCCAGAAGCT
GGACAGAGCCTTGGGTCATCTATGAGGGCCCTAGCGGCTACAGCGATCTGGCCTCTATTGGCCC
AGCTCCTGAAGGCGGACTGGTGTTCGCTTGTCTGTATGAGAGCGGCGCCAGAACCAGCTACGAC
GAGATCAGCTTCTGCACCTTCAGCCTGCGCGAGGTGCTGGAAAATGTGCCCGCCTCTCCTAAGC
CTCCTAACCTGGGCGATAAGCCTAGAGGCTGTTGCTGGCCATCT
SEQ ID NO: 27:
MTGERPSTALPDRRWGPRILGFWGGCRVWVFAAIFLLLSLAASWSKA
SEQ ID NO: 28:
MDMRVPAQLLGLLLLWLPGARC
SEQ ID NO: 29:
YGTL
SEQ ID NO: 30:
MTVEKSVVFKAEGEHFTDQKGNTIVGSGSGGTTKYFRIPAMCTTSKGTIVVFADARHNTASDQS
FIDTAAARSTDGGKTWNKKIAIYNDRVNSKLSRVMDPTCIVANIQGRETILVMVGKWNNNDKTW
GAYRDKAPDTDWDLVLYKSTDDGVTFSKVETNIHDIVTKNGTISAMLGGVGSGLQLNDGKLVFP
VQMVRTKNITTVLNTSFIYSTDGITWSLPSGYCEGFGSENNIIEFNASLVNNIRNSGLRRSFET
KDFGKTWTEFPPMDKKVDNRNHGVQGSTITIPSGNKLVAAHSSAQNKNNDYTRSDISLYAHNLY
SGEVKLIDDFYPKVGNASGAGYSCLSYRKNVDKETLYVVYEANGSIEFQDLSRHLPVIKSYN
SEQ ID NO: 31:
EPKSCDKTHTCPPCPAPELLGGPSVELFPPKPKDTLMISRTPEVTCVVVDVSHEDPEVKENWYV
DGVEVHNAKTKPREEQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK
SEQ ID NO: 32:
DKTHTCPPCPAPELLGGPSVFLFPPKPKDTLMISRTPEVTCVVVDVSHEDPEVKENWYVDGVEV
HNAKTKPREEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFELTSKLTV
DKSRWQQGNVFSCSVMHEALHNHYTQKSLSLSPGK
SEQ ID NO: 33:
EPKSCDKTHTCPPCPAPELLGGPSVFLFPPKPKDTLMISRTPEVTCVVVDVSHEDPEVKENWYV
DGVEVHNAKTKPREEQYNSTYRVVSVLTVLHQDWLNGKEYKCKVSNKALPAPIEKTISKAKGQP
REPQVYTLPPSREEMTKNQVSLYCLVKGFYPSDIAVEWESNGQPENNYKTTPPVLDSDGSFFLY
SKLTVDKSRWQQGNVFSCSVMHEALHNHYTQKSLSLSPGK
SEQ ID NO: 34:
ATGAGACCTGCGGACCTGCCCCCGCGCCCCATGGAAGAATCCCCGGCGTCCAGCTCTGCCCCGA
CAGAGACGGAGGAGCCGGGGTCCAGTGCAGAGGTCATGGAAGAAGTGACAACATGCTCCTTCAA
CAGCCCTCTGTTCCGGCAGGAAGATGACAGAGGGATTACCTACCGGATCCCAGCCCTGCTCTAC
ATACCCCCCACCCACACCTTCCTGGCCTTTGCAGAGAAGCGTTCTACGAGGAGAGATGAGGATG
CTCTCCACCTGGTGCTGAGGCGAGGGTTGAGGATTGGGCAGTTGGTACAGTGGGGGCCCCTGAA
GCCACTGATGGAAGCCACACTACCGGGGCATCGGACCATGAACCCCTGTCCTGTATGGGAGCAG
AAGAGTGGTTGTGTGTTCCTGTTCTTCATCTGTGTGCGGGGCCATGTCACAGAGCGTCAACAGA
TTGTGTCAGGCAGGAATGCTGCCCGCCTTTGCTTCATCTACAGTCAGGATGCTGGATGTTCATG
GAGTGAGGTGAGGGACTTGACTGAGGAGGTCATTGGCTCAGAGCTGAAGCACTGGGCCACATTT
GCTGTGGGCCCAGGTCATGGCATCCAGCTGCAGTCAGGGAGACTGGTCATCCCTGCGTATACCT
ACTACATCCCTTCCTGGTTCTTTTGCTTCCAGCTACCATGTAAAACCAGGCCTCATTCTCTGAT
GATCTACAGTGATGACCTAGGGGTCACATGGCACCATGGTAGACTCATTAGGCCCATGGTTACA
GTAGAATGTGAAGTGGCAGAGGTGACTGGGAGGGCTGGCCACCCTGTGCTATATTGCAGTGCCC
GGACACCAAACAGGTGCCGGGCAGAGGCGCTCAGCACTGACCATGGTGAAGGCTTTCAGAGACT
GGCCCTGAGTCGACAGCTCTGTGAGCCCCCACATGGTTGCCAAGGGAGTGTGGTAAGTTTCCGG
CCCCTGGAGATCCCACATAGGTGCCAGGACTCTAGCAGCAAAGATGCACCCACCATTCAGCAGA
GCTCTCCAGGCAGTTCACTGAGGCTGGAGGAGGAAGCTGGAACACCGTCAGAATCATGGCTCTT
GTACTCACACCCAACCAGTAGGAAACAGAGGGTTGACCTAGGTATCTATCTCAACCAGACCCCC
TTGGAGGCTGCCTGCTGGTCCCGCCCCTGGATCTTGCACTGTGGGCCCTGTGGCTACTCTGATC
TGGCTGCTCTGGAGGAGGAGGGCTTGTTTGGGTGTTTGTTTGAATGTGGGACCAAGCAAGAGTG
TGAGCAGATTGCCTTCCGCCTGTTTACACACCGGGAGATCCTGAGTCACCTGCAGGGGGACTGC
ACCAGCCCTGGTAGGAACCCAAGCCAATTCAAAAGCAAT
SEQ ID NO: 35:
ATGATGAGCTCTGCAGCCTTCCCAAGGTGGCTGAGCATGGGGGTCCCTCGTACCCCTTCACGGA
CAGTGCTCTTCGAGCGGGAGAGGACGGGCCTGACCTACCGCGTGCCCTCGCTGCTCCCCGTGCC
CCCCGGGCCCACCCTGCTGGCCTTTGTGGAGCAGCGGCTCAGCCCTGACGACTCCCACGCCCAC
CGCCTGGTGCTGAGGAGGGGCACGCTGGCCGGGGGCTCCGTGCGGTGGGGTGCCCTGCACGTGC
TGGGGACAGCAGCCCTGGCGGAGCACCGGTCCATGAACCCCTGCCCTGTGCACGATGCTGGCAC
GGGCACCGTCTTCCTCTTCTTCATCGCGGTGCTGGGCCACACGCCTGAGGCCGTGCAGATCGCC
ACGGGAAGGAACGCCGCGCGCCTCTGCTGTGTGGCCAGCCGTGACGCCGGCCTCTCGTGGGGCA
GCGCCCGGGACCTCACCGAGGAGGCCATCGGTGGTGCCGTGCAGGACTGGGCCACATTCGCTGT
GGGTCCCGGCCACGGTGTGCAGCTGCCCTCAGGCCGCCTGCTGGTACCCGCCTACACCTACCGC
GTGGACCGCCGAGAGTGTTTTGGCAAGATCTGCCGGACCAGCCCTCACTCCTTCGCCTTCTACA
GCGATGACCACGGCCGCACCTGGCGCTGTGGAGGCCTCGTGCCCAACCTGCGCTCAGGCGAGTG
CCAGCTGGCAGCGGTGGACGGTGGGCAGGCCGGCAGCTTCCTCTACTGCAATGCCCGGAGCCCA
CTGGGCAGCCGTGTGCAGGCGCTCAGCACTGACGAGGGCACCTCCTTCCTGCCCGCAGAGCGCG
TGGCTTCCCTGCCCGAGACTGCCTGGGGCTGCCAGGGCAGCATCGTGGGCTTCCCAGCCCCCGC
CCCCAACAGGCCACGGGATGACAGTTGGTCAGTGGGCCCCGGGAGTCCCCTCCAGCCTCCACTC
CTCGGTCCTGGAGTCCACGAACCCCCAGAGGAGGCTGCTGTAGACCCCCGTGGAGGCCAGGTGC
CTGGTGGGCCCTTCAGCCGTCTGCAGCCTCGGGGGGATGGCCCCAGGCAGCCTGGCCCCAGGCC
TGGGGTCAGTGGGGATGTGGGGTCCTGGACCCTGGCACTCCCCATGCCCTTTGCTGCCCCGCCC
CAGAGCCCCACGTGGCTGCTGTACTCCCACCCAGTGGGGCGCAGGGCTCGGCTACACATGGGTA
TCCGCCTGAGCCAGTCCCCGCTGGACCCGCGCAGCTGGACAGAGCCCTGGGTGATCTACGAGGG
CCCCAGCGGCTACTCCGACCTGGCGTCCATCGGGCCGGCCCCTGAGGGGGGCCTGGTTTTTGCC
TGCCTGTACGAGAGCGGGGCCAGGACCTCCTATGATGAGATTTCCTTTTGTACATTCTCCCTGC
GTGAGGTCCTGGAGAACGTGCCCGCCAGCCCCAAACCGCCCAACCTTGGGGACAAGCCTCGGGG
GTGCTGCTGGCCCTCC
SEQ ID NO: 36:
MRFKNVKKTALMLAMFGMATSSNAALFDYNATGDTEFDSPAKQGWMQDNTNNGSGVLTNADGMP
AWLVQGIGGRAQWTYSLSTNQHAQASSFGWRMTTEMKVLSGGMITNYYANGTQRVLPIISLDSS
GNLVVEFEGQTGRTVLATGTAATEYHKFELVELPGSNPSASFYFDGKLIRDNIQPTASKQNMIV
WGNGSSNTDGVAAYRDIKFEIQGDVIFRGPDRIPSIVASSVTPGVVTAFAEKRVGGGDPGALSN
TNDIITRTSRDGGITWDTELNLTEQINVSDEFDESDPRPIYDPSSNTVLVSYARWPTDAAQNGD
RIKPWMPNGIFYSVYDVASGNWQAPIDVTDQVKERSFQIAGWGGSELYRRNTSLNSQQDWQSNA
KIRIVDGAANQIQVADGSRKYVVTLSIDESGGLVANLNGVSAPIILQSEHAKVHSFHDYELQYS
ALNHTTTLFVDGQQITTWAGEVSQENNIQFGNADAQIDGRLHVQKIVLTQQGHNLVEFDAFYLA
QQTPEVEKDLEKLGWTKIKTGNTMSLYGNASVNPGPGHGITLTRQQNISGSQNGRLIYPAIVLD
RFFLNVMSIYSDDGGSNWQTGSTLPIPFRWKSSSILETLEPSEADMVELQNGDLLLTARLDENQ
IVNGVNYSPRQQFLSKDGGITWSLLEANNANVESNISTGTVDASITRFEQSDGSHELLFTNPQG
NPAGTNGRQNLGLWFSFDEGVTWKGPIQLVNGASAYSDIYQLDSENAIVIVETDNSNMRILRMP
ITLLKQKLTLSQN
SEQ ID NO: 37:
TTGTCAATCAAGATGACTTCACAACGAAGAAGAGCATCGATTCACAAGGAAACAGATTCTAATA
TAAAGGGAGTAGATATGCGTTTCAAAAACGTAAAGAAAACCGCTTTAATGCTTGCAATGTTCGG
TATGGCGACAAGCTCAAACGCCGCACTTTTTGACTATAACGCAACGGGTGACACTGAGTTTGAC
AGTCCAGCCAAACAGGGATGGATGCAAGACAACACGAATAATGGCAGCGGCGTTTTAACCAATG
CAGATGGAATGCCCGCTTGGTTGGTGCAAGGTATTGGAGGGAGAGCTCAATGGACATATTCTCT
CTCTACTAATCAACATGCCCAAGCATCAAGTTTCGGTTGGCGAATGACGACAGAAATGAAAGTG
CTCAGTGGTGGAATGATCACAAACTACTACGCCAACGGCACTCAGCGTGTCTTACCCATCATTT
CATTAGATAGCAGTGGTAACTTAGTTGTTGAGTTTGAAGGGCAAACTGGACGCACCGTTTTGGC
AACCGGCACAGCAGCAACGGAATATCATAAATTTGAATTGGTATTCCTTCCTGGAAGTAACCCA
TCCGCTAGCTTTTACTTCGATGGCAAACTCATTCGTGACAACATCCAGCCGACTGCATCAAAAC
AAAATATGATCGTATGGGGGAATGGCTCATCAAATACGGATGGTGTCGCCGCTTATCGTGATAT
TAAGTTTGAAATTCAAGGCGACGTCATCTTCAGAGGCCCAGACCGTATACCGTCCATTGTAGCA
AGTAGCGTAACACCAGGGGTGGTAACCGCATTTGCAGAGAAACGTGTGGGGGGAGGAGATCCCG
GTGCTCTGAGTAATACCAATGACATAATCACTCGTACCTCACGAGATGGCGGTATAACTTGGGA
TACCGAGCTCAACCTCACTGAGCAAATCAATGTCAGTGATGAGTTTGATTTCTCCGATCCTCGG
CCTATCTATGATCCTTCCTCCAATACGGTTCTTGTCTCTTATGCTCGATGGCCGACCGATGCCG
CTCAAAACGGAGATCGAATAAAACCATGGATGCCAAACGGTATTTTTTACAGCGTCTATGATGT
TGCATCAGGGAACTGGCAAGCGCCTATCGATGTTACCGATCAGGTGAAAGAACGCAGTTTCCAA
ATCGCTGGTTGGGGTGGTTCAGAGCTGTATCGCCGAAATACCAGCCTAAATAGCCAGCAAGACT
GGCAATCAAACGCTAAGATCCGAATTGTTGATGGTGCAGCGAACCAGATACAAGTTGCCGATGG
TAGCCGAAAATATGTTGTCACACTGAGTATTGATGAATCAGGTGGTCTAGTCGCTAATCTAAAC
GGTGTTAGTGCTCCGATTATCCTGCAATCTGAACACGCAAAGGTACACTCTTTCCATGACTACG
AACTTCAATATTCGGCGTTAAACCACACCACAACGTTATTCGTGGATGGTCAGCAAATCACAAC
TTGGGCTGGCGAAGTATCGCAGGAGAACAACATTCAGTTTGGTAATGCGGATGCCCAAATTGAC
GGCAGACTGCATGTGCAAAAAATTGTTCTCACACAGCAAGGCCATAACCTCGTGGAGTTTGATG
CTTTCTATTTAGCACAGCAAACCCCTGAAGTAGAGAAAGACCTTGAAAAGCTTGGTTGGACAAA
AATTAAAACGGGCAACACCATGAGTTTGTATGGAAATGCCAGTGTCAACCCAGGACCGGGTCAT
GGCATCACCCTTACTCGACAACAAAATATCAGTGGCAGCCAAAACGGCCGCTTGATCTACCCAG
CGATTGTGCTTGATCGTTTCTTCTTGAACGTCATGTCTATTTACAGTGATGATGGCGGTTCAAA
CTGGCAAACCGGTTCAACACTCCCTATCCCCTTTCGCTGGAAGAGTTCGAGTATCCTAGAAACT
CTCGAACCTAGTGAAGCTGATATGGTTGAACTCCAAAACGGTGATCTACTCCTTACTGCACGCC
TTGATTTTAACCAAATCGTTAATGGTGTGAACTATAGCCCACGCCAGCAATTTTTGAGTAAAGA
TGGTGGAATCACGTGGAGCCTACTTGAGGCTAACAACGCTAACGTCTTTAGCAATATCAGTACT
GGTACCGTTGATGCTTCTATTACTCGGTTCGAGCAAAGTGACGGTAGCCATTTCTTACTCTTTA
CTAACCCACAAGGAAACCCTGCGGGGACAAATGGCAGGCAAAATCTAGGCTTATGGTTTAGCTT
CGATGAAGGGGTGACATGGAAAGGACCAATTCAACTTGTTAATGGTGCATCGGCATATTCTGAT
ATTTATCAATTGGATTCGGAAAATGCGATTGTCATTGTTGAAACGGATAATTCAAATATGCGAA
TTCTTCGTATGCCTATCACATTGCTAAAACAGAAGCTGACCTTATCGCAAAACTAA
SEQ ID NO: 38:
MVGADPTRPRGPLSYWAGRRGQGLAAIFLLLVSAAESEARAEDDESLVQPLVTMEQLLWVSGKQ
IGSVDTFRIPLITATPRGTLLAFAEARKKSASDEGAKFIAMRRSTDQGSTWSSTAFIVDDGEAS
DGLNLGAVVNDVDTGIVELIYTLCAHKVNCQVASTMLVWSKDDGISWSPPRNLSVDIGTEMFAP
GPGSGIQKQREPGKGRLIVCGHGTLERDGVFCLLSDDHGASWHYGTGVSGIPFGQPKHDHDENP
DECQPYELPDGSVIINARNQNNYHCRCRIVLRSYDACDTLRPRDVTEDPELVDPVVAAGALATS
SGIVEFSNPAHPEFRVNLTLRWSFSNGTSWLKERVQVWPGPSGYSSLTALENSTDGKKQPPQLF
VLYEKGLNRYTESISMVKISVYGTL
SEQ ID NO: 39:
MTVQPSPWFSDLRPMATCPVLQKETLERTGVHAYRIPALLYLKKQKTLLAFAEKRASKTDEHAE
LIVLRRGSYNEATNRVKWQPEEVVTQAQLEGHRSMNPCPLYDKQTKTLFLFFIAVPGRVSEHHQ
LHTKVNVTRLCCVSSTDHGRTWSPIQDLTETTIGSTHQEWATFAVGPGHCLQLRNPAGSLLVPA
YAYRKLHPAQKPTPFAFCFISLDHGHTWKLGNFVAENSLECQVAEVGTGAQRMVYLNARSELGA
RVQAQSPNDGLDFQDNRVVSKLVEPPHGCHGSVVAFHNPISKPHALDTWLLYTHPTDSRNRTNL
GVYLNQMPLDPTAWSEPTLLAMGICAYSDLQNMGQGPDGSPQFGCLYESGNYEEIIFLIFTLKQ
AFPTVEDAQ
SEQ ID NO: 40:
MEEVPPYSLSSTLFQQEEQSGVTYRIPALLYLPPTHTFLAFAEKRTSVRDEDAACLVLRRGLMK
GRSVQWGPQRLLMEATLPGHRTMNPCPVWEKNTGRVYLFFICVRGHVTERCQIVWGKNAARLCF
LCSEDAGCSWGEVKDLTEEVIGSEVKRWATFAVGPGHGIQLHSGRLIIPAYAYYVSRWFLCFAC
SVKPHSLMIYSDDFGVTWHHGKFIEPQVTGECQVAEVAGTAGNPVLYCSARTPSRFRAEAFSTD
SGGCFQKPTLNPQLHEPRTGCQGSVVSFRPLKMPNTYQDSIGKGAPATQKCPLLDSPLEVEKGA
ETPSATWLLYSHPTSKRKRINLGIYYNRNPLEVNCWSRPWILNRGPSGYSDLAVVEEQDLVACL
FECGEKNEYERIDFCLESDHEVLSCEDCTSPSSD
SEQ ID NO: 41:
METAGAPFCFHVDSLVPCSYWKVMGPTRVPRRTVLFQRERTGLTYRVPALLCVPPRPTLLAFAE
QRLSPDDSHAHRLVLRRGTLTRGSVRWGTLSVLETAVLEEHRSMNPCPVLDEHSGTIFLFFIAV
LGHTPEAVQIATGKNAARLCCVTSCDAGLTWGSVRDLTEEAIGAALQDWATFAVGPGHGVQLRS
GRLLVPAYTYHVDRRECFGKICWTSPHSLAFYSDDHGISWHCGGLVPNLRSGECQLAAVDGDEL
YCNARSPLGNRVQALSADEGTSFLPGELVPTLAETARGCQGSIVGFLAPPSIEPQDDRWTGSPR
NTPHSPCFNLRVQESSGEGARGLLERWMPRLPLCYPQSRSPENHGLEPGSDGDKTSWTPECPMS
SDSMLQSPTWLLYSHPAGRRARLHMGIYLSRSPLDPHSWTEPWVIYEGPSGYSDLAFLGPMPGA
SLVFACLFESGTRTSYEDISFCLESLADVLENVPTGLEMLSLRDKAQGHCWPS
SEQ ID NO: 42:
GGGTCACATGCTGATGGACTAATTGGAGTCGCGGCAGCGCGGGCTGCGGCCCCCAAGGGGAGGG
GTCGGAGTGACGTGCGCGCTTTTAAAGGGCCGAGGTCAGCTGACGGCTTGCCACCGGTGACCAG
TTCCTGGACAGGGATCGCCGGGAGCTATGGTGGGGGCAGACCCGACCAGACCCCGGGGACCGCT
GAGCTATTGGGCGGGCCGTCGGGGTCAGGGGCTCGCAGCGATCTTCCTGCTCCTGGTGTCCGCG
GCGGAATCCGAGGCCAGGGCAGAGGATGACTTCAGCCTGGTGCAGCCGCTGGTGACCATGGAGC
AGCTGCTGTGGGTGAGCGGGAAGCAGATCGGCTCTGTAGACACTTTCCGCATCCCGCTCATCAC
AGCCACCCCTCGGGGCACGCTCCTGGCCTTCGCTGAGGCCAGGAAAAAATCTGCATCCGATGAG
GGGGCCAAGTTCATCGCCATGAGGAGGTCCACGGACCAGGGTAGCACGTGGTCCTCTACAGCCT
TCATCGTAGACGATGGGGAGGCCTCCGATGGCCTGAACCTGGGCGCTGTGGTGAACGATGTAGA
CACAGGGATAGTGTTCCTTATCTATACCCTCTGTGCTCACAAGGTCAACTGCCAGGTGGCCTCT
ACCATGTTGGTTTGGAGTAAGGACGACGGCATTTCCTGGAGCCCACCCCGGAATCTCTCTGTGG
ATATTGGCACAGAGATGTTTGCCCCTGGACCTGGCTCAGGCATTCAGAAACAGCGGGAGCCTGG
GAAGGGCCGGCTCATTGTGTGTGGACACGGGACGCTGGAGCGAGATGGGGTCTTCTGTCTCCTC
AGTGATGACCACGGTGCCTCCTGGCACTACGGCACTGGAGTGAGCGGCATTCCCTTTGGCCAGC
CCAAACACGATCACGATTTCAACCCCGACGAGTGCCAGCCCTACGAGCTTCCAGATGGCTCGGT
CATCATCAACGCCCGGAACCAGAATAACTACCATTGCCGCTGCAGGATCGTCCTCCGCAGCTAT
GACGCCTGTGACACCCTCAGGCCCCGGGATGTGACCTTCGACCCTGAGCTCGTGGACCCTGTGG
TAGCTGCAGGAGCACTAGCCACCAGCTCCGGCATTGTCTTCTTCTCCAATCCAGCCCACCCTGA
GTTCCGAGTGAACCTGACCCTGCGCTGGAGTTTCAGCAATGGTACATCCTGGCAGAAGGAGAGG
GTCCAGGTGTGGCCGGGACCCAGCGGCTACTCGTCCCTGACAGCCCTGGAAAACAGCACGGATG
GAAAGAAGCAGCCCCCGCAGCTGTTCGTTCTGTACGAGAAAGGCCTGAACCGGTACACCGAGAG
CATCTCCATGGTCAAAATCAGCGTCTACGGCACGCTCTGAGCCCCGTGCCCAAAGGACACCAAG
TCCTGGTCGCTGACTTCACAGCTCTCTGGACCATCTGCAGAGGGTGCCTGAAACACAGCTCTTC
CTCTGAACTCTGACCTTTTGCAACTTCTCATCAACAGGGAAGTCTCTTCGTTATGACTTAACAC
CCAGCTTCCTCTCGGGGCAGGAAGTCCCTCCGTCACCAAGAGCACTTTTTTCCAGTATGCTGGG
GATGGCCCCTGTCCATTCTCTTCCAGGACAACGGAGCTGTGCCTTTCTGGGACAGGATGGGGGA
GGGGCTCCCCCTGGAGAGATGAACAGATACGAACTCAGGGAACTGAGAAGGCCCGGTGTCCTAG
GGTACAAAGGCAGGTACTAGATGTGATTGCTGAAAGTCCCCAGGGCAGAGTGTCCTTTCAGAGC
AAGGATAAGCACACCTACGTGTGCACCTTTGATTATTTATGAATCGAAATATTTGTAACTTAAA
ATTTTTGATGCAGAAAAAGCGTTTGTGGAGTCTGTGGTTCTGTCTGCTCACGCCTTCCCAATTG
CCTCCTGGAGAGACAGGAAGGCAGCTGGAAGAGGAGCCGATGTACTTACTGGGAAGCAGAAACC
CCTAGATTCCATCCTGGCTGCTGCTGTTTGCAAGTGTCAAAGATGGGGGGGCGTGTTTATATTT
TATATTTCTAAGATGGGGTGGCATAGGAAATAGGGAACAGATGTGTAAAACCAGATGGGAAGGA
CAGTCTGTGAGAAAGGAGCAAGCAGTTGCTGCAGGTGTGGGAGAGCAAAGCCCTTCTCCACGTG
GAAAGAGCCCAGATGGACGCTAAGCATGTTGGGCACCTGTAACCCCGCACTCGCTGGACTGACG
GTGTAGCTCAGTGGTGGAGCTAGTACTTGGAACGCCTAAGACTCTGGGTTCAGTCCTTGGGGGG
GGGGGTATGTGTTTATTGAGAGGAAGGTGTACGTACTGTAGGTCAGAGGACAGCTTACTGGAGT
TGTCTCTCTCCTTCACGCTGTGAGTCCTGTGGAATGACCTCAGGTGTCAGAGTTGGGGGCAGGT
GCCTTTGCCAGCTGAGCCATCTTGCTGTCTCTGCTTTAATTTAAAAAAAAAAAAAAAAAAGAAT
ATTAAGGTCTGAGGGATTCGGGCTGCGTTCATTTCAATTAGAGGGTCATATTTCTTTTGACATT
TCTTCTCTAAGAAATGTTAAGATCATTTGTTCTGTGTGATAGAGGTATAGCTCCATTGTATGTC
AGCAGTGAGGGATCCTGTGCATTTTATCCAGAGTTTGTACGGTGTTCTAGGGGCTGCTAGTGCA
GCCCAGTGCTAAACACTTCAGCATGCACAAGGCCTCAATCAGTGCATGCATGTGCACACACACA
CAGACACACACGTACACACTGACACAGGTACACAAATACACACTGGCCCACATGTACACATCGA
CTCACAGGTACACAGACCCACTTTGACACACATATACACAGACACAAACGCACTGGCACACACA
TATACACAGGCACACATGGATAGATGGACACACGTGTACACATACACACACACACAGAAATACA
AATGTTCAGGTTTTCTAAAAAAAAAAAAATTAGAGACGTGTTGACTTCATTTTTAGCAAAAATC
CTGTCATGTATCTTAAAGTGGATTGAACCCACTATGTAGCCCAGGCTGGCCTCCAAATGGGCAT
CCTTCTGCCTCAGTCTCCCGAGGGCTAGGATAACAGGAGTATGCCATCACACCTGGCTAATAGA
AATTTTCAAAATTGTTTGTTTGAAGGTGACTCTTACTATATTGCCTAACTGATCTCCAGTTCGT
GAAATCCTCCTGCCTCAGAACCAGGACTGTCAATATAACCCACCAAGACAGGCCAACATTCACA
ATTGATTGTTAGTTTGTGGTCTGAATCAAGGTCTTATACTGTAGCCCAGGCTAGCCCGGAATAC
ACGATATCTCCAGTGCTTCAGATCCTCAGTTCTAACTAAGCATGGCCACATCCATGTTTAACTG
CAAATTTGATGTTACCATGGTTTGGTTTGGTTTGGTTTGGTTTGGTTTGGTTTGGTTTGGTTTT
TTGGCCATTTTTTTTTTCTCATGCTGAGGCCTTGTGCTCTCAAGTTGGGGAGACAGCATGGAGG
GTAGCTGCAACTGTAACCCCAGTTCCAGGGGACCTGACACCCTCTGGCCTCCACAAGTATTAGG
CACATCTGTGGTGCACAGACATACAATCAGGCAAAATATTCATACACATAAAATAAAATAATTT
AAAACAAAAGCAAAAATCAGGACCTAAGAAAAAAATCTATTCCTGATTCTTTTATGTTTTGTTT
GTATTTTATCAAGACAGGGTTGTTTCTCTGTATAGCCCTGGCTGTCTTGGAATTCACTCTGTAG
ACCAGGCTGGCCTCAAACTCAGAAATCCTCCTGCCTTTGCCTTCCAAGTGCTGGAATTAAAGGC
ATGCGCCACC
SEQ ID NO: 43:
GACATGACCCAAACGGCCCCTGGCTGCAAGGTAATATCGGAAGTTGACTAAGAATGGACGCCCC
ACCACTGACTGACCCGCCCCCTGAGTCTGAGATTGGACTTGTCTCTGGATACAGTCATACTTTG
AGGTACTACAAGTTAGAAACTGTTAGGTTACTCAGTTCAGTCCATGACAGTCCAACCTTCTCCA
TGGTTTTCCGATCTCAGGCCCATGGCGACCTGCCCTGTCCTGCAGAAGGAGACACTGTTCCGCA
CAGGCGTCCATGCTTACAGAATCCCTGCTCTGCTCTACCTGAAGAAGCAGAAGACCCTGCTGGC
CTTTGCGGAAAAGCGAGCCAGCAAGACGGATGAGCACGCAGAGTTGATTGTCCTGAGAAGAGGA
AGCTACAACGAAGCCACCAACCGTGTCAAGTGGCAGCCTGAGGAAGTGGTGACCCAAGCCCAGC
TGGAAGGCCACCGCTCCATGAATCCATGTCCCTTGTATGACAAGCAAACAAAGACCCTCTTCCT
TTTCTTCATCGCTGTCCCTGGGCGTGTATCAGAACATCATCAGCTCCACACTAAGGTTAATGTC
ACACGGCTGTGCTGTGTCAGCAGCACTGACCATGGGAGGACCTGGAGCCCCATCCAGGACCTCA
CAGAGACCACCATTGGCAGCACTCATCAGGAATGGGCCACATTTGCTGTGGGTCCTGGGCATTG
TCTGCAGCTGCGGAACCCAGCTGGGAGCCTGCTGGTACCTGCTTATGCCTACCGGAAACTGCAC
CCTGCTCAGAAGCCTACCCCCTTTGCCTTCTGCTTCATCAGCCTTGACCATGGGCACACATGGA
AACTAGGCAACTTTGTGGCTGAAAACTCACTGGAGTGCCAGGTGGCTGAGGTTGGCACTGGAGC
TCAGAGGATGGTATATCTCAATGCTAGGAGCTTCCTGGGAGCCAGGGTCCAGGCACAAAGTCCT
AATGATGGTCTGGATTTCCAGGACAACCGGGTAGTGAGTAAGCTTGTAGAGCCCCCCCACGGGT
GTCATGGAAGTGTGGTTGCCTTCCACAACCCCATCTCTAAGCCACATGCCTTAGACACATGGCT
TCTTTATACACACCCTACAGACTCCAGGAATAGAACCAACCTGGGTGTGTACCTAAACCAGATG
CCACTAGATCCCACAGCCTGGTCAGAGCCCACCCTGCTGGCCATGGGCATCTGTGCCTACTCAG
ACTTACAGAACATGGGGCAAGGCCCTGATGGCTCCCCACAGTTTGGGTGTCTGTATGAATCAGG
TAACTATGAAGAGATCATTTTCCTCATATTCACCCTGAAGCAAGCTTTCCCCACTGTATTTGAT
GCCCAGTGATCTCAGTGCACGTGGCCCAAAGGGCTTCCTTGTGCTTCAAAACACCCATCTCTCT
TTGCTTCCAGCATCCTCTGGACTCTTGAGTCCAGCTCTTGGGTAACTTCCTCAGGAGGATGCAG
AGAATTTGGTCTCTTGACTCTCTGCAGGCCTTATTGTTTCAGCCTCTGGTTCTCTTTTCAGCCC
AGAAATCAAAGGAGCCTGGCTTTCCTCAGCCTGTTGGCAGGGCAGGTGGGGACAGTATATATAG
AGGCTGCCATTCTGCATGTCGGTTGTCACTATGCTAGTTTAACCTGCCTGTTTCCCCATGCCTA
GTGTTTGAATGAGTATTAATAAAATATCCAACCCAGCCCATTTCTTCCTGGAAAAAAA
SEQ ID NO: 44:
ACTGCGCGGTGAAGGGGCGTGGCCTGGCCGGGGAGGTTGACACCCAGACGCTGCTCTCAGTCCT
CTGGCGCCTGCTCCCCAGCGCATTCCTTCTGCTCCTGGGATATTTGTCTCATTACTGCCAGTTC
TTGCGCAGCGGTCACTGGGTTCGTTTCAGCGTCTGTGGTTTCTGTCGCTGTTATCCAGTCTCCA
TCGCCCCAGCTCAGCTTCAGGCCTTCTTCCGAGACTCCACGGGAGAGCCCAGAGAGCCTCCGGA
GCCGAAGCCATGGAGGAAGTCCCACCCTACTCCCTCAGCAGCACCCTGTTCCAGCAGGAAGAAC
AGAGTGGGGTGACCTACCGGATCCCAGCCCTGCTGTACCTTCCTCCCACCCACACCTTCCTGGC
CTTTGCAGAGAAGCGGACCTCAGTCAGAGATGAGGATGCTGCCTGCCTGGTGCTCAGACGAGGG
CTGATGAAGGGGCGCTCTGTACAGTGGGGCCCCCAACGGCTACTGATGGAGGCCACATTACCTG
GGCATCGCACCATGAACCCCTGCCCTGTGTGGGAGAAAAATACTGGCCGTGTGTACCTGTTTTT
CATCTGTGTGCGGGGCCATGTTACTGAGAGGTGCCAGATTGTGTGGGGCAAAAATGCCGCCCGT
CTCTGCTTCCTTTGCAGTGAAGATGCCGGCTGCTCTTGGGGTGAAGTGAAAGACTTGACCGAGG
AGGTCATTGGCTCAGAGGTGAAGCGCTGGGCCACATTTGCTGTGGGCCCAGGTCATGGCATCCA
GCTACACTCGGGAAGGCTGATCATCCCCGCCTATGCCTACTATGTCTCACGTTGGTTTCTCTGC
TTTGCGTGTTCAGTCAAGCCCCATTCCCTGATGATCTACAGTGATGACTTTGGAGTCACATGGC
ACCATGGCAAGTTCATTGAGCCCCAGGTGACAGGGGAGTGCCAAGTGGCCGAAGTGGCTGGGAC
GGCTGGTAACCCTGTGCTCACTGCAGTGCCCGAACACCAAGCCGATTTCGAGCAGAGGCTTTTA
GTACTGATAGTGGTGGCTGCTTTCAGAAGCCAACCCTGAACCCACAACTCCATGAGCCTCGAAC
CGGCTGCCAAGGTAGTGTAGTGAGCTTCCGGCCTTTGAAGATGCCAAATACCTATCAAGACTCA
ATTGGCAAAGGTGCTCCCGCTACTCAGAAGTGCCCTCTGCTGGACAGTCCTCTGGAGGTGGAGA
AAGGAGCTGAAACACCATCAGCAACATGGCTCTTGTACTCACATCCAACTAGCAAGAGGAAGAG
GATTAACCTAGGCATCTACTACAACCGGAACCCCTTGGAGGTGAACTGCTGGTCCCGCCCGTGG
ATCTTGAACCGTGGGCCCAGTGGCTACTCTGATCTGGCTGTTGTGGAAGAACAGGACTTGGTGG
CGTGTTTGTTTGAGTGTGGGGAGAAGAATGAGTATGAGCGGATTGACTTCTGTCTGTTTTCAGA
CCATGAGGTCCTGAGCTGTGAAGACTGTACCAGCCCTAGTAGCGACTAAAGCCAAATCAAGACG
GATGAGTGAGGCCCAGCTTCCCACAGAAAGGAATGGCAGCTACAGCCAGGGTAACAGAGGTCTC
TGATGTCTAGAGAAAACTCTAAAAACTAATAATCTGCTCCTTGAATTTTTTCACTTTTCCCTTC
AATGAGCATGGTGAAAATTGTGCCATATCTTACATAACGAGGCTCTTGAACTGGGAGTTTGAAT
CTCTTCTCTTCCCATTAAAAGGAGAGGCCATGTGCTCGCTTCGCGTTCGACAAAGCCTGGATTC
TGATCTTGAGTGGAAGCCACAGGCTTGTCTTTTCCAATGGTTCACTGCTCACCTGAGTATTAGG
TGATGTGTAGGTGCCTTGGCCAGAAGAAAGATCTGTGTTGTTGTATTTTTTTAAATTTATTTAT
TTACTATATGTAAGTACACTGCAGCTGTCTTCAGACACACCAGAAGAGGGCGTCAGATCTCATT
AGAGATGGTTGTGAGCCACCATGTGGTTGCTGGGATTTGAACTCAGGACCTTCAGAAGAGCAGT
CAGTGCTCTTAACTACTGAGCCATCTCTCAAGCCCCGCATTGCTGTATTTTTAATAAGAAAAAT
GCCCTTATCCTTCCAATAATGCCTGGAGCTGTACAAATTCTCTGTCTTAGAAGACTTGAGAAAG
CAGAACTGTAAGGTCAGATGCTTTCTCCAGCCTTGATGCTGTGTTCCACCTTCCCTTCCTCATC
CAGAAAACAGTTACTAGGGAGAAAATGAGAAACCCATGCCAGCTGCCCTTGATGATGGTTGATA
ACGGTGCTTATTGCTTTTGATGTCATTACCTCTGTTAGAGATGAATCAGAGTCAGAGGTCCTTA
GCTGCATCCACCCATTTCCAGGGGGACATTCTAACACTGCTGAACAGTCAGCTAAAATGAGAGC
TGTGTGTCCTAGCCTGATTCCAGGTTAGTCATGATGCTTCCTGGAGCTGGGCTTTTATCTAATC
CCAGGAGCCATCTAGGGGAGGCTCAGAGCTAGCAGGTGATCTTCCTGAGATGGTTTCACCGTGA
CAGGTGAACCATGAGCCCTTCCAAGCAAGGCCAAAGGACAACATTATAGGAAAGATTTCTAGTA
TTAATATGCCTTTTCTCTGTGTGTGTACTGTCTTGTAGTGATGCTATATAGACAAATAGATGAT
TTCTTATTTTTTGTTTGTTTGTTTGTTTTTTTGTTTTTCTGTAGCCCTAGCTGTCCTGGAACTC
ACTTTGTAAACCAGGCTGGCCTCGATCTCAGAAATCCGCCTGCCTCTGCCTCCCGAGTGCTGGG
ATTAAAGGTGTGCACCACCACACCTTAATGATGATCCTATAAGTATTCCTAAAATTATACTAGT
AATTATTAACTCCTTTATAATAGGACTGCTATTAAAGCCCTCGCTGATATGAAAACTACAGTGA
GAACTCTGCCAGTCTTCACATGTCATAATTACTTCTGAGATAGAAAGCAGGCATTTACAACTTA
GAACACATTTCTTAGAGCTGTAAAACAATTAACTAGAGGTCATAAAAGGGAATGAAAGATTTAT
TGTAGGTGCTAGGACAGAACATAAAATATTGACTGGGCTTATCTATATGAAACTTCATTGTTAA
CTTTTACACAAGAATTATGGTTTTTAACTTTCAGTGAACCTGCGGAGCTAGTGACAGAAGAGAA
ATGTCTAGTTAGATAACTACTCTTAATGGAAATTCACATAAACATCTGTTGCCATCTTCTTTTT
GAATTTATGTTTAAACTTGTGAATGTTTGAATTAGACACTACGCGAGCACATAGAAAATAAAGA
ACTAAGCGTGAA
SEQ ID NO: 45:
GGACAGTGTGCATCACGGAGCTTGTGGCCCAGACTGTGCCTGGCAGACCCAGAGGACCTAAGGC
TTGGCTCTAGTGGTGGTCAGCACAGCCCTCGGTGGTCTGCGGAGCCTGATATTGCTTTACGTAA
GGGCTGTTCTGCTGTGCATCTCCTGTGTCTGAAGCTATTCGCCATGGAGACTGCTGGAGCTCCC
TTCTGCTTCCATGTGGACTCCCTGGTACCTTGCTCCTACTGGAAGGTTATGGGGCCCACGCGTG
TTCCCAGGAGAACGGTGCTCTTCCAGAGGGAAAGGACGGGCCTGACCTACCGTGTGCCTGCGTT
ACTCTGTGTGCCTCCCAGGCCTACTCTGCTGGCCTTCGCGGAACAGCGACTTAGCCCTGATGAC
TCCCATGCCCACCGCCTGGTGCTACGGAGGGGCACGCTGACCAGGGGCTCAGTGCGGTGGGGCA
CTCTGAGTGTACTGGAGACTGCAGTACTGGAGGAGCACAGGTCTATGAACCCTTGCCCGGTGCT
GGATGAGCACTCTGGTACCATCTTCCTCTTCTTCATTGCCGTGCTGGGCCACACACCGGAGGCC
GTGCAAATCGCCACTGGCAAGAACGCTGCTCGCCTCTGCTGTGTGACCAGCTGTGACGCTGGCC
TCACCTGGGGCAGTGTTCGAGATCTCACTGAGGAAGCCATTGGTGCTGCATTGCAGGACTGGGC
CACCTTTGCTGTGGGTCCGGGCCATGGAGTTCAGCTGCGCTCGGGTCGCCTGCTTGTTCCTGCT
TACACCTATCATGTGGACCGACGGGAATGTTTTGGCAAGATCTGCTGGACCAGTCCCCACTCCT
TGGCATTCTACAGTGATGATCATGGGATCTCCTGGCATTGTGGAGGCCTTGTGCCCAACCTACG
CTCTGGAGAGTGCCAACTGGCTGCGGTAGATGGAGACTTTCTCTACTGTAATGCTCGAAGCCCT
CTGGGTAACCGTGTGCAGGCACTGAGTGCTGATGAAGGCACGTCCTTCCTACCAGGGGAGCTGG
TGCCTACATTGGCAGAGACGGCTCGTGGTTGCCAGGGTAGCATTGTGGGCTTCCTAGCTCCACC
CTCAATCGAGCCTCAGGATGACCGGTGGACAGGGAGTCCTAGGAACACCCCACATTCCCCATGC
TTCAATCTCAGAGTACAGGAGTCTTCGGGGGAAGGTGCCAGAGGTCTTCTTGAACGTTGGATGC
CCAGGTTGCCTCTCTGCTACCCACAGTCCCGGAGCCCAGAGAATCATGGCCTAGAGCCTGGGTC
AGATGGAGATAAGACATCCTGGACTCCGGAATGTCCTATGTCCTCTGATTCCATGCTTCAGAGC
CCCACATGGCTACTATATTCCCACCCAGCAGGGCGTAGAGCTCGGCTCCACATGGGAATCTACC
TGAGCCGATCCCCCTTGGATCCCCACAGCTGGACAGAGCCCTGGGTGATCTATGAGGGCCCCAG
TGGCTACTCTGACCTTGCCTTTCTTGGGCCTATGCCTGGGGCATCCCTGGTTTTTGCCTGTCTG
TTTGAGAGCGGGACCAGGACTTCCTATGAAGACATTTCTTTTTGCTTGTTCTCACTGGCGGATG
TCCTGGAGAATGTGCCCACTGGCTTAGAGATGCTAAGTCTCAGGGATAAGGCTCAGGGGCATTG
CTGGCCCTCTTGATGGCCTCACCCTCTCGTAGCCGCCTGGAGAGGAAGGGTAGACTATATAGAG
GAGGTTAGGGGTAGGTCAGCATGATGCTAGGATGGAGAGAGCTCTGTCCCCTCGTGGATGGTGG
TGGTGACTCACCCGGGGGGCCAGCTGCTTTCTGAGTGCAAATGAGAAAAATAAAGAGCTGCGCT
GTGACTTTTCTTTCCACATCAAAGCTTGGGTGTCAGTGCTTTAGCTTGATGCTCTGATCACCAT
GCAAATCTTCCACCGGCGCCTTGCTCAGCTTTCATATCCCAAGGGTGCCTGGGAGGAAGGCAAC
AGGGACAGTGGACATCACTGCACCACTTTCCACGACCCTGTGTGCCAACCTCAGCCACTTTGAA
ACATGCTGATGACTGAGGTCTGTTCACTTTCTTAATTTCAAGCAGGAGAAGCAGGTTGGGGAGC
CAGCCTCCCCAGCTAGAGGGGACAGAACTTGACTTGAGCAGGGGGGTACCTCCTAGGACCTGCT
CCATGTGCCTACTTCTTTACCCTTCTCTAGAGAGGGCTCTTGTCCTGTCAGAGCTGTTTTCTCC
CTTCTCTTGTTTTTTCTTTTTCAAGACTGTTTCTCTGTGTTAGCCCTGGCTGTCCTGGATCTCA
CTCTGTAGATCAGGCTGACCTTGAGTTCAAAGCTCCATCTGCCTCTACTTCTCACATTACTGTG
ATTAAAGGCATATACTACCACTGCCTGGTGCCCTTTTGTATTTCTTATTAAAGTCCTAATGTCT
GATTATAAAAACAGTCTGTGTGGGCTGGAGTGATGGCTTACTCAGTAAAGCACTTGCCATGGAA
TCTGGGCAATCTGAGTTTCATTTTTAGCATCCTGTAAAAATCCCAATTTGATGGTGTACTTGTA
ATGTCAGCATGGAGAGGCAGAGATAGGTAAGTTCCCCAAGACTCTTTGAACCGACAGCTTGGCC
TCACTGGCACATTCCAGGTCTCAGTGAGAGACCCTGCCTCAAAATACAAAGAAAGAGCTGCTGA
AGAGTGGGTCAGAGTTGACCTCTGATCTCCGGAAGTATATGATACACACCCGTGCATGCACTCT
TCCTTACAAAATAAAAAGCAAAACAAAACCCCAACAGGTATATGGCCATTTTAGAAAAATTAGA
AGATTTAGAAAGCTATACATAAAAAAAAATGACCTAAAGAAAAATCTTTACTGTTCTGGGCACT
ATCCCTATCAAACCACTGTGTTCTTTGGCCAAGCCTTGGGGTGGACACTGTTTTGAGGTGGGTC
CTGTTATCTCCACTAGGTAGTGGAGTTTTGTGTCAGACTAACTGGGTCTTAAAGCTGTCTTTAA
GGCCATCAGGAGCTACTGACTTGCCTGCCTCAGCAGAGCATATCCTGAAGGTCGGGGTTAAGTC
TCCTTCCCGAGCGAGTTGCCTTCCAGTGGGCCCCTGGACTCCTAGGTCCTCAGCGCTCATCAGC
TGCCAAGGACTCTGAGGGAATGTCCTCTGACTGTGGCCCCGAAAGGTAGGGGAGGGGGATGTGC
TTAGGCTTAGGACAGGGTCCTGTTTCAGTCTGCCTTCACTGTTAGTAGCACTGTGCCACATGGC
ACAGACTGGGCGAGCTTTAAAGGAAGGAGGTTGATATTGGTTCCCACTTCTGGGGATCATGGTT
GAGCAGCCTTGTCTGATGATGGTTGTCTTGATGGTAGATCGTGAGGTAGTTGATGAAGGTATGA
CATGGTGAGAAACTCTGTGTGTGTGTGTTATTTTCTCTGTGTTCTACCTATACATCTATCTATG
TATATATGTATCTATCTATCTACCTGGAGGCTGGAGAGATAGCTTAGTGGTTAAGAACATTTGT
TGTTCTTGCATAGTCCTGGATTTAAATTTTCAGCACCCACATGGCAGCTCACAACAACCCATAA
ATCCAGTTTCAGAGGATCCAACCTCTGATATACCATGTCAGCCAGAGCAGACACGGCTGAAGGT
GGTTTGATCCCCGTATGGAGAGGTGACAATTGGGAAGAGAGAAAGATCAACTTAACCATGCAAG
GAACAGGAAGTTAAATACTGAACAGGGAAGGTAAAGGCAGGAAGTAGATGTAGAGGGCAAATCA
ATGAAACCCAAACATACCCAAATTACGCTAAACACACACTGACATGCCAATTAAAAGGACAAAT
TGGCTCCACTGGCAAAACCAAAACAGACACTGAAGATCCAAACAGTCACATGCCAACTACCGCG
GAGGGAGACAGACACAGAGAAGACCGTGACAGACACTTGGACACTCTTGAGAGTGGATGTGCAG
GAAGAGAGCTCTGCCAGTGGAGAAGAAAGCACTCAGAAGAAAGTGACAGCAGCTGTAAATTTGT
ATTCTGCTAATGTTATGTTCCAAAGTTGAAAGCAAAATTGTACCAATTCATAAGAACAAACAGG
CTGACTCTCAGTTGTGACTGAACGTCTCTCAGTAACTGACGGGGCGAGCAGGCCAAAGGAGAGT
CGGCTCAGAAGGGTGCATAGCCACGCCAAATCAAATAAGCAAGTACAACCGGCAGGCTCTATTT
CTAGCACAAAGGGGTCTGTGCCTCATTCTGTGCTTGGGTCAGAGCTTGGGTCTCTCATTTGGAT
GTAAGTGGTGTAGTGGAGAAGCAGGAAATAATCCGGAGCGCATATTTTGATTTTAACATAAGTG
CTGATTTGGGAGGGAGTTTTGTCAAATTGTGTTTTTACAATGTTTTTTTTTTTTTAAATGATGC
TTTTTTGTAAAGTGTACAAATGTGATATAAGATTGGTTCTGCTACATTCAGTTTCTATAAAAGT
GGTTCTAAAATATTGTACTGTCAATCATCTCATGATTATTCTACTGTACACATTACTGACTTTG
TATGTAATAATTAATATTAGAAGAAAATATAATTTATTTGAATATAAAAAAAAAAAAAAAAAAA
SEQ ID NO: 46:
X₁ASLPX₂LQX₃ESVFQSGAHAYRIPALLYLPGQQSLLAFAEQRASKKDEHAELIVLRRGDYDAX₄
THQVQWQAQEVVAQARLDGHRSMNPCPLYDX₅QTGTLFLFFIAIPGQVTEQQQLQTRANVTRLCQ
VTSTDHGRTWSSPRDLTDAAIGPAYREWSTFAVGPGHCLQLHDRARSLVVPAYAYRKLHPX₆QRP
IPSAFCFLSHDHGRTWARGHFVAQDTLECQVAEVETGEQRVVTLNARSHLRARVQAQSTNDGLD
FQESQLVKKLVEPPPX₇GCQGSVISFPSPRSGPGSPAQWLLYTHPTHX₈X₉QRADLGAYLNPRPP
APEAWSEPVLLAKGSX₁₀AYSDLQSMGTGPDGSPLFGCLYEANDYEEIX₁₁FX₁₂MFTLKQAFPAE
YLPQ
SEQ ID NO: 47:
X₁X₂SX₃X₄X₅LQX₆ESVFQSGAHAYRIPALLYLPGQQSLLAFAEQRASX₇X₈DEHAELIVX₉RRGD
YDAX₁₀THQVQWX₁₁AQEVVAQAX₁₂LDGHRSMNPCPLYDX₁₃QTGTLFLFFIAIPX₁₄X₁₅VTEX₁₆Q
QLQTRANVTRLX₁₇X₁₈VTSTDHGRTWSSPRDLTDAAIGPX₁₉YREWSTFAVGPGHX₂₀LQLHDRX₂₁
RSLVVPAYAYRKLHPX₂₂QRPIPSAFX₂₃FLSHDHGRTWARGHFVAQDTX₂₄ECQVAEVETGEQRV
VTLNARSHLRARVQAQSX₂₅NX₂₆GLDFQX₂₇SQLVKKLVEPPPX₂₈GX₂₉QGSVISFPSPRSGPGSP
AQX₃₀LLYTHPTHX₃₁X₃₂QRADLGAYLNPRPPAPEAWSEPX₃₃LLAKGSX₃₄AYSDLQSMGTGPDGS
PLFGX₃₅LYEANDYEEIX₃₆FX₃₇MFTLKQAFPAEYLPQ
SEQ ID NO: 48:
DASLPYLQKESVFQSGAHAYRIPALLYLPGQQSLLAFAEQRASKKDEHAELIVLRRGDYDAGTH
QVQWQAQEVVAQARLDGHRSMNPCPLYDEQTGTLFLFFIAIPGQVTEQQQLQTRANVTRLCQVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSTNDGLDFQ
ESQLVKKLVEPPPQGCQGSVISFPSPRSGPGSPAQWLLYTHPTHSWQRADLGAYLNPRPPAPEA
WSEPVLLAKGSAAYSDLQSMGTGPDGSPLFGCLYEANDYEEIVELMFTLKQAFPAEYLPQ
SEQ ID NO: 49:
DASLPYLQDESVFQSGAHAYRIPALLYLPGQQSLLAFAEQRASKKDEHAELIVLRRGDYDAPTH
QVQWQAQEVVAQARLDGHRSMNPCPLYDEQTGTLFLFFIAIPGQVTEQQQLQTRANVTRLCQVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSINDGLDFQ
ESQLVKKLVEPPPQGCQGSVISFPSPRSGPGSPAQWLLYTHPTHSWQRADLGAYLNPRPPAPEA
WSEPVLLAKGSAAYSDLQSMGTGPDGSPLFGCLYEANDYEEIRFIMFTLKQAFPAEYLPQ
SEQ ID NO: 50:
DASLPYLQKESVFQSGAHAYRIPALLYLPGQQSLLAFAEQRASKKDEHAELIVLRRGDYDANTH
QVQWQAQEVVAQARLDGHRSMNPCPLYDAQTGTLFLFFIAIPGQVTEQQQLQTRANVTRLCQVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSTNDGLDFQ
ESQLVKKLVEPPPQGCQGSVISFPSPRSGPGSPAQWLLYTHPTHSWQRADLGAYLNPRPPAPEA
WSEPVLLAKGSAAYSDLQSMGTGPDGSPLFGCLYEANDYEEIVELMFTLKQAFPAEYLPQ
SEQ ID NO: 51:
DASLPYLQKESVFQSGAHAYRIPALLYLPGQQSLLAFAEQRASKKDEHAELIVLRRGDYDAPTH
QVQWQAQEVVAQARLDGHRSMNPCPLYDAQTGTLFLFFIAIPGQVTEQQQLQTRANVTRLCQVT
STDHGRTWSSPRDLTDAAIGPAYREWSTFAVGPGHCLQLHDRARSLVVPAYAYRKLHPKQRPIP
SAFCFLSHDHGRTWARGHEVAQDTLECQVAEVETGEQRVVTLNARSHLRARVQAQSTNDGLDFQ
ESQLVKKLVEPPPAGCQGSVISFPSPRSGPGSPAQWLLYTHPTHRKQRADLGAYLNPRPPAPEA
WSEPVLLAKGSAAYSDLQSMGTGPDGSPLFGCLYEANDYEEIVELMFTLKQAFPAEYLPQ
SEQ ID NO: 52:
DASLPYLQKESVFQSGAHAYRIPALLYLPGQQSLLAFAEQRASKKDEHAELIVLRRGDYDASTH
QVQWQAQEVVAQARLDGHRSMNPCPLYDAQTGTLFLFFIAIPGQVTEQQQLQTRANVTRLCQVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSTNDGLDFQ
ESQLVKKLVEPPPAGCQGSVISFPSPRSGPGSPAQWLLYTHPTHRKQRADLGAYLNPRPPAPEA
WSEPVLLAKGSAAYSDLQSMGTGPDGSPLFGCLYEANDYEEIVELMFTLKQAFPAEYLPQ
SEQ ID NO: 53:
DASLPYLQKESVFQSGAHAYRIPALLYLPGQQSLLAFAEQRASKKDEHAELIVLRRGDYDATTH
QVQWQAQEVVAQARLDGHRSMNPCPLYDAQTGTLFLFFIAIPGQVTEQQQLQTRANVTRLCQVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSTNDGLDFQ
ESQLVKKLVEPPPAGCQGSVISFPSPRSGPGSPAQWLLYTHPTHRKQRADLGAYLNPRPPAPEA
WSEPVLLAKGSAAYSDLQSMGTGPDGSPLFGCLYEANDYEEIVELMFTLKQAFPAEYLPQ
SEQ ID NO: 54:
DASLPYLQKESVFQSGAHAYRIPALLYLPGQQSLLAFAEQRASKKDEHAELIVLRRGDYDANTH
QVQWQAQEVVAQARLDGHRSMNPCPLYDAQTGTLFLFFIAIPGQVTEQQQLQTRANVTRLCQVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSINDGLDFQ
ESQLVKKLVEPPPAGCQGSVISFPSPRSGPGSPAQWLLYTHPTHRKQRADLGAYLNPRPPAPEA
WSEPVLLAKGSAAYSDLQSMGTGPDGSPLFGCLYEANDYEEIVELMFTLKQAFPAEYLPQ
SEQ ID NO: 55:
ASLPYLQKESVFQSGAHAYRIPALLYLPGQQSLLAFAEQRASKKDEHAELIVLRRGDYDAPTHQ
VQWQAQEVVAQARLDGHRSMNPCPLYDAQTGTLFLFFIAIPGQVTEQQQLQTRANVTRLCQVTS
TDHGRTWSSPRDLTDAAIGPAYREWSTFAVGPGHCLQLHDRARSLVVPAYAYRKLHPKQRPIPS
AFCFLSHDHGRTWARGHEVAQDTLECQVAEVETGEQRVVTLNARSHLRARVQAQSTNDGLDEQE
SQLVKKLVEPPPQGCQGSVISFPSPRSGPGSPAQWLLYTHPTHSWQRADLGAYLNPRPPAPEAW
SEPVLLAKGSCAYSDLQSMGTGPDGSPLFGCLYEANDYEEIVELMFTLKQAFPAEYLPQ
SEQ ID NO: 56:
MASLPYLQKESVFQSGAHAYRIPALLYLPGQQSLLAFAEQRASKKDEHAELIVLRRGDYDAPTH
QVQWQAQEVVAQARLDGHRSMNPCPLYDAQTGTLFLFFIAIPGQVTEQQQLQTRANVTRLCQVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSINDGLDFQ
ESQLVKKLVEPPPQGCQGSVISFPSPRSGPGSPAQWLLYTHPTHSWQRADLGAYLNPRPPAPEA
WSEPVLLAKGSCAYSDLQSMGTGPDGSPLFGCLYEANDYEEIVELMFTLKQAFPAEYLPQ
SEQ ID NO: 57:
ASLPYLQKESVFQSGAHAYRIPALLYLPGQQSLLAFAEQRASKKDEHAELIVLRRGDYDAPTHQ
VQWQAQEVVAQARLDGHRSMNPCPLYDAQTGTLFLFFIAIPGQVTEQQQLQTRANVTRLCQVTS
TDHGRTWSSPRDLTDAAIGPAYREWSTFAVGPGHCLQLHDRARSLVVPAYAYRKLHPKQRPIPS
AFCFLSHDHGRTWARGHEVAQDTLECQVAEVETGEQRVVTLNARSHLRARVQAQSTNDGLDEQE
SQLVKKLVEPPPQGCQGSVISFPSPRSGPGSPAQWLLYTHPTHSWQRADLGAYLNPRPPAPEAW
SEPVLLAKGSAAYSDLQSMGTGPDGSPLFGCLYEANDYEEIVELMFTLKQAFPAEYLPQ
SEQ ID NO: 58:
DASLPYLQKESVFQSGAHAYRIPALLYLPGQQSLLAFAEQRASKKDEHAELIVLRRGDYDAPTH
QVQWQAQEVVAQARLDGHRSMNPCPLYDAQTGTLFLFFIAIPGQVTEQQQLQTRANVTRLCQVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSINDGLDFQ
ESQLVKKLVEPPPQGCQGSVISFPSPRSGPGSPAQWLLYTHPTHSWQRADLGAYLNPRPPAPEA
WSEPVLLAKGSAAYSDLQSMGTGPDGSPLFGCLYEANDYEEIVELMFTLKQAFPAEYLPQ
SEQ ID NO: 59:
DASLPYLQKESVFQSGAHAYRIPALLYLPGQQSLLAFAEQRASKKDEHAELIVLRRGDYDAPTH
QVQWQAQEVVAQARLDGHRSMNPCPLYDAQTGTLFLFFIAIPGQVTEQQQLQTRANVTRLCQVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSTNDGLDFQ
ESQLVKKLVEPPPQGCQGSVISFPSPRSGPGSPAQWLLYTHPTHSWQRADLGAYLNPRPPAPEA
WSEPVLLAKGSCAYSDLQSMGTGPDGSPLFGCLYEANDYEEIVELMFTLKQAFPAEYLPQ
SEQ ID NO: 60:
AASLPYLQKESVFQSGAHAYRIPALLYLPGQQSLLAFAEQRASKKDEHAELIVLRRGDYDAPTH
QVQWQAQEVVAQARLDGHRSMNPCPLYDAQTGTLFLFFIAIPGQVTEQQQLQTRANVTRLCQVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSTNDGLDFQ
ESQLVKKLVEPPPQGCQGSVISFPSPRSGPGSPAQWLLYTHPTHSWQRADLGAYLNPRPPAPEA
WSEPVLLAKGSAAYSDLQSMGTGPDGSPLFGCLYEANDYEEIVELMFTLKQAFPAEYLPQ
SEQ ID NO: 61:
MASLPYLQKESVFQSGAHAYRIPALLYLPGQQSLLAFAEQRASKKDEHAELIVLRRGDYDAPTH
QVQWQAQEVVAQARLDGHRSMNPCPLYDAQTGTLFLFFIAIPGQVTEQQQLQTRANVTRLCQVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSTNDGLDFQ
ESQLVKKLVEPPPQGCQGSVISFPSPRSGPGSPAQWLLYTHPTHSWQRADLGAYLNPRPPAPEA
WSEPVLLAKGSAAYSDLQSMGTGPDGSPLFGCLYEANDYEEIVELMFTLKQAFPAEYLPQ
SEQ ID NO: 62:
AASLPYLQKESVFQSGAHAYRIPALLYLPGQQSLLAFAEQRASKKDEHAELIVLRRGDYDAPTH
QVQWQAQEVVAQARLDGHRSMNPCPLYDAQTGTLFLFFIAIPGQVTEQQQLQTRANVTRLCQVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSTNDGLDFQ
ESQLVKKLVEPPPQGCQGSVISFPSPRSGPGSPAQWLLYTHPTHSWQRADLGAYLNPRPPAPEA
WSEPVLLAKGSCAYSDLQSMGTGPDGSPLFGCLYEANDYEEIVELMFTLKQAFPAEYLPQ
SEQ ID NO: 63:
DASLPYLQKESVFQSGAHAYRIPALLYLPGQQSLLAFAEQRRSKKDEHAELIVLRRGDYDAGTH
QVQWQAQEVVAQARLDGHRSMNPCPLYDEQTGTLFLFFIAIPGQVTEQQQLQTRANVTRLCYVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRERVQAQSTNDGLDFQ
ESQLVKKLVEPPPTGCQGSVISFPSPRSGPGSPAQWLLYTHPTHSWQRADLGAYLNPRPPAPEA
WSEPVLLAKGSAAYSDLQSMGTGPDGSPLFGCLYEANDYEEIVELMFTLKQAFPAEYLPQ
SEQ ID NO: 64:
X₁X₂SX₃X₄X₅LQX₆ESVFQSGAHAYRIPALLYLPGQQSLLAFAEQRX₇SX₈X₉DEHAELIVX₁₀RRG
DYDAX₁₁THQVQWX₁₂AQEVVAQAX₁₃LX₁₄GHRSMNPCPLYDX₁₅QTGTLFLFFIAIPX₁₆X₁₇VTE
X₁₈QQLQTRANVTRLX₁₉X₂₀VTSTDHGRTWSSPRDLTDAAIGPX₂₁YREWSTFAVGPGHX₂₂LQLHD
X₂₃X₂₄RSLVVPAYAYRKLHPX₂₅X₂₆X₂₇PIPSAFX₂₈FLSHDHGRTWARGHFVX₂₉QDTX₃₀ECQVAEV
X₃₁TGEQRVVTLNARSX₃₂X₃₃X₃₄X₃₅RX₃₆QAQSX₃₇NX₃₈GLDFQX₃₉X₄₀QX₄₁VKKLX₄₂EPPPX₄₃G
X₄₄QGSVISFPSPRSGPGSPAQX₄₅LLYTHPTHX₄₆X₄₇QRADLGAYLNPRPPAPEAWSEPX₄₈LLAK
GSX₄₉AYSDLQSMGTGPDGSPLFGX₅₀LYEANDYEEIX₅₁FX₅₂MFTLKQAFPAEYLPQ
SEQ ID NO: 65:
X₁ASLPX₂LQX₃ESVFQSGAHAYRIPALLYLPGQQSLLAFAEQRX₄SKKDEHAELIVLRRGDYDAX₅THQVQWQAQEVV
AQARLDGHRSMNPCPLYDX₆QTGTLFLFFIAIPGQVTEQQQLQTRANVTRLCX₇VTSTDHGRTWSSPRDLTDAAIGPA
YREWSTFAVGPGHCLQLHDRARSLVVPAYAYRKLHPX₈QRPIPSAFCFLSHDHGRTWARGHFVAQDTLECQVAEVETG
EQRVVTLNARSHLRX₉RVQAQSTNDGLDFQESQLVKKLVEPPPX₁₀GCQGSVISFPSPRSGPGSPAQWLLYTHPTHX₁₁
X₁₂QRADLGAYLNPRPPAPEAWSEPVLLAKGSX₁₃AYSDLQSMGTGPDGSPLFGCLYEANDYEEIX₁₄FX₁₅MFTLKQAF
PAEYLPQ
SEQ ID NO: 66:
X₁X₂SX₃X₄X₅LQX₆ESVFQSGAHAYRIPALLYLPGQQSLLAFAEQRX₇SX₈X₉DEHAELIVX₁₀RRG
DYDAX₁₁THQVQWX₁₂AQEVVAQAX₁₃LX₁₄GHRSMNPCPLYDX₁₅QTGTLFLFFIAIPX₁₆X₁₇VTE
X₁₈QQLQTRANVTRLX₁₉X₂₀VTSTDHGRTWSSPRDLTDAAIGPX₂₁YREWSTFAVGPGHX₂₂LQLHD
X₂₃X₂₄RSLVVPAYAYRKLHPX₂₅X₂₆X₂₇PIPSAFX₂₈FLSHDHGRTWARGHFVX₂₉QDTX₃₀ECQVAEV
X₃₁TGEQRVVTLNARSX₃₂X₃₃X₃₄X₃₅RX₃₆QAQSX₃₇NX₃₈GLDFQX₃₉X₄₀QX₄₁VKKLX₄₂EPPPX₄₃G
X₄₄QGSVISFPSPRSGPGSPAQX₄₅LLYTHPTHX₄₆X₄₇QRADLGAYLNPRPPAPEAWSEPX₄₈LLAK
GSX₄₉AYSDLQSMGTGPDGSPLFGX₅₀LYEANDYEEIX₅₁FX₅₂MFTLKQAFPAEYLPQX₅₃DKTHTC
PPCPAPELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPS
REEMTKNQVSLTCLVKGFYPSDIAVEWESNGQPENNYKTTPPVLDSDGSFFLTSKLTVDKSRWQ
QGNVFSCSVMHEALHNHYTQKSLSLSPGK
SEQ ID NO: 67:
DASLPYLQKESVFQSGAHAYRIPALLYLPGQQSLLAFAEQRASKKDEHAELIVLRRGDYDAGTH
QVQWQAQEVVAQARLDGHRSMNPCPLYDEQTGTLFLFFIAIPGQVTEQQQLQTRANVTRLCQVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSTNDGLDFQ
ESQLVKKLVEPPPQGCQGSVISFPSPRSGPGSPAQWLLYTHPTHSWQRADLGAYLNPRPPAPEA
WSEPVLLAKGSAAYSDLQSMGTGPDGSPLFGCLYEANDYEEIVELMFTLKQAFPAEYLPQGGGG
SGGGGSDKTHTCPPCPAPELLGGPSVFLFPPKPKDTLMISRTPEVTCVVVDVSHEDPEVKENWY
VDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFEL
TSKLTVDKSRWQQGNVFSCSVMHEALHNHYTQKSLSLSPGK
SEQ ID NO: 68:
DASLPYLQDESVFQSGAHAYRIPALLYLPGQQSLLAFAEQRASKKDEHAELIVLRRGDYDAPTH
QVQWQAQEVVAQARLDGHRSMNPCPLYDEQTGTLFLFFIAIPGQVTEQQQLQTRANVTRLCQVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSTNDGLDFQ
ESQLVKKLVEPPPQGCQGSVISFPSPRSGPGSPAQWLLYTHPTHSWQRADLGAYLNPRPPAPEA
WSEPVLLAKGSAAYSDLQSMGTGPDGSPLFGCLYEANDYEEIRFIMFTLKQAFPAEYLPQGGGG
SGGGGSDKTHTCPPCPAPELLGGPSVFLFPPKPKDTLMISRTPEVTCVVVDVSHEDPEVKENWY
VDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFEL
TSKLTVDKSRWQQGNVFSCSVMHEALHNHYTQKSLSLSPGK
SEQ ID NO: 69:
DASLPYLQKESVFQSGAHAYRIPALLYLPGQQSLLAFAEQRASKKDEHAELIVLRRGDYDANTH
QVQWQAQEVVAQARLDGHRSMNPCPLYDAQTGTLFLFFIAIPGQVTEQQQLQTRANVTRLCQVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSTNDGLDFQ
ESQLVKKLVEPPPQGCQGSVISFPSPRSGPGSPAQWLLYTHPTHSWQRADLGAYLNPRPPAPEA
WSEPVLLAKGSAAYSDLQSMGTGPDGSPLFGCLYEANDYEEIVELMFTLKQAFPAEYLPQGGGG
SGGGGSDKTHTCPPCPAPELLGGPSVFLFPPKPKDTLMISRTPEVTCVVVDVSHEDPEVKENWY
VDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFEL
TSKLTVDKSRWQQGNVFSCSVMHEALHNHYTQKSLSLSPGK
SEQ ID NO: 70:
DASLPYLQKESVFQSGAHAYRIPALLYLPGQQSLLAFAEQRASKKDEHAELIVLRRGDYDAPTH
QVQWQAQEVVAQARLDGHRSMNPCPLYDAQTGTLFLFFIAIPGQVTEQQQLQTRANVTRLCQVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSTNDGLDFQ
ESQLVKKLVEPPPAGCQGSVISFPSPRSGPGSPAQWLLYTHPTHRKQRADLGAYLNPRPPAPEA
WSEPVLLAKGSAAYSDLQSMGTGPDGSPLFGCLYEANDYEEIVELMFTLKQAFPAEYLPQGGGG
SGGGGSDKTHTCPPCPAPELLGGPSVFLFPPKPKDTLMISRTPEVTCVVVDVSHEDPEVKENWY
VDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFFL
TSKLTVDKSRWQQGNVFSCSVMHEALHNHYTQKSLSLSPGK
SEQ ID NO: 71:
DASLPYLQKESVFQSGAHAYRIPALLYLPGQQSLLAFAEQRASKKDEHAELIVLRRGDYDASTH
QVQWQAQEVVAQARLDGHRSMNPCPLYDAQTGTLFLFFIAIPGQVTEQQQLQTRANVTRLCQVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSTNDGLDFQ
ESQLVKKLVEPPPAGCQGSVISFPSPRSGPGSPAQWLLYTHPTHRKQRADLGAYLNPRPPAPEA
WSEPVLLAKGSAAYSDLQSMGTGPDGSPLFGCLYEANDYEEIVELMFTLKQAFPAEYLPQGGGG
SGGGGSDKTHTCPPCPAPELLGGPSVFLFPPKPKDTLMISRTPEVTCVVVDVSHEDPEVKENWY
VDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFEL
TSKLTVDKSRWQQGNVFSCSVMHEALHNHYTQKSLSLSPGK
SEQ ID NO: 72:
DASLPYLQKESVFQSGAHAYRIPALLYLPGQQSLLAFAEQRASKKDEHAELIVLRRGDYDATTH
QVQWQAQEVVAQARLDGHRSMNPCPLYDAQTGTLFLFFIAIPGQVTEQQQLQTRANVTRLCQVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSINDGLDFQ
ESQLVKKLVEPPPAGCQGSVISFPSPRSGPGSPAQWLLYTHPTHRKQRADLGAYLNPRPPAPEA
WSEPVLLAKGSAAYSDLQSMGTGPDGSPLFGCLYEANDYEEIVELMFTLKQAFPAEYLPQGGGG
SGGGGSDKTHTCPPCPAPELLGGPSVFLFPPKPKDTLMISRTPEVTCVVVDVSHEDPEVKENWY
VDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFEL
TSKLTVDKSRWQQGNVFSCSVMHEALHNHYTQKSLSLSPGK
SEQ ID NO: 73:
DASLPYLQKESVFQSGAHAYRIPALLYLPGQQSLLAFAEQRASKKDEHAELIVLRRGDYDANTH
QVQWQAQEVVAQARLDGHRSMNPCPLYDAQTGTLFLFFIAIPGQVTEQQQLQTRANVTRLCQVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSINDGLDFQ
ESQLVKKLVEPPPAGCQGSVISFPSPRSGPGSPAQWLLYTHPTHRKQRADLGAYLNPRPPAPEA
WSEPVLLAKGSAAYSDLQSMGTGPDGSPLFGCLYEANDYEEIVELMFTLKQAFPAEYLPQGGGG
SGGGGSDKTHTCPPCPAPELLGGPSVELFPPKPKDTLMISRTPEVTCVVVDVSHEDPEVKENWY
VDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFEL
TSKLTVDKSRWQQGNVFSCSVMHEALHNHYTQKSLSLSPGK
SEQ ID NO: 74:
X₁ASLPX₂LQX₃ESVFQSGAHAYRIPALLYLPGQQSLLAFAEQRASKKDEHAELIVLRRGDYDAX₄
THQVQWQAQEVVAQARLDGHRSMNPCPLYDX₅QTGTLFLFFIAIPGQVTEQQQLQTRANVTRLCQ
VTSTDHGRTWSSPRDLTDAAIGPAYREWSTFAVGPGHCLQLHDRARSLVVPAYAYRKLHPX₆QRP
IPSAFCFLSHDHGRTWARGHFVAQDTLECQVAEVETGEQRVVTLNARSHLRARVQAQSTNDGLD
FQESQLVKKLVEPPPX₇GCQGSVISFPSPRSGPGSPAQWLLYTHPTHX₈X₉QRADLGAYLNPRPP
APEAWSEPVLLAKGSX₁₀AYSDLQSMGTGPDGSPLFGCLYEANDYEEIX₁₁FX₁₂MFTLKQAFPAE
YLPQGGGGSGGGGSDKTHTCPPCPAPELLGGPSVFLFPPKPKDTLMISRTPEVTCVVVDVSHED
PEVKENWYVDGVEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPENNYKTTPPVL
DSDGSFFLTSKLTVDKSRWQQGNVFSCSVMHEALHNHYTQKSLSLSPGK
SEQ ID NO: 75:
X₁X₂SX₃X₄X₅LQX₆ESVFQSGAHAYRIPALLYLPGQQSLLAFAEQRASX₇X₈DEHAELIVX₉RRGD
YDAX₁₀THQVQWX₁₁AQEVVAQAX₁₂LDGHRSMNPCPLYDX₁₃QTGTLFLFFIAIPX₁₄X₁₅VTEX₁₆Q
QLQTRANVTRLX₁₇X₁₈VTSTDHGRTWSSPRDLTDAAIGPX₁₉YREWSTFAVGPGHX₂₀LQLHDRX₂₁
RSLVVPAYAYRKLHPX₂₂QRPIPSAFX₂₃FLSHDHGRTWARGHFVAQDTX₂₄ECQVAEVETGEQRV
VTLNARSHLRARVQAQSX₂₅NX₂₆GLDFQX₂₇SQLVKKLVEPPPX₂₈GX₂₉QGSVISFPSPRSGPGSP
AQX₃₀LLYTHPTHX₃₁X₃₂QRADLGAYLNPRPPAPEAWSEPX₃₃LLAKGSX₃₄AYSDLQSMGTGPDGS
PLFGX₃₅LYEANDYEEIX₃₆FX₃₇MFTLKQAFPAEYLPQGGGGSGGGGSDKTHTCPPCPAPELLGG
PSVFLFPPKPKDTLMISRTPEVTCVVVDVSHEDPEVKENWYVDGVEVHNAKTKPREEQYNSTYR
VVSVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLTSKLTVDKSRWQQGNVFSCSVMH
EALHNHYTQKSLSLSPGK
SEQ ID NO: 76:
X₁ASLPX₂LQX₃ESVFQSGAHAYRIPALLYLPGQQSLLAFAEQRX₄SKKDEHAELIVLRRGDYDA
X₅THQVQWQAQEVVAQARLDGHRSMNPCPLYDX₆QTGTLFLFFIAIPGQVTEQQQLQTRANVTRLC
X₇VTSTDHGRTWSSPRDLTDAAIGPAYREWSTFAVGPGHCLQLHDRARSLVVPAYAYRKLHPX₈Q
RPIPSAFCFLSHDHGRTWARGHFVAQDTLECQVAEVETGEQRVVTLNARSHLRX₉RVQAQSTNDG
LDFQESQLVKKLVEPPPX₁₀GCQGSVISFPSPRSGPGSPAQWLLYTHPTHX₁₁X₁₂QRADLGAYLN
PRPPAPEAWSEPVLLAKGSX₁₃AYSDLQSMGTGPDGSPLFGCLYEANDYEEIX₁₄FX₁₅MFTLKQA
FPAEYLPQX₁₆DKTHTCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPVLDSDG
SFFLTSKLTVDKSRWQQGNVFSCSVMHEALHNHYTQKSLSLSPGK
SEQ ID NO: 77:
EIVLTQSPATLSLSPGERATLSCRASKGVSTSGYSYLHWYQQKPGQAPRLLIYLASYLESGVPA
RFSGSGSGTDFTLTISSLEPEDFAVYYCQHSRDLPLTFGGGTKVEIKRTVAAPSVFIFPPSDEQ
LKSGTASVVCLLNNFYPREAKVQWKVDNALQSGNSQESVTEQDSKDSTYSLSSTLTLSKADYEK
HKVYACEVTHQGLSSPVTKSENRGEC
SEQ ID NO: 78:
DASLPYLQKESVFQSGAHAYRIPALLYLPGQQSLLAFAEQRASKKDEHAELIVLRRGDYDAGTH
QVQWQAQEVVAQARLDGHRSMNPCPLYDEQTGTLFLFFIAIPGQVTEQQQLQTRANVTRLCQVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSINDGLDFQ
ESQLVKKLVEPPPQGCQGSVISFPSPRSGPGSPAQWLLYTHPTHSWQRADLGAYLNPRPPAPEA
WSEPVLLAKGSAAYSDLQSMGTGPDGSPLFGCLYEANDYEEIVELMFTLKQAFPAEYLPQGGGG
SDKTHTCPPCPAPELLGGPSVFLFPPKPKDTLMISRTPEVTCVVVDVSHEDPEVKENWYVDGVE
VHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFELTSKLT
VDKSRWQQGNVFSCSVMHEALHNHYTQKSLSLSPGK
SEQ ID NO: 79:
GATGCATCTCTGCCTTACCTGCAGAAAGAAAGCGTGTTCCAGTCTGGCGCCCACGCCTACAGAA
TTCCCGCTCTGCTGTATCTGCCAGGCCAGCAGTCTCTGCTGGCTTTCGCTGAACAGCGGGCCAG
CAAGAAGGATGAGCACGCCGAACTGATCGTGCTGCGGAGAGGCGATTACGACGCCGGCACACAT
CAGGTGCAGTGGCAGGCTCAAGAGGTGGTGGCTCAGGCTAGACTGGACGGCCACAGATCTATGA
ACCCCTGTCCTCTGTACGATGAACAGACCGGCACACTGTTTCTGTTCTTTATCGCTATCCCCGG
CCAAGTGACCGAGCAGCAGCAGCTGCAGACAAGAGCCAACGTGACCAGACTGTGTCAAGTGACC
TCCACCGACCACGGCAGAACCTGGTCTAGCCCTAGAGATCTGACCGACGCCGCCATCGGACCTG
CCTATAGAGAGTGGTCCACCTTCGCCGTTGGACCTGGACACTGTCTCCAGCTGCACGACAGGGC
TAGATCTCTGGTGGTGCCTGCCTACGCCTATAGAAAGCTGCACCCCAAACAGCGGCCTATTCCT
AGCGCCTTCTGCTTTCTGAGCCACGATCACGGCAGGACATGGGCCAGAGGACATTTCGTGGCCC
AGGACACACTGGAATGCCAGGTGGCCGAAGTGGAAACCGGCGAGCAGAGAGTCGTGACCCTGAA
CGCCAGATCTCACCTGAGAGCCAGAGTGCAGGCCCAGAGCACAAACGACGGCCTGGATTTCCAA
GAGAGCCAGCTGGTCAAGAAACTGGTGGAACCTCCTCCACAGGGCTGTCAGGGAAGCGTGATCA
GCTTTCCATCTCCTAGAAGCGGCCCTGGCTCTCCTGCTCAGTGGCTGCTGTATACACACCCCAC
ACACAGCTGGCAGAGAGCCGATCTGGGCGCCTACCTGAATCCTAGACCTCCTGCTCCTGAGGCT
TGGAGCGAACCTGTTCTGCTGGCCAAGGGCAGCGCTGCCTACAGCGATCTGCAGTCTATGGGCA
CAGGCCCTGATGGCAGCCCTCTGTTTGGCTGTCTGTACGAGGCCAACGACTACGAAGAGATCGT
GTTCCTGATGTTCACCCTGAAGCAGGCCTTTCCAGCCGAGTACCTGCCTCAAGGCGGAGGCGGA
TCCGACAAAACTCACACATGCCCACCGTGCCCAGCACCTGAACTCCTGGGGGGACCGTCAGTCT
TCCTCTTCCCCCCAAAACCCAAGGACACCCTCATGATCTCCCGGACCCCTGAGGTCACATGCGT
GGTGGTGGACGTGAGCCACGAAGACCCTGAGGTCAAGTTCAACTGGTACGTGGACGGCGTGGAG
GTGCATAATGCCAAGACAAAGCCGCGGGAGGAGCAGTACAACAGCACGTACCGTGTGGTCAGCG
TCCTCACCGTCCTGCACCAGGACTGGCTGAATGGCAAGGAGTACAAGTGCAAGGTCTCCAACAA
AGCCCTCCCAGCCCCCATCGAGAAAACCATCTCCAAAGCCAAAGGGCAGCCCCGAGAACCACAG
GTCTACACCCTGCCCCCATCCCGGGAGGAGATGACCAAGAACCAGGTCAGCCTGACCTGCCTGG
TCAAAGGCTTCTATCCCAGCGACATCGCCGTGGAGTGGGAGAGCAATGGGCAGCCGGAGAACAA
CTACAAGACCACGCCTCCCGTGCTGGACTCCGACGGCTCCTTCTTCCTCACTAGCAAGCTCACC
GTGGACAAGAGCAGGTGGCAGCAGGGGAACGTCTTCTCATGCTCCGTGATGCATGAGGCTCTGC
ACAACCACTACACGCAGAAGAGCCTCTCCCTGTCTCCGGGTAAA
SEQ ID NO: 80:
GATGCATCTCTGCCTTACCTGCAGAAAGAAAGCGTGTTCCAGTCTGGCGCCCACGCCTACAGAA
TTCCCGCTCTGCTGTATCTGCCAGGCCAGCAGTCTCTGCTGGCTTTCGCTGAACAGCGGGCCAG
CAAGAAGGATGAGCACGCCGAACTGATCGTGCTGCGGAGAGGCGATTACGACGCCGGCACACAT
CAGGTGCAGTGGCAGGCTCAAGAGGTGGTGGCTCAGGCTAGACTGGACGGCCACAGATCTATGA
ACCCCTGTCCTCTGTACGATGAACAGACCGGCACACTGTTTCTGTTCTTTATCGCTATCCCCGG
CCAAGTGACCGAGCAGCAGCAGCTGCAGACAAGAGCCAACGTGACCAGACTGTGTCAAGTGACC
TCCACCGACCACGGCAGAACCTGGTCTAGCCCTAGAGATCTGACCGACGCCGCCATCGGACCTG
CCTATAGAGAGTGGTCCACCTTCGCCGTTGGACCTGGACACTGTCTCCAGCTGCACGACAGGGC
TAGATCTCTGGTGGTGCCTGCCTACGCCTATAGAAAGCTGCACCCCAAACAGCGGCCTATTCCT
AGCGCCTTCTGCTTTCTGAGCCACGATCACGGCAGGACATGGGCCAGAGGACATTTCGTGGCCC
AGGACACACTGGAATGCCAGGTGGCCGAAGTGGAAACCGGCGAGCAGAGAGTCGTGACCCTGAA
CGCCAGATCTCACCTGAGAGCCAGAGTGCAGGCCCAGAGCACAAACGACGGCCTGGATTTCCAA
GAGAGCCAGCTGGTCAAGAAACTGGTGGAACCTCCTCCACAGGGCTGTCAGGGAAGCGTGATCA
GCTTTCCATCTCCTAGAAGCGGCCCTGGCTCTCCTGCTCAGTGGCTGCTGTATACACACCCCAC
ACACAGCTGGCAGAGAGCCGATCTGGGCGCCTACCTGAATCCTAGACCTCCTGCTCCTGAGGCT
TGGAGCGAACCTGTTCTGCTGGCCAAGGGCAGCGCTGCCTACAGCGATCTGCAGTCTATGGGCA
CAGGCCCTGATGGCAGCCCTCTGTTTGGCTGTCTGTACGAGGCCAACGACTACGAAGAGATCGT
GTTCCTGATGTTCACCCTGAAGCAGGCCTTTCCAGCCGAGTACCTGCCTCAA
SEQ ID NO: 81:
DASLPYLQKESVFQSGAHAYRIPALLYLPGQQSLLAFAEQRASKKDEHAELIVLRRGDYDAGTH
QVQWQAQEVVAQARLDGHRSMNPCPLYDEQTGTLFLFFIAIPGQVTEQQQLQTRANVTRLCQVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSTNDGLDFQ
ESQLVKKLVEPPPQGCQGSVISFPSPRSGPGSPAQWLLYTHPTHSWQRADLGAYLNPRPPAPEA
WSEPVLLAKGSAAYSDLQSMGTGPDGSPLFGCLYEANDYEEIVELMFTLKQAFPAEYLPQEPKS
SDKTHTCPPCPAPELLGGPSVFLFPPKPKDTLMISRTPEVTCVVVDVSHEDPEVKENWYVDGVE
VHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFELTSKLT
VDKSRWQQGNVFSCSVMHEALHNHYTQKSLSLSPGK
SEQ ID NO: 82:
DASLPYLQDESVFQSGAHAYRIPALLYLPGQQSLLAFAEQRASKKDEHAELIVLRRGDYDAPTH
QVQWQAQEVVAQARLDGHRSMNPCPLYDEQTGTLFLFFIAIPGQVTEQQQLQTRANVTRLCQVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSINDGLDFQ
ESQLVKKLVEPPPQGCQGSVISFPSPRSGPGSPAQWLLYTHPTHSWQRADLGAYLNPRPPAPEA
WSEPVLLAKGSAAYSDLQSMGTGPDGSPLFGCLYEANDYEEIRFIMFTLKQAFPAEYLPQEPKS
SDKTHTCPPCPAPELLGGPSVFLFPPKPKDTLMISRTPEVTCVVVDVSHEDPEVKENWYVDGVE
VHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFELTSKLT
VDKSRWQQGNVFSCSVMHEALHNHYTQKSLSLSPGK
SEQ ID NO: 83:
DASLPYLQKESVFQSGAHAYRIPALLYLPGQQSLLAFAEQRASKKDEHAELIVLRRGDYDANTH
QVQWQAQEVVAQARLDGHRSMNPCPLYDAQTGTLFLFFIAIPGQVTEQQQLQTRANVTRLCQVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSTNDGLDFQ
ESQLVKKLVEPPPQGCQGSVISFPSPRSGPGSPAQWLLYTHPTHSWQRADLGAYLNPRPPAPEA
WSEPVLLAKGSAAYSDLQSMGTGPDGSPLFGCLYEANDYEEIVELMFTLKQAFPAEYLPQEPKS
SDKTHTCPPCPAPELLGGPSVFLFPPKPKDTLMISRTPEVTCVVVDVSHEDPEVKENWYVDGVE
VHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFELTSKLT
VDKSRWQQGNVFSCSVMHEALHNHYTQKSLSLSPGK
SEQ ID NO: 84:
DASLPYLQKESVFQSGAHAYRIPALLYLPGQQSLLAFAEQRASKKDEHAELIVLRRGDYDAPTH
QVQWQAQEVVAQARLDGHRSMNPCPLYDAQTGTLFLFFIAIPGQVTEQQQLQTRANVTRLCQVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSTNDGLDFQ
ESQLVKKLVEPPPAGCQGSVISFPSPRSGPGSPAQWLLYTHPTHRKQRADLGAYLNPRPPAPEA
WSEPVLLAKGSAAYSDLQSMGTGPDGSPLFGCLYEANDYEEIVELMFTLKQAFPAEYLPQEPKS
SDKTHTCPPCPAPELLGGPSVFLFPPKPKDTLMISRTPEVTCVVVDVSHEDPEVKENWYVDGVE
VHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLTSKLT
VDKSRWQQGNVFSCSVMHEALHNHYTQKSLSLSPGK
SEQ ID NO: 85:
DASLPYLQKESVFQSGAHAYRIPALLYLPGQQSLLAFAEQRASKKDEHAELIVLRRGDYDASTH
QVQWQAQEVVAQARLDGHRSMNPCPLYDAQTGTLFLFFIAIPGQVTEQQQLQTRANVTRLCQVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSTNDGLDFQ
ESQLVKKLVEPPPAGCQGSVISFPSPRSGPGSPAQWLLYTHPTHRKQRADLGAYLNPRPPAPEA
WSEPVLLAKGSAAYSDLQSMGTGPDGSPLFGCLYEANDYEEIVELMFTLKQAFPAEYLPQEPKS
SDKTHTCPPCPAPELLGGPSVELFPPKPKDTLMISRTPEVTCVVVDVSHEDPEVKENWYVDGVE
VHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFELTSKLT
VDKSRWQQGNVFSCSVMHEALHNHYTQKSLSLSPGK
SEQ ID NO: 86:
DASLPYLQKESVFQSGAHAYRIPALLYLPGQQSLLAFAEQRASKKDEHAELIVLRRGDYDATTH
QVQWQAQEVVAQARLDGHRSMNPCPLYDAQTGTLFLFFIAIPGQVTEQQQLQTRANVTRLCQVT
STDHGRTWSSPRDLTDAAIGPAYREWSTFAVGPGHCLQLHDRARSLVVPAYAYRKLHPKQRPIP
SAFCFLSHDHGRTWARGHEVAQDTLECQVAEVETGEQRVVTLNARSHLRARVQAQSTNDGLDFQ
ESQLVKKLVEPPPAGCQGSVISFPSPRSGPGSPAQWLLYTHPTHRKQRADLGAYLNPRPPAPEA
WSEPVLLAKGSAAYSDLQSMGTGPDGSPLFGCLYEANDYEEIVELMFTLKQAFPAEYLPQEPKS
SDKTHTCPPCPAPELLGGPSVFLFPPKPKDTLMISRTPEVTCVVVDVSHEDPEVKENWYVDGVE
VHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFELTSKLT
VDKSRWQQGNVFSCSVMHEALHNHYTQKSLSLSPGK
SEQ ID NO: 87:
DASLPYLQKESVFQSGAHAYRIPALLYLPGQQSLLAFAEQRASKKDEHAELIVLRRGDYDANTH
QVQWQAQEVVAQARLDGHRSMNPCPLYDAQTGTLFLFFIAIPGQVTEQQQLQTRANVTRLCQVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSINDGLDFQ
ESQLVKKLVEPPPAGCQGSVISFPSPRSGPGSPAQWLLYTHPTHRKQRADLGAYLNPRPPAPEA
WSEPVLLAKGSAAYSDLQSMGTGPDGSPLFGCLYEANDYEEIVELMFTLKQAFPAEYLPQEPKS
SDKTHTCPPCPAPELLGGPSVFLFPPKPKDTLMISRTPEVTCVVVDVSHEDPEVKENWYVDGVE
VHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFELTSKLT
VDKSRWQQGNVFSCSVMHEALHNHYTQKSLSLSPGK
SEQ ID NO: 88:
X₁ASLPX₂LQX₃ESVFQSGAHAYRIPALLYLPGQQSLLAFAEQRASKKDEHAELIVLRRGDYDAX₄
THQVQWQAQEVVAQARLDGHRSMNPCPLYDX₅QTGTLFLFFIAIPGQVTEQQQLQTRANVTRLCQ
VTSTDHGRTWSSPRDLTDAAIGPAYREWSTFAVGPGHCLQLHDRARSLVVPAYAYRKLHPX₆QRP
IPSAFCFLSHDHGRTWARGHFVAQDTLECQVAEVETGEQRVVTLNARSHLRARVQAQSTNDGLD
FQESQLVKKLVEPPPX₇GCQGSVISFPSPRSGPGSPAQWLLYTHPTHX₈X₉QRADLGAYLNPRPP
APEAWSEPVLLAKGSX₁₀AYSDLQSMGTGPDGSPLFGCLYEANDYEEIX₁₁FX₁₂MFTLKQAFPAE
YLPQX₁₃DKTHTCPPCPAPELLGGPSVFLFPPKPKDTLMISRTPEVTCVVVDVSHEDPEVKENWY
VDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFFL
TSKLTVDKSRWQQGNVFSCSVMHEALHNHYTQKSLSLSPGK
SEQ ID NO: 89:
X₁X₂SX₃X₄X₅LQX₆ESVFQSGAHAYRIPALLYLPGQQSLLAFAEQRASX₇X₈DEHAELIVX₉RRGD
YDAX₁₀THQVQWX₁₁AQEVVAQAX₁₂LDGHRSMNPCPLYDX₁₃QTGTLFLFFIAIPX₁₄X₁₅VTEX₁₆Q
QLQTRANVTRLX₁₇X₁₈VTSTDHGRTWSSPRDLTDAAIGPX₁₉YREWSTFAVGPGHX₂₀LQLHDRX₂₁
RSLVVPAYAYRKLHPX₂₂QRPIPSAFX₂₃FLSHDHGRTWARGHFVAQDTX₂₄ECQVAEVETGEQRV
VTLNARSHLRARVQAQSX₂₅NX₂₆GLDFQX₂₇SQLVKKLVEPPPX₂₈GX₂₉QGSVISFPSPRSGPGSP
AQX₃₀LLYTHPTHX₃₁X₃₂QRADLGAYLNPRPPAPEAWSEPX₃₃LLAKGSX₃₄AYSDLQSMGTGPDGS
PLFGX₃₅LYEANDYEEIX₃₆FX₃₇MFTLKQAFPAEYLPQX₃₈DKTHTCPPCPAPELLGGPSVELEPP
KPKDTLMISRTPEVTCVVVDVSHEDPEVKENWYVDGVEVHNAKTKPREEQYNSTYRVVSVLTVL
HQDWLNGKEYKCKVSNKALPAPIEKTISKAKGQPREPQVYTLPPSREEMTKNQVSLTCLVKGFY
PSDIAVEWESNGQPENNYKTTPPVLDSDGSFFLTSKLTVDKSRWQQGNVFSCSVMHEALHNHYT
QKSLSLSPGK
SEQ ID NO: 90:
GGGGSGGGGS
SEQ ID NO: 91:
EPKSS
SEQ ID NO: 92:
EPKSCDKTHTCPPCPAPELLGGPSVFLFPPKPKDTLMISRTPEVTCVVVDVSHEDPEVKENWYV
DGVEVHNAKTKPREEQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKTTPPVLDSDGSFFLT
SKLTVDKSRWQQGNVFSCSVMHEALHNHYTQKSLSLSPGK
SEQ ID NO: 93:
DKTHTCPPCPAPELLGGPSVELFPPKPKDTLMISRTPEVTCVVVDVSHEDPEVKENWYVDGVEV
HNAKTKPREEQYNSTYRVVSVLTVLHQDWLNGKEYKCKVSNKALPAPIEKTISKAKGQPREPQV
YTLPPSREEMTKNQVSLYCLVKGFYPSDIAVEWESNGQPENNYKTTPPVLDSDGSFFLYSKLTV
DKSRWQQGNVFSCSVMHEALHNHYTQKSLSLSPGK
SEQ ID NO: 94:
DASLPYLQKESVFQSGAHAYRIPALLYLPGQQSLLAFAEQRASKKDEHAELIVLRRGDYDAGTH
QVQWQAQEVVAQARLDGHRSMNPCPLYDEQTGTLFLFFIAIPGQVTEQQQLQTRANVTRLCQVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSTNDGLDFQ
ESQLVKKLVEPPPQGCQGSVISFPSPRSGPGSPAQWLLYTHPTHARQRADLGAYLNPRPPAPEA
WSEPVLLAKGSAAYSDLQSMGTGPDGSPLFGCLYEANDYEEIVELMFTLKQAFPAEYLPQ
SEQ ID NO: 95:
DASLPYLQKESVFQSGAHAYRIPALLYLPGQQSLLAFAEQRASKKDEHAELIVLRRGDYDAGTH
QVQWQAQEVVAQARLDGHRSMNPCPLYDEQTGTLFLFFIAIPGQVTEQQQLQTRANVTRLCQVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSTNDGLDFQ
ESQLVKKLVEPPPQGCQGSVISFPSPRSGPGSPAQWLLYTHPTHARQRADLGAYLNPRPPAPEA
WSEPVLLAKGSAAYSDLQSMGTGPDGSPLFGCLYEANDYEEIVELMFTLKQAFPAEYLPQGGGG
SGGGGSDKTHTCPPCPAPELLGGPSVELFPPKPKDTLMISRTPEVTCVVVDVSHEDPEVKENWY
VDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFFL
TSKLTVDKSRWQQGNVFSCSVMHEALHNHYTQKSLSLSPGK
SEQ ID NO: 96:
DASLPYLQKESVFQSGAHAYRIPALLYLPGQQSLLAFAEQRASKKDEHAELIVLRRGDYDAGTH
QVQWQAQEVVAQARLDGHRSMNPCPLYDEQTGTLFLFFIAIPGQVTEQQQLQTRANVTRLCQVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSTNDGLDFQ
ESQLVKKLVEPPPQGCQGSVISFPSPRSGPGSPAQWLLYTHPTHARQRADLGAYLNPRPPAPEA
WSEPVLLAKGSAAYSDLQSMGTGPDGSPLFGCLYEANDYEEIVELMFTLKQAFPAEYLPQEPKS
SDKTHTCPPCPAPELLGGPSVFLFPPKPKDTLMISRTPEVTCVVVDVSHEDPEVKENWYVDGVE
VHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFELTSKLT
VDKSRWQQGNVFSCSVMHEALHNHYTQKSLSLSPGK
SEQ ID NO: 97:
DASLPYLQKESVFQSGAHAYRIPALLYLPGQQSLLAFAEQRASKKDEHAELIVLRRGDYDAGTH
QVQWQAQEVVAQARLDGHRSMNPCPLYDEQTGTLFLFFIAIPGQVTEQQQLQTRANVTRLCYVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSTNDGLDFQ
ESQLVKKLVEPPPTGCQGSVISFPSPRSGPGSPAQWLLYTHPTHSWQRADLGAYLNPRPPAPEA
WSEPVLLAKGSAAYSDLQSMGTGPDGSPLFGCLYEANDYEEIVELMFTLKQAFPAEYLPQ
SEQ ID NO: 98:
DASLPYLQKESVFQSGAHAYRIPALLYLPGQQSLLAFAEQRASKKDEHAELIVLRRGDYDAGTH
QVQWQAQEVVAQARLDGHRSMNPCPLYDEQTGTLFLFFIAIPGQVTEQQQLQTRANVTRLCYVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSTNDGLDFQ
ESQLVKKLVEPPPTGCQGSVISFPSPRSGPGSPAQWLLYTHPTHSWQRADLGAYLNPRPPAPEA
WSEPVLLAKGSAAYSDLQSMGTGPDGSPLFGCLYEANDYEEIVELMFTLKQAFPAEYLPQGGGG
SGGGGSDKTHTCPPCPAPELLGGPSVELFPPKPKDTLMISRTPEVTCVVVDVSHEDPEVKENWY
VDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFFL
TSKLTVDKSRWQQGNVFSCSVMHEALHNHYTQKSLSLSPGK
SEQ ID NO: 99:
DASLPYLQKESVFQSGAHAYRIPALLYLPGQQSLLAFAEQRASKKDEHAELIVLRRGDYDAGTH
QVQWQAQEVVAQARLDGHRSMNPCPLYDEQTGTLFLFFIAIPGQVTEQQQLQTRANVTRLCYVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSTNDGLDFQ
ESQLVKKLVEPPPTGCQGSVISFPSPRSGPGSPAQWLLYTHPTHSWQRADLGAYLNPRPPAPEA
WSEPVLLAKGSAAYSDLQSMGTGPDGSPLFGCLYEANDYEEIVELMFTLKQAFPAEYLPQEPKS
SDKTHTCPPCPAPELLGGPSVFLFPPKPKDTLMISRTPEVTCVVVDVSHEDPEVKENWYVDGVE
VHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFELTSKLT
VDKSRWQQGNVFSCSVMHEALHNHYTQKSLSLSPGK
SEQ ID NO: 100:
DASLPYLQKESVFQSGAHAYRIPALLYLPGQQSLLAFAEQRASKKDEHAELIVLRRGDYDAGTH
QVQWQAQEVVAQARLDGHRSMNPCPLYDEQTGTLFLFFIAIPGQVTEQQQLQTRANVTRLCYVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRERVQAQSTNDGLDFQ
ESQLVKKLVEPPPTGCQGSVISFPSPRSGPGSPAQWLLYTHPTHSWQRADLGAYLNPRPPAPEA
WSEPVLLAKGSAAYSDLQSMGTGPDGSPLFGCLYEANDYEEIVELMFTLKQAFPAEYLPQ
SEQ ID NO: 101:
DASLPYLQKESVFQSGAHAYRIPALLYLPGQQSLLAFAEQRASKKDEHAELIVLRRGDYDAGTH
QVQWQAQEVVAQARLDGHRSMNPCPLYDEQTGTLFLFFIAIPGQVTEQQQLQTRANVTRLCYVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRERVQAQSTNDGLDFQ
ESQLVKKLVEPPPTGCQGSVISFPSPRSGPGSPAQWLLYTHPTHSWQRADLGAYLNPRPPAPEA
WSEPVLLAKGSAAYSDLQSMGTGPDGSPLFGCLYEANDYEEIVELMFTLKQAFPAEYLPQGGGG
SGGGGSDKTHTCPPCPAPELLGGPSVFLFPPKPKDTLMISRTPEVTCVVVDVSHEDPEVKENWY
VDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFFL
TSKLTVDKSRWQQGNVFSCSVMHEALHNHYTQKSLSLSPGK
SEQ ID NO: 102:
DASLPYLQKESVFQSGAHAYRIPALLYLPGQQSLLAFAEQRASKKDEHAELIVLRRGDYDAGTH
QVQWQAQEVVAQARLDGHRSMNPCPLYDEQTGTLFLFFIAIPGQVTEQQQLQTRANVTRLCYVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRERVQAQSTNDGLDFQ
ESQLVKKLVEPPPTGCQGSVISFPSPRSGPGSPAQWLLYTHPTHSWQRADLGAYLNPRPPAPEA
WSEPVLLAKGSAAYSDLQSMGTGPDGSPLFGCLYEANDYEEIVELMETLKQAFPAEYLPQEPKS
SDKTHTCPPCPAPELLGGPSVELFPPKPKDTLMISRTPEVTCVVVDVSHEDPEVKENWYVDGVE
VHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLTSKLT
VDKSRWQQGNVFSCSVMHEALHNHYTQKSLSLSPGK
SEQ ID NO: 103:
GATGCATCTCTGCCTTACCTGCAGAAAGAAAGCGTGTTCCAGTCTGGCGCCCACGCCTACAGAA
TTCCCGCTCTGCTGTATCTGCCAGGCCAGCAGTCTCTGCTGGCTTTCGCTGAACAGCGGGCCAG
CAAGAAGGATGAGCACGCCGAACTGATCGTGCTGCGGAGAGGCGATTACGACGCCGGCACACAT
CAGGTGCAGTGGCAGGCTCAAGAGGTGGTGGCTCAGGCTAGACTGGACGGCCACAGATCTATGA
ACCCCTGTCCTCTGTACGATGAACAGACCGGCACACTGTTTCTGTTCTTTATCGCTATCCCCGG
CCAAGTGACCGAGCAGCAGCAGCTGCAGACAAGAGCCAACGTGACCAGACTGTGTCAAGTGACC
TCCACCGACCACGGCAGAACCTGGTCTAGCCCTAGAGATCTGACCGACGCCGCCATCGGACCTG
CCTATAGAGAGTGGTCCACCTTCGCCGTTGGACCTGGACACTGTCTCCAGCTGCACGACAGGGC
TAGATCTCTGGTGGTGCCTGCCTACGCCTATAGAAAGCTGCACCCCAAACAGCGGCCTATTCCT
AGCGCCTTCTGCTTTCTGAGCCACGATCACGGCAGGACATGGGCCAGAGGACATTTCGTGGCCC
AGGACACACTGGAATGCCAGGTGGCCGAAGTGGAAACCGGCGAGCAGAGAGTCGTGACCCTGAA
CGCCAGATCTCACCTGAGAGCCAGAGTGCAGGCCCAGAGCACAAACGACGGCCTGGATTTCCAA
GAGAGCCAGCTGGTCAAGAAACTGGTGGAACCTCCTCCACAGGGCTGTCAGGGAAGCGTGATCA
GCTTTCCATCTCCTAGAAGCGGCCCTGGCTCTCCTGCTCAGTGGCTGCTGTATACACACCCCAC
ACACAGCTGGCAGAGAGCCGATCTGGGCGCCTACCTGAATCCTAGACCTCCTGCTCCTGAGGCT
TGGAGCGAACCTGTTCTGCTGGCCAAGGGCAGCGCTGCCTACAGCGATCTGCAGTCTATGGGCA
CAGGCCCTGATGGCAGCCCTCTGTTTGGCTGTCTGTACGAGGCCAACGACTACGAAGAGATCGT
GTTCCTGATGTTCACCCTGAAGCAGGCCTTTCCAGCCGAGTACCTGCCTCAAGGCGGAGGCGGA
TCCGACAAAACTCACACATGCCCACCGTGCCCAGCACCTGAACTCCTGGGGGGACCGTCAGTCT
TCCTCTTCCCCCCAAAACCCAAGGACACCCTCATGATCTCCCGGACCCCTGAGGTCACATGCGT
GGTGGTGGACGTGAGCCACGAAGACCCTGAGGTCAAGTTCAACTGGTACGTGGACGGCGTGGAG
GTGCATAATGCCAAGACAAAGCCGCGGGAGGAGCAGTACAACAGCACGTACCGTGTGGTCAGCG
TCCTCACCGTCCTGCACCAGGACTGGCTGAATGGCAAGGAGTACAAGTGCAAGGTCTCCAACAA
AGCCCTCCCAGCCCCCATCGAGAAAACCATCTCCAAAGCCAAAGGGCAGCCCCGAGAACCACAG
GTCTACACCCTGCCCCCATCCCGGGAGGAGATGACCAAGAACCAGGTCAGCCTGACCTGCCTGG
TCAAAGGCTTCTATCCCAGCGACATCGCCGTGGAGTGGGAGAGCAATGGGCAGCCGGAGAACAA
CTACAAGACCACGCCTCCCGTGCTGGACTCCGACGGCTCCTTCTTCCTCACTAGCAAGCTCACC
GTGGACAAGAGCAGGTGGCAGCAGGGGAACGTCTTCTCATGCTCCGTGATGCATGAGGCTCTGC
ACAACCACTACACGCAGAAGAGCCTCTCCCTGTCTCCGGGTAAA
SEQ ID NO: 104:
GAGATCGTGCTGACACAGAGCCCTGCCACACTGTCACTGTCTCCAGGCGAGAGAGCCACACTGA
GCTGTAGAGCTTCTAAGGGCGTGTCCACCAGCGGCTACTCTTACCTGCACTGGTATCAGCAGAA
GCCCGGCCAGGCTCCTAGACTGCTGATCTACCTGGCCAGCTACCTGGAAAGCGGAGTGCCTGCT
AGATTCAGCGGCTCTGGCTCTGGCACCGACTTCACCCTGACAATCAGCAGCCTGGAACCTGAGG
ACTTCGCCGTGTACTACTGCCAGCACAGCAGAGATCTGCCCCTGACATTTGGCGGAGGCACCAA
GGTGGAAATCAAGCGTACGGTGGCTGCACCATCTGTCTTCATCTTCCCGCCATCTGATGAGCAG
TTGAAATCTGGAACTGCCTCTGTTGTGTGCCTGCTGAATAACTTCTATCCCAGAGAGGCCAAAG
TACAGTGGAAGGTGGATAACGCCCTCCAATCGGGTAACTCCCAGGAGAGTGTCACAGAGCAGGA
CAGCAAGGACAGCACCTACAGCCTCAGCAGCACCCTGACGCTGAGCAAAGCAGACTACGAGAAA
CACAAAGTCTACGCCTGCGAAGTCACCCATCAGGGCCTGAGCTCGCCCGTCACAAAGAGCTTCA
ACAGGGGAGAGTGT
SEQ ID NO: 105:
QVQLVQSGVEVKKPGASVKVSCKASGYTFTNYYMYWVRQAPGQGLEWMGGINPSNGGTNENEKE
KNRVTLTTDSSTTTAYMELKSLQFDDTAVYYCARRDYRFDMGFDYWGQGTTVTVSSASTKGPSV
FPLAPSSKSTSGGTAALGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGGPSVELFPPKPKDTLMI
SRTPEVTCVVVDVSHEDPEVKENWYVDGVEVHNAKTKPREEQYASTYRVVSVLTVLHQDWLNGK
EYKCKVSNKALPAPIEKTISKAKGQPREPQVYTLPPSREEMTKNQVSLTCLVKGFYPSDIAVEW
ESNGQPENNYKTTPPVLDSDGSFFLTSKLTVDKSRWQQGNVFSCSVMHEALHNHYTQKSLSLSP
GK
SEQ ID NO: 106:
TVEKSVVFKAEGEHFTDQKGNTIVGSGSGGTTKYFRIPAMCTTSKGTIVVFADARHNTASDQSE
IDTAAARSTDGGKTWNKKIAIYNDRVNSKLSRVMDPTCIVANIQGRETILVMVGKWNNNDKTWG
AYRDKAPDTDWDLVLYKSTDDGVTFSKVETNIHDIVTKNGTISAMLGGVGSGLQLNDGKLVFPV
QMVRTKNITTVLNTSFIYSTDGITWSLPSGYCEGFGSENNIIEFNASLVNNIRNSGLRRSFETK
DFGKTWTEFPPMDKKVDNRNHGVQGSTITIPSGNKLVAAHSSAQNKNNDYTRSDISLYAHNLYS
GEVKLIDDFYPKVGNASGAGYSCLSYRKNVDKETLYVVYEANGSIEFQDLSRHLPVIKSYNGGG
GSGGGGSDKTHTCPPCPAPELLGGPSVELFPPKPKDTLMISRTPEVTCVVVDVSHEDPEVKENW
YVDGVEVHNAKTKPREEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENNYKTTPPVLDSDGSFF
LTSKLTVDKSRWQQGNVFSCSVMHEALHNHYTQKSLSLSPGKSGGGGSHHHHHHHH
SEQ ID NO: 107:
ACAGTGGAAAAGTCCGTGGTGTTCAAGGCCGAGGGCGAGCACTTCACCGACCAGAAAGGCAATA
CCATCGTCGGCTCTGGCAGCGGCGGCACCACCAAGTACTTTAGAATCCCCGCCATGTGCACCAC
CAGCAAGGGCACCATTGTGGTGTTCGCCGACGCCAGACACAACACCGCCAGCGATCAGAGCTTC
ATCGATACCGCTGCCGCCAGAAGTACAGACGGCGGCAAGACCTGGAACAAGAAGATCGCCATCT
ACAACGACCGCGTGAACAGCAAGCTGAGCAGAGTGATGGACCCTACCTGCATCGTGGCCAACAT
CCAGGGCAGAGAAACCATCCTGGTCATGGTCGGAAAGTGGAACAACAACGATAAGACCTGGGGC
GCCTACAGAGACAAGGCCCCTGATACCGATTGGGACCTCGTGCTGTATAAGAGCACCGACGACG
GCGTGACCTTCAGCAAGGTGGAAACAAACATCCACGACATCGTGACCAAGAACGGCACCATCTC
TGCCATGCTCGGCGGCGTTGGATCTGGCCTGCAACTGAATGATGGCAAGCTGGTGTTCCCCGTG
CAGATGGTCCGAACAAAGAACATCACCACCGTGCTGAATACCAGCTTCATCTACTCCACCGACG
GCATCACATGGTCCCTGCCTAGCGGCTACTGTGAAGGCTTTGGCAGCGAGAACAACATCATCGA
GTTCAACGCCAGCCTGGTCAACAACATCCGGAACAGCGGCCTGCGGAGAAGCTTCGAGACAAAG
GACTTCGGAAAGACGTGGACCGAGTTTCCTCCAATGGACAAGAAGGTGGACAACCGGAACCACG
GCGTGCAGGGCAGCACAATCACAATCCCTAGCGGCAACAAACTGGTGGCCGCTCACTCTAGCGC
CCAGAACAAGAACAACGATTACACCAGAAGCGACATCAGCCTGTACGCCCACAACCTGTACTCC
GGCGAAGTGAAGCTGATCGACGACTTCTACCCCAAAGTGGGCAATGCCAGCGGAGCCGGCTACA
GCTGTCTGAGCTACCGGAAAAATGTGGACAAAGAAACCCTGTACGTGGTGTACGAGGCCAACGG
CAGCATCGAGTTTCAGGACCTGAGCAGACATCTGCCCGTGATCAAGAGCTACAATGGCGGAGGT
GGAAGTGGCGGAGGCGGATCCGACAAAACTCACACATGCCCACCGTGCCCAGCACCTGAACTCC
TGGGGGGACCGTCAGTCTTCCTCTTCCCCCCAAAACCCAAGGACACCCTCATGATCTCCCGGAC
CCCTGAGGTCACATGCGTGGTGGTGGACGTGAGCCACGAAGACCCTGAGGTCAAGTTCAACTGG
TACGTGGACGGCGTGGAGGTGCATAATGCCAAGACAAAGCCGCGGGAGGAGCAGTACAACAGCA
CGTACCGTGTGGTCAGCGTCCTCACCGTCCTGCACCAGGACTGGCTGAATGGCAAGGAGTACAA
GTGCAAGGTCTCCAACAAAGCCCTCCCAGCCCCCATCGAGAAAACCATCTCCAAAGCCAAAGGG
CAGCCCCGAGAACCACAGGTCTACACCCTGCCCCCATCCCGGGAGGAGATGACCAAGAACCAGG
TCAGCCTGACCTGCCTGGTCAAAGGCTTCTATCCCAGCGACATCGCCGTGGAGTGGGAGAGCAA
TGGGCAGCCGGAGAACAACTACAAGACCACGCCTCCCGTGCTGGACTCCGACGGCTCCTTCTTC
CTCACTAGCAAGCTCACCGTGGACAAGAGCAGGTGGCAGCAGGGGAACGTCTTCTCATGCTCCG
TGATGCATGAGGCTCTGCACAACCACTACACGCAGAAGAGCCTCTCCCTGTCTCCGGGTAAAAG
CGGCGGAGGCGGATCTCATCATCACCATCATCACCATCAC
SEQ ID NO: 108:
TVEKSVVFKAEGEHFTDQKGNTIVGSGSGGTTKYFRIPAMCTTSKGTIVVFADARHNTASDQSF
IDTAAARSTDGGKTWNKKIAIYNDRVNSKLSRVMDPTCIVANIQGRETILVMVGKWNNNDKTWG
AYRDKAPDTDWDLVLYKSTDDGVTFSKVETNIHDIVTKNGTISAMLGGVGSGLQLNDGKLVFPV
QMVRTKNITTVLNTSFIYSTDGITWSLPSGYCEGFGSENNIIEFNASLVNNIRNSGLRRSFETK
DFGKTWTEFPPMDKKVDNRNHGVQGSTITIPSGNKLVAAHSSAQNKNNDYTRSDISLYAHNLYS
GEVKLIDDFYPKVGNASGAGYSCLSYRKNVDKETLYVVYEANGSIEFQDLSRHLPVIKSYNGGG
GSGGGGSDKTHTCPPCPAPELLGGPSVELFPPKPKDTLMISRTPEVTCVVVDVSHEDPEVKENW
YVDGVEVHNAKTKPREEQYGSTYRVVSVLTVLHQDWLNGKEYKCKVSNKALPAPIEKTISKAKG
QPREPQVYTLPPSREEMTKNQVSLTCLVKGFYPSDIAVEWESNGQPENNYKTTPPVLDSDGSFF
LTSKLTVDKSRWQQGNVFSCSVMHEALHNHYTQKSLSLSPGKSGGGGSHHHHHHHH
SEQ ID NO: 109:
ACAGTGGAAAAGTCCGTGGTGTTCAAGGCCGAGGGCGAGCACTTCACCGACCAGAAAGGCAATA
CCATCGTCGGCTCTGGCAGCGGCGGCACCACCAAGTACTTTAGAATCCCCGCCATGTGCACCAC
CAGCAAGGGCACCATTGTGGTGTTCGCCGACGCCAGACACAACACCGCCAGCGATCAGAGCTTC
ATCGATACCGCTGCCGCCAGAAGTACAGACGGCGGCAAGACCTGGAACAAGAAGATCGCCATCT
ACAACGACCGCGTGAACAGCAAGCTGAGCAGAGTGATGGACCCTACCTGCATCGTGGCCAACAT
CCAGGGCAGAGAAACCATCCTGGTCATGGTCGGAAAGTGGAACAACAACGATAAGACCTGGGGC
GCCTACAGAGACAAGGCCCCTGATACCGATTGGGACCTCGTGCTGTATAAGAGCACCGACGACG
GCGTGACCTTCAGCAAGGTGGAAACAAACATCCACGACATCGTGACCAAGAACGGCACCATCTC
TGCCATGCTCGGCGGCGTTGGATCTGGCCTGCAACTGAATGATGGCAAGCTGGTGTTCCCCGTG
CAGATGGTCCGAACAAAGAACATCACCACCGTGCTGAATACCAGCTTCATCTACTCCACCGACG
GCATCACATGGTCCCTGCCTAGCGGCTACTGTGAAGGCTTTGGCAGCGAGAACAACATCATCGA
GTTCAACGCCAGCCTGGTCAACAACATCCGGAACAGCGGCCTGCGGAGAAGCTTCGAGACAAAG
GACTTCGGAAAGACGTGGACCGAGTTTCCTCCAATGGACAAGAAGGTGGACAACCGGAACCACG
GCGTGCAGGGCAGCACAATCACAATCCCTAGCGGCAACAAACTGGTGGCCGCTCACTCTAGCGC
CCAGAACAAGAACAACGATTACACCAGAAGCGACATCAGCCTGTACGCCCACAACCTGTACTCC
GGCGAAGTGAAGCTGATCGACGACTTCTACCCCAAAGTGGGCAATGCCAGCGGAGCCGGCTACA
GCTGTCTGAGCTACCGGAAAAATGTGGACAAAGAAACCCTGTACGTGGTGTACGAGGCCAACGG
CAGCATCGAGTTTCAGGACCTGAGCAGACATCTGCCCGTGATCAAGAGCTACAATGGCGGAGGT
GGAAGTGGCGGAGGCGGATCCGACAAAACTCACACATGCCCACCGTGCCCAGCACCTGAACTCC
TGGGGGGACCGTCAGTCTTCCTCTTCCCCCCAAAACCCAAGGACACCCTCATGATCTCCCGGAC
CCCTGAGGTCACATGCGTGGTGGTGGACGTGAGCCACGAAGACCCTGAGGTCAAGTTCAACTGG
TACGTGGACGGCGTGGAGGTGCATAATGCCAAGACAAAGCCGCGGGAGGAGCAGTACGGTAGCA
CGTACCGTGTGGTCAGCGTCCTCACCGTCCTGCACCAGGACTGGCTGAATGGCAAGGAGTACAA
GTGCAAGGTCTCCAACAAAGCCCTCCCAGCCCCCATCGAGAAAACCATCTCCAAAGCCAAAGGG
CAGCCCCGAGAACCACAGGTCTACACCCTGCCCCCATCCCGGGAGGAGATGACCAAGAACCAGG
TCAGCCTGACCTGCCTGGTCAAAGGCTTCTATCCCAGCGACATCGCCGTGGAGTGGGAGAGCAA
TGGGCAGCCGGAGAACAACTACAAGACCACGCCTCCCGTGCTGGACTCCGACGGCTCCTTCTTC
CTCACTAGCAAGCTCACCGTGGACAAGAGCAGGTGGCAGCAGGGGAACGTCTTCTCATGCTCCG
TGATGCATGAGGCTCTGCACAACCACTACACGCAGAAGAGCCTCTCCCTGTCTCCGGGTAAAAG
CGGCGGAGGCGGATCTCATCATCACCATCATCACCATCAC
SEQ ID NO: 110:
CAGGTTCAGCTGGTGCAGTCTGGCGTGGAAGTGAAGAAACCTGGGGCCAGCGTGAAGGTGTCCT
GTAAAGCCAGCGGCTACACCTTTACCAACTACTACATGTACTGGGTCCGACAGGCCCCAGGCCA
AGGACTTGAATGGATGGGCGGCATCAACCCCAGCAACGGCGGCACCAACTTCAACGAGAAGTTC
AAGAACAGAGTGACCCTGACCACCGACAGCAGCACCACCACAGCCTACATGGAACTGAAGTCCC
TGCAGTTCGACGACACCGCCGTGTACTACTGCGCCAGACGGGACTACAGATTCGACATGGGCTT
CGACTATTGGGGCCAGGGCACCACCGTGACAGTCTCTTCTGCtagcACCAAGGGCCCATCcGTC
TTCCCCCTGGCACCCTCCTCCAAGAGCACCTCTGGGGGCACAGCGGCCCTGGGCTGCCTGGTCA
AGGACTACTTCCCCGAACCGGTGACGGTGTCCTGGAACTCAGGCGCtCTGACCAGCGGCGTGCA
CACCTTCCCGGCTGTCCTACAGTCCTCAGGACTCTACTCCCTCAGCAGCGTGGTGACCGTGCCC
TCCAGCAGCTTGGGCACCCAGACCTACATCTGCAACGTGAATCACAAGCCCAGCAACACCAAGG
TGGACAAGAAAGTTGAGCCCAAATCTTGTGACAAAACTCACACATGCCCACCGTGCCCAGCACC
TGAACTCCTGGGGGGACCGTCAGTCTTCCTCTTCCCCCCAAAACCCAAGGACACCCTCATGATC
TCCCGGACCCCTGAGGTCACATGCGTGGTGGTGGACGTGAGCCACGAAGACCCTGAGGTCAAGT
TCAACTGGTACGTGGACGGCGTGGAGGTGCATAATGCCAAGACAAAGCCGCGGGAGGAGCAGTA
CgccAGCACGTACCGTGTGGTCAGCGTCCTCACCGTCCTGCACCAGGACTGGCTGAATGGCAAG
GAGTACAAGTGCAAGGTCTCCAACAAAGCCCTCCCAGCCCCCATCGAGAAAACCATCTCCAAAG
CCAAAGGGCAGCCCCGAGAACCACAGGTcTACACCCTGCCCCCATCCCGGGAGGAGATGACCAA
GAACCAGGTCAGCCTGACCTGCCTGGTCAAAGGCTTCTATCCCAGCGACATCGCCGTGGAGTGG
GAGAGCAATGGGCAGCCGGAGAACAACTACAAGACCACGCCTCCCGTGCTGGACTCCGACGGCT
CCTTCTTCCTCaccAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCG
GGTAAA
SEQ ID NO: 111:
DASLPYLQKESVFQSGAHAYRIPALLYLPGQQSLLAFAEQRASKKDEHAELIVLRRGDYDAGTH
QVQWQAQEVVAQARLDGHRSMNPCPLYDEQTGTLFLFFIAIPGQVTEQQQLQTRANVTRLCQVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSTNDGLDFQ
ESQLVKKLVEPPPQGCQGSVISFPSPRSGPGSPAQWLLYTHPTHSWQRADLGAYLNPRPPAPEA
WSEPVLLAKGSAAYSDLQSMGTGPDGSPLFGCLYEANDYEEIVELMFTLKQAFPAEYLPQEPKS
SDKTHTCPPCPAPELLGGPSVFLFPPKPKDTLMISRTPEVTCVVVDVSHEDPEVKENWYVDGVE
VHNAKTKPREEQYASTYRVVSVLTVLHQDWLNGKEYKCKVSNKALPAPIEKTISKAKGQPREPQ
VYTLPPSREEMTKNQVSLYCLVKGFYPSDIAVEWESNGQPENNYKTTPPVLDSDGSFFLYSKLT
VDKSRWQQGNVFSCSVMHEALHNHYTQKSLSLSPGK
SEQ ID NO: 112:
DASLPYLQKESVFQSGAHAYRIPALLYLPGQQSLLAFAEQRASKKDEHAELIVLRRGDYDAGTH
QVQWQAQEVVAQARLDGHRSMNPCPLYDEQTGTLFLFFIAIPGQVTEQQQLQTRANVTRLCQVT
STDHGRTWSSPRDLTDAAIGPAYREWSTFAVGPGHCLQLHDRARSLVVPAYAYRKLHPKQRPIP
SAFCFLSHDHGRTWARGHEVAQDTLECQVAEVETGEQRVVTLNARSHLRARVQAQSTNDGLDFQ
ESQLVKKLVEPPPQGCQGSVISFPSPRSGPGSPAQWLLYTHPTHSWQRADLGAYLNPRPPAPEA
WSEPVLLAKGSAAYSDLQSMGTGPDGSPLFGCLYEANDYEEIVELMFTLKQAFPAEYLPQGGGG
SGGGGSDKTHTCPPCPAPELLGGPSVELFPPKPKDTLMISRTPEVTCVVVDVSHEDPEVKENWY
VDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFEL
TSKLTVDKSRWQQGNVFSCSVMHEALHNHYTQKSLSLSPGK
SEQ ID NO: 113:
GATGCATCTCTGCCTTACCTGCAGAAAGAAAGCGTGTTCCAGTCTGGCGCCCACGCCTACAGAA
TTCCCGCTCTGCTGTATCTGCCAGGCCAGCAGTCTCTGCTGGCTTTCGCTGAACAGCGGGCCAG
CAAGAAGGATGAGCACGCCGAACTGATCGTGCTGCGGAGAGGCGATTACGACGCCGGCACACAT
CAGGTGCAGTGGCAGGCTCAAGAGGTGGTGGCTCAGGCTAGACTGGACGGCCACAGATCTATGA
ACCCCTGTCCTCTGTACGATGAACAGACCGGCACACTGTTTCTGTTCTTTATCGCTATCCCCGG
CCAAGTGACCGAGCAGCAGCAGCTGCAGACAAGAGCCAACGTGACCAGACTGTGTCAAGTGACC
TCCACCGACCACGGCAGAACCTGGTCTAGCCCTAGAGATCTGACCGACGCCGCCATCGGACCTG
CCTATAGAGAGTGGTCCACCTTCGCCGTTGGACCTGGACACTGTCTCCAGCTGCACGACAGGGC
TAGATCTCTGGTGGTGCCTGCCTACGCCTATAGAAAGCTGCACCCCAAACAGCGGCCTATTCCT
AGCGCCTTCTGCTTTCTGAGCCACGATCACGGCAGGACATGGGCCAGAGGACATTTCGTGGCCC
AGGACACACTGGAATGCCAGGTGGCCGAAGTGGAAACCGGCGAGCAGAGAGTCGTGACCCTGAA
CGCCAGATCTCACCTGAGAGCCAGAGTGCAGGCCCAGAGCACAAACGACGGCCTGGATTTCCAA
GAGAGCCAGCTGGTCAAGAAACTGGTGGAACCTCCTCCACAGGGCTGTCAGGGAAGCGTGATCA
GCTTTCCATCTCCTAGAAGCGGCCCTGGCTCTCCTGCTCAGTGGCTGCTGTATACACACCCCAC
ACACAGCTGGCAGAGAGCCGATCTGGGCGCCTACCTGAATCCTAGACCTCCTGCTCCTGAGGCT
TGGAGCGAACCTGTTCTGCTGGCCAAGGGCAGCGCTGCCTACAGCGATCTGCAGTCTATGGGCA
CAGGCCCTGATGGCAGCCCTCTGTTTGGCTGTCTGTACGAGGCCAACGACTACGAAGAGATCGT
GTTCCTGATGTTCACCCTGAAGCAGGCCTTTCCAGCCGAGTACCTGCCTCAAGGCGGAGGTGGA
AGTGGCGGAGGCGGATCCGACAAAACTCACACATGCCCACCGTGCCCAGCACCTGAACTCCTGG
GGGGACCGTCAGTCTTCCTCTTCCCCCCAAAACCCAAGGACACCCTCATGATCTCCCGGACCCC
TGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACAACAGCACGT
ACCGTGTGGTCAGCGTCCTCACCGTCCTGCACCAGGACTGGCTGAATGGCAAGGAGTACAAGTG
CAAGGTCTCCAACAAAGCCCTCCCAGCCCCCATCGAGAAAACCATCTCCAAAGCCAAAGGGCAG
CCCCGAGAACCACAGGTCTACACCCTGCCCCCATCCCGGGAGGAGATGACCAAGAACCAGGTCA
GCCTGACCTGCCTGGTCAAAGGCTTCTATCCCAGCGACATCGCCGTGGAGTGGGAGAGCAATGG
GCAGCCGGAGAACAACTACAAGACCACGCCTCCCGTGCTGGACTCCGACGGCTCCTTCTTCCTC
ACTAGCAAGCTCACCGTGGACAAGAGCAGGTGGCAGCAGGGGAACGTCTTCTCATGCTCCGTGA
TGCATGAGGCTCTGCACAACCACTACACGCAGAAGAGCCTCTCCCTGTCTCCGGGTAAA
SEQ ID NO: 114:
gatGCATCTCTGCCTTACCTGCAGAAAGAAAGCGTGTTCCAGTCTGGCGCCCACGCCTACAGAA
TTCCCGCTCTGCTGTATCTGCCAGGCCAGCAGTCTCTGCTGGCTTTCGCTGAACAGCGGGCCAG
CAAGAAGGATGAGCACGCCGAACTGATCGTGCTGCGGAGAGGCGATTACGACGCCggcACACAT
CAGGTGCAGTGGCAGGCTCAAGAGGTGGTGGCTCAGGCTAGACTGGACGGCCACAGATCTATGA
ACCCCTGTCCTCTGTACGATgaaCAGACCGGCACACTGTTTCTGTTCTTTATCGCTATCCCCGG
CCAAGTGACCGAGCAGCAGCAGCTGCAGACAAGAGCCAACGTGACCAGACTGTGTCAAGTGACC
TCCACCGACCACGGCAGAACCTGGTCTAGCCCTAGAGATCTGACCGACGCCGCCATCGGACCTG
CCTATAGAGAGTGGTCCACCTTCGCCGTTGGACCTGGACACTGTCTCCAGCTGCACGACAGGGC
TAGATCTCTGGTGGTGCCTGCCTACGCCTATAGAAAGCTGCACCCCAAACAGCGGCCTATTCCT
AGCGCCTTCTGCTTTCTGAGCCACGATCACGGCAGGACATGGGCCAGAGGACATTTCGTGGCCC
AGGACACACTGGAATGCCAGGTGGCCGAAGTGGAAACCGGCGAGCAGAGAGTCGTGACCCTGAA
CGCCAGATCTCACCTGAGAGCCAGAGTGCAGGCCCAGAGCACAAACGACGGCCTGGATTTCCAA
GAGAGCCAGCTGGTCAAGAAACTGGTGGAACCTCCTCCACAGGGCTGTCAGGGAAGCGTGATCA
GCTTTCCATCTCCTAGAAGCGGCCCTGGCTCTCCTGCTCAGTGGCTGCTGTATACACACCCCAC
ACACAGCTGGCAGAGAGCCGATCTGGGCGCCTACCTGAATCCTAGACCTCCTGCTCCTGAGGCT
TGGAGCGAACCTGTTCTGCTGGCCAAGGGCAGCgctGCCTACAGCGATCTGCAGTCTATGGGCA
CAGGCCCTGATGGCAGCCCTCTGTTTGGCTGTCTGTACGAGGCCAACGACTACGAAGAGATCGT
GTTCCTGATGTTCACCCTGAAGCAGGCCTTTCCAGCCGAGTACCTGCCTCAAGAGCCCAAATCT
TcTGACAAAACTCACACATGCCCACCGTGCCCAGCACCTGAACTCCTGGGGGGACCGTCAGTCT
TCCTCTTCCCCCCAAAACCCAAGGACACCCTCATGATCTCCCGGACCCCTGAGGTCACATGCGT
GGTGGTGGACGTGAGCCACGAAGACCCTGAGGTCAAGTTCAACTGGTACGTGGACGGCGTGGAG
GTGCATAATGCCAAGACAAAGCCGCGGGAGGAGCAGTACgccAGCACGTACCGTGTGGTCAGCG
TCCTCACCGTCCTGCACCAGGACTGGCTGAATGGCAAGGAGTACAAGTGCAAGGTCTCCAACAA
AGCCCTCCCAGCCCCCATCGAGAAAACCATCTCCAAAGCCAAAGGGCAGCCCCGAGAACCACAG
GTcTACACCCTGCCCCCATCCCGGGAGGAGATGACCAAGAACCAGGTCAGCCTGtacTGCCTGG
TCAAAGGCTTCTATCCCAGCGACATCGCCGTGGAGTGGGAGAGCAATGGGCAGCCGGAGAACAA
CTACAAGACCACGCCTCCCGTGCTGGACTCCGACGGCTCCTTCTTCCTCTATAGCAAGCTCACC
GTGGACAAGAGCAGGTGGCAGCAGGGGAACGTCTTCTCATGCTCCGTGATGCATGAGGCTCTGC
ACAACCACTACACGCAGAAGAGCCTAAGCTTGTCTCCGGGTAAA
SEQ ID NO: 115:
DASLPYLQKESVFQSGAHAYRIPALLYLPGQQSLLAFAEQRASKKDEHAELIVLRRGDYDAGTH
QVQWQAQEVVAQARLDGHRSMNPCPLYDEQTGTLFLFFIAIPGQVTEQQQLQTRANVTRLCYVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRERVQAQSTNDGLDFQ
ESQLVKKLVEPPPTGCQGSVISFPSPRSGPGSPAQWLLYTHPTHSWQRADLGAYLNPRPPAPEA
WSEPVLLAKGSAAYSDLQSMGTGPDGSPLFGCLYEANDYEEIVELMFTLKQAFPAEYLPQEPKS
SDKTHTCPPCPAPELLGGPSVELFPPKPKDTLMISRTPEVTCVVVDVSHEDPEVKENWYVDGVE
VHNAKTKPREEQYASTYRVVSVLTVLHQDWLNGKEYKCKVSNKALPAPIEKTISKAKGQPREPQ
VYTLPPSREEMTKNQVSLYCLVKGFYPSDIAVEWESNGQPENNYKTTPPVLDSDGSFFLYSKLT
VDKSRWQQGNVFSCSVMHEALHNHYTQKSLSLSPGK
SEQ ID NO: 116:
X₁ASLPX₂LQX₃ESVFQSGAHAYRIPALLYLPGQQSLLAFAEQRASKKDEHAELIVLRRGDYDAX₄
THQVQWQAQEVVAQARLDGHRSMNPCPLYDX₅QTGTLFLFFIAIPGQVTEQQQLQTRANVTRLC
X₆VTSTDHGRTWSSPRDLTDAAIGPAYREWSTFAVGPGHCLQLHDRARSLVVPAYAYRKLHPX₇QR
PIPSAFCFLSHDHGRTWARGHEVAQDTLECQVAEVETGEQRVVTLNARSHLRX₈RVQAQSTNDGL
DFQESQLVKKLVEPPPX₉GCQGSVISFPSPRSGPGSPAQWLLYTHPTHX₁₀X₁₁QRADLGAYLNPR
PPAPEAWSEPVLLAKGSX₁₂AYSDLQSMGTGPDGSPLFGCLYEANDYEEIX₁₃FX₁₄MFTLKQAFP
AEYLPQX₁₅DKTHTCPPCPAPELLGGPSVFLFPPKPKDTLMISRTPEVTCVVVDVSHEDPEVKEN
WYVDGVEVHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVLDSDGSF
FLTSKLTVDKSRWQQGNVFSCSVMHEALHNHYTQKSLSLSPGK
SEQ ID NO: 117:
X₁X₂SX₃X₄X₅LQX₆ESVFQSGAHAYRIPALLYLPGQQSLLAFAEQRASX₇X₈DEHAELIVX₉RRGD
YDAX₁₀THQVQWX₁₁AQEVVAQAX₁₂LX₁₃GHRSMNPCPLYDX₁₄QTGTLFLFFIAIPX₁₅X₁₆VTEX₁₇
QQLQTRANVTRLX₁₈X₁₉VTSTDHGRTWSSPRDLTDAAIGPX₂₀YREWSTFAVGPGHX₂₁LQLHDX₂₂
X₂₃RSLVVPAYAYRKLHPX₂₄X₂₅X₂₆PIPSAFX₂₇FLSHDHGRTWARGHFVX₂₈QDTX₂₉ECQVAEV
X₃₀TGEQRVVTLNARSX₃₁X₃₂X₃₃X₃₄RX₃₅QAQSX₃₆NX₃₇GLDFQX₃₈X₃₉QX₄₀VKKL
X₄₁EPPPX₄₂GX₄₃QGSVISFPSPRSGPGSPAQX₄₄LLYTHPTHX₄₅X₄₆QRADLGAYLNPRPPAPEA
WSEPX₄₇LLAKGSX₄₈AYSDLQSMGTGPDGSPLFGX₄₉LYEANDYEEIX₅₀FX₅₁MFTLKQAFPAEYL
PQX₅₂DKTHTCPPCPAPELLGGPSVELFPPKPKDTLMISRTPEVTCVVVDVSHEDPEVKENWYVD
GVEVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFELTS
KLTVDKSRWQQGNVFSCSVMHEALHNHYTQKSLSLSPGK
SEQ ID NO: 118:
gatGCATCTCTGCCTTACCTGCAGAAAGAAAGCGTGTTCCAGTCTGGCGCCCACGCCTACAGAA
TTCCCGCTCTGCTGTATCTGCCAGGCCAGCAGTCTCTGCTGGCTTTCGCTGAACAGCGGGCCAG
CAAGAAGGATGAGCACGCCGAACTGATCGTGCTGCGGAGAGGCGATTACGACGCCggcACACAT
CAGGTGCAGTGGCAGGCTCAAGAGGTGGTGGCTCAGGCTAGACTGGACGGCCACAGATCTATGA
ACCCCTGTCCTCTGTACGATgaaCAGACCGGCACACTGTTTCTGTTCTTTATCGCTATCCCCGG
CCAAGTGACCGAGCAGCAGCAGCTGCAGACAAGAGCCAACGTGACCAGACTGTGTtacGTGACC
TCCACCGACCACGGCAGAACCTGGTCTAGCCCTAGAGATCTGACCGACGCCGCCATCGGACCTG
CCTATAGAGAGTGGTCCACCTTCGCCGTTGGACCTGGACACTGTCTCCAGCTGCACGACAGGGC
TAGATCTCTGGTGGTGCCTGCCTACGCCTATAGAAAGCTGCACCCCAAACAGCGGCCTATTCCT
AGCGCCTTCTGCTTTCTGAGCCACGATCACGGCAGGACATGGGCCAGAGGACATTTCGTGGCCC
AGGACACACTGGAATGCCAGGTGGCCGAAGTGGAAACCGGCGAGCAGAGAGTCGTGACCCTGAA
CGCCAGATCTCACCTGAGATTCAGAGTGCAGGCCCAGAGCACAAACGACGGCCTGGATTTCCAA
GAGAGCCAGCTGGTCAAGAAACTGGTGGAACCTCCTCCAaccGGCTGTCAGGGAAGCGTGATCA
GCTTTCCATCTCCTAGAAGCGGCCCTGGCTCTCCTGCTCAGTGGCTGCTGTATACACACCCCAC
ACACAGCTGGCAGAGAGCCGATCTGGGCGCCTACCTGAATCCTAGACCTCCTGCTCCTGAGGCT
TGGAGCGAACCTGTTCTGCTGGCCAAGGGCAGCgctGCCTACAGCGATCTGCAGTCTATGGGCA
CAGGCCCTGATGGCAGCCCTCTGTTTGGCTGTCTGTACGAGGCCAACGACTACGAAGAGATCGT
GTTCCTGATGTTCACCCTGAAGCAGGCCTTTCCAGCCGAGTACCTGCCTCAA
SEQ ID NO: 119:
X₁X₂SX₃X₄X₅LQX₆ESVFQSGAHAYRIPALLYLPGQQSLLAFAEQRASX₇X₈DEHAELIVX₉RRGD
YDAX₁₀THQVQWX₁₁AQEVVAQAX₁₂LX₁₃GHRSMNPCPLYDX₁₄QTGTLFLFFIAIPX₁₅X₁₆VTEX₁₇
QQLQTRANVTRLX₁₈X₁₉VTSTDHGRTWSSPRDLTDAAIGPX₂₀YREWSTFAVGPGHX₂₁LQLHDX₂₂
X₂₃RSLVVPAYAYRKLHPX₂₄X₂₅X₂₆PIPSAFX₂₇FLSHDHGRTWARGHFVX₂₈QDTX₂₉ECQVAEV
X₃₀TGEQRVVTLNARSX₃₁X₃₂X₃₃X₃₄RX₃₅QAQSX₃₆NX₃₇GLDFQX₃₈X₃₉QX₄₀VKKL
X₄₁EPPPX₄₂GX₄₃QGSVISFPSPRSGPGSPAQX₄₄LLYTHPTHX₄₅X₄₆QRADLGAYLNPRPPAPEA
WSEPX₄₇LLAKGSX₄₈AYSDLQSMGTGPDGSPLFGX₄₉LYEANDYEEIX₅₀FX₅₁MFTLKQAFPAEYL
PQ
SEQ ID NO: 120:
X₁ASLPX₂LQX₃ESVFQSGAHAYRIPALLYLPGQQSLLAFAEQRASKKDEHAELIVLRRGDYDAX₄
THQVQWQAQEVVAQARLDGHRSMNPCPLYDX₅QTGTLFLFFIAIPGQVTEQQQLQTRANVTRLCX₆
VTSTDHGRTWSSPRDLTDAAIGPAYREWSTFAVGPGHCLQLHDRARSLVVPAYAYRKLHPX₇QR
PIPSAFCFLSHDHGRTWARGHFVAQDTLECQVAEVETGEQRVVTLNARSHLRX₈RVQAQSTNDGL
DFQESQLVKKLVEPPPX₉GCQGSVISFPSPRSGPGSPAQWLLYTHPTHX₁₀X₁₁QRADLGAYLNPR
PPAPEAWSEPVLLAKGSX₁₂AYSDLQSMGTGPDGSPLFGCLYEANDYEEIX₁₃FX₁₄MFTLKQAFP
AEYLPQ
SEQ ID NO: 121:
GGGGS
SEQ ID NO: 122:
DASLPYLQKESVFQSGAHAYRIPALLYLPGQQSLLAFAEQRASKKDEHAELIVLRRGDYDAGTH
QVQWQAQEVVAQARLDGHRSMNPCPLYDEQTGTLFLFFIAIPGQVTEQQQLQTRANVTRLCYVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSTNDGLDFQ
ESQLVKKLVEPPPTGCQGSVISFPSPRSGPGSPAQWLLYTHPTHSWQRADLGAYLNPRPPAPEA
WSEPVLLAKGSAAYSDLQSMGTGPDGSPLFGCLYEANDYEEIVELMFTLKQAFPAEYLPQEPKS
SDKTHTCPPCPAPELLGGPSVFLFPPKPKDTLMISRTPEVTCVVVDVSHEDPEVKENWYVDGVE
VHNAKTKPREEQYNSTYRVVSVLTVLHQDWLNGKEYKCKVSNKALPAPIEKTISKAKGQPREPQ
VYTLPPSREEMTKNQVSLYCLVKGFYPSDIAVEWESNGQPENNYKTTPPVLDSDGSFFLYSKLT
VDKSRWQQGNVFSCSVMHEALHNHYTQKSLSLSPGK
SEQ ID NO: 123:
DASLPYLQKESVFQSGAHAYRIPALLYLPGQQSLLAFAEQRASKKDEHAELIVLRRGDYDAGTH
QVQWQAQEVVAQARLDGHRSMNPCPLYDEQTGTLFLFFIAIPGQVTEQQQLQTRANVTRLCYVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRERVQAQSTNDGLDFQ
ESQLVKKLVEPPPTGCQGSVISFPSPRSGPGSPAQWLLYTHPTHSWQRADLGAYLNPRPPAPEA
WSEPVLLAKGSAAYSDLQSMGTGPDGSPLFGCLYEANDYEEIVELMFTLKQAFPAEYLPQEPKS
SDKTHTCPPCPAPELLGGPSVFLFPPKPKDTLMISRTPEVTCVVVDVSHEDPEVKENWYVDGVE
VHNAKTKPREEQYNSTYRVVSVLTVLHQDWLNGKEYKCKVSNKALPAPIEKTISKAKGQPREPQ
VYTLPPSREEMTKNQVSLYCLVKGFYPSDIAVEWESNGQPENNYKTTPPVLDSDGSFFLYSKLT
VDKSRWQQGNVFSCSVMHEALHNHYTQKSLSLSPGK
SEQ ID NO: 124:
gatGCATCTCTGCCTTACCTGCAGAAAGAAAGCGTGTTCCAGTCTGGCGCCCACGCCTACAGAA
TTCCCGCTCTGCTGTATCTGCCAGGCCAGCAGTCTCTGCTGGCTTTCGCTGAACAGCGGGCCAG
CAAGAAGGATGAGCACGCCGAACTGATCGTGCTGCGGAGAGGCGATTACGACGCCggcACACAT
CAGGTGCAGTGGCAGGCTCAAGAGGTGGTGGCTCAGGCTAGACTGGACGGCCACAGATCTATGA
ACCCCTGTCCTCTGTACGATgaaCAGACCGGCACACTGTTTCTGTTCTTTATCGCTATCCCCGG
CCAAGTGACCGAGCAGCAGCAGCTGCAGACAAGAGCCAACGTGACCAGACTGTGTtacGTGACC
TCCACCGACCACGGCAGAACCTGGTCTAGCCCTAGAGATCTGACCGACGCCGCCATCGGACCTG
CCTATAGAGAGTGGTCCACCTTCGCCGTTGGACCTGGACACTGTCTCCAGCTGCACGACAGGGC
TAGATCTCTGGTGGTGCCTGCCTACGCCTATAGAAAGCTGCACCCCAAACAGCGGCCTATTCCT
AGCGCCTTCTGCTTTCTGAGCCACGATCACGGCAGGACATGGGCCAGAGGACATTTCGTGGCCC
AGGACACACTGGAATGCCAGGTGGCCGAAGTGGAAACCGGCGAGCAGAGAGTCGTGACCCTGAA
CGCCAGATCTCACCTGAGATTCAGAGTGCAGGCCCAGAGCACAAACGACGGCCTGGATTTCCAA
GAGAGCCAGCTGGTCAAGAAACTGGTGGAACCTCCTCCAaccGGCTGTCAGGGAAGCGTGATCA
GCTTTCCATCTCCTAGAAGCGGCCCTGGCTCTCCTGCTCAGTGGCTGCTGTATACACACCCCAC
ACACAGCTGGCAGAGAGCCGATCTGGGCGCCTACCTGAATCCTAGACCTCCTGCTCCTGAGGCT
TGGAGCGAACCTGTTCTGCTGGCCAAGGGCAGCgctGCCTACAGCGATCTGCAGTCTATGGGCA
CAGGCCCTGATGGCAGCCCTCTGTTTGGCTGTCTGTACGAGGCCAACGACTACGAAGAGATCGT
GTTCCTGATGTTCACCCTGAAGCAGGCCTTTCCAGCCGAGTACCTGCCTCAAGAGCCCAAATCT
TcTGACAAAACTCACACATGCCCACCGTGCCCAGCACCTGAACTCCTGGGGGGACCGTCAGTCT
TCCTCTTCCCCCCAAAACCCAAGGACACCCTCATGATCTCCCGGACCCCTGAGGTCACATGCGT
GGTGGTGGACGTGAGCCACGAAGACCCTGAGGTCAAGTTCAACTGGTACGTGGACGGCGTGGAG
GTGCATAATGCCAAGACAAAGCCGCGGGAGGAGCAGTACgccAGCACGTACCGTGTGGTCAGCG
TCCTCACCGTCCTGCACCAGGACTGGCTGAATGGCAAGGAGTACAAGTGCAAGGTCTCCAACAA
AGCCCTCCCAGCCCCCATCGAGAAAACCATCTCCAAAGCCAAAGGGCAGCCCCGAGAACCACAG
GTCTACACCCTGCCCCCATCCCGGGAGGAGATGACCAAGAACCAGGTCAGCCTGtacTGCCTGG
TCAAAGGCTTCTATCCCAGCGACATCGCCGTGGAGTGGGAGAGCAATGGGCAGCCGGAGAACAA
CTACAAGACCACGCCTCCCGTGCTGGACTCCGACGGCTCCTTCTTCCTCTATAGCAAGCTCACC
GTGGACAAGAGCAGGTGGCAGCAGGGGAACGTCTTCTCATGCTCCGTGATGCATGAGGCTCTGC
ACAACCACTACACGCAGAAGAGCCTAAGCTTGTCTCCGGGTAAA
SEQ ID NO: 125:
DASLPYLQKESVFQSGAHAYRIPALLYLPGQQSLLAFAEQRRSKKDEHAELIVLRRGDYDAGTH
QVQWQAQEVVAQARLDGHRSMNPCPLYDEQTGTLFLFFIAIPGQVTEQQQLQTRANVTRLCYVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRERVQAQSTNDGLDFQ
ESQLVKKLVEPPPTGCQGSVISFPSPRSGPGSPAQWLLYTHPTHSWQRADLGAYLNPRPPAPEA
WSEPVLLAKGSAAYSDLQSMGTGPDGSPLFGCLYEANDYEEIVELMFTLKQAFPAEYLPQEPKS
SDKTHTCPPCPAPELLGGPSVELFPPKPKDTLMISRTPEVTCVVVDVSHEDPEVKENWYVDGVE
VHNAKTKPREEQYASTYRVVSVLTVLHQDWLNGKEYKCKVSNKALPAPIEKTISKAKGQPREPQ
VYTLPPSREEMTKNQVSLYCLVKGFYPSDIAVEWESNGQPENNYKTTPPVLDSDGSFFLYSKLT
VDKSRWQQGNVFSCSVMHEALHNHYTQKSLSLSPGK
SEQ ID NO: 126:
DASLPYLQKESVFQSGAHAYRIPALLYLPGQQSLLAFAEQRASKKDEHAELIVLRRGDYDAGTH
QVQWQAQEVVAQARLDGHRSMNPCPLYDEQTGTLFLFFIAIPGQVTEQQQLQTRANVTRLCYVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSTNDGLDFQ
ESQLVKKLVEPPPQGCQGSVISFPSPRSGPGSPAQWLLYTHPTHSWQRADLGAYLNPRPPAPEA
WSEPVLLAKGSAAYSDLQSMGTGPDGSPLFGCLYEANDYEEIVELMFTLKQAFPAEYLPQ
SEQ ID NO: 127:
DASLPYLQKESVFQSGAHAYRIPALLYLPGQQSLLAFAEQRASKKDEHAELIVLRRGDYDAGTH
QVQWQAQEVVAQARLDGHRSMNPCPLYDEQTGTLFLFFIAIPGQVTEQQQLQTRANVTRLCYVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSTNDGLDFQ
ESQLVKKLVEPPPQGCQGSVISFPSPRSGPGSPAQWLLYTHPTHSWQRADLGAYLNPRPPAPEA
WSEPVLLAKGSAAYSDLQSMGTGPDGSPLFGCLYEANDYEEIVELMFTLKQAFPAEYLPQEPKS
SDKTHTCPPCPAPELLGGPSVFLFPPKPKDTLMISRTPEVTCVVVDVSHEDPEVKENWYVDGVE
VHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFELTSKLT
VDKSRWQQGNVFSCSVMHEALHNHYTQKSLSLSPGK
SEQ ID NO: 128:
DASLPYLQKESVFQSGAHAYRIPALLYLPGQQSLLAFAEQRASKKDEHAELIVLRRGDYDAGTH
QVQWQAQEVVAQARLDGHRSMNPCPLYDEQTGTLFLFFIAIPGQVTEQQQLQTRANVTRLCYVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRARVQAQSTNDGLDFQ
ESQLVKKLVEPPPQGCQGSVISFPSPRSGPGSPAQWLLYTHPTHSWQRADLGAYLNPRPPAPEA
WSEPVLLAKGSAAYSDLQSMGTGPDGSPLFGCLYEANDYEEIVELMFTLKQAFPAEYLPQGGGG
SGGGGSDKTHTCPPCPAPELLGGPSVELFPPKPKDTLMISRTPEVTCVVVDVSHEDPEVKENWY
VDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFFL
TSKLTVDKSRWQQGNVFSCSVMHEALHNHYTQKSLSLSPGK
SEQ ID NO: 129:
gatGCATCTCTGCCTTACCTGCAGAAAGAAAGCGTGTTCCAGTCTGGCGCCCACGCCTACAGAA
TTCCCGCTCTGCTGTATCTGCCAGGCCAGCAGTCTCTGCTGGCTTTCGCTGAACAGCGGcggAG
CAAGAAGGATGAGCACGCCGAACTGATCGTGCTGCGGAGAGGCGATTACGACGCCggcACACAT
CAGGTGCAGTGGCAGGCTCAAGAGGTGGTGGCTCAGGCTAGACTGGACGGCCACAGATCTATGA
ACCCCTGTCCTCTGTACGATgaaCAGACCGGCACACTGTTTCTGTTCTTTATCGCTATCCCCGG
CCAAGTGACCGAGCAGCAGCAGCTGCAGACAAGAGCCAACGTGACCAGACTGTGTtacGTGACC
TCCACCGACCACGGCAGAACCTGGTCTAGCCCTAGAGATCTGACCGACGCCGCCATCGGACCTG
CCTATAGAGAGTGGTCCACCTTCGCCGTTGGACCTGGACACTGTCTCCAGCTGCACGACAGGGC
TAGATCTCTGGTGGTGCCTGCCTACGCCTATAGAAAGCTGCACCCCAAACAGCGGCCTATTCCT
AGCGCCTTCTGCTTTCTGAGCCACGATCACGGCAGGACATGGGCCAGAGGACATTTCGTGGCCC
AGGACACACTGGAATGCCAGGTGGCCGAAGTGGAAACCGGCGAGCAGAGAGTCGTGACCCTGAA
CGCCAGATCTCACCTGAGATTCAGAGTGCAGGCCCAGAGCACAAACGACGGCCTGGATTTCCAA
GAGAGCCAGCTGGTCAAGAAACTGGTGGAACCTCCTCCAaccGGCTGTCAGGGAAGCGTGATCA
GCTTTCCATCTCCTAGAAGCGGCCCTGGCTCTCCTGCTCAGTGGCTGCTGTATACACACCCCAC
ACACAGCTGGCAGAGAGCCGATCTGGGCGCCTACCTGAATCCTAGACCTCCTGCTCCTGAGGCT
TGGAGCGAACCTGTTCTGCTGGCCAAGGGCAGCgctGCCTACAGCGATCTGCAGTCTATGGGCA
CAGGCCCTGATGGCAGCCCTCTGTTTGGCTGTCTGTACGAGGCCAACGACTACGAAGAGATCGT
GTTCCTGATGTTCACCCTGAAGCAGGCCTTTCCAGCCGAGTACCTGCCTCAAGAGCCCAAATCT
TcTGACAAAACTCACACATGCCCACCGTGCCCAGCACCTGAACTCCTGGGGGGACCGTCAGTCT
TCCTCTTCCCCCCAAAACCCAAGGACACCCTCATGATCTCCCGGACCCCTGAGGTCACATGCGT
GGTGGTGGACGTGAGCCACGAAGACCCTGAGGTCAAGTTCAACTGGTACGTGGACGGCGTGGAG
GTGCATAATGCCAAGACAAAGCCGCGGGAGGAGCAGTACgccAGCACGTACCGTGTGGTCAGCG
TCCTCACCGTCCTGCACCAGGACTGGCTGAATGGCAAGGAGTACAAGTGCAAGGTCTCCAACAA
AGCCCTCCCAGCCCCCATCGAGAAAACCATCTCCAAAGCCAAAGGGCAGCCCCGAGAACCACAG
GTcTACACCCTGCCCCCATCCCGGGAGGAGATGACCAAGAACCAGGTCAGCCTGtacTGCCTGG
TCAAAGGCTTCTATCCCAGCGACATCGCCGTGGAGTGGGAGAGCAATGGGCAGCCGGAGAACAA
CTACAAGACCACGCCTCCCGTGCTGGACTCCGACGGCTCCTTCTTCCTCTATAGCAAGCTCACC
GTGGACAAGAGCAGGTGGCAGCAGGGGAACGTCTTCTCATGCTCCGTGATGCATGAGGCTCTGC
ACAACCACTACACGCAGAAGAGCCTAAGCTTGTCTCCGGGTAAA
SEQ ID NO: 130:
DASLPYLQKESVFQSGAHAYRIPALLYLPGQQSLLAFAEQRRSKKDEHAELIVLRRGDYDAGTH
QVQWQAQEVVAQARLDGHRSMNPCPLYDEQTGTLFLFFIAIPGQVTEQQQLQTRANVTRLCYVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRERVQAQSINDGLDFQ
ESQLVKKLVEPPPTGCQGSVISFPSPRSGPGSPAQWLLYTHPTHSWQRADLGAYLNPRPPAPEA
WSEPVLLAKGSAAYSDLQSMGTGPDGSPLFGCLYEANDYEEIVELMFTLKQAFPAEYLPQEPKS
SDKTHTCPPCPAPELLGGPSVELFPPKPKDTLMISRTPEVTCVVVDVSHEDPEVKENWYVDGVE
VHNAKTKPREEQYASTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGKGGGGSGGGGSGGGGSQVQLVQSGVEVKK
PGASVKVSCKASGYTFTNYYMYWVRQAPGQGLEWMGGINPSNGGTNENEKFKNRVTLTTDSSTT
TAYMELKSLQFDDTAVYYCARRDYRFDMGFDYWGQGTTVTVSSASTKGPSVFPLAPSSKSTSGG
TAALGCLVKDYFPEPVTVSWNSGALTSGVHTFPAVLQSSGLYSLSSVVTVPSSSLGTQTYICNV
NHKPSNTKVDKKVEPKSCDKTS
SEQ ID NO: 131:
gatGCATCTCTGCCTTACCTGCAGAAAGAAAGCGTGTTCCAGTCTGGCGCCCACGCCTACAGAA
TTCCCGCTCTGCTGTATCTGCCAGGCCAGCAGTCTCTGCTGGCTTTCGCTGAACAGCGGcggAG
CAAGAAGGATGAGCACGCCGAACTGATCGTGCTGCGGAGAGGCGATTACGACGCCggcACACAT
CAGGTGCAGTGGCAGGCTCAAGAGGTGGTGGCTCAGGCTAGACTGGACGGCCACAGATCTATGA
ACCCCTGTCCTCTGTACGATgaaCAGACCGGCACACTGTTTCTGTTCTTTATCGCTATCCCCGG
CCAAGTGACCGAGCAGCAGCAGCTGCAGACAAGAGCCAACGTGACCAGACTGTGTtacGTGACC
TCCACCGACCACGGCAGAACCTGGTCTAGCCCTAGAGATCTGACCGACGCCGCCATCGGACCTG
CCTATAGAGAGTGGTCCACCTTCGCCGTTGGACCTGGACACTGTCTCCAGCTGCACGACAGGGC
TAGATCTCTGGTGGTGCCTGCCTACGCCTATAGAAAGCTGCACCCCAAACAGCGGCCTATTCCT
AGCGCCTTCTGCTTTCTGAGCCACGATCACGGCAGGACATGGGCCAGAGGACATTTCGTGGCCC
AGGACACACTGGAATGCCAGGTGGCCGAAGTGGAAACCGGCGAGCAGAGAGTCGTGACCCTGAA
CGCCAGATCTCACCTGAGATTCAGAGTGCAGGCCCAGAGCACAAACGACGGCCTGGATTTCCAA
GAGAGCCAGCTGGTCAAGAAACTGGTGGAACCTCCTCCAaccGGCTGTCAGGGAAGCGTGATCA
GCTTTCCATCTCCTAGAAGCGGCCCTGGCTCTCCTGCTCAGTGGCTGCTGTATACACACCCCAC
ACACAGCTGGCAGAGAGCCGATCTGGGCGCCTACCTGAATCCTAGACCTCCTGCTCCTGAGGCT
TGGAGCGAACCTGTTCTGCTGGCCAAGGGCAGCgctGCCTACAGCGATCTGCAGTCTATGGGCA
CAGGCCCTGATGGCAGCCCTCTGTTTGGCTGTCTGTACGAGGCCAACGACTACGAAGAGATCGT
GTTCCTGATGTTCACCCTGAAGCAGGCCTTTCCAGCCGAGTACCTGCCTCAAGAGCCCAAATCT
TcTGACAAAACTCACACATGCCCACCGTGCCCAGCACCTGAACTCCTGGGGGGACCGTCAGTCT
TCCTCTTCCCCCCAAAACCCAAGGACACCCTCATGATCTCCCGGACCCCTGAGGTCACATGCGT
GGTGGTGGACGTGAGCCACGAAGACCCTGAGGTCAAGTTCAACTGGTACGTGGACGGCGTGGAG
GTGCATAATGCCAAGACAAAGCCGCGGGAGGAGCAGTACgccAGCACGTACCGTGTGGTCAGCG
TCCTCACCGTCCTGCACCAGGACTGGCTGAATGGCAAGGAGTACAAGTGCAAGGTCTCCAACAA
AGCCCTCCCAGCCCCCATCGAGAAAACCATCTCCAAAGCCAAAGGGCAGCCCCGAGAACCACAG
GTcTACACCCTGCCCCCATCCCGGGAGGAGATGACCAAGAACCAGGTCAGCCTGACCTGCCTGG
TCAAAGGCTTCTATCCCAGCGACATCGCCGTGGAGTGGGAGAGCAATGGGCAGCCGGAGAACAA
CTACAAGACCACGCCTCCCGTGCTGGACTCCGACGGCTCCTTCTTCCTCTATAGCAAGCTCACC
GTGGACAAGAGCAGGTGGCAGCAGGGGAACGTCTTCTCATGCTCCGTGATGCATGAGGCTCTGC
ACAACCACTACACGCAGAAGAGCCTAAGCTTGTCTCCGGGTAAAGGAGGCGGAGGATCTGGCGG
AGGTGGAAGTGGCGGAGGCGGATCTCAGGTTCAGCTGGTGCAGTCTGGCGTGGAAGTGAAGAAA
CCTGGGGCCAGCGTGAAGGTGTCCTGTAAAGCCAGCGGCTACACCTTTACCAACTACTACATGT
ACTGGGTCCGACAGGCCCCAGGCCAAGGACTTGAATGGATGGGCGGCATCAACCCCAGCAACGG
CGGCACCAACTTCAACGAGAAGTTCAAGAACAGAGTGACCCTGACCACCGACAGCAGCACCACC
ACAGCCTACATGGAACTGAAGTCCCTGCAGTTCGACGACACCGCCGTGTACTACTGCGCCAGAC
GGGACTACAGATTCGACATGGGCTTCGACTATTGGGGCCAGGGCACCACCGTGACAGTCTCTTC
TGCtagcACCAAGGGCCCATCcGTCTTCCCCCTGGCACCCTCCTCCAAGAGCACCTCTGGGGGC
ACAGCGGCCCTGGGCTGCCTGGTCAAGGACTACTTCCCCGAACCGGTGACGGTGTCCTGGAACT
CAGGCGCtCTGACCAGCGGCGTGCACACCTTCCCGGCTGTCCTACAGTCCTCAGGACTCTACTC
CCTCAGCAGCGTGGTGACCGTGCCCTCCAGCAGCTTGGGCACCCAGACCTACATCTGCAACGTG
AATCACAAGCCCAGCAACACCAAGGTGGACAAGAAAGTTGAGCCCAAATCTTGTGACAAAACTa
gc
SEQ ID NO: 132:
QVQLVQSGVEVKKPGASVKVSCKASGYTFTNYYMYWVRQAPGQGLEWMGGINPSNGGTNENEKE
KNRVTLTTDSSTTTAYMELKSLQFDDTAVYYCARRDYRFDMGFDYWGQGTTVTVSSASTKGPSV
FPLAPSSKSTSGGTAALGCLVKDYFPEPVTVSWNSGALTSGVHTFPAVLQSSGLYSLSSVVTVP
SSSLGTQTYICNVNHKPSNTKVDKKVEPKSCDKTS
SEQ ID NO: 133:
CAGGTTCAGCTGGTGCAGTCTGGCGTGGAAGTGAAGAAACCTGGGGCCAGCGTGAAGGTGTCCT
GTAAAGCCAGCGGCTACACCTTTACCAACTACTACATGTACTGGGTCCGACAGGCCCCAGGCCA
AGGACTTGAATGGATGGGCGGCATCAACCCCAGCAACGGCGGCACCAACTTCAACGAGAAGTTC
AAGAACAGAGTGACCCTGACCACCGACAGCAGCACCACCACAGCCTACATGGAACTGAAGTCCC
TGCAGTTCGACGACACCGCCGTGTACTACTGCGCCAGACGGGACTACAGATTCGACATGGGCTT
CGACTATTGGGGCCAGGGCACCACCGTGACAGTCTCTTCTGCtagcACCAAGGGCCCATCcGTC
TTCCCCCTGGCACCCTCCTCCAAGAGCACCTCTGGGGGCACAGCGGCCCTGGGCTGCCTGGTCA
AGGACTACTTCCCCGAACCGGTGACGGTGTCCTGGAACTCAGGCGCtCTGACCAGCGGCGTGCA
CACCTTCCCGGCTGTCCTACAGTCCTCAGGACTCTACTCCCTCAGCAGCGTGGTGACCGTGCCC
TCCAGCAGCTTGGGCACCCAGACCTACATCTGCAACGTGAATCACAAGCCCAGCAACACCAAGG
TGGACAAGAAAGTTGAGCCCAAATCTTGTGACAAAACTagc
SEQ ID NO: 134:
DASLPYLQKESVFQSGAHAYRIPALLYLPGQQSLLAFAEQRRSKKDEHAELIVLRRGDYDAGTH
QVQWQAQEVVAQARLDGHRSMNPCPLYDEQTGTLFLFFIAIPGQVTEQQQLQTRANVTRLCYVT
STDHGRTWSSPRDLTDAAIGPAYREWSTFAVGPGHCLQLHDRARSLVVPAYAYRKLHPKQRPIP
SAFCFLSHDHGRTWARGHFVAQDTLECQVAEVETGEQRVVTLNARSHLRFRVQAQSTNDGLDFQ
ESQLVKKLVEPPPTGCQGSVISFPSPRSGPGSPAQWLLYTHPTHSWQRADLGAYLNPRPPAPEA
WSEPVLLAKGSAAYSDLQSMGTGPDGSPLFGCLYEANDYEEIVELMFTLKQAFPAEYLPQEPKS
SDKTHTCPPCPAPELLGGPSVELFPPKPKDTLMISRTPEVTCVVVDVSHEDPEVKENWYVDGVE
VHNAKTKPREEQYASTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGKGGGGSGGGGSGGGGSEIVLTQSPATLSL
SPGERATLSCRASKGVSTSGYSYLHWYQQKPGQAPRLLIYLASYLESGVPARESGSGSGTDETL
TISSLEPEDFAVYYCQHSRDLPLTFGGGTKVEIKRTVAAPSVFIFPPSDEQLKSGTASVVCLLN
NFYPREAKVQWKVDNALQSGNSQESVTEQDSKDSTYSLSSTLTLSKADYEKHKVYACEVTHQGL
SSPVTKSENRGEC
SEQ ID NO: 135:
gatGCATCTCTGCCTTACCTGCAGAAAGAAAGCGTGTTCCAGTCTGGCGCCCACGCCTACAGAA
TTCCCGCTCTGCTGTATCTGCCAGGCCAGCAGTCTCTGCTGGCTTTCGCTGAACAGCGGcggAG
CAAGAAGGATGAGCACGCCGAACTGATCGTGCTGCGGAGAGGCGATTACGACGCCggcACACAT
CAGGTGCAGTGGCAGGCTCAAGAGGTGGTGGCTCAGGCTAGACTGGACGGCCACAGATCTATGA
ACCCCTGTCCTCTGTACGATgaaCAGACCGGCACACTGTTTCTGTTCTTTATCGCTATCCCCGG
CCAAGTGACCGAGCAGCAGCAGCTGCAGACAAGAGCCAACGTGACCAGACTGTGTtacGTGACC
TCCACCGACCACGGCAGAACCTGGTCTAGCCCTAGAGATCTGACCGACGCCGCCATCGGACCTG
CCTATAGAGAGTGGTCCACCTTCGCCGTTGGACCTGGACACTGTCTCCAGCTGCACGACAGGGC
TAGATCTCTGGTGGTGCCTGCCTACGCCTATAGAAAGCTGCACCCCAAACAGCGGCCTATTCCT
AGCGCCTTCTGCTTTCTGAGCCACGATCACGGCAGGACATGGGCCAGAGGACATTTCGTGGCCC
AGGACACACTGGAATGCCAGGTGGCCGAAGTGGAAACCGGCGAGCAGAGAGTCGTGACCCTGAA
CGCCAGATCTCACCTGAGATTCAGAGTGCAGGCCCAGAGCACAAACGACGGCCTGGATTTCCAA
GAGAGCCAGCTGGTCAAGAAACTGGTGGAACCTCCTCCAaccGGCTGTCAGGGAAGCGTGATCA
GCTTTCCATCTCCTAGAAGCGGCCCTGGCTCTCCTGCTCAGTGGCTGCTGTATACACACCCCAC
ACACAGCTGGCAGAGAGCCGATCTGGGCGCCTACCTGAATCCTAGACCTCCTGCTCCTGAGGCT
TGGAGCGAACCTGTTCTGCTGGCCAAGGGCAGCgctGCCTACAGCGATCTGCAGTCTATGGGCA
CAGGCCCTGATGGCAGCCCTCTGTTTGGCTGTCTGTACGAGGCCAACGACTACGAAGAGATCGT
GTTCCTGATGTTCACCCTGAAGCAGGCCTTTCCAGCCGAGTACCTGCCTCAAGAGCCCAAATCT
TcTGACAAAACTCACACATGCCCACCGTGCCCAGCACCTGAACTCCTGGGGGGACCGTCAGTCT
TCCTCTTCCCCCCAAAACCCAAGGACACCCTCATGATCTCCCGGACCCCTGAGGTCACATGCGT
GGTGGTGGACGTGAGCCACGAAGACCCTGAGGTCAAGTTCAACTGGTACGTGGACGGCGTGGAG
GTGCATAATGCCAAGACAAAGCCGCGGGAGGAGCAGTACgccAGCACGTACCGTGTGGTCAGCG
TCCTCACCGTCCTGCACCAGGACTGGCTGAATGGCAAGGAGTACAAGTGCAAGGTCTCCAACAA
AGCCCTCCCAGCCCCCATCGAGAAAACCATCTCCAAAGCCAAAGGGCAGCCCCGAGAACCACAG
GTcTACACCCTGCCCCCATCCCGGGAGGAGATGACCAAGAACCAGGTCAGCCTGACCTGCCTGG
TCAAAGGCTTCTATCCCAGCGACATCGCCGTGGAGTGGGAGAGCAATGGGCAGCCGGAGAACAA
CTACAAGACCACGCCTCCCGTGCTGGACTCCGACGGCTCCTTCTTCCTCTATAGCAAGCTCACC
GTGGACAAGAGCAGGTGGCAGCAGGGGAACGTCTTCTCATGCTCCGTGATGCATGAGGCTCTGC
ACAACCACTACACGCAGAAGAGCCTAAGCTTGTCTCCGGGTAAAGGAGGCGGAGGATCTGGCGG
AGGTGGAAGTGGCGGAGGCGGATCTGAGATCGTGCTGACACAGAGCCCTGCCACACTGTCACTG
TCTCCAGGCGAGAGAGCCACACTGAGCTGTAGAGCTTCTAAGGGCGTGTCCACCAGCGGCTACT
CTTACCTGCACTGGTATCAGCAGAAGCCCGGCCAGGCTCCTAGACTGCTGATCTACCTGGCCAG
CTACCTGGAAAGCGGAGTGCCTGCTAGATTCAGCGGCTCTGGCTCTGGCACCGACTTCACCCTG
ACAATCAGCAGCCTGGAACCTGAGGACTTCGCCGTGTACTACTGCCAGCACAGCAGAGATCTGC
CCCTGACATTTGGCGGAGGCACCAAGGTGGAAATCAAGCGTACGGTGGCTGCACCATCTGTCTT
CATCTTCCCGCCATCTGATGAGCAGTTGAAATCTGGAACTGCCTCTGTTGTGTGCCTGCTGAAT
AACTTCTATCCCAGAGAGGCCAAAGTACAGTGGAAGGTGGATAACGCCCTCCAATCGGGTAACT
CCCAGGAGAGTGTCACAGAGCAGGACAGCAAGGACAGCACCTACAGCCTCAGCAGCACCCTGAC
GCTGAGCAAAGCAGACTACGAGAAACACAAAGTCTACGCCTGCGAAGTCACCCATCAGGGCCTG
AGCTCGCCCGTCACAAAGAGCTTCAACAGGGGAGAGTGT
SEQ ID NO: 136:
QVQLVQSGVEVKKPGASVKVSCKASGYTFTNYYMYWVRQAPGQGLEWMGGINPSNGGTNENEKF
KNRVTLTTDSSTTTAYMELKSLQFDDTAVYYCARRDYRFDMGFDYWGQGTTVTVSSASTKGPSV
FPLAPCSRSTSESTAALGCLVKDYFPEPVTVSWNSGALTSGVHTFPAVLQSSGLYSLSSVVTVP
SSSLGTKTYTCNVDHKPSNTKVDKRVESKYGPPCPPCPAPEFLGGPSVELFPPKPKDTLMISRT
PEVTCVVVDVSQEDPEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVKGFYPSDIAVEWESN
GQPENNYKTTPPVLDSDGSFFLYSRLTVDKSRWQEGNVFSCSVMHEALHNHYTQKSLSLSLGK
SEQ ID NO: 137:
QVQLVQSGVEVKKPGASVKVSCKASGYTFTNYYMYWVRQAPGQGLEWMGGINPSNGGTNENEKE
KNRVTLTTDSSTTTAYMELKSLQFDDTAVYYCARRDYRFDMGEDYWGQGTTVTVSS
SEQ ID NO: 138:
EIVLTQSPATLSLSPGERATLSCRASKGVSTSGYSYLHWYQQKPGQAPRLLIYLASYLESGVPA
RFSGSGSGTDFTLTISSLEPEDFAVYYCQHSRDLPLTFGGGTKVEIK
SEQ ID NO: 139:
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVELFPP
KPKDTLMISRTPEVTCVVVDVSHEDPEVKENWYVDGVEVHNAKTKPREEQYNSTYRVVSVLTVL
HQDWLNGKEYKCKVSNKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNVESCSVMHEALHNHYT
QKSLSLSPGK
SEQ ID NO: 140:
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVELEPP
KPKDTLMISRTPEVTCVVVDVSHEDPEVKENWYVDGVEVHNAKTKPREEQYASTYRVVSVLTVL
HQDWLNGKEYKCKVSNKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNVFSCSVMHEALHNHYT
QKSLSLSPGK
SEQ ID NO: 141:
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVELEPP
KPKDTLMISRTPEVTCVVVDVSHEDPEVKENWYVDGVEVHNAKTKPREEQYNSTYRVVSVLTVL
HQDWLNGKEYKCKVSNKALPAPIEKTISKAKGQPREPQVYTLPPSREEMTKNQVSLTCLVKGFY
PSDIAVEWESNGQPENNYKTTPPVLDSDGSFFLTSKLTVDKSRWQQGNVFSCSVMHEALHNHYT
QKSLSLSPGK
SEQ ID NO: 142:
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVELEPP
KPKDTLMISRTPEVTCVVVDVSHEDPEVKENWYVDGVEVHNAKTKPREEQYNSTYRVVSVLTVL
HQDWLNGKEYKCKVSNKALPAPIEKTISKAKGQPREPQVYTLPPSREEMTKNQVSLYCLVKGFY
PSDIAVEWESNGQPENNYKTTPPVLDSDGSFFLYSKLTVDKSRWQQGNVFSCSVMHEALHNHYT
QKSLSLSPGK
SEQ ID NO: 143:
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVELEPP
KPKDTLMISRTPEVTCVVVDVSHEDPEVKENWYVDGVEVHNAKTKPREEQYASTYRVVSVLTVL
HQDWLNGKEYKCKVSNKALPAPIEKTISKAKGQPREPQVYTLPPSREEMTKNQVSLTCLVKGFY
PSDIAVEWESNGQPENNYKTTPPVLDSDGSFFLTSKLTVDKSRWQQGNVFSCSVMHEALHNHYT
QKSLSLSPGK
SEQ ID NO: 144:
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVELEPP
KPKDTLMISRTPEVTCVVVDVSHEDPEVKENWYVDGVEVHNAKTKPREEQYASTYRVVSVLTVL
HQDWLNGKEYKCKVSNKALPAPIEKTISKAKGQPREPQVYTLPPSREEMTKNQVSLYCLVKGFY
PSDIAVEWESNGQPENNYKTTPPVLDSDGSFFLYSKLTVDKSRWQQGNVFSCSVMHEALHNHYT
QKSLSLSPGK
SEQ ID NO: 145:
MGWSCIILFLVATATGVHSQVQLVQSGVEVKKPGASVKVSCKASGYTFTNYYMYWVRQAPGQGL
EWMGGINPSNGGTNFNEKFKNRVTLTTDSSTTTAYMELKSLQFDDTAVYYCARRDYRFDMGEDY
WGQGTTVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPKSCDKTHTCPPCPAPEL
LGGPSVFLFPPKPKDTLMISRTPEVTCVVVDVSHEDPEVKENWYVDGVEVHNAKTKPREEQYAS
TYRVVSVLTVLHQDWLNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLTVDKSRWQQGNVESCS
VMHEALHNHYTQKSLSLSPGKGGGGSGGGGSGGGGSDASLPYLQKESVFQSGAHAYRIPALLYL
PGQQSLLAFAEQRASKKDEHAELIVLRRGDYDAGTHQVQWQAQEVVAQARLDGHRSMNPCPLYD
EQTGTLFLFFIAIPGQVTEQQQLQTRANVTRLCYVTSTDHGRTWSSPRDLTDAAIGPAYREWST
FAVGPGHCLQLHDRARSLVVPAYAYRKLHPKQRPIPSAFCFLSHDHGRTWARGHFVAQDTLECQ
VAEVETGEQRVVTLNARSHLRERVQAQSTNDGLDFQESQLVKKLVEPPPTGCQGSVISFPSPRS
GPGSPAQWLLYTHPTHSWQRADLGAYLNPRPPAPEAWSEPVLLAKGSAAYSDLQSMGTGPDGSP
LFGCLYEANDYEEIVELMFTLKQAFPAEYLPQ
SEQ ID NO: 146:
atgGGATGGAGCTGTATCATCCTCTTCTTGGTAGCAACAGCTACAGGTGTaCACTCCCAGGTTC
AGCTGGTGCAGTCTGGCGTGGAAGTGAAGAAACCTGGGGCCAGCGTGAAGGTGTCCTGTAAAGC
CAGCGGCTACACCTTTACCAACTACTACATGTACTGGGTCCGACAGGCCCCAGGCCAAGGACTT
GAATGGATGGGCGGCATCAACCCCAGCAACGGCGGCACCAACTTCAACGAGAAGTTCAAGAACA
GAGTGACCCTGACCACCGACAGCAGCACCACCACAGCCTACATGGAACTGAAGTCCCTGCAGTT
CGACGACACCGCCGTGTACTACTGCGCCAGACGGGACTACAGATTCGACATGGGCTTCGACTAT
TGGGGCCAGGGCACCACCGTGACAGTCTCTTCTGCtagcACCAAGGGCCCATCcGTCTTCCCCC
TGGCACCCTCCTCCAAGAGCACCTCTGGGGGCACAGCGGCCCTGGGCTGCCTGGTCAAGGACTA
CTTCCCCGAACCGGTGACGGTGTCCTGGAACTCAGGCGCtCTGACCAGCGGCGTGCACACCTTC
CCGGCTGTCCTACAGTCCTCAGGACTCTACTCCCTCAGCAGCGTGGTGACCGTGCCCTCCAGCA
GCTTGGGCACCCAGACCTACATCTGCAACGTGAATCACAAGCCCAGCAACACCAAGGTGGACAA
GAAAGTTGAGCCCAAATCTTGTGACAAAACTCACACATGCCCACCGTGCCCAGCACCTGAACTC
CTGGGGGGACCGTCAGTCTTCCTCTTCCCCCCAAAACCCAAGGACACCCTCATGATCTCCCGGA
CCCCTGAGGTCACATGCGTGGTGGTGGACGTGAGCCACGAAGACCCTGAGGTCAAGTTCAACTG
GTACGTGGACGGCGTGGAGGTGCATAATGCCAAGACAAAGCCGCGGGAGGAGCAGTACgccAGC
ACGTACCGTGTGGTCAGCGTCCTCACCGTCCTGCACCAGGACTGGCTGAATGGCAAGGAGTACA
AGTGCAAGGTCTCCAACAAAGCCCTCCCAGCCCCCATCGAGAAAACCATCTCCAAAGCCAAAGG
GCAGCCCCGAGAACCACAGGTcTACACCCTGCCCCCATCCCGGGAGGAGATGACCAAGAACCAG
GTCAGCCTGACCTGCCTGGTCAAAGGCTTCTATCCCAGCGACATCGCCGTGGAGTGGGAGAGCA
ATGGGCAGCCGGAGAACAACTACAAGACCACGCCTCCCGTGCTGGACTCCGACGGCTCCTTCTT
CCTCTATAGCAAGCTCACCGTGGACAAGAGCAGGTGGCAGCAGGGGAACGTCTTCTCATGCTCC
GTGATGCATGAGGCTCTGCACAACCACTACACGCAGAAGAGCCTAAGCTTGTCTCCGGGTAAAG
GAGGCGGAGGATCTGGCGGAGGTGGAAGTGGCGGAGGCGGATCTgatGCATCTCTGCCTTACCT
GCAGAAAGAAAGCGTGTTCCAGTCTGGCGCCCACGCCTACAGAATTCCCGCTCTGCTGTATCTG
CCAGGCCAGCAGTCTCTGCTGGCTTTCGCTGAACAGCGGGCCAGCAAGAAGGATGAGCACGCCG
AACTGATCGTGCTGCGGAGAGGCGATTACGACGCCggcACACATCAGGTGCAGTGGCAGGCTCA
AGAGGTGGTGGCTCAGGCTAGACTGGACGGCCACAGATCTATGAACCCCTGTCCTCTGTACGAT
gaaCAGACCGGCACACTGTTTCTGTTCTTTATCGCTATCCCCGGCCAAGTGACCGAGCAGCAGC
AGCTGCAGACAAGAGCCAACGTGACCAGACTGTGTtacGTGACCTCCACCGACCACGGCAGAAC
CTGGTCTAGCCCTAGAGATCTGACCGACGCCGCCATCGGACCTGCCTATAGAGAGTGGTCCACC
TTCGCCGTTGGACCTGGACACTGTCTCCAGCTGCACGACAGGGCTAGATCTCTGGTGGTGCCTG
CCTACGCCTATAGAAAGCTGCACCCCAAACAGCGGCCTATTCCTAGCGCCTTCTGCTTTCTGAG
CCACGATCACGGCAGGACATGGGCCAGAGGACATTTCGTGGCCCAGGACACACTGGAATGCCAG
GTGGCCGAAGTGGAAACCGGCGAGCAGAGAGTCGTGACCCTGAACGCCAGATCTCACCTGAGAT
TCAGAGTGCAGGCCCAGAGCACAAACGACGGCCTGGATTTCCAAGAGAGCCAGCTGGTCAAGAA
ACTGGTGGAACCTCCTCCAaccGGCTGTCAGGGAAGCGTGATCAGCTTTCCATCTCCTAGAAGC
GGCCCTGGCTCTCCTGCTCAGTGGCTGCTGTATACACACCCCACACACAGCTGGCAGAGAGCCG
ATCTGGGCGCCTACCTGAATCCTAGACCTCCTGCTCCTGAGGCTTGGAGCGAACCTGTTCTGCT
GGCCAAGGGCAGCgctGCCTACAGCGATCTGCAGTCTATGGGCACAGGCCCTGATGGCAGCCCT
CTGTTTGGCTGTCTGTACGAGGCCAACGACTACGAAGAGATCGTGTTCCTGATGTTCACCCTGA
AGCAGGCCTTTCCAGCCGAGTACCTGCCTCAATAGTGA

What is claimed is:

1. A fusion protein comprising:
(a) a sialidase enzyme; and
(b) an anti-PD-1 immunoglobulin antigen-binding domain.

2. The fusion protein of claim 1, wherein the sialidase is a human sialidase.

3. The fusion protein of claim 1 or 2, wherein the sialidase is a recombinant mutant human sialidase.

4. The fusion protein of any one of claims 1-3, wherein the sialidase comprises:
(a) a substitution or deletion of a methionine residue at a position corresponding to position 1 of wild-type human Neu2 (M1);
(b) a substitution of a valine residue at a position corresponding to position 6 of wild-type human Neu2 (V6);
(c) a substitution of a lysine residue at a position corresponding to position 9 of wild-type human Neu2 (K9);
(d) a substitution of an alanine residue at a position corresponding to position 42 of wild-type human Neu2 (A42);
(e) a substitution of a proline residue at a position corresponding to position 62 of wild-type human Neu2 (P62);
(f) a substitution of an alanine residue at a position corresponding to position 93 of wild-type human Neu2 (A93);
(g) a substitution of a glutamine residue at a position corresponding to position 126 of wild-type human Neu2 (Q126);
(h) a substitution of an isoleucine residue at a position corresponding to position 187 of wild-type human Neu2 (I187);
(i) a substitution of an alanine residue at a position corresponding to position 242 of wild-type human Neu2 (A242);
(j) a substitution of a glutamine residue at a position corresponding to position 270 of wild-type human Neu2 (Q270);
(k) a substitution of a serine residue at a position corresponding to position 301 of wild-type human Neu2 (S301);
(l) a substitution of a tryptophan residue at a position corresponding to position 302 of wild-type human Neu2 (W302);
(m) a substitution of a cysteine residue at a position corresponding to position 332 of wild-type human Neu2 (C332);
(n) a substitution of a valine residue at a position corresponding to position 363 of wild-type human Neu2 (V363); or
(o) a substitution of a leucine residue at a position corresponding to position 365 of wild-type human Neu2 (L365);
or a combination of any of the foregoing substitutions.

5. The fusion protein of claim 4, wherein, in the sialidase:
(a) the methionine residue at a position corresponding to position 1 of wild-type human Neu2 is deleted (ΔM1), is substituted by alanine (M1A), or is substituted by aspartic acid (M1D);
(b) the valine residue at a position corresponding to position 6 of wild-type human Neu2 is substituted by tyrosine (V6Y);
(c) the alanine residue at a position corresponding to position 42 of wild-type human Neu2 is substituted by arginine (A42R)
(d) the lysine residue at a position corresponding to position 9 of wild-type human Neu2 is substituted by aspartic acid (K9D);
(e) the proline residue at a position corresponding to position 62 of wild-type human Neu2 is substituted by asparagine (P62N), aspartic acid (P62D), histidine (P62H), glutamic acid (P62E), glycine (P62G), serine (P62S), or threonine (P62T);
(f) the alanine residue at a position corresponding to position 93 of wild-type human Neu2 is substituted by glutamic acid (A93E) or lysine (A93K);
(g) the glutamine residue at a position corresponding to position 126 of wild-type human Neu2 is substituted by leucine (Q126L), glutamic acid (Q126E), phenylalanine (Q126F), histidine (Q126H), isoleucine (Q126I), or tyrosine (Q126Y);
(h) the isoleucine residue at a position corresponding to position 187 of wild-type human Neu2 is substituted by lysine (I187K);
(i) the alanine residue at a position corresponding to position 242 of wild-type human Neu2 is substituted by cysteine (A242C), phenylalanine (A242F), glycine (A242G), histidine (A242H), isoleucine (A242I), lysine (A242K), leucine (A242L), methionine (A242M), asparagine (A242N), glutamine (A242Q), arginine (A242R), serine (A242S), valine (A242V), tryptophan (A242W), or tyrosine (A242Y);
(j) the glutamine residue at a position corresponding to position 270 of wild-type human Neu2 is substituted by alanine (Q270A), histidine (Q270H), phenylalanine (Q270F), proline (Q270P), serine (Q270S), or threonine (Q270T);
(k) the serine residue at a position corresponding to position 301 of wild-type human Neu2 is substituted by alanine (S301A), aspartic acid (S301D), glutamic acid (S301E), phenylalanine (S301F), histidine (S301H), lysine (S301K), leucine (S301L), methionine (S301M), asparagine (S301N), proline (S301P), glutamine (S301Q), arginine (S301R), threonine (S301T), valine (S301V), tryptophan (S301W), or tyrosine (S301Y);
(1) the tryptophan residue at a position corresponding to position 302 of wild-type human Neu2 is substituted by alanine (W302A), aspartic acid (W302D), phenylalanine (W302F), glycine (W302G), histidine (W302H), isoleucine (W3021), lysine (W302K), leucine (W302L), methionine (W302M), asparagine (W302N), proline (W302P), glutamine (W302Q), arginine (W302R), serine (W302S), threonine (W302T), valine (W302V), or tyrosine (W302Y);
(m) the cysteine residue at a position corresponding to position 332 of wild-type human Neu2 is substituted by alanine (C332A);
(n) the valine residue at a position corresponding to position 363 of wild-type human Neu2 is substituted by arginine (V363R); or
(o) the leucine residue at a position corresponding to position 365 of wild-type human Neu2 is substituted by glutamine (L365Q), histidine (L365H), isoleucine (L365I), lysine (L365K) or serine (L365S);
or the sialidase comprises a combination of any of the foregoing substitutions.

6. The fusion protein of claim 5, wherein the sialidase comprises a substitution selected from ΔM1, M1A, M1D, V6Y, K9D, A42R, P62G, P62N, P62S, P62T, A93E, Q126Y, I187K, A242F, A242W, A242Y, Q270A, Q270T, S301A, S301R, W302K, W302R, C332A, V363R, and L365I, or a combination of any of the foregoing substitutions.

7. The fusion protein of claim 6, wherein the sialidase comprises:
(a) the M1D, V6Y, P62G, A93E, I187K, and C332A substitutions;
(b) the M1D, V6Y, K9D, A93E, I187K, C332A, V363R, and L365I substitutions;
(c) the M1D, V6Y, P62N, I187K, and C332A substitutions;
(d) the M1D, V6Y, I187K, Q270A, S301R, W302K, and C332A substitutions;
(e) the M1D, V6Y, P62S, I187K, Q270A, S301R, W302K, and C332A substitutions;
(f) the M1D, V6Y, P62T, I187K, Q270A, S301R, W302K, and C332A substitutions;
(g) the M1D, V6Y, P62N, I187K, Q270A, S301R, W302K, and C332A substitutions;
(h) the M1D, V6Y, P62G, A93E, I187K, S301A, W302R, and C332A substitutions;
(i) the M1D, V6Y, P62G, A93E, Q126Y, I187K, Q270T, and C332A substitutions;
(j) the M1D, V6Y, P62G, A93E, Q126Y, I187K, and C332A substitutions;
(k) the M1D, V6Y, P62G, A93E, Q126Y, I187K, A242F, Q270T, and C332A substitutions; or
(l) the M1D, V6Y, A42R, P62G, A93E, Q126Y, I187K, A242F, Q270T, and C332A mutations.

8. The fusion protein of any one of claims 1-7, wherein the sialidase is selected from Neu1, Neu2, Neu3, and Neu4.

9. The fusion protein of claim 8, wherein the sialidase is Neu2.

10. The fusion protein of any one of claims 1-9, wherein the sialidase has a different substrate specificity than the corresponding wild-type sialidase.

11. The fusion protein of claim 10, wherein the sialidase can cleave α2,3, α2,6, and/or α2,8 linkages.

12. The fusion protein of claim 11, wherein the sialidase can cleave α2,3 and α2,8 linkages.

13. The fusion protein of any one of claims 1-12, wherein the sialidase comprises any one of SEQ ID NOs: 48-63, 94, 97, 100, or 126.

14. The fusion protein of any one of claims 1-13, wherein the sialidase comprises a mutation set forth in any one of Tables 1-9.

15. The fusion protein of any one of claims 1-14, wherein the fusion protein further comprises an immunoglobulin Fc domain.

16. The fusion protein of claim 15, wherein the immunoglobulin Fc domain is derived from a human IgG1, IgG2, IgG3, IgG4, IgA1, IgA2, IgD, IgE, or IgM Fc domain.

17. The fusion protein of claim 16, wherein the immunoglobulin Fc domain is derived from a human IgG1, IgG2, IgG3, or IgG4 Fc domain.

18. The fusion protein of claim 17, wherein the immunoglobulin Fc domain is derived from a human IgG1 Fc domain.

19. The fusion protein of any one of claims 1-18, wherein the anti-PD-1 immunoglobulin antigen-binding domain is associated with a second anti-PD-1 immunoglobulin antigen-binding domain to produce an anti-PD-1 antigen-binding site.

20. The fusion protein of any one of claims 1-19, wherein the anti-PD-1 immunoglobulin antigen-binding domain is derived from an antibody selected from nivolumab, pembrolizumab, cemiplimab, PDR001, and pidilizumab.

21. The fusion protein of claim 20, wherein the anti-PD-1 immunoglobulin antigen-binding domain is derived from pembrolizumab.

22. The fusion protein of any one of claims 1-21, wherein the sialidase and the immunoglobulin Fc domain and/or the anti-PD-1 immunoglobulin antigen-binding domain are linked by a peptide bond or an amino acid linker.

23. The fusion protein of any one of claims 1-22, wherein the fusion protein comprises any one of SEQ ID NOs: 67-73, 78, 81-87, 95, 96, 98, 99, 101, 102, 105, 106, 108, 111, 112, 115, 122, 123, 125, 127, 128, 130, 132, or 134.

24. An antibody conjugate comprising the fusion protein of any one of claims 1-23.

25. The antibody conjugate of claim 24, wherein the antibody conjugate comprises a single sialidase.

26. The antibody conjugate of claim 24, wherein the antibody conjugate comprises two sialidases.

27. The antibody conjugate of claim 26, wherein the two sialidases are identical.

28. The antibody conjugate of any one of claims 24-27, wherein the antibody conjugate comprises a single anti-PD-1 antigen-binding site.

29. The antibody conjugate of any one of claims 24-27, wherein the antibody conjugate comprises two anti-PD-1 antigen-binding sites.

30. The antibody conjugate of claim 29, wherein the two anti-PD-1 antigen-binding sites are identical.

31. The antibody conjugate of any one of claims 24-30, wherein the antibody conjugate has a molecular weight from about 135 kDa to about 165 kDa.

32. The antibody conjugate of any one of claims 24-30, wherein the antibody conjugate has a molecular weight from about 215 kDa to about 245 kDa.

33. The antibody conjugate of any one of claims 24-32, wherein the antibody conjugate comprises:
(a) a first polypeptide comprising an immunoglobulin light chain;
(b) a second polypeptide comprising an immunoglobulin heavy chain; and
(c) a third polypeptide comprising an immunoglobulin Fc domain and a sialidase;
wherein the first and second polypeptides are covalently linked together and the second and third polypeptides are linked together, and wherein the first polypeptide and the second polypeptide together define an anti-PD-1 antigen-binding site.

34. The antibody conjugate of claim 33, wherein the third polypeptide comprises the sialidase and the immunoglobulin Fc domain in an N- to C-terminal orientation.

35. The antibody conjugate of claim 33 or 34, wherein the first polypeptide comprises SEQ ID NO: 77.

36. The antibody conjugate of any one of claims 33-35, wherein the second polypeptide comprises SEQ ID NO: 105.

37. The antibody conjugate of any one of claims 33-36, wherein the third polypeptide comprises any one of SEQ ID NOs: 67-73, 78, 81-87, 95, 96, 98, 99, 101, 102, 106, 108, 111, 112, 115, 122, 123, 125, 127, or 128.

38. The antibody conjugate of any one of claims 24-32, wherein the fusion protein comprises:
(a) a first polypeptide comprising a first immunoglobulin light chain;
(b) a second polypeptide comprising a first immunoglobulin heavy chain and a first sialidase;
(c) a third polypeptide comprising a second immunoglobulin heavy chain and a second sialidase; and
(d) a fourth polypeptide comprising a second immunoglobulin light chain;
wherein the first and second polypeptides are covalently linked together, the third and fourth polypeptides are covalently linked together, and the second and third polypeptides are covalently linked together, and wherein the first polypeptide and the second polypeptide together define a first anti-PD-1 antigen-binding site, and the third polypeptide and the fourth polypeptide together define a second anti-PD-1 antigen-binding site.

39. The antibody conjugate of claim 38, wherein the second and third polypeptides comprise the first and second immunoglobulin heavy chain and the first and second sialidase, respectively, in an N- to C-terminal orientation.

40. The antibody conjugate of any one of claims 24-32, wherein the fusion protein comprises:
(a) a first polypeptide comprising a first sialidase, a first immunoglobulin Fc domain, and a first single chain variable fragment (scFv); and
(b) a second polypeptide comprising a second sialidase, a second immunoglobulin Fc domain, and a second single chain variable fragment (scFv);
wherein the first and second polypeptides are covalently linked together, and wherein the first scFv defines a first anti-PD-1 antigen-binding site, and the second scFv defines a second anti-PD-1 antigen-binding site.

41. The antibody conjugate of claim 40, wherein the first polypeptide comprises the first sialidase, the first immunoglobulin Fc domain, and the first scFv in an N- to C-terminal orientation, and the second polypeptide comprises the second sialidase, the second immunoglobulin Fc domain, and the second scFv in an N- to C-terminal orientation.

42. The antibody conjugate of any one of claims 24-32, wherein the antibody conjugate comprises:
(a) a first polypeptide comprising an immunoglobulin light chain;
(b) a second polypeptide comprising an immunoglobulin heavy chain and a single chain variable fragment (scFv); and
(c) a third polypeptide comprising an immunoglobulin Fc domain and a sialidase;
wherein the first and second polypeptides are covalently linked together and the second and third polypeptides are covalently linked together, and wherein the immunoglobulin light chain and immunoglobulin heavy chain together define a first anti-PD-1 antigen-binding site and the scFv defines a second anti-PD-1 antigen-binding site.

43. The antibody conjugate of claim 42, wherein the second polypeptide comprises the immunoglobulin heavy chain and the scFv in an N- to C-terminal orientation, and the third polypeptide comprises the sialidase and the immunoglobulin Fc domain in an N- to C-terminal orientation.

44. An isolated nucleic acid comprising a nucleotide sequence encoding the fusion protein of any one of claims 1-23, or at least a portion of the antibody conjugate of any one of claims 24-43.

45. An expression vector comprising the nucleic acid of claim 44.

46. A host cell comprising the expression vector of claim 45.

47. A pharmaceutical composition comprising the fusion protein of any one of claims 1-23 or the antibody conjugate of any one of claims 24-32.

48. A method of treating cancer in a subject in need thereof, the method comprising administering to the subject an effective amount of the fusion protein of any one of claims 1-23, the antibody conjugate of any one of claims 24-32, or the pharmaceutical composition of claim 47.

49. The method of claim 48, wherein the cancer is selected from melanoma, bladder cancer, breast cancer, cervical cancer, esophageal cancer, gastric cancer, kidney cancer, lung cancer (e.g., NSCLC), ovarian cancer, metastatic Merkel cell carcinoma (MCC), metastatic urothelial carcinoma (UC), pancreatic cancer, colon cancer, stomach cancer, AML, and multiple myeloma.


SEQUENCE LISTING (150 sequences)

SEQ ID NO: 1 - protein, 380 aa, Homo sapiens
MASLPVLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPIQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQ

SEQ ID NO: 2 - protein, 385 aa, Mus musculus
MEDLRPMATCPVLQKETLFRTGVHAYRIPALLYLKKQKTLLAFAEKRASKTDEHAELIVLRRGSYNEATNRVKWQPEEVVTQAQLEGHRSMNPCPLYDKQTKTLFLFFIAVPGRVSEHHQLHTKVNVTRLCCVSSTDHGRTWSPIQDLTETTIGSTHQEWATFAVGPGHCLQLRNPAGSLLVPAYAYRKLHPAQKPTPFAFCFISLDHGHTWKLGNFVAENSLECQVAEVGTGAQRMVYLNARSFLGARVQAQSPNDGLDFQDNRVVSKLVEPPHGCHGSVVAFHNPISKPHALDTWLLYTHPTDSRNRTNLGVYLNQMPLDPTAWSEPTLLAMGICAYSDLQNMGQGPDGSPQFGCLYESGNYEEIIFLIFTLKQAFPTVFDAQ

SEQ ID NO: 3 - protein, 5 aa, Mus musculus
EDLRP

SEQ ID NO: 4 - protein, 6 aa, Mus musculus
MEDLRP

SEQ ID NO: 5 - protein, 227 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - DNA, 1143 bp, Salmonella typhimurium
acagtggaaaagtccgtggtgttcaaggccgagggcgagcacttcaccgaccagaaaggcaataccatcgtcggctctggcagcggcggcaccaccaagtactttagaatccccgccatgtgcaccaccagcaagggcaccattgtggtgttcgccgacgccagacacaacaccgccagcgatcagagcttcatcgataccgctgccgccagatctaccgatggcggcaagacctggaacaagaagatcgccatctacaacgaccgcgtgaacagcaagctgagcagagtgatggaccctacctgcatcgtggccaacatccagggcagagaaaccatcctggtcatggtcggaaagtggaacaacaacgataagacctggggcgcctacagagacaaggcccctgataccgattgggacctcgtgctgtacaagagcaccgatgacggcgtgaccttcagcaaggtggaaacaaacatccacgacatcgtgaccaagaacggcaccatctctgccatgctcggcggcgttggatctggcctgcaactgaatgatggcaagctggtgttccccgtgcagatggtccgaacaaagaatatcaccaccgtgctgaataccagcttcatctacagcaccgacggcatcacatggtccctgcctagcggctactgtgaaggctttggcagcgagaacaacatcatcgagttcaacgccagcctggtcaacaacatccggaacagcggcctgcggagaagcttcgagacaaaggacttcggaaagacgtggaccgagtttcctccaatggacaagaaggtggacaaccggaaccacggcgtgcagggcagcacaatcacaatccctagcggcaacaaactggtggccgctcactctagcgcccagaacaagaacaacgactacaccagaagcgacatcagcctgtacgcccacaacctgtacagcggcgaagtgaagctgatcgacgacttctaccccaaagtgggcaatgccagcggagccggctacagctgtctgagctaccggaaaaatgtggacaaagaaaccctgtacgtggtgtacgaggccaacggcagcatcgagtttcaggacctgagcagacatctgcccgtgatcaagagctacaac

SEQ ID NO: 7 - protein, 364 aa, Homo sapiens
ENDFGLVQPLVTMEQLLWVSGRQIGSVDTFRIPLITATPRGTLLAFAEARKMSSSDEGAKFIALRRSMDQGSTWSPTAFIVNDGDVPDGLNLGAVVSDVETGVVFLFYSLCAHKAGCQVASTMLVWSKDDGVSWSTPRNLSLDIGTEVFAPGPGSGIQKQREPRKGRLIVCGHGTLERDGVFCLLSDDHGASWRYGSGVSGIPYGQPKQENDFNPDECQPYELPDGSVVINARNQNNYHCHCRIVLRSYDACDTLRPRDVTFDPELVDPVVAAGAVVTSSGIVFFSNPAHPEFRVNLTLRWSFSNGTSWRKETVQLWPGPSGYSSLATLEGSMDGEEQAPQLYVLYEKGRNHYTESISVAKISV

SEQ ID NO: 8 - protein, 428 aa, Homo sapiens
MEEVTTCSFNSPLFRQEDDRGITYRIPALLYIPPTHTFLAFAEKRSTRRDEDALHLVLRRGLRIGQLVQWGPLKPLMEATLPGHRTMNPCPVWEQKSGCVFLFFICVRGHVTERQQIVSGRNAARLCFIYSQDAGCSWSEVRDLTEEVIGSELKHWATFAVGPGHGIQLQSGRLVIPAYTYYIPSWFFCFQLPCKTRPHSLMIYSDDLGVTWHHGRLIRPMVTVECEVAEVTGRAGHPVLYCSARTPNRCRAEALSTDHGEGFQRLALSRQLCEPPHGCQGSVVSFRPLEIPHRCQDSSSKDAPTIQQSSPGSSLRLEEEAGTPSESWLLYSHPTSRKQRVDLGIYLNQTPLEAACWSRPWILHCGPCGYSDLAALEEEGLFGCLFECGTKQECEQIAFRLFTHREILSHLQGDCTSPGRNPSQFKSN

SEQ ID NO: 9 - protein, 461 aa, Homo sapiens
MRPADLPPRPMEESPASSSAPTETEEPGSSAEVMEEVTTCSFNSPLFRQEDDRGITYRIPALLYIPPTHTFLAFAEKRSTRRDEDALHLVLRRGLRIGQLVQWGPLKPLMEATLPGHRTMNPCPVWEQKSGCVFLFFICVRGHVTERQQIVSGRNAARLCFIYSQDAGCSWSEVRDLTEEVIGSELKHWATFAVGPGHGIQLQSGRLVIPAYTYYIPSWFFCFQLPCKTRPHSLMIYSDDLGVTWHHGRLIRPMVTVECEVAEVTGRAGHPVLYCSARTPNRCRAEALSTDHGEGFQRLALSRQLCEPPHGCQGSVVSFRPLEIPHRCQDSSSKDAPTIQQSSPGSSLRLEEEAGTPSESWLLYSHPTSRKQRVDLGIYLNQTPLEAACWSRPWILHCGPCGYSDLAALEEEGLFGCLFECGTKQECEQIAFRLFTHREILSHLQGDCTSPGRNPSQFKSN

SEQ ID NO: 10 - protein, 484 aa, Homo sapiens
MGVPRTPSRTVLFERERTGLTYRVPSLLPVPPGPTLLAFVEQRLSPDDSHAHRLVLRRGTLAGGSVRWGALHVLGTAALAEHRSMNPCPVHDAGTGTVFLFFIAVLGHTPEAVQIATGRNAARLCCVASRDAGLSWGSARDLTEEAIGGAVQDWATFAVGPGHGVQLPSGRLLVPAYTYRVDRRECFGKICRTSPHSFAFYSDDHGRTWRCGGLVPNLRSGECQLAAVDGGQAGSFLYCNARSPLGSRVQALSTDEGTSFLPAERVASLPETAWGCQGSIVGFPAPAPNRPRDDSWSVGPGSPLQPPLLGPGVHEPPEEAAVDPRGGQVPGGPFSRLQPRGDGPRQPGPRPGVSGDVGSWTLALPMPFAAPPQSPTWLLYSHPVGRRARLHMGIRLSQSPLDPRSWTEPWVIYEGPSGYSDLASIGPAPEGGLVFACLYESGARTSYDEISFCTFSLREVLENVPASPKPPNLGDKPRGCCWPS

SEQ ID NO: 11 - protein, 496 aa, Homo sapiens
MMSSAAFPRWLSMGVPRTPSRTVLFERERTGLTYRVPSLLPVPPGPTLLAFVEQRLSPDDSHAHRLVLRRGTLAGGSVRWGALHVLGTAALAEHRSMNPCPVHDAGTGTVFLFFIAVLGHTPEAVQIATGRNAARLCCVASRDAGLSWGSARDLTEEAIGGAVQDWATFAVGPGHGVQLPSGRLLVPAYTYRVDRRECFGKICRTSPHSFAFYSDDHGRTWRCGGLVPNLRSGECQLAAVDGGQAGSFLYCNARSPLGSRVQALSTDEGTSFLPAERVASLPETAWGCQGSIVGFPAPAPNRPRDDSWSVGPGSPLQPPLLGPGVHEPPEEAAVDPRGGQVPGGPFSRLQPRGDGPRQPGPRPGVSGDVGSWTLALPMPFAAPPQSPTWLLYSHPVGRRARLHMGIRLSQSPLDPRSWTEPWVIYEGPSGYSDLASIGPAPEGGLVFACLYESGARTSYDEISFCTFSLREVLENVPASPKPPNLGDKPRGCCWPS

SEQ ID NO: 12 - protein, 5 aa, Homo sapiens
MASLP

SEQ ID NO: 13 - protein, 4 aa, Homo sapiens
ASLP

SEQ ID NO: 14 - protein, 8 aa, Salmonella typhimurium
TVEKSVVF

SEQ ID NO: 15 - protein, 12 aa, Homo sapiens
GDYDAPTHQVQW

SEQ ID NO: 16 - protein, 8 aa, Homo sapiens
SMDQGSTW

SEQ ID NO: 17 - protein, 8 aa, Salmonella typhimurium
STDGGKTW

SEQ ID NO: 18 - protein, 8 aa, Homo sapiens
PRPPAPEA

SEQ ID NO: 19 - protein, 8 aa, Homo sapiens
QTPLEAAC

SEQ ID NO: 20 - protein, 9 aa, Homo sapiens
NPRPPAPEA

SEQ ID NO: 21 - protein, 7 aa, Unknown
  Description of Unknown Recombinant mutant human sialidase substitution sequence
SQNDGES

SEQ ID NO: 22 - protein, 7 aa, Unknown
  Description of Unknown Recombinant mutant human sialidase substitution sequence
LSHSLST

SEQ ID NO: 23 - DNA, 1092 bp, Homo sapiens
gagaacgactttggactggtgcagcctctggtcaccatggaacagctgctgtgggtttccggcagacagatcggcagcgtggacaccttcagaatccctctgatcaccgccacacctagaggcaccctgctggcctttgccgaggccagaaagatgagcagctctgacgagggcgccaagtttattgccctgaggcggtctatggaccagggctctacatggtcccctaccgccttcatcgtgaacgatggcgacgtgcccgatggcctgaatctgggagctgtggtgtccgatgtggaaaccggcgtggtgttcctgttctacagcctgtgtgcccacaaggccggttgtcaggtggccagcacaatgctcgtgtggtccaaggacgacggcgtgtcctggtctacccctagaaacctgagcctggacatcggcaccgaagtgtttgctccaggacctggctctggcatccagaagcagagagagcccagaaagggcagactgatcgtgtgtggccacggcacccttgagagagatggcgttttctgcctgctgagcgacgatcatggcgcctcttggagatacggcagcggagtgtctggaatcccttacggccagcctaagcaagagaacgatttcaaccccgacgagtgccagccttacgagctgcctgatggcagcgtcgtgatcaacgcccggaaccagaacaactaccactgccactgccggatcgtgctgagaagctacgacgcctgcgataccctgcggcctagagatgtgaccttcgatcctgagctggtggaccctgttgttgccgctggtgccgtcgtgacatctagcggcatcgtgttcttcagcaaccctgctcaccccgagttcagagtgaatctgaccctgcggtggtccttcagcaatggcacaagctggcggaaagaaaccgtgcagctttggcctggacctagcggctactcttctctggctacactggaaggcagcatggacggcgaagaacaggcccctcagctgtacgtgctgtacgagaagggcagaaaccactacaccgagagcatcagcgtggccaagatcagcgtt

SEQ ID NO: 24 - DNA, 1140 bp, Homo sapiens
atggccagcctgcctgtgctgcagaaagaaagcgtgttccagtctggcgcccacgcctacagaattcccgctctgctgtatctgccaggccagcagtctctgctggctttcgctgaacagcgggccagcaagaaggatgagcacgccgaactgatcgtgctgcggagaggcgattacgacgcccctacacatcaggtgcagtggcaggctcaagaggtggtggctcaggctagactggacggccacagatctatgaacccctgtcctctgtacgatgcccagaccggcacactgtttctgttctttatcgctatccccggccaagtgaccgagcagcagcagctgcagacaagagccaacgtgaccagactgtgtcaagtgacctccaccgaccacggcagaacctggtctagccctagagatctgaccgacgccgccatcggacctgcctatagagagtggtccaccttcgccgttggacctggacactgtctccagctgcacgacagggctagatctctggtggtgcctgcctacgcctatagaaagctgcaccccatccagcggcctattcctagcgccttctgctttctgagccacgatcacggcaggacatgggccagaggacatttcgtggcccaggacacactggaatgccaggtggccgaagtggaaaccggcgagcagagagtcgtgaccctgaacgccagatctcacctgagagccagagtgcaggcccagagcacaaacgacggcctggatttccaagagagccagctggtcaagaaactggtggaacctcctccacagggctgtcagggaagcgtgatcagctttccatctcctagaagcggccctggctctcctgctcagtggctgctgtatacacaccccacacacagctggcagagagccgatctgggcgcctacctgaatcctagacctcctgctcctgaggcttggagcgaacctgttctgctggccaagggcagctgtgcctacagcgatctgcagtctatgggcacaggccctgatggcagccctctgtttggctgtctgtacgaggccaacgactacgaagagatcgtgttcctgatgttcaccctgaagcaggcctttccagccgagtacctgcctcaa

SEQ ID NO: 25 - DNA, 1284 bp, Homo sapiens
atggaggaagtgaccacctgtagcttcaacagccctctgttccggcaagaggacgaccggggcatcacctacagaatccctgctctgctgtacatccctcctacacacacctttctggccttcgccgagaagcggagcaccagacgagatgaagatgccctgcacctggtgctgagaagaggcctgagaatcggacagctggtgcagtggggacctctgaagcctctgatggaagccacactgcccggccacagaaccatgaatccttgtcctgtgtgggagcagaaaagcggctgcgtgttcctgttcttcatctgcgtgcggggccacgtgaccgagagacagcaaatcgtgtccggcagaaacgccgccagactgtgcttcatctacagccaggatgccggctgctcttggagcgaagttcgggatctgaccgaagaagtgatcggcagcgagctgaagcactgggccacatttgctgttggccctggccacggaatccagctgcaatctggcagactggtcatccccgcctacacctactatatccccagctggttcttctgcttccaactgccttgcaagacccggcctcacagcctgatgatctacagcgacgatctgggcgtgacatggcaccacggcagactgatcagacccatggtcaccgtggaatgcgaggtggccgaagtgacaggcagagctggacaccctgtgctgtactgctctgccagaacacccaaccggtgtagagccgaggctctgtctacagatcacggcgagggctttcagagactggccctctctagacagctgtgcgaacctcctcatggctgtcagggcagcgtggtgtccttcagacctctggaaatccctcaccggtgccaggacagcagctctaaggatgcccctaccatccagcagtctagccctggcagcagcctgagactggaagaggaagccggaacacctagcgagagctggctgctgtactctcaccccaccagcagaaagcagagagtggacctgggcatctacctgaatcagacccctctggaagccgcctgttggagcagaccttggattctgcactgtggcccttgcggctactctgatctggccgctctggaagaagagggcctgttcggctgcctgtttgagtgcggcacaaagcaagagtgcgagcagatcgccttccggctgttcacccacagagagatcctgagccatctgcagggcgactgcacaagcccaggcagaaatcccagccagttcaagagcaac

SEQ ID NO: 26 - DNA, 1452 bp, Homo sapiens
atgggcgtgcccagaacacccagcagaaccgtgctgttcgagagagagaggaccggcctgacctacagagtgccttctctgctgcctgtgcctcctggacctacactgctggccttcgtggaacagagactgagccccgatgattctcacgcccacagactggtgctgagaagaggaacactggctggcggctctgttagatggggagcactgcatgtgctgggcacagctgctcttgccgagcacagatccatgaatccctgtcctgtgcacgacgccggaaccggcacagtgtttctgttctttatcgccgtgctgggccacacacctgaggccgttcaaattgccaccggcagaaatgccgccagactgtgttgtgtggcctccagagatgccggcctgtcttggggatctgccagagatctgaccgaggaagccattggcggagccgttcaggattgggccacatttgctgttggacctggacacggcgtgcagctgccaagtggtagactgctggtgcctgcctacacatacagagtggatcggagagagtgcttcggaaagatctgccggacaagccctcacagcttcgccttctactccgacgatcacggccggacttggagatgtggtggcctggtgcctaatctgagaagcggcgaatgtcaactggccgccgttgatggtggacaggctggcagcttcctgtactgcaacgccagatctcctctgggctctagagtgcaggccctgtctaccgatgagggcaccagttttctgcccgccgaaagagttgcctctctgcctgaaacagcctggggctgtcagggctctatcgtgggatttcctgctcctgctccaaacagaccccgggacgattcttggagtgtcggccctggatctccactgcagcctccattgcttggaccaggcgttcacgagccacctgaagaggctgccgttgatcctagaggcggacaagttcctggcggcccttttagcagactgcagccaagaggcgacggccctagacaacctggaccaagacctggcgtcagcggagatgttggctcttggacactggccctgcctatgccttttgccgctcctcctcagtctcctacctggctgctgtactctcaccctgttggcagacgggccagactgcacatgggcatcagactgtctcagagccctctggaccccagaagctggacagagccttgggtcatctatgagggccctagcggctacagcgatctggcctctattggcccagctcctgaaggcggactggtgttcgcttgtctgtatgagagcggcgccagaaccagctacgacgagatcagcttctgcaccttcagcctgcgcgaggtgctggaaaatgtgcccgcctctcctaagcctcctaacctgggcgataagcctagaggctgttgctggccatct

SEQ ID NO: 27 - protein, 47 aa, Homo sapiens
MTGERPSTALPDRRWGPRILGFWGGCRVWVFAAIFLLLSLAASWSKA

SEQ ID NO: 28 - protein, 22 aa, Unknown
  Description of Unknown N-terminal signal peptide sequence
MDMRVPAQLLGLLLLWLPGARC

SEQ ID NO: 29 - protein, 4 aa, Unknown
  Description of Unknown C-terminal lysosomal signal motif sequence
YGTL

SEQ ID NO: 30 - protein, 382 aa, Salmonella typhimurium
MTVEKSVVFKAEGEHFTDQKGNTIVGSGSGGTTKYFRIPAMCTTSKGTIVVFADARHNTASDQSFIDTAAARSTDGGKTWNKKIAIYNDRVNSKLSRVMDPTCIVANIQGRETILVMVGKWNNNDKTWGAYRDKAPDTDWDLVLYKSTDDGVTFSKVETNIHDIVTKNGTISAMLGGVGSGLQLNDGKLVFPVQMVRTKNITTVLNTSFIYSTDGITWSLPSGYCEGFGSENNIIEFNASLVNNIRNSGLRRSFETKDFGKTWTEFPPMDKKVDNRNHGVQGSTITIPSGNKLVAAHSSAQNKNNDYTRSDISLYAHNLYSGEVKLIDDFYPKVGNASGAGYSCLSYRKNVDKETLYVVYEANGSIEFQDLSRHLPVIKSYN

SEQ ID NO: 31 - protein, 232 aa, Artificial Sequence
  Description of Artificial Sequence Synthetic polypeptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 227 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 33 - protein, 232 aa, Artificial Sequence
  Description of Artificial Sequence Synthetic polypeptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - DNA, 1383 bp, Homo sapiens
atgagacctgcggacctgcccccgcgccccatggaagaatccccggcgtccagctctgccccgacagagacggaggagccggggtccagtgcagaggtcatggaagaagtgacaacatgctccttcaacagccctctgttccggcaggaagatgacagagggattacctaccggatcccagccctgctctacataccccccacccacaccttcctggcctttgcagagaagcgttctacgaggagagatgaggatgctctccacctggtgctgaggcgagggttgaggattgggcagttggtacagtgggggcccctgaagccactgatggaagccacactaccggggcatcggaccatgaacccctgtcctgtatgggagcagaagagtggttgtgtgttcctgttcttcatctgtgtgcggggccatgtcacagagcgtcaacagattgtgtcaggcaggaatgctgcccgcctttgcttcatctacagtcaggatgctggatgttcatggagtgaggtgagggacttgactgaggaggtcattggctcagagctgaagcactgggccacatttgctgtgggcccaggtcatggcatccagctgcagtcagggagactggtcatccctgcgtatacctactacatcccttcctggttcttttgcttccagctaccatgtaaaaccaggcctcattctctgatgatctacagtgatgacctaggggtcacatggcaccatggtagactcattaggcccatggttacagtagaatgtgaagtggcagaggtgactgggagggctggccaccctgtgctatattgcagtgcccggacaccaaacaggtgccgggcagaggcgctcagcactgaccatggtgaaggctttcagagactggccctgagtcgacagctctgtgagcccccacatggttgccaagggagtgtggtaagtttccggcccctggagatcccacataggtgccaggactctagcagcaaagatgcacccaccattcagcagagctctccaggcagttcactgaggctggaggaggaagctggaacaccgtcagaatcatggctcttgtactcacacccaaccagtaggaaacagagggttgacctaggtatctatctcaaccagacccccttggaggctgcctgctggtcccgcccctggatcttgcactgtgggccctgtggctactctgatctggctgctctggaggaggagggcttgtttgggtgtttgtttgaatgtgggaccaagcaagagtgtgagcagattgccttccgcctgtttacacaccgggagatcctgagtcacctgcagggggactgcaccagccctggtaggaacccaagccaattcaaaagcaat

SEQ ID NO: 35 - DNA, 1488 bp, Homo sapiens
atgatgagctctgcagccttcccaaggtggctgagcatgggggtccctcgtaccccttcacggacagtgctcttcgagcgggagaggacgggcctgacctaccgcgtgccctcgctgctccccgtgccccccgggcccaccctgctggcctttgtggagcagcggctcagccctgacgactcccacgcccaccgcctggtgctgaggaggggcacgctggccgggggctccgtgcggtggggtgccctgcacgtgctggggacagcagccctggcggagcaccggtccatgaacccctgccctgtgcacgatgctggcacgggcaccgtcttcctcttcttcatcgcggtgctgggccacacgcctgaggccgtgcagatcgccacgggaaggaacgccgcgcgcctctgctgtgtggccagccgtgacgccggcctctcgtggggcagcgcccgggacctcaccgaggaggccatcggtggtgccgtgcaggactgggccacattcgctgtgggtcccggccacggtgtgcagctgccctcaggccgcctgctggtacccgcctacacctaccgcgtggaccgccgagagtgttttggcaagatctgccggaccagccctcactccttcgccttctacagcgatgaccacggccgcacctggcgctgtggaggcctcgtgcccaacctgcgctcaggcgagtgccagctggcagcggtggacggtgggcaggccggcagcttcctctactgcaatgcccggagcccactgggcagccgtgtgcaggcgctcagcactgacgagggcacctccttcctgcccgcagagcgcgtggcttccctgcccgagactgcctggggctgccagggcagcatcgtgggcttcccagcccccgcccccaacaggccacgggatgacagttggtcagtgggccccgggagtcccctccagcctccactcctcggtcctggagtccacgaacccccagaggaggctgctgtagacccccgtggaggccaggtgcctggtgggcccttcagccgtctgcagcctcggggggatggccccaggcagcctggccccaggcctggggtcagtggggatgtggggtcctggaccctggcactccccatgccctttgctgccccgccccagagccccacgtggctgctgtactcccacccagtggggcgcagggctcggctacacatgggtatccgcctgagccagtccccgctggacccgcgcagctggacagagccctgggtgatctacgagggccccagcggctactccgacctggcgtccatcgggccggcccctgaggggggcctggtttttgcctgcctgtacgagagcggggccaggacctcctatgatgagatttccttttgtacattctccctgcgtgaggtcctggagaacgtgcccgccagccccaaaccgcccaaccttggggacaagcctcgggggtgctgctggccctcc

SEQ ID NO: 36 - protein, 781 aa, Vibrio cholerae
MRFKNVKKTALMLAMFGMATSSNAALFDYNATGDTEFDSPAKQGWMQDNTNNGSGVLTNADGMPAWLVQGIGGRAQWTYSLSTNQHAQASSFGWRMTTEMKVLSGGMITNYYANGTQRVLPIISLDSSGNLVVEFEGQTGRTVLATGTAATEYHKFELVFLPGSNPSASFYFDGKLIRDNIQPTASKQNMIVWGNGSSNTDGVAAYRDIKFEIQGDVIFRGPDRIPSIVASSVTPGVVTAFAEKRVGGGDPGALSNTNDIITRTSRDGGITWDTELNLTEQINVSDEFDFSDPRPIYDPSSNTVLVSYARWPTDAAQNGDRIKPWMPNGIFYSVYDVASGNWQAPIDVTDQVKERSFQIAGWGGSELYRRNTSLNSQQDWQSNAKIRIVDGAANQIQVADGSRKYVVTLSIDESGGLVANLNGVSAPIILQSEHAKVHSFHDYELQYSALNHTTTLFVDGQQITTWAGEVSQENNIQFGNADAQIDGRLHVQKIVLTQQGHNLVEFDAFYLAQQTPEVEKDLEKLGWTKIKTGNTMSLYGNASVNPGPGHGITLTRQQNISGSQNGRLIYPAIVLDRFFLNVMSIYSDDGGSNWQTGSTLPIPFRWKSSSILETLEPSEADMVELQNGDLLLTARLDFNQIVNGVNYSPRQQFLSKDGGITWSLLEANNANVFSNISTGTVDASITRFEQSDGSHFLLFTNPQGNPAGTNGRQNLGLWFSFDEGVTWKGPIQLVNGASAYSDIYQLDSENAIVIVETDNSNMRILRMPITLLKQKLTLSQN

SEQ ID NO: 37 - DNA, 2424 bp, Vibrio cholerae
ttgtcaatcaagatgacttcacaacgaagaagagcatcgattcacaaggaaacagattctaatataaagggagtagatatgcgtttcaaaaacgtaaagaaaaccgctttaatgcttgcaatgttcggtatggcgacaagctcaaacgccgcactttttgactataacgcaacgggtgacactgagtttgacagtccagccaaacagggatggatgcaagacaacacgaataatggcagcggcgttttaaccaatgcagatggaatgcccgcttggttggtgcaaggtattggagggagagctcaatggacatattctctctctactaatcaacatgcccaagcatcaagtttcggttggcgaatgacgacagaaatgaaagtgctcagtggtggaatgatcacaaactactacgccaacggcactcagcgtgtcttacccatcatttcattagatagcagtggtaacttagttgttgagtttgaagggcaaactggacgcaccgttttggcaaccggcacagcagcaacggaatatcataaatttgaattggtattccttcctggaagtaacccatccgctagcttttacttcgatggcaaactcattcgtgacaacatccagccgactgcatcaaaacaaaatatgatcgtatgggggaatggctcatcaaatacggatggtgtcgccgcttatcgtgatattaagtttgaaattcaaggcgacgtcatcttcagaggcccagaccgtataccgtccattgtagcaagtagcgtaacaccaggggtggtaaccgcatttgcagagaaacgtgtggggggaggagatcccggtgctctgagtaataccaatgacataatcactcgtacctcacgagatggcggtataacttgggataccgagctcaacctcactgagcaaatcaatgtcagtgatgagtttgatttctccgatcctcggcctatctatgatccttcctccaatacggttcttgtctcttatgctcgatggccgaccgatgccgctcaaaacggagatcgaataaaaccatggatgccaaacggtattttttacagcgtctatgatgttgcatcagggaactggcaagcgcctatcgatgttaccgatcaggtgaaagaacgcagtttccaaatcgctggttggggtggttcagagctgtatcgccgaaataccagcctaaatagccagcaagactggcaatcaaacgctaagatccgaattgttgatggtgcagcgaaccagatacaagttgccgatggtagccgaaaatatgttgtcacactgagtattgatgaatcaggtggtctagtcgctaatctaaacggtgttagtgctccgattatcctgcaatctgaacacgcaaaggtacactctttccatgactacgaacttcaatattcggcgttaaaccacaccacaacgttattcgtggatggtcagcaaatcacaacttgggctggcgaagtatcgcaggagaacaacattcagtttggtaatgcggatgcccaaattgacggcagactgcatgtgcaaaaaattgttctcacacagcaaggccataacctcgtggagtttgatgctttctatttagcacagcaaacccctgaagtagagaaagaccttgaaaagcttggttggacaaaaattaaaacgggcaacaccatgagtttgtatggaaatgccagtgtcaacccaggaccgggtcatggcatcacccttactcgacaacaaaatatcagtggcagccaaaacggccgcttgatctacccagcgattgtgcttgatcgtttcttcttgaacgtcatgtctatttacagtgatgatggcggttcaaactggcaaaccggttcaacactccctatcccctttcgctggaagagttcgagtatcctagaaactctcgaacctagtgaagctgatatggttgaactccaaaacggtgatctactccttactgcacgccttgattttaaccaaatcgttaatggtgtgaactatagcccacgccagcaatttttgagtaaagatggtggaatcacgtggagcctacttgaggctaacaacgctaacgtctttagcaatatcagtactggtaccgttgatgcttctattactcggttcgagcaaagtgacggtagccatttcttactctttactaacccacaaggaaaccctgcggggacaaatggcaggcaaaatctaggcttatggtttagcttcgatgaaggggtgacatggaaaggaccaattcaacttgttaatggtgcatcggcatattctgatatttatcaattggattcggaaaatgcgattgtcattgttgaaacggataattcaaatatgcgaattcttcgtatgcctatcacattgctaaaacagaagctgaccttatcgcaaaactaa

SEQ ID NO: 38 - protein, 409 aa, Mus musculus
MVGADPTRPRGPLSYWAGRRGQGLAAIFLLLVSAAESEARAEDDFSLVQPLVTMEQLLWVSGKQIGSVDTFRIPLITATPRGTLLAFAEARKKSASDEGAKFIAMRRSTDQGSTWSSTAFIVDDGEASDGLNLGAVVNDVDTGIVFLIYTLCAHKVNCQVASTMLVWSKDDGISWSPPRNLSVDIGTEMFAPGPGSGIQKQREPGKGRLIVCGHGTLERDGVFCLLSDDHGASWHYGTGVSGIPFGQPKHDHDFNPDECQPYELPDGSVIINARNQNNYHCRCRIVLRSYDACDTLRPRDVTFDPELVDPVVAAGALATSSGIVFFSNPAHPEFRVNLTLRWSFSNGTSWLKERVQVWPGPSGYSSLTALENSTDGKKQPPQLFVLYEKGLNRYTESISMVKISVYGTL

SEQ ID NO: 39 - protein, 393 aa, Mus musculus
MTVQPSPWFSDLRPMATCPVLQKETLFRTGVHAYRIPALLYLKKQKTLLAFAEKRASKTDEHAELIVLRRGSYNEATNRVKWQPEEVVTQAQLEGHRSMNPCPLYDKQTKTLFLFFIAVPGRVSEHHQLHTKVNVTRLCCVSSTDHGRTWSPIQDLTETTIGSTHQEWATFAVGPGHCLQLRNPAGSLLVPAYAYRKLHPAQKPTPFAFCFISLDHGHTWKLGNFVAENSLECQVAEVGTGAQRMVYLNARSFLGARVQAQSPNDGLDFQDNRVVSKLVEPPHGCHGSVVAFHNPISKPHALDTWLLYTHPTDSRNRTNLGVYLNQMPLDPTAWSEPTLLAMGICAYSDLQNMGQGPDGSPQFGCLYESGNYEEIIFLIFTLKQAFPTVFDAQ

SEQ ID NO: 40 - protein, 418 aa, Mus musculus
MEEVPPYSLSSTLFQQEEQSGVTYRIPALLYLPPTHTFLAFAEKRTSVRDEDAACLVLRRGLMKGRSVQWGPQRLLMEATLPGHRTMNPCPVWEKNTGRVYLFFICVRGHVTERCQIVWGKNAARLCFLCSEDAGCSWGEVKDLTEEVIGSEVKRWATFAVGPGHGIQLHSGRLIIPAYAYYVSRWFLCFACSVKPHSLMIYSDDFGVTWHHGKFIEPQVTGECQVAEVAGTAGNPVLYCSARTPSRFRAEAFSTDSGGCFQKPTLNPQLHEPRTGCQGSVVSFRPLKMPNTYQDSIGKGAPATQKCPLLDSPLEVEKGAETPSATWLLYSHPTSKRKRINLGIYYNRNPLEVNCWSRPWILNRGPSGYSDLAVVEEQDLVACLFECGEKNEYERIDFCLFSDHEVLSCEDCTSPSSD

SEQ ID NO: 41 - protein, 501 aa, Mus musculus
METAGAPFCFHVDSLVPCSYWKVMGPTRVPRRTVLFQRERTGLTYRVPALLCVPPRPTLLAFAEQRLSPDDSHAHRLVLRRGTLTRGSVRWGTLSVLETAVLEEHRSMNPCPVLDEHSGTIFLFFIAVLGHTPEAVQIATGKNAARLCCVTSCDAGLTWGSVRDLTEEAIGAALQDWATFAVGPGHGVQLRSGRLLVPAYTYHVDRRECFGKICWTSPHSLAFYSDDHGISWHCGGLVPNLRSGECQLAAVDGDFLYCNARSPLGNRVQALSADEGTSFLPGELVPTLAETARGCQGSIVGFLAPPSIEPQDDRWTGSPRNTPHSPCFNLRVQESSGEGARGLLERWMPRLPLCYPQSRSPENHGLEPGSDGDKTSWTPECPMSSDSMLQSPTWLLYSHPAGRRARLHMGIYLSRSPLDPHSWTEPWVIYEGPSGYSDLAFLGPMPGASLVFACLFESGTRTSYEDISFCLFSLADVLENVPTGLEMLSLRDKAQGHCWPS

SEQ ID NO: 42 - DNA, 3850 bp, Mus musculus
gggtcacatgctgatggactaattggagtcgcggcagcgcgggctgcggcccccaaggggaggggtcggagtgacgtgcgcgcttttaaagggccgaggtcagctgacggcttgccaccggtgaccagttcctggacagggatcgccgggagctatggtgggggcagacccgaccagaccccggggaccgctgagctattgggcgggccgtcggggtcaggggctcgcagcgatcttcctgctcctggtgtccgcggcggaatccgaggccagggcagaggatgacttcagcctggtgcagccgctggtgaccatggagcagctgctgtgggtgagcgggaagcagatcggctctgtagacactttccgcatcccgctcatcacagccacccctcggggcacgctcctggccttcgctgaggccaggaaaaaatctgcatccgatgagggggccaagttcatcgccatgaggaggtccacggaccagggtagcacgtggtcctctacagccttcatcgtagacgatggggaggcctccgatggcctgaacctgggcgctgtggtgaacgatgtagacacagggatagtgttccttatctataccctctgtgctcacaaggtcaactgccaggtggcctctaccatgttggtttggagtaaggacgacggcatttcctggagcccaccccggaatctctctgtggatattggcacagagatgtttgcccctggacctggctcaggcattcagaaacagcgggagcctgggaagggccggctcattgtgtgtggacacgggacgctggagcgagatggggtcttctgtctcctcagtgatgaccacggtgcctcctggcactacggcactggagtgagcggcattccctttggccagcccaaacacgatcacgatttcaaccccgacgagtgccagccctacgagcttccagatggctcggtcatcatcaacgcccggaaccagaataactaccattgccgctgcaggatcgtcctccgcagctatgacgcctgtgacaccctcaggccccgggatgtgaccttcgaccctgagctcgtggaccctgtggtagctgcaggagcactagccaccagctccggcattgtcttcttctccaatccagcccaccctgagttccgagtgaacctgaccctgcgctggagtttcagcaatggtacatcctggcagaaggagagggtccaggtgtggccgggacccagcggctactcgtccctgacagccctggaaaacagcacggatggaaagaagcagcccccgcagctgttcgttctgtacgagaaaggcctgaaccggtacaccgagagcatctccatggtcaaaatcagcgtctacggcacgctctgagccccgtgcccaaaggacaccaagtcctggtcgctgacttcacagctctctggaccatctgcagagggtgcctgaaacacagctcttcctctgaactctgaccttttgcaacttctcatcaacagggaagtctcttcgttatgacttaacacccagcttcctctcggggcaggaagtccctccgtcaccaagagcacttttttccagtatgctggggatggcccctgtccattctcttccaggacaacggagctgtgcctttctgggacaggatgggggaggggctccccctggagagatgaacagatacgaactcagggaactgagaaggcccggtgtcctagggtacaaaggcaggtactagatgtgattgctgaaagtccccagggcagagtgtcctttcagagcaaggataagcacacctacgtgtgcacctttgattatttatgaatcgaaatatttgtaacttaaaatttttgatgcagaaaaagcgtttgtggagtctgtggttctgtctgctcacgccttcccaattgcctcctggagagacaggaaggcagctggaagaggagccgatgtacttactgggaagcagaaacccctagattccatcctggctgctgctgtttgcaagtgtcaaagatgggggggcgtgtttatattttatatttctaagatggggtggcataggaaatagggaacagatgtgtaaaaccagatgggaaggacagtctgtgagaaaggagcaagcagttgctgcaggtgtgggagagcaaagcccttctccacgtggaaagagcccagatggacgctaagcatgttgggcacctgtaaccccgcactcgctggactgacggtgtagctcagtggtggagctagtacttggaacgcctaagactctgggttcagtccttgggggggggggtatgtgtttattgagaggaaggtgtacgtactgtaggtcagaggacagcttactggagttgtctctctccttcacgctgtgagtcctgtggaatgacctcaggtgtcagagttgggggcaggtgcctttgccagctgagccatcttgctgtctctgctttaatttaaaaaaaaaaaaaaaaaagaatattaaggtctgagggattcgggctgcgttcatttcaattagagggtcatatttcttttgacatttcttctctaagaaatgttaagatcatttgttctgtgtgatagaggtatagctccattgtatgtcagcagtgagggatcctgtgcattttatccagagtttgtacggtgttctaggggctgctagtgcagcccagtgctaaacacttcagcatgcacaaggcctcaatcagtgcatgcatgtgcacacacacacagacacacacgtacacactgacacaggtacacaaatacacactggcccacatgtacacatcgactcacaggtacacagacccactttgacacacatatacacagacacaaacgcactggcacacacatatacacaggcacacatggatagatggacacacgtgtacacatacacacacacacagaaatacaaatgttcaggttttctaaaaaaaaaaaaattagagacgtgttgacttcatttttagcaaaaatcctgtcatgtatcttaaagtggattgaacccactatgtagcccaggctggcctccaaatgggcatccttctgcctcagtctcccgagggctaggataacaggagtatgccatcacacctggctaatagaaattttcaaaattgtttgtttgaaggtgactcttactatattgcctaactgatctccagttcgtgaaatcctcctgcctcagaaccaggactgtcaatataacccaccaagacaggccaacattcacaattgattgttagtttgtggtctgaatcaaggtcttatactgtagcccaggctagcccggaatacacgatatctccagtgcttcagatcctcagttctaactaagcatggccacatccatgtttaactgcaaatttgatgttaccatggtttggtttggtttggtttggtttggtttggtttggtttggttttttggccattttttttttctcatgctgaggccttgtgctctcaagttggggagacagcatggagggtagctgcaactgtaaccccagttccaggggacctgacaccctctggcctccacaagtattaggcacatctgtggtgcacagacatacaatcaggcaaaatattcatacacataaaataaaataatttaaaacaaaagcaaaaatcaggacctaagaaaaaaatctattcctgattcttttatgttttgtttgtattttatcaagacagggttgtttctctgtatagccctggctgtcttggaattcactctgtagaccaggctggcctcaaactcagaaatcctcctgcctttgccttccaagtgctggaattaaaggcatgcgccacc

SEQ ID NO: 43 - DNA, 1722 bp, Mus musculus
gacatgacccaaacggcccctggctgcaaggtaatatcggaagttgactaagaatggacgccccaccactgactgacccgccccctgagtctgagattggacttgtctctggatacagtcatactttgaggtactacaagttagaaactgttaggttactcagttcagtccatgacagtccaaccttctccatggttttccgatctcaggcccatggcgacctgccctgtcctgcagaaggagacactgttccgcacaggcgtccatgcttacagaatccctgctctgctctacctgaagaagcagaagaccctgctggcctttgcggaaaagcgagccagcaagacggatgagcacgcagagttgattgtcctgagaagaggaagctacaacgaagccaccaaccgtgtcaagtggcagcctgaggaagtggtgacccaagcccagctggaaggccaccgctccatgaatccatgtcccttgtatgacaagcaaacaaagaccctcttccttttcttcatcgctgtccctgggcgtgtatcagaacatcatcagctccacactaaggttaatgtcacacggctgtgctgtgtcagcagcactgaccatgggaggacctggagccccatccaggacctcacagagaccaccattggcagcactcatcaggaatgggccacatttgctgtgggtcctgggcattgtctgcagctgcggaacccagctgggagcctgctggtacctgcttatgcctaccggaaactgcaccctgctcagaagcctaccccctttgccttctgcttcatcagccttgaccatgggcacacatggaaactaggcaactttgtggctgaaaactcactggagtgccaggtggctgaggttggcactggagctcagaggatggtatatctcaatgctaggagcttcctgggagccagggtccaggcacaaagtcctaatgatggtctggatttccaggacaaccgggtagtgagtaagcttgtagagcccccccacgggtgtcatggaagtgtggttgccttccacaaccccatctctaagccacatgccttagacacatggcttctttatacacaccctacagactccaggaatagaaccaacctgggtgtgtacctaaaccagatgccactagatcccacagcctggtcagagcccaccctgctggccatgggcatctgtgcctactcagacttacagaacatggggcaaggccctgatggctccccacagtttgggtgtctgtatgaatcaggtaactatgaagagatcattttcctcatattcaccctgaagcaagctttccccactgtatttgatgcccagtgatctcagtgcacgtggcccaaagggcttccttgtgcttcaaaacacccatctctctttgcttccagcatcctctggactcttgagtccagctcttgggtaacttcctcaggaggatgcagagaatttggtctcttgactctctgcaggccttattgtttcagcctctggttctcttttcagcccagaaatcaaaggagcctggctttcctcagcctgttggcagggcaggtggggacagtatatatagaggctgccattctgcatgtcggttgtcactatgctagtttaacctgcctgtttccccatgcctagtgtttgaatgagtattaataaaatatccaacccagcccatttcttcctggaaaaaaa

SEQ ID NO: 44 - DNA, 3340 bp, Mus musculus
actgcgcggtgaaggggcgtggcctggccggggaggttgacacccagacgctgctctcagtcctctggcgcctgctccccagcgcattccttctgctcctgggatatttgtctcattactgccagttcttgcgcagcggtcactgggttcgtttcagcgtctgtggtttctgtcgctgttatccagtctccatcgccccagctcagcttcaggccttcttccgagactccacgggagagcccagagagcctccggagccgaagccatggaggaagtcccaccctactccctcagcagcaccctgttccagcaggaagaacagagtggggtgacctaccggatcccagccctgctgtaccttcctcccacccacaccttcctggcctttgcagagaagcggacctcagtcagagatgaggatgctgcctgcctggtgctcagacgagggctgatgaaggggcgctctgtacagtggggcccccaacggctactgatggaggccacattacctgggcatcgcaccatgaacccctgccctgtgtgggagaaaaatactggccgtgtgtacctgtttttcatctgtgtgcggggccatgttactgagaggtgccagattgtgtggggcaaaaatgccgcccgtctctgcttcctttgcagtgaagatgccggctgctcttggggtgaagtgaaagacttgaccgaggaggtcattggctcagaggtgaagcgctgggccacatttgctgtgggcccaggtcatggcatccagctacactcgggaaggctgatcatccccgcctatgcctactatgtctcacgttggtttctctgctttgcgtgttcagtcaagccccattccctgatgatctacagtgatgactttggagtcacatggcaccatggcaagttcattgagccccaggtgacaggggagtgccaagtggccgaagtggctgggacggctggtaaccctgtgctcactgcagtgcccgaacaccaagccgatttcgagcagaggcttttagtactgatagtggtggctgctttcagaagccaaccctgaacccacaactccatgagcctcgaaccggctgccaaggtagtgtagtgagcttccggcctttgaagatgccaaatacctatcaagactcaattggcaaaggtgctcccgctactcagaagtgccctctgctggacagtcctctggaggtggagaaaggagctgaaacaccatcagcaacatggctcttgtactcacatccaactagcaagaggaagaggattaacctaggcatctactacaaccggaaccccttggaggtgaactgctggtcccgcccgtggatcttgaaccgtgggcccagtggctactctgatctggctgttgtggaagaacaggacttggtggcgtgtttgtttgagtgtggggagaagaatgagtatgagcggattgacttctgtctgttttcagaccatgaggtcctgagctgtgaagactgtaccagccctagtagcgactaaagccaaatcaagacggatgagtgaggcccagcttcccacagaaaggaatggcagctacagccagggtaacagaggtctctgatgtctagagaaaactctaaaaactaataatctgctccttgaattttttcacttttcccttcaatgagcatggtgaaaattgtgccatatcttacataacgaggctcttgaactgggagtttgaatctcttctcttcccattaaaaggagaggccatgtgctcgcttcgcgttcgacaaagcctggattctgatcttgagtggaagccacaggcttgtcttttccaatggttcactgctcacctgagtattaggtgatgtgtaggtgccttggccagaagaaagatctgtgttgttgtatttttttaaatttatttatttactatatgtaagtacactgcagctgtcttcagacacaccagaagagggcgtcagatctcattagagatggttgtgagccaccatgtggttgctgggatttgaactcaggaccttcagaagagcagtcagtgctcttaactactgagccatctctcaagccccgcattgctgtatttttaataagaaaaatgcccttatccttccaataatgcctggagctgtacaaattctctgtcttagaagacttgagaaagcagaactgtaaggtcagatgctttctccagccttgatgctgtgttccaccttcccttcctcatccagaaaacagttactagggagaaaatgagaaacccatgccagctgcccttgatgatggttgataacggtgcttattgcttttgatgtcattacctctgttagagatgaatcagagtcagaggtccttagctgcatccacccatttccagggggacattctaacactgctgaacagtcagctaaaatgagagctgtgtgtcctagcctgattccaggttagtcatgatgcttcctggagctgggcttttatctaatcccaggagccatctaggggaggctcagagctagcaggtgatcttcctgagatggtttcaccgtgacaggtgaaccatgagcccttccaagcaaggccaaaggacaacattataggaaagatttctagtattaatatgccttttctctgtgtgtgtactgtcttgtagtgatgctatatagacaaatagatgatttcttattttttgtttgtttgtttgtttttttgtttttctgtagccctagctgtcctggaactcactttgtaaaccaggctggcctcgatctcagaaatccgcctgcctctgcctcccgagtgctgggattaaaggtgtgcaccaccacaccttaatgatgatcctataagtattcctaaaattatactagtaattattaactcctttataataggactgctattaaagccctcgctgatatgaaaactacagtgagaactctgccagtcttcacatgtcataattacttctgagatagaaagcaggcatttacaacttagaacacatttcttagagctgtaaaacaattaactagaggtcataaaagggaatgaaagatttattgtaggtgctaggacagaacataaaatattgactgggcttatctatatgaaacttcattgttaacttttacacaagaattatggtttttaactttcagtgaacctgcggagctagtgacagaagagaaatgtctagttagataactactcttaatggaaattcacataaacatctgttgccatcttctttttgaatttatgtttaaacttgtgaatgtttgaattagacactacgcgagcacatagaaaataaagaactaagcgtgaa

SEQ ID NO: 45 - DNA, 4608 bp, Mus musculus
ggacagtgtgcatcacggagcttgtggcccagactgtgcctggcagacccagaggacctaaggcttggctctagtggtggtcagcacagccctcggtggtctgcggagcctgatattgctttacgtaagggctgttctgctgtgcatctcctgtgtctgaagctattcgccatggagactgctggagctcccttctgcttccatgtggactccctggtaccttgctcctactggaaggttatggggcccacgcgtgttcccaggagaacggtgctcttccagagggaaaggacgggcctgacctaccgtgtgcctgcgttactctgtgtgcctcccaggcctactctgctggccttcgcggaacagcgacttagccctgatgactcccatgcccaccgcctggtgctacggaggggcacgctgaccaggggctcagtgcggtggggcactctgagtgtactggagactgcagtactggaggagcacaggtctatgaacccttgcccggtgctggatgagcactctggtaccatcttcctcttcttcattgccgtgctgggccacacaccggaggccgtgcaaatcgccactggcaagaacgctgctcgcctctgctgtgtgaccagctgtgacgctggcctcacctggggcagtgttcgagatctcactgaggaagccattggtgctgcattgcaggactgggccacctttgctgtgggtccgggccatggagttcagctgcgctcgggtcgcctgcttgttcctgcttacacctatcatgtggaccgacgggaatgttttggcaagatctgctggaccagtccccactccttggcattctacagtgatgatcatgggatctcctggcattgtggaggccttgtgcccaacctacgctctggagagtgccaactggctgcggtagatggagactttctctactgtaatgctcgaagccctctgggtaaccgtgtgcaggcactgagtgctgatgaaggcacgtccttcctaccaggggagctggtgcctacattggcagagacggctcgtggttgccagggtagcattgtgggcttcctagctccaccctcaatcgagcctcaggatgaccggtggacagggagtcctaggaacaccccacattccccatgcttcaatctcagagtacaggagtcttcgggggaaggtgccagaggtcttcttgaacgttggatgcccaggttgcctctctgctacccacagtcccggagcccagagaatcatggcctagagcctgggtcagatggagataagacatcctggactccggaatgtcctatgtcctctgattccatgcttcagagccccacatggctactatattcccacccagcagggcgtagagctcggctccacatgggaatctacctgagccgatcccccttggatccccacagctggacagagccctgggtgatctatgagggccccagtggctactctgaccttgcctttcttgggcctatgcctggggcatccctggtttttgcctgtctgtttgagagcgggaccaggacttcctatgaagacatttctttttgcttgttctcactggcggatgtcctggagaatgtgcccactggcttagagatgctaagtctcagggataaggctcaggggcattgctggccctcttgatggcctcaccctctcgtagccgcctggagaggaagggtagactatatagaggaggttaggggtaggtcagcatgatgctaggatggagagagctctgtcccctcgtggatggtggtggtgactcacccggggggccagctgctttctgagtgcaaatgagaaaaataaagagctgcgctgtgacttttctttccacatcaaagcttgggtgtcagtgctttagcttgatgctctgatcaccatgcaaatcttccaccggcgccttgctcagctttcatatcccaagggtgcctgggaggaaggcaacagggacagtggacatcactgcaccactttccacgaccctgtgtgccaacctcagccactttgaaacatgctgatgactgaggtctgttcactttcttaatttcaagcaggagaagcaggttggggagccagcctccccagctagaggggacagaacttgacttgagcaggggggtacctcctaggacctgctccatgtgcctacttctttacccttctctagagagggctcttgtcctgtcagagctgttttctcccttctcttgttttttctttttcaagactgtttctctgtgttagccctggctgtcctggatctcactctgtagatcaggctgaccttgagttcaaagctccatctgcctctacttctcacattactgtgattaaaggcatatactaccactgcctggtgcccttttgtatttcttattaaagtcctaatgtctgattataaaaacagtctgtgtgggctggagtgatggcttactcagtaaagcacttgccatggaatctgggcaatctgagtttcatttttagcatcctgtaaaaatcccaatttgatggtgtacttgtaatgtcagcatggagaggcagagataggtaagttccccaagactctttgaaccgacagcttggcctcactggcacattccaggtctcagtgagagaccctgcctcaaaatacaaagaaagagctgctgaagagtgggtcagagttgacctctgatctccggaagtatatgatacacacccgtgcatgcactcttccttacaaaataaaaagcaaaacaaaaccccaacaggtatatggccattttagaaaaattagaagatttagaaagctatacataaaaaaaaatgacctaaagaaaaatctttactgttctgggcactatccctatcaaaccactgtgttctttggccaagccttggggtggacactgttttgaggtgggtcctgttatctccactaggtagtggagttttgtgtcagactaactgggtcttaaagctgtctttaaggccatcaggagctactgacttgcctgcctcagcagagcatatcctgaaggtcggggttaagtctccttcccgagcgagttgccttccagtgggcccctggactcctaggtcctcagcgctcatcagctgccaaggactctgagggaatgtcctctgactgtggccccgaaaggtaggggagggggatgtgcttaggcttaggacagggtcctgtttcagtctgccttcactgttagtagcactgtgccacatggcacagactgggcgagctttaaaggaaggaggttgatattggttcccacttctggggatcatggttgagcagccttgtctgatgatggttgtcttgatggtagatcgtgaggtagttgatgaaggtatgacatggtgagaaactctgtgtgtgtgtgttattttctctgtgttctacctatacatctatctatgtatatatgtatctatctatctacctggaggctggagagatagcttagtggttaagaacatttgttgttcttgcatagtcctggatttaaattttcagcacccacatggcagctcacaacaacccataaatccagtttcagaggatccaacctctgatataccatgtcagccagagcagacacggctgaaggtggtttgatccccgtatggagaggtgacaattgggaagagagaaagatcaacttaaccatgcaaggaacaggaagttaaatactgaacagggaaggtaaaggcaggaagtagatgtagagggcaaatcaatgaaacccaaacatacccaaattacgctaaacacacactgacatgccaattaaaaggacaaattggctccactggcaaaaccaaaacagacactgaagatccaaacagtcacatgccaactaccgcggagggagacagacacagagaagaccgtgacagacacttggacactcttgagagtggatgtgcaggaagagagctctgccagtggagaagaaagcactcagaagaaagtgacagcagctgtaaatttgtattctgctaatgttatgttccaaagttgaaagcaaaattgtaccaattcataagaacaaacaggctgactctcagttgtgactgaacgtctctcagtaactgacggggcgagcaggccaaaggagagtcggctcagaagggtgcatagccacgccaaatcaaataagcaagtacaaccggcaggctctatttctagcacaaaggggtctgtgcctcattctgtgcttgggtcagagcttgggtctctcatttggatgtaagtggtgtagtggagaagcaggaaataatccggagcgcatattttgattttaacataagtgctgatttgggagggagttttgtcaaattgtgtttttacaatgtttttttttttttaaatgatgcttttttgtaaagtgtacaaatgtgatataagattggttctgctacattcagtttctataaaagtggttctaaaatattgtactgtcaatcatctcatgattattctactgtacacattactgactttgtatgtaataattaatattagaagaaaatataatttatttgaatataaaaaaaaaaaaaaaaaaa

SEQ ID NO: 46 - protein, 380 aa, Artificial Sequence
  Description of Artificial Sequence Synthetic polypeptide
  MOD_RES (1)..(1): Any amino acid
  MOD_RES (6)..(6): Any amino acid
  MOD_RES (9)..(9): Any amino acid
  MOD_RES (62)..(62): Any amino acid
  MOD_RES (93)..(93): Any amino acid
  MOD_RES (187)..(187): Any amino acid
  MOD_RES (270)..(270): Any amino acid
  MOD_RES (301)..(302): Any amino acid
  MOD_RES (332)..(332): Any amino acid
  MOD_RES (363)..(363): Any amino acid
  MOD_RES (365)..(365): Any amino acid
XASLPXLQXESVFQSGAHAYRIPALLYLPGQQSLLAFAEQRASKKDEHAELIVLRRGDYDAXTHQVQWQAQEVVAQARLDGHRSMNPCPLYDXQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPXQRPIPSAFCFLSHDHGRTWARGHFVAQDTLECQVAEVETGEQRVVTLNARSHLRARVQAQSTNDGLDFQESQLVKKLVEPPPXGCQGSVISFPSPRSGPGSPAQWLLYTHPTHXXQRADLGAYLNPRPPAPEAWSEPVLLAKGSXAYSDLQSMGTGPDGSPLFGCLYEANDYEEIXFXMFTLKQAFPAEYLPQ

SEQ ID NO: 47 - protein, 380 aa, Artificial Sequence
  Description of Artificial Sequence Synthetic polypeptide
  MOD_RES (1)..(2): Any amino acid
  MOD_RES (4)..(6): Any amino acid
  MOD_RES (9)..(9): Any amino acid
  MOD_RES (44)..(45): Any amino acid
  MOD_RES (54)..(54): Any amino acid
  MOD_RES (62)..(62): Any amino acid
  MOD_RES (69)..(69): Any amino acid
  MOD_RES (78)..(78): Any amino acid
  MOD_RES (93)..(93): Any amino acid
  MOD_RES (107)..(108): Any amino acid
  MOD_RES (112)..(112): Any amino acid
  MOD_RES (125)..(126): Any amino acid
  MOD_RES (150)..(150): Any amino acid
  MOD_RES (164)..(164): Any amino acid
  MOD_RES (171)..(171): Any amino acid
  MOD_RES (187)..(187): Any amino acid
  MOD_RES (196)..(196): Any amino acid
  MOD_RES (217)..(217): Any amino acid
  MOD_RES (249)..(249): Any amino acid
  MOD_RES (251)..(251): Any amino acid
  MOD_RES (257)..(257): Any amino acid
  MOD_RES (270)..(270): Any amino acid
  MOD_RES (272)..(272): Any amino acid
  MOD_RES (292)..(292): Any amino acid
  MOD_RES (301)..(302): Any amino acid
  MOD_RES (325)..(325): Any amino acid
  MOD_RES (332)..(332): Any amino acid
  MOD_RES (352)..(352): Any amino acid
  MOD_RES (363)..(363): Any amino acid
  MOD_RES (365)..(365): Any amino acid
XXSXXXLQXESVFQSGAHAYRIPALLYLPGQQSLLAFAEQRASXXDEHAELIVXRRGDYDAXTHQVQWXAQEVVAQAXLDGHRSMNPCPLYDXQTGTLFLFFIAIPXXVTEXQQLQTRANVTRLXXVTSTDHGRTWSSPRDLTDAAIGPXYREWSTFAVGPGHXLQLHDRXRSLVVPAYAYRKLHPXQRPIPSAFXFLSHDHGRTWARGHFVAQDTXECQVAEVETGEQRVVTLNARSHLRARVQAQSXNXGLDFQXSQLVKKLVEPPPXGXQGSVISFPSPRSGPGSPAQXLLYTHPTHXXQRADLGAYLNPRPPAPEAWSEPXLLAKGSXAYSDLQSMGTGPDGSPLFGXLYEANDYEEIXFXMFTLKQAFPAEYLPQ

SEQ ID NO: 48 - protein, 380 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQ

SEQ ID NO: 49 - protein, 380 aa, Artificial Sequence
  Description of Artificial Sequence Synthetic polypeptide
DASLPYLQDESVFQSGAHAYRIPALLYLPGQQSLLAFAEQRASKKDEHAELIVLRRGDYDAPTHQVQWQAQEVVAQARLDGHRSMNPCPLYDEQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIRFIMFTLKQAFPAEYLPQ

SEQ ID NO: 50 - protein, 380 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDANTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQ

SEQ ID NO: 51 - protein, 380 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPAGCQGSVISFPSPRSGPGSPAQWLLYTHPTHRKQRADLGAYLNPRPPAPEAWSEPVLLAKGSAAYSDLQSMGTGPDGSPLFGCLYEANDYEEIVFLMFTLKQAFPAEYLPQ

SEQ ID NO: 52 - protein, 380 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDASTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPAGCQGSVISFPSPRSGPGSPAQWLLYTHPTHRKQRADLGAYLNPRPPAPEAWSEPVLLAKGSAAYSDLQSMGTGPDGSPLFGCLYEANDYEEIVFLMFTLKQAFPAEYLPQ

SEQ ID NO: 53 - protein, 380 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDATTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPAGCQGSVISFPSPRSGPGSPAQWLLYTHPTHRKQRADLGAYLNPRPPAPEAWSEPVLLAKGSAAYSDLQSMGTGPDGSPLFGCLYEANDYEEIVFLMFTLKQAFPAEYLPQ

SEQ ID NO: 54 - protein, 380 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDANTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPAGCQGSVISFPSPRSGPGSPAQWLLYTHPTHRKQRADLGAYLNPRPPAPEAWSEPVLLAKGSAAYSDLQSMGTGPDGSPLFGCLYEANDYEEIVFLMFTLKQAFPAEYLPQ

SEQ ID NO: 55 - protein, 379 aa, Artificial Sequence
  Description of Artificial Sequence Synthetic polypeptide
ASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQ

SEQ ID NO: 56 - protein, 380 aa, Artificial Sequence
  Description of Artificial Sequence Synthetic polypeptide
MASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQ

SEQ ID NO: 57 - protein, 379 aa, Artificial Sequence
  Description of Artificial Sequence Synthetic polypeptide
ASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQ

SEQ ID NO: 58 - protein, 380 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQ

SEQ ID NO: 59 - protein, 380 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQ

SEQ ID NO: 60 - protein, 380 aa, Artificial Sequence
  Description of Artificial Sequence Synthetic polypeptide
AASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQ

SEQ ID NO: 61 - protein, 380 aa, Artificial Sequence
  Description of Artificial Sequence Synthetic polypeptide
MASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQ

SEQ ID NO: 62 - protein, 380 aa, Artificial Sequence
  Description of Artificial Sequence Synthetic polypeptide
AASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQ

SEQ ID NO: 63 - protein, 380 aa, Artificial Sequence
  Description of Artificial Sequence Synthetic polypeptide
DASLPYLQKESVFQSGAHAYRIPALLYLPGQQSLLAFAEQRRSKKDEHAELIVLRRGDYDAGTHQVQWQAQEVVAQARLDGHRSMNPCPLYDEQTGTLFLFFIAIPGQVTEQQQLQTRANVTRLCYVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRFRVQAQSTNDGLDFQESQLVKKLVEPPPTGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQ

SEQ ID NO: 64 - protein, 380 aa, Artificial Sequence
  Description of Artificial Sequence Synthetic polypeptide
  MOD_RES (1)..(1): Ala, Arg, Asn, Asp, Gln, Glu, Gly, His, Leu, Lys, Met, Phe, Thr, Val, or not present
  MOD_RES (2)..(2): Ala or Lys
  MOD_RES (4)..(4): Asn or Leu
  MOD_RES (5)..(5): Pro or His
  MOD_RES (6)..(6): Phe, Trp, Tyr or Val
  MOD_RES (9)..(9): Lys or Asp
  MOD_RES (42)..(42): Ala or Arg
  MOD_RES (44)..(44): Lys, Arg, or Glu
  MOD_RES (45)..(45): Lys, Ala, Arg, or Glu
  MOD_RES (54)..(54): Leu or Met
  MOD_RES (62)..(62): Pro, Asn, Asp, His, Glu, Gly, Ser or Thr
  MOD_RES (69)..(69): Gln or His
  MOD_RES (78)..(78): Arg or Lys
  MOD_RES (80)..(80): Asp or Pro
  MOD_RES (93)..(93): Ala, Glu or Lys
  MOD_RES (107)..(107): Gly or Asp
  MOD_RES (108)..(108): Gln or His
  MOD_RES (112)..(112): Gln, Arg, or Lys
  MOD_RES (125)..(125): Ala, Cys, Ile, Ser, Val, or Leu
  MOD_RES (126)..(126): Gln, Leu, Glu, Phe, His, Ile, Leu, or Tyr
  MOD_RES (150)..(150): Ala or Val
  MOD_RES (164)..(164): Cys or Gly
  MOD_RES (170)..(170): Arg or Pro
  MOD_RES (171)..(171): Ala or Gly
  MOD_RES (187)..(187): Arg, Ile, or Lys
  MOD_RES (188)..(188): Gln or Pro
  MOD_RES (189)..(189): Arg or Pro
  MOD_RES (196)..(196): Ala, Cys, Leu, or Val
  MOD_RES (213)..(213): Ala, Cys, Asn, Ser, or Thr
  MOD_RES (217)..(217): Leu, Ala, or Val
  MOD_RES (225)..(225): Glu or Pro
  MOD_RES (239)..(239): His or Pro
  MOD_RES (240)..(240): Leu, Asp, Asn, or Tyr
  MOD_RES (241)..(241): Arg, Ala, Asp, Leu, Gln, or Tyr
  MOD_RES (242)..(242): Ala, Cys, Phe, Gly, His, Ile, Lys, Leu, Met, Asn, Gln, Arg, Ser, Val, Trp, or Tyr
  MOD_RES (244)..(244): Val, Ile, or Lys
  MOD_RES (249)..(249): Thr or Ala
  MOD_RES (251)..(251): Asp or Gly
  MOD_RES (257)..(257): Glu, Lys, or Pro
  MOD_RES (258)..(258): Ser or Cys
  MOD_RES (260)..(260): Leu, Asp, Phe, Gln, or Thr
  MOD_RES (265)..(265): Val or Phe
  MOD_RES (270)..(270): Gln, Ala, His, Phe, Pro, Ser, or Thr
  MOD_RES (272)..(272): Cys or Val
  MOD_RES (292)..(292): Trp or Arg
  MOD_RES (301)..(301): Ser, Arg, Ala, Asp, Glu, Phe, Gly, His, Ile, Lys, Leu, Met, Asn, Pro, Gln, Thr, Val, Trp, or Tyr
  MOD_RES (302)..(302): Trp, Lys, Ala, Asp, Glu, Phe, Gly, His, Ile, Lys, Leu, Met, Asn, Pro, Gln, Arg, Ser, Thr, Val, or Tyr
  MOD_RES (325)..(325): Lys or Val
  MOD_RES (332)..(332): Ala, Cys, Ser, or Val
  MOD_RES (352)..(352): Cys, Leu, or Val
  MOD_RES (363)..(363): Val or Arg
  MOD_RES (365)..(365): Leu, Gln, His, Ile, Lys, or Ser
  See specification as filed for detailed description of substitutions and preferred embodiments
XXSXXXLQXESVFQSGAHAYRIPALLYLPGQQSLLAFAEQRXSXXDEHAELIVXRRGDYDAXTHQVQWXAQEVVAQAXLXGHRSMNPCPLYDXQTGTLFLFFIAIPXXVTEXQQLQTRANVTRLXXVTSTDHGRTWSSPRDLTDAAIGPXYREWSTFAVGPGHXLQLHDXXRSLVVPAYAYRKLHPXXXPIPSAFXFLSHDHGRTWARGHFVXQDTXECQVAEVXTGEQRVVTLNARSXXXXRXQAQSXNXGLDFQXXQXVKKLXEPPPXGXQGSVISFPSPRSGPGSPAQXLLYTHPTHXXQRADLGAYLNPRPPAPEAWSEPXLLAKGSXAYSDLQSMGTGPDGSPLFGXLYEANDYEEIXFXMFTLKQAFPAEYLPQ

SEQ ID NO: 65 - protein, 380 aa, Artificial Sequence
  Description of Artificial Sequence Synthetic polypeptide
  MOD_RES (1)..(1): Ala, Arg, Asn, Asp, Gln, Glu, Gly, His, Leu, Lys, Met, Phe, Thr, Val, or not present
  MOD_RES (6)..(6): Phe, Trp, Tyr or Val
  MOD_RES (9)..(9): Lys or Asp
  MOD_RES (42)..(42): Arg or Ala
  MOD_RES (62)..(62): Pro, Asn, Asp, His, Glu, Gly, Ser or Thr
  MOD_RES (93)..(93): Ala, Glu, or Lys
  MOD_RES (126)..(126): Gln, Leu, Glu, Phe, His, Ile, Leu, or Tyr
  MOD_RES (187)..(187): Arg, Ile, or Lys
  MOD_RES (242)..(242): Ala, Cys, Phe, Gly, His, Ile, Lys, Leu, Met, Asn, Gln, Arg, Ser, Val, Trp, or Tyr
  MOD_RES (270)..(270): Gln, Ala, His, Phe, Pro, Ser, or Thr
  MOD_RES (301)..(301): Ser, Arg, Ala, Asp, Glu, Phe, Gly, His, Ile, Lys, Leu, Met, Asn, Pro, Gln, Thr, Val, Trp, or Tyr
  MOD_RES (302)..(302): Trp, Lys, Ala, Asp, Glu, Phe, Gly, His, Ile, Lys, Leu, Met, Asn, Pro, Gln, Arg, Ser, Thr, Val, or Tyr
  MOD_RES (332)..(332): Ala, Cys, Ser, or Val
  MOD_RES (363)..(363): Val or Arg
  MOD_RES (365)..(365): Leu, Gln, His, Ile, Lys, or Ser
  See specification as filed for detailed description of substitutions and preferred embodiments
XASLPXLQXESVFQSGAHAYRIPALLYLPGQQSLLAFAEQRXSKKDEHAELIVLRRGDYDAXTHQVQWQAQEVVAQARLDGHRSMNPCPLYDXQTGTLFLFFIAIPGQVTEQQQLQTRANVTRLCXVTSTDHGRTWSSPRDLTDAAIGPAYREWSTFAVGPGHCLQLHDRARSLVVPAYAYRKLHPXQRPIPSAFCFLSHDHGRTWARGHFVAQDTLECQVAEVETGEQRVVTLNARSHLRXRVQAQSTNDGLDFQESQLVKKLVEPPPXGCQGSVISFPSPRSGPGSPAQWLLYTHPTHXXQRADLGAYLNPRPPAPEAWSEPVLLAKGSXAYSDLQSMGTGPDGSPLFGCLYEANDYEEIXFXMFTLKQAFPAEYLPQ

SEQ ID NO: 66 - protein, 617 aa, Artificial Sequence
  Description of Artificial Sequence Synthetic polypeptide
  MOD_RES (1)..(1): Ala, Arg, Asn, Asp, Gln, Glu, Gly, His, Leu, Lys, Met, Phe, Thr, Val, or not present
  MOD_RES (2)..(2): Ala or Lys
  MOD_RES (4)..(4): Asn or Leu
  MOD_RES (5)..(5): Pro or His
  MOD_RES (6)..(6): Phe, Trp, Tyr or Val
  MOD_RES (9)..(9): Lys or Asp
  MOD_RES (42)..(42): Ala or Arg
  MOD_RES (44)..(44): Lys, Arg, or Glu
  MOD_RES (45)..(45): Lys, Ala, Arg, or Glu
  MOD_RES (54)..(54): Leu or Met
  MOD_RES (62)..(62): Pro, Asn, Asp, His, Glu, Gly, Ser or Thr
  MOD_RES (69)..(69): Gln or His
  MOD_RES (78)..(78): Arg or Lys
  MOD_RES (80)..(80): Asp or Pro
  MOD_RES (93)..(93): Ala, Glu or Lys
  MOD_RES (107)..(107): Gly or Asp
  MOD_RES (108)..(108): Gln or His
  MOD_RES (112)..(112): Gln, Arg, or Lys
  MOD_RES (125)..(125): Ala, Cys, Ile, Ser, Val, or Leu
  MOD_RES (126)..(126): Gln, Leu, Glu, Phe, His, Ile, Leu, or Tyr
  MOD_RES (150)..(150): Ala or Val
  MOD_RES (164)..(164): Cys or Gly
  MOD_RES (170)..(170): Arg or Pro
  MOD_RES (171)..(171): Ala or Gly
  MOD_RES (187)..(187): Arg, Ile, or Lys
  MOD_RES (188)..(188): Gln or Pro
  MOD_RES (189)..(189): Arg or Pro
  MOD_RES (196)..(196): Ala, Cys, Leu, or Val
  MOD_RES (213)..(213): Ala, Cys, Asn, Ser, or Thr
  MOD_RES (217)..(217): Leu, Ala, or Val
  MOD_RES (225)..(225): Glu or Pro
  MOD_RES (239)..(239): His or Pro
  MOD_RES (240)..(240): Leu, Asp, Asn, or Tyr
  MOD_RES (241)..(241): Arg, Ala, Asp, Leu, Gln, or Tyr
  MOD_RES (242)..(242): Ala, Cys, Phe, Gly, His, Ile, Lys, Leu, Met, Asn, Gln, Arg, Ser, Val, Trp, or Tyr
  MOD_RES (244)..(244): Val, Ile, or Lys
  MOD_RES (249)..(249): Thr or Ala
  MOD_RES (251)..(251): Asp or Gly
  MOD_RES (257)..(257): Glu, Lys, or Pro
  MOD_RES (258)..(258): Ser or Cys
  MOD_RES (260)..(260): Leu, Asp, Phe, Gln, or Thr
  MOD_RES (265)..(265): Val or Phe
  MOD_RES (270)..(270): Gln, Ala, His, Phe, Pro, Ser, or Thr
  MOD_RES (272)..(272): Cys or Val
  MOD_RES (292)..(292): Trp or Arg
  MOD_RES (301)..(301): Ser, Arg, Ala, Asp, Glu, Phe, Gly, His, Ile, Lys, Leu, Met, Asn, Pro, Gln, Thr, Val, Trp, or Tyr
  MOD_RES (302)..(302): Trp, Lys, Ala, Asp, Glu, Phe, Gly, His, Ile, Lys, Leu, Met, Asn, Pro, Gln, Arg, Ser, Thr, Val, or Tyr
  MOD_RES (325)..(325): Lys or Val
  MOD_RES (332)..(332): Ala, Cys, Ser, or Val
  MOD_RES (352)..(352): Cys, Leu, or Val
  MOD_RES (363)..(363): Val or Arg
  MOD_RES (365)..(365): Leu, Gln, His, Ile, Lys, or Ser
  MOD_RES (381)..(390): This region may encompass one of the following sequences “GGGGS” or “GGGGSGGGGS” or “EPKSS”
  See specification as filed for detailed description of substitutions and preferred embodiments
XXSXXXLQXESVFQSGAHAYRIPALLYLPGQQSLLAFAEQRXSXXDEHAELIVXRRGDYDAXTHQVQWXAQEVVAQAXLXGHRSMNPCPLYDXQTGTLFLFFIAIPXXVTEXQQLQTRANVTRLXXVTSTDHGRTWSSPRDLTDAAIGPXYREWSTFAVGPGHXLQLHDXXRSLVVPAYAYRKLHPXXXPIPSAFXFLSHDHGRTWARGHFVXQDTXECQVAEVXTGEQRVVTLNARSXXXXRXQAQSXNXGLDFQXXQXVKKLXEPPPXGXQGSVISFPSPRSGPGSPAQXLLYTHPTHXXQRADLGAYLNPRPPAPEAWSEPXLLAKGSXAYSDLQSMGTGPDGSPLFGXLYEANDYEEIXFXMFTLKQAFPAEYLPQXXXXXXXXXXDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 67 - protein, 617 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 68 - protein, 617 aa, Artificial Sequence
  Description of Artificial Sequence Synthetic polypeptide
DASLPYLQDESVFQSGAHAYRIPALLYLPGQQSLLAFAEQRASKKDEHAELIVLRRGDYDAPTHQVQWQAQEVVAQARLDGHRSMNPCPLYDEQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIRFIMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 69 - protein, 617 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDANTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 70 - protein, 617 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPAGCQGSVISFPSPRSGPGSPAQWLLYTHPTHRKQRADLGAYLNPRPPAPEAWSEPVLLAKGSAAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 71 - protein, 617 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDASTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPAGCQGSVISFPSPRSGPGSPAQWLLYTHPTHRKQRADLGAYLNPRPPAPEAWSEPVLLAKGSAAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 72 - protein, 617 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDATTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPAGCQGSVISFPSPRSGPGSPAQWLLYTHPTHRKQRADLGAYLNPRPPAPEAWSEPVLLAKGSAAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 73 - protein, 617 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDANTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPAGCQGSVISFPSPRSGPGSPAQWLLYTHPTHRKQRADLGAYLNPRPPAPEAWSEPVLLAKGSAAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 74 - protein, 617 aa, Artificial Sequence
  Description of Artificial Sequence Synthetic polypeptide
  MOD_RES (1)..(1): Any amino acid
  MOD_RES (6)..(6): Any amino acid
  MOD_RES (9)..(9): Any amino acid
  MOD_RES (62)..(62): Any amino acid
  MOD_RES (93)..(93): Any amino acid
  MOD_RES (187)..(187): Any amino acid
  MOD_RES (270)..(270): Any amino acid
  MOD_RES (301)..(302): Any amino acid
  MOD_RES (332)..(332): Any amino acid
  MOD_RES (363)..(363): Any amino acid
  MOD_RES (365)..(365): Any amino acid
XASLPXLQXESVFQSGAHAYRIPALLYLPGQQSLLAFAEQRASKKDEHAELIVLRRGDYDAXTHQVQWQAQEVVAQARLDGHRSMNPCPLYDXQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPXQRPIPSAFCFLSHDHGRTWARGHFVAQDTLECQVAEVETGEQRVVTLNARSHLRARVQAQSTNDGLDFQESQLVKKLVEPPPXGCQGSVISFPSPRSGPGSPAQWLLYTHPTHXXQRADLGAYLNPRPPAPEAWSEPVLLAKGSXAYSDLQSMGTGPDGSPLFGCLYEANDYEEIXFXMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 75 - protein, 617 aa, Artificial Sequence
  Description of Artificial Sequence Synthetic polypeptide
  MOD_RES (1)..(2): Any amino acid
  MOD_RES (4)..(6): Any amino acid
  MOD_RES (9)..(9): Any amino acid
  MOD_RES (44)..(45): Any amino acid
  MOD_RES (54)..(54): Any amino acid
  MOD_RES (62)..(62): Any amino acid
  MOD_RES (69)..(69): Any amino acid
  MOD_RES (78)..(78): Any amino acid
  MOD_RES (93)..(93): Any amino acid
  MOD_RES (107)..(108): Any amino acid
  MOD_RES (112)..(112): Any amino acid
  MOD_RES (125)..(126): Any amino acid
  MOD_RES (150)..(150): Any amino acid
  MOD_RES (164)..(164): Any amino acid
  MOD_RES (171)..(171): Any amino acid
  MOD_RES (187)..(187): Any amino acid
  MOD_RES (196)..(196): Any amino acid
  MOD_RES (217)..(217): Any amino acid
  MOD_RES (249)..(249): Any amino acid
  MOD_RES (251)..(251): Any amino acid
  MOD_RES (257)..(257): Any amino acid
  MOD_RES (270)..(270): Any amino acid
  MOD_RES (272)..(272): Any amino acid
  MOD_RES (292)..(292): Any amino acid
  MOD_RES (301)..(302): Any amino acid
  MOD_RES (325)..(325): Any amino acid
  MOD_RES (332)..(332): Any amino acid
  MOD_RES (352)..(352): Any amino acid
  MOD_RES (363)..(363): Any amino acid
  MOD_RES (365)..(365): Any amino acid
XXSXXXLQXESVFQSGAHAYRIPALLYLPGQQSLLAFAEQRASXXDEHAELIVXRRGDYDAXTHQVQWXAQEVVAQAXLDGHRSMNPCPLYDXQTGTLFLFFIAIPXXVTEXQQLQTRANVTRLXXVTSTDHGRTWSSPRDLTDAAIGPXYREWSTFAVGPGHXLQLHDRXRSLVVPAYAYRKLHPXQRPIPSAFXFLSHDHGRTWARGHFVAQDTXECQVAEVETGEQRVVTLNARSHLRARVQAQSXNXGLDFQXSQLVKKLVEPPPXGXQGSVISFPSPRSGPGSPAQXLLYTHPTHXXQRADLGAYLNPRPPAPEAWSEPXLLAKGSXAYSDLQSMGTGPDGSPLFGXLYEANDYEEIXFXMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 76 - protein, 617 aa, Artificial Sequence
  Description of Artificial Sequence Synthetic polypeptide
  MOD_RES (1)..(1): Ala, Arg, Asn, Asp, Gln, Glu, Gly, His, Leu, Lys, Met, Phe, Thr, Val, or not present
  MOD_RES (6)..(6): Phe, Trp, Tyr or Val
  MOD_RES (9)..(9): Lys or Asp
  MOD_RES (42)..(42): Arg or Ala
  MOD_RES (62)..(62): Pro, Asn, Asp, His, Glu, Gly, Ser or Thr
  MOD_RES (93)..(93): Ala, Glu, or Lys
  MOD_RES (126)..(126): Gln, Leu, Glu, Phe, His, Ile, Leu, or Tyr
  MOD_RES (187)..(187): Arg, Ile, or Lys
  MOD_RES (242)..(242): Ala, Cys, Phe, Gly, His, Ile, Lys, Leu, Met, Asn, Gln, Arg, Ser, Val, Trp, or Tyr
  MOD_RES (270)..(270): Gln, Ala, His, Phe, Pro, Ser, or Thr
  MOD_RES (301)..(301): Ser, Arg, Ala, Asp, Glu, Phe, Gly, His, Ile, Lys, Leu, Met, Asn, Pro, Gln, Thr, Val, Trp, or Tyr
  MOD_RES (302)..(302): Trp, Lys, Ala, Asp, Glu, Phe, Gly, His, Ile, Lys, Leu, Met, Asn, Pro, Gln, Arg, Ser, Thr, Val, or Tyr
  MOD_RES (332)..(332): Ala, Cys, Ser, or Val
  MOD_RES (363)..(363): Val or Arg
  MOD_RES (365)..(365): Leu, Gln, His, Ile, Lys, or Ser
  MOD_RES (381)..(390): This region may encompass one of the following sequences “GGGGS” or “GGGGSGGGGS” or “EPKSS”
  See specification as filed for detailed description of substitutions and preferred embodiments
XASLPXLQXESVFQSGAHAYRIPALLYLPGQQSLLAFAEQRXSKKDEHAELIVLRRGDYDAXTHQVQWQAQEVVAQARLDGHRSMNPCPLYDXQTGTLFLFFIAIPGQVTEQQQLQTRANVTRLCXVTSTDHGRTWSSPRDLTDAAIGPAYREWSTFAVGPGHCLQLHDRARSLVVPAYAYRKLHPXQRPIPSAFCFLSHDHGRTWARGHFVAQDTLECQVAEVETGEQRVVTLNARSHLRXRVQAQSTNDGLDFQESQLVKKLVEPPPXGCQGSVISFPSPRSGPGSPAQWLLYTHPTHXXQRADLGAYLNPRPPAPEAWSEPVLLAKGSXAYSDLQSMGTGPDGSPLFGCLYEANDYEEIXFXMFTLKQAFPAEYLPQXXXXXXXXXXDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 77 - protein, 218 aa, Artificial Sequence
  Description of Artificial Sequence Synthetic polypeptide
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 612 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 79 - DNA, 1836 bp, Artificial Sequence
  Description of Artificial Sequence Synthetic polynucleotide
gatgcatctctgccttacctgcagaaagaaagcgtgttccagtctggcgcccacgcctacagaattcccgctctgctgtatctgccaggccagcagtctctgctggctttcgctgaacagcgggccagcaagaaggatgagcacgccgaactgatcgtgctgcggagaggcgattacgacgccggcacacatcaggtgcagtggcaggctcaagaggtggtggctcaggctagactggacggccacagatctatgaacccctgtcctctgtacgatgaacagaccggcacactgtttctgttctttatcgctatccccggccaagtgaccgagcagcagcagctgcagacaagagccaacgtgaccagactgtgtcaagtgacctccaccgaccacggcagaacctggtctagccctagagatctgaccgacgccgccatcggacctgcctatagagagtggtccaccttcgccgttggacctggacactgtctccagctgcacgacagggctagatctctggtggtgcctgcctacgcctatagaaagctgcaccccaaacagcggcctattcctagcgccttctgctttctgagccacgatcacggcaggacatgggccagaggacatttcgtggcccaggacacactggaatgccaggtggccgaagtggaaaccggcgagcagagagtcgtgaccctgaacgccagatctcacctgagagccagagtgcaggcccagagcacaaacgacggcctggatttccaagagagccagctggtcaagaaactggtggaacctcctccacagggctgtcagggaagcgtgatcagctttccatctcctagaagcggccctggctctcctgctcagtggctgctgtatacacaccccacacacagctggcagagagccgatctgggcgcctacctgaatcctagacctcctgctcctgaggcttggagcgaacctgttctgctggccaagggcagcgctgcctacagcgatctgcagtctatgggcacaggccctgatggcagccctctgtttggctgtctgtacgaggccaacgactacgaagagatcgtgttcctgatgttcaccctgaagcaggcctttccagccgagtacctgcctcaaggcggaggcggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctcactagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 80 - DNA, 1140 bp, Artificial Sequence
  Description of Artificial Sequence Synthetic polynucleotide
gatgcatctctgccttacctgcagaaagaaagcgtgttccagtctggcgcccacgcctacagaattcccgctctgctgtatctgccaggccagcagtctctgctggctttcgctgaacagcgggccagcaagaaggatgagcacgccgaactgatcgtgctgcggagaggcgattacgacgccggcacacatcaggtgcagtggcaggctcaagaggtggtggctcaggctagactggacggccacagatctatgaacccctgtcctctgtacgatgaacagaccggcacactgtttctgttctttatcgctatccccggccaagtgaccgagcagcagcagctgcagacaagagccaacgtgaccagactgtgtcaagtgacctccaccgaccacggcagaacctggtctagccctagagatctgaccgacgccgccatcggacctgcctatagagagtggtccaccttcgccgttggacctggacactgtctccagctgcacgacagggctagatctctggtggtgcctgcctacgcctatagaaagctgcaccccaaacagcggcctattcctagcgccttctgctttctgagccacgatcacggcaggacatgggccagaggacatttcgtggcccaggacacactggaatgccaggtggccgaagtggaaaccggcgagcagagagtcgtgaccctgaacgccagatctcacctgagagccagagtgcaggcccagagcacaaacgacggcctggatttccaagagagccagctggtcaagaaactggtggaacctcctccacagggctgtcagggaagcgtgatcagctttccatctcctagaagcggccctggctctcctgctcagtggctgctgtatacacaccccacacacagctggcagagagccgatctgggcgcctacctgaatcctagacctcctgctcctgaggcttggagcgaacctgttctgctggccaagggcagcgctgcctacagcgatctgcagtctatgggcacaggccctgatggcagccctctgtttggctgtctgtacgaggccaacgactacgaagagatcgtgttcctgatgttcaccctgaagcaggcctttccagccgagtacctgcctcaa

SEQ ID NO: 81 - protein, 612 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 82 - protein, 612 aa, Artificial Sequence
  Description of Artificial Sequence Synthetic polypeptide
DASLPYLQDESVFQSGAHAYRIPALLYLPGQQSLLAFAEQRASKKDEHAELIVLRRGDYDAPTHQVQWQAQEVVAQARLDGHRSMNPCPLYDEQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIRFIMFTLKQAFPAEYLPQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 83 - protein, 612 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDANTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 84 - protein, 612 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPAGCQGSVISFPSPRSGPGSPAQWLLYTHPTHRKQRADLGAYLNPRPPAPEAWSEPVLLAKGSAAYSDLQSMGTGPDGSPLFGCLYEANDYEEIVFLMFTLKQAFPAEYLPQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 85 - protein, 612 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDASTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPAGCQGSVISFPSPRSGPGSPAQWLLYTHPTHRKQRADLGAYLNPRPPAPEAWSEPVLLAKGSAAYSDLQSMGTGPDGSPLFGCLYEANDYEEIVFLMFTLKQAFPAEYLPQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 86 - protein, 612 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDATTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPAGCQGSVISFPSPRSGPGSPAQWLLYTHPTHRKQRADLGAYLNPRPPAPEAWSEPVLLAKGSAAYSDLQSMGTGPDGSPLFGCLYEANDYEEIVFLMFTLKQAFPAEYLPQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 87 - protein, 612 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDANTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPAGCQGSVISFPSPRSGPGSPAQWLLYTHPTHRKQRADLGAYLNPRPPAPEAWSEPVLLAKGSAAYSDLQSMGTGPDGSPLFGCLYEANDYEEIVFLMFTLKQAFPAEYLPQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 88 - protein, 608 aa, Artificial Sequence
  Description of Artificial Sequence Synthetic polypeptide
  MOD_RES (1)..(1): Any amino acid
  MOD_RES (6)..(6): Any amino acid
  MOD_RES (9)..(9): Any amino acid
  MOD_RES (62)..(62): Any amino acid
  MOD_RES (93)..(93): Any amino acid
  MOD_RES (187)..(187): Any amino acid
  MOD_RES (270)..(270): Any amino acid
  MOD_RES (301)..(302): Any amino acid
  MOD_RES (332)..(332): Any amino acid
  MOD_RES (363)..(363): Any amino acid
  MOD_RES (365)..(365): Any amino acid
  MOD_RES (381)..(381): Any amino acid
XASLPXLQXESVFQSGAHAYRIPALLYLPGQQSLLAFAEQRASKKDEHAELIVLRRGDYDAXTHQVQWQAQEVVAQARLDGHRSMNPCPLYDXQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPXQRPIPSAFCFLSHDHGRTWARGHFVAQDTLECQVAEVETGEQRVVTLNARSHLRARVQAQSTNDGLDFQESQLVKKLVEPPPXGCQGSVISFPSPRSGPGSPAQWLLYTHPTHXXQRADLGAYLNPRPPAPEAWSEPVLLAKGSXAYSDLQSMGTGPDGSPLFGCLYEANDYEEIXFXMFTLKQAFPAEYLPQXDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 89 - protein, 608 aa, Artificial Sequence
  Description of Artificial Sequence Synthetic polypeptide
  MOD_RES (1)..(2): Any amino acid
  MOD_RES (4)..(6): Any amino acid
  MOD_RES (9)..(9): Any amino acid
  MOD_RES (44)..(45): Any amino acid
  MOD_RES (54)..(54): Any amino acid
  MOD_RES (62)..(62): Any amino acid
  MOD_RES (69)..(69): Any amino acid
  MOD_RES (78)..(78): Any amino acid
  MOD_RES (93)..(93): Any amino acid
  MOD_RES (107)..(108): Any amino acid
  MOD_RES (112)..(112): Any amino acid
  MOD_RES (125)..(126): Any amino acid
  MOD_RES (150)..(150): Any amino acid
  MOD_RES (164)..(164): Any amino acid
  MOD_RES (171)..(171): Any amino acid
  MOD_RES (187)..(187): Any amino acid
  MOD_RES (196)..(196): Any amino acid
  MOD_RES (217)..(217): Any amino acid
  MOD_RES (249)..(249): Any amino acid
  MOD_RES (251)..(251): Any amino acid
  MOD_RES (257)..(257): Any amino acid
  MOD_RES (270)..(270): Any amino acid
  MOD_RES (272)..(272): Any amino acid
  MOD_RES (292)..(292): Any amino acid
  MOD_RES (301)..(302): Any amino acid
  MOD_RES (325)..(325): Any amino acid
  MOD_RES (332)..(332): Any amino acid
  MOD_RES (352)..(352): Any amino acid
  MOD_RES (363)..(363): Any amino acid
  MOD_RES (365)..(365): Any amino acid
  MOD_RES (381)..(381): Any amino acid
XXSXXXLQXESVFQSGAHAYRIPALLYLPGQQSLLAFAEQRASXXDEHAELIVXRRGDYDAXTHQVQWXAQEVVAQAXLDGHRSMNPCPLYDXQTGTLFLFFIAIPXXVTEXQQLQTRANVTRLXXVTSTDHGRTWSSPRDLTDAAIGPXYREWSTFAVGPGHXLQLHDRXRSLVVPAYAYRKLHPXQRPIPSAFXFLSHDHGRTWARGHFVAQDTXECQVAEVETGEQRVVTLNARSHLRARVQAQSXNXGLDFQXSQLVKKLVEPPPXGXQGSVISFPSPRSGPGSPAQXLLYTHPTHXXQRADLGAYLNPRPPAPEAWSEPXLLAKGSXAYSDLQSMGTGPDGSPLFGXLYEANDYEEIXFXMFTLKQAFPAEYLPQXDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 90 - protein, 10 aa, Artificial Sequence
  Description of Artificial Sequence Synthetic peptide
GGGGSGGGGS

SEQ ID NO: 91 - protein, 5 aa, Artificial Sequence
  Description of Artificial Sequence Synthetic peptide
EPKSS

SEQ ID NO: 92 - protein, 232 aa, Artificial Sequence
  Description of Artificial Sequence Synthetic polypeptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 93 - protein, 227 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 380 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHARQRADLGAYLNPRPPAPEAWSEPVLLAKGSAAYSDLQSMGTGPDGSPLFGCLYEANDYEEIVFLMFTLKQAFPAEYLPQ

SEQ ID NO: 95 - protein, 617 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHARQRADLGAYLNPRPPAPEAWSEPVLLAKGSAAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 96 - protein, 612 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHARQRADLGAYLNPRPPAPEAWSEPVLLAKGSAAYSDLQSMGTGPDGSPLFGCLYEANDYEEIVFLMFTLKQAFPAEYLPQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 97 - protein, 380 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCYVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPTGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQ

SEQ ID NO: 98 - protein, 617 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCYVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPTGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 99 - protein, 612 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCYVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPTGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 100 - protein, 380 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCYVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRFRVQAQSTNDGLDFQESQLVKKLVEPPPTGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQ

SEQ ID NO: 101 - protein, 617 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCYVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRFRVQAQSTNDGLDFQESQLVKKLVEPPPTGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 102 - protein, 612 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCYVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRFRVQAQSTNDGLDFQESQLVKKLVEPPPTGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 103 - DNA, 1836 bp, Artificial Sequence; identical to SEQ ID NO: 79
  Description of Artificial Sequence Synthetic polynucleotide

SEQ ID NO: 104 - DNA, 654 bp, Artificial Sequence
  Description of Artificial Sequence Synthetic polynucleotide
gagatcgtgctgacacagagccctgccacactgtcactgtctccaggcgagagagccacactgagctgtagagcttctaagggcgtgtccaccagcggctactcttacctgcactggtatcagcagaagcccggccaggctcctagactgctgatctacctggccagctacctggaaagcggagtgcctgctagattcagcggctctggctctggcaccgacttcaccctgacaatcagcagcctggaacctgaggacttcgccgtgtactactgccagcacagcagagatctgcccctgacatttggcggaggcaccaaggtggaaatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 105 - protein, 450 aa, Artificial Sequence
  Description of Artificial Sequence Synthetic polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 106 - protein, 632 aa, Artificial Sequence
  Description of Artificial Sequence Synthetic polypeptide
TVEKSVVFKAEGEHFTDQKGNTIVGSGSGGTTKYFRIPAMCTTSKGTIVVFADARHNTASDQSFIDTAAARSTDGGKTWNKKIAIYNDRVNSKLSRVMDPTCIVANIQGRETILVMVGKWNNNDKTWGAYRDKAPDTDWDLVLYKSTDDGVTFSKVETNIHDIVTKNGTISAMLGGVGSGLQLNDGKLVFPVQMVRTKNITTVLNTSFIYSTDGITWSLPSGYCEGFGSENNIIEFNASLVNNIRNSGLRRSFETKDFGKTWTEFPPMDKKVDNRNHGVQGSTITIPSGNKLVAAHSSAQNKNNDYTRSDISLYAHNLYSGEVKLIDDFYPKVGNASGAGYSCLSYRKNVDKETLYVVYEANGSIEFQDLSRHLPVIKSYNGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGKSGGGGSHHHHHHHH

SEQ ID NO: 107 - DNA, 1896 bp, Artificial Sequence
  Description of Artificial Sequence Synthetic polynucleotide
acagtggaaaagtccgtggtgttcaaggccgagggcgagcacttcaccgaccagaaaggcaataccatcgtcggctctggcagcggcggcaccaccaagtactttagaatccccgccatgtgcaccaccagcaagggcaccattgtggtgttcgccgacgccagacacaacaccgccagcgatcagagcttcatcgataccgctgccgccagaagtacagacggcggcaagacctggaacaagaagatcgccatctacaacgaccgcgtgaacagcaagctgagcagagtgatggaccctacctgcatcgtggccaacatccagggcagagaaaccatcctggtcatggtcggaaagtggaacaacaacgataagacctggggcgcctacagagacaaggcccctgataccgattgggacctcgtgctgtataagagcaccgacgacggcgtgaccttcagcaaggtggaaacaaacatccacgacatcgtgaccaagaacggcaccatctctgccatgctcggcggcgttggatctggcctgcaactgaatgatggcaagctggtgttccccgtgcagatggtccgaacaaagaacatcaccaccgtgctgaataccagcttcatctactccaccgacggcatcacatggtccctgcctagcggctactgtgaaggctttggcagcgagaacaacatcatcgagttcaacgccagcctggtcaacaacatccggaacagcggcctgcggagaagcttcgagacaaaggacttcggaaagacgtggaccgagtttcctccaatggacaagaaggtggacaaccggaaccacggcgtgcagggcagcacaatcacaatccctagcggcaacaaactggtggccgctcactctagcgcccagaacaagaacaacgattacaccagaagcgacatcagcctgtacgcccacaacctgtactccggcgaagtgaagctgatcgacgacttctaccccaaagtgggcaatgccagcggagccggctacagctgtctgagctaccggaaaaatgtggacaaagaaaccctgtacgtggtgtacgaggccaacggcagcatcgagtttcaggacctgagcagacatctgcccgtgatcaagagctacaatggcggaggtggaagtggcggaggcggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctcactagcaagctcaccgtggacaagagcaggtggcagcaggggaacgtcttctcatgctccgtgatgcatgaggctctgcacaaccactacacgcagaagagcctctccctgtctccgggtaaaagcggcggaggcggatctcatcatcaccatcatcaccatcac

SEQ ID NO: 108 - protein, 632 aa, Artificial Sequence
  Description of Artificial Sequence Synthetic polypeptide
TVEKSVVFKAEGEHFTDQKGNTIVGSGSGGTTKYFRIPAMCTTSKGTIVVFADARHNTASDQSFIDTAAARSTDGGKTWNKKIAIYNDRVNSKLSRVMDPTCIVANIQGRETILVMVGKWNNNDKTWGAYRDKAPDTDWDLVLYKSTDDGVTFSKVETNIHDIVTKNGTISAMLGGVGSGLQLNDGKLVFPVQMVRTKNITTVLNTSFIYSTDGITWSLPSGYCEGFGSENNIIEFNASLVNNIRNSGLRRSFETKDFGKTWTEFPPMDKKVDNRNHGVQGSTITIPSGNKLVAAHSSAQNKNNDYTRSDISLYAHNLYSGEVKLIDDFYPKVGNASGAGYSCLSYRKNVDKETLYVVYEANGSIEFQDLSRHLPVIKSYNGGGGSGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGKSGGGGSHHHHHHHH

SEQ ID NO: 109 - DNA, 1896 bp, Artificial Sequence
  Description of Artificial Sequence Synthetic polynucleotide
acagtggaaaagtccgtggtgttcaaggccgagggcgagcacttcaccgaccagaaaggcaataccatcgtcggctctggcagcggcggcaccaccaagtactttagaatccccgccatgtgcaccaccagcaagggcaccattgtggtgttcgccgacgccagacacaacaccgccagcgatcagagcttcatcgataccgctgccgccagaagtacagacggcggcaagacctggaacaagaagatcgccatctacaacgaccgcgtgaacagcaagctgagcagagtgatggaccctacctgcatcgtggccaacatccagggcagagaaaccatcctggtcatggtcggaaagtggaacaacaacgataagacctggggcgcctacagagacaaggcccctgataccgattgggacctcgtgctgtataagagcaccgacgacggcgtgaccttcagcaaggtggaaacaaacatccacgacatcgtgaccaagaacggcaccatctctgccatgctcggcggcgttggatctggcctgcaactgaatgatggcaagctggtgttccccgtgcagatggtccgaacaaagaacatcaccaccgtgctgaataccagcttcatctactccaccgacggcatcacatggtccctgcctagcggctactgtgaaggctttggcagcgagaacaacatcatcgagttcaacgccagcctggtcaacaacatccggaacagcggcctgcggagaagcttcgagacaaaggacttcggaaagacgtggaccgagtttcctccaatggacaagaaggtggacaaccggaaccacggcgtgcagggcagcacaatcacaatccctagcggcaacaaactggtggccgctcactctagcgcccagaacaagaacaacgattacaccagaagcgacatcagcctgtacgcccacaacctgtactccggcgaagtgaagctgatcgacgacttctaccccaaagtgggcaatgccagcggagccggctacagctgtctgagctaccggaaaaatgtggacaaagaaaccctgtacgtggtgtacgaggccaacggcagcatcgagtttcaggacctgagcagacatctgcccgtgatcaagagctacaatggcggaggtggaagtggcggaggcggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggtagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctcactagcaagctcaccgtggacaagagcaggtggcagcaggggaacgtcttctcatgctccgtgatgcatgaggctctgcacaaccactacacgcagaagagcctctccctgtctccgggtaaaagcggcggaggcggatctcatcatcaccatcatcaccatcac

SEQ ID NO: 110 - DNA, 1350 bp, Artificial Sequence
  Description of Artificial Sequence Synthetic polynucleotide
caggttcagctggtgcagtctggcgtggaagtgaagaaacctggggccagcgtgaaggtgtcctgtaaagccagcggctacacctttaccaactactacatgtactgggtccgacaggccccaggccaaggacttgaatggatgggcggcatcaaccccagcaacggcggcaccaacttcaacgagaagttcaagaacagagtgaccctgaccaccgacagcagcaccaccacagcctacatggaactgaagtccctgcagttcgacgacaccgccgtgtactactgcgccagacgggactacagattcgacatgggcttcgactattggggccagggcaccaccgtgacagtctcttctgctagcaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctcaccagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 111 - protein, 612 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 617 aa, Artificial Sequence; identical to SEQ ID NO: 67
  Description of Artificial Sequence Synthetic polypeptide

SEQ ID NO: 113 - DNA, 1851 bp, Artificial Sequence
  Description of Artificial Sequence Synthetic polynucleotide
gatgcatctctgccttacctgcagaaagaaagcgtgttccagtctggcgcccacgcctacagaattcccgctctgctgtatctgccaggccagcagtctctgctggctttcgctgaacagcgggccagcaagaaggatgagcacgccgaactgatcgtgctgcggagaggcgattacgacgccggcacacatcaggtgcagtggcaggctcaagaggtggtggctcaggctagactggacggccacagatctatgaacccctgtcctctgtacgatgaacagaccggcacactgtttctgttctttatcgctatccccggccaagtgaccgagcagcagcagctgcagacaagagccaacgtgaccagactgtgtcaagtgacctccaccgaccacggcagaacctggtctagccctagagatctgaccgacgccgccatcggacctgcctatagagagtggtccaccttcgccgttggacctggacactgtctccagctgcacgacagggctagatctctggtggtgcctgcctacgcctatagaaagctgcaccccaaacagcggcctattcctagcgccttctgctttctgagccacgatcacggcaggacatgggccagaggacatttcgtggcccaggacacactggaatgccaggtggccgaagtggaaaccggcgagcagagagtcgtgaccctgaacgccagatctcacctgagagccagagtgcaggcccagagcacaaacgacggcctggatttccaagagagccagctggtcaagaaactggtggaacctcctccacagggctgtcagggaagcgtgatcagctttccatctcctagaagcggccctggctctcctgctcagtggctgctgtatacacaccccacacacagctggcagagagccgatctgggcgcctacctgaatcctagacctcctgctcctgaggcttggagcgaacctgttctgctggccaagggcagcgctgcctacagcgatctgcagtctatgggcacaggccctgatggcagccctctgtttggctgtctgtacgaggccaacgactacgaagagatcgtgttcctgatgttcaccctgaagcaggcctttccagccgagtacctgcctcaaggcggaggtggaagtggcggaggcggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctcactagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 114 - DNA, 1836 bp, Artificial Sequence
  Description of Artificial Sequence Synthetic polynucleotide
gatgcatctctgccttacctgcagaaagaaagcgtgttccagtctggcgcccacgcctacagaattcccgctctgctgtatctgccaggccagcagtctctgctggctttcgctgaacagcgggccagcaagaaggatgagcacgccgaactgatcgtgctgcggagaggcgattacgacgccggcacacatcaggtgcagtggcaggctcaagaggtggtggctcaggctagactggacggccacagatctatgaacccctgtcctctgtacgatgaacagaccggcacactgtttctgttctttatcgctatccccggccaagtgaccgagcagcagcagctgcagacaagagccaacgtgaccagactgtgtcaagtgacctccaccgaccacggcagaacctggtctagccctagagatctgaccgacgccgccatcggacctgcctatagagagtggtccaccttcgccgttggacctggacactgtctccagctgcacgacagggctagatctctggtggtgcctgcctacgcctatagaaagctgcaccccaaacagcggcctattcctagcgccttctgctttctgagccacgatcacggcaggacatgggccagaggacatttcgtggcccaggacacactggaatgccaggtggccgaagtggaaaccggcgagcagagagtcgtgaccctgaacgccagatctcacctgagagccagagtgcaggcccagagcacaaacgacggcctggatttccaagagagccagctggtcaagaaactggtggaacctcctccacagggctgtcagggaagcgtgatcagctttccatctcctagaagcggccctggctctcctgctcagtggctgctgtatacacaccccacacacagctggcagagagccgatctgggcgcctacctgaatcctagacctcctgctcctgaggcttggagcgaacctgttctgctggccaagggcagcgctgcctacagcgatctgcagtctatgggcacaggccctgatggcagccctctgtttggctgtctgtacgaggccaacgactacgaagagatcgtgttcctgatgttcaccctgaagcaggcctttccagccgagtacctgcctcaagagcccaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgtactgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctaagcttgtctccgggtaaa

SEQ ID NO: 115 - protein, 612 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCYVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRFRVQAQSTNDGLDFQESQLVKKLVEPPPTGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 608 aa, Artificial Sequence
  Description of Artificial Sequence Synthetic polypeptide
  MOD_RES (1)..(1): Any amino acid
  MOD_RES (6)..(6): Any amino acid
  MOD_RES (9)..(9): Any amino acid
  MOD_RES (62)..(62): Any amino acid
  MOD_RES (93)..(93): Any amino acid
  MOD_RES (126)..(126): Any amino acid
  MOD_RES (187)..(187): Any amino acid
  MOD_RES (242)..(242): Any amino acid
  MOD_RES (270)..(270): Any amino acid
  MOD_RES (301)..(302): Any amino acid
  MOD_RES (332)..(332): Any amino acid
  MOD_RES (363)..(363): Any amino acid
  MOD_RES (365)..(365): Any amino acid
  MOD_RES (381)..(381): Any amino acid
XASLPXLQXESVFQSGAHAYRIPALLYLPGQQSLLAFAEQRASKKDEHAELIVLRRGDYDAXTHQVQWQAQEVVAQARLDGHRSMNPCPLYDXQTGTLFLFFIAIPGQVTEQQQLQTRANVTRLCXVTSTDHGRTWSSPRDLTDAAIGPAYREWSTFAVGPGHCLQLHDRARSLVVPAYAYRKLHPXQRPIPSAFCFLSHDHGRTWARGHFVAQDTLECQVAEVETGEQRVVTLNARSHLRXRVQAQSTNDGLDFQESQLVKKLVEPPPXGCQGSVISFPSPRSGPGSPAQWLLYTHPTHXXQRADLGAYLNPRPPAPEAWSEPVLLAKGSXAYSDLQSMGTGPDGSPLFGCLYEANDYEEIXFXMFTLKQAFPAEYLPQXDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 117 - protein, 608 aa, Artificial Sequence
  Description of Artificial Sequence Synthetic polypeptide
  MOD_RES (1)..(2): Any amino acid
  MOD_RES (4)..(6): Any amino acid
  MOD_RES (9)..(9): Any amino acid
  MOD_RES (44)..(45): Any amino acid
  MOD_RES (54)..(54): Any amino acid
  MOD_RES (62)..(62): Any amino acid
  MOD_RES (69)..(69): Any amino acid
  MOD_RES (78)..(78): Any amino acid
  MOD_RES (80)..(80): Any amino acid
  MOD_RES (93)..(93): Any amino acid
  MOD_RES (107)..(108): Any amino acid
  MOD_RES (112)..(112): Any amino acid
  MOD_RES (125)..(126): Any amino acid
  MOD_RES (150)..(150): Any amino acid
  MOD_RES (164)..(164): Any amino acid
  MOD_RES (170)..(171): Any amino acid
  MOD_RES (187)..(189): Any amino acid
  MOD_RES (196)..(196): Any amino acid
  MOD_RES (213)..(213): Any amino acid
  MOD_RES (217)..(217): Any amino acid
  MOD_RES (225)..(225): Any amino acid
  MOD_RES (239)..(242): Any amino acid
  MOD_RES (244)..(244): Any amino acid
  MOD_RES (249)..(249): Any amino acid
  MOD_RES (251)..(251): Any amino acid
  MOD_RES (257)..(258): Any amino acid
  MOD_RES (260)..(260): Any amino acid
  MOD_RES (265)..(265): Any amino acid
  MOD_RES (270)..(270): Any amino acid
  MOD_RES (272)..(272): Any amino acid
  MOD_RES (292)..(292): Any amino acid
  MOD_RES (301)..(302): Any amino acid
  MOD_RES (325)..(325): Any amino acid
  MOD_RES (332)..(332): Any amino acid
  MOD_RES (352)..(352): Any amino acid
  MOD_RES (363)..(363): Any amino acid
  MOD_RES (365)..(365): Any amino acid
  MOD_RES (381)..(381): Any amino acid
XXSXXXLQXESVFQSGAHAYRIPALLYLPGQQSLLAFAEQRASXXDEHAELIVXRRGDYDAXTHQVQWXAQEVVAQAXLXGHRSMNPCPLYDXQTGTLFLFFIAIPXXVTEXQQLQTRANVTRLXXVTSTDHGRTWSSPRDLTDAAIGPXYREWSTFAVGPGHXLQLHDXXRSLVVPAYAYRKLHPXXXPIPSAFXFLSHDHGRTWARGHFVXQDTXECQVAEVXTGEQRVVTLNARSXXXXRXQAQSXNXGLDFQXXQXVKKLXEPPPXGXQGSVISFPSPRSGPGSPAQXLLYTHPTHXXQRADLGAYLNPRPPAPEAWSEPXLLAKGSXAYSDLQSMGTGPDGSPLFGXLYEANDYEEIXFXMFTLKQAFPAEYLPQXDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 118 - DNA, 1140 bp, Artificial Sequence
  Description of Artificial Sequence Synthetic polynucleotide
gatgcatctctgccttacctgcagaaagaaagcgtgttccagtctggcgcccacgcctacagaattcccgctctgctgtatctgccaggccagcagtctctgctggctttcgctgaacagcgggccagcaagaaggatgagcacgccgaactgatcgtgctgcggagaggcgattacgacgccggcacacatcaggtgcagtggcaggctcaagaggtggtggctcaggctagactggacggccacagatctatgaacccctgtcctctgtacgatgaacagaccggcacactgtttctgttctttatcgctatccccggccaagtgaccgagcagcagcagctgcagacaagagccaacgtgaccagactgtgttacgtgacctccaccgaccacggcagaacctggtctagccctagagatctgaccgacgccgccatcggacctgcctatagagagtggtccaccttcgccgttggacctggacactgtctccagctgcacgacagggctagatctctggtggtgcctgcctacgcctatagaaagctgcaccccaaacagcggcctattcctagcgccttctgctttctgagccacgatcacggcaggacatgggccagaggacatttcgtggcccaggacacactggaatgccaggtggccgaagtggaaaccggcgagcagagagtcgtgaccctgaacgccagatctcacctgagattcagagtgcaggcccagagcacaaacgacggcctggatttccaagagagccagctggtcaagaaactggtggaacctcctccaaccggctgtcagggaagcgtgatcagctttccatctcctagaagcggccctggctctcctgctcagtggctgctgtatacacaccccacacacagctggcagagagccgatctgggcgcctacctgaatcctagacctcctgctcctgaggcttggagcgaacctgttctgctggccaagggcagcgctgcctacagcgatctgcagtctatgggcacaggccctgatggcagccctctgtttggctgtctgtacgaggccaacgactacgaagagatcgtgttcctgatgttcaccctgaagcaggcctttccagccgagtacctgcctcaa

SEQ ID NO: 119 - protein, 380 aa, Artificial Sequence
  Description of Artificial Sequence Synthetic polypeptide
  MOD_RES (1)..(2): Any amino acid
  MOD_RES (4)..(6): Any amino acid
  MOD_RES (9)..(9): Any amino acid
  MOD_RES (44)..(45): Any amino acid
  MOD_RES (54)..(54): Any amino acid
  MOD_RES (62)..(62): Any amino acid
  MOD_RES (69)..(69): Any amino acid
  MOD_RES (78)..(78): Any amino acid
  MOD_RES (80)..(80): Any amino acid
  MOD_RES (93)..(93): Any amino acid
  MOD_RES (107)..(108): Any amino acid
  MOD_RES (112)..(112): Any amino acid
  MOD_RES (125)..(126): Any amino acid
  MOD_RES (150)..(150): Any amino acid
  MOD_RES (164)..(164): Any amino acid
  MOD_RES (170)..(171): Any amino acid
  MOD_RES (187)..(189): Any amino acid
  MOD_RES (196)..(196): Any amino acid
  MOD_RES (213)..(213): Any amino acid
  MOD_RES (217)..(217): Any amino acid
  MOD_RES (225)..(225): Any amino acid
  MOD_RES (239)..(242): Any amino acid
  MOD_RES (244)..(244): Any amino acid
  MOD_RES (249)..(249): Any amino acid
  MOD_RES (251)..(251): Any amino acid
  MOD_RES (257)..(258): Any amino acid
  MOD_RES (260)..(260): Any amino acid
  MOD_RES (265)..(265): Any amino acid
  MOD_RES (270)..(270): Any amino acid
  MOD_RES (272)..(272): Any amino acid
  MOD_RES (292)..(292): Any amino acid
  MOD_RES (301)..(302): Any amino acid
  MOD_RES (325)..(325): Any amino acid
  MOD_RES (332)..(332): Any amino acid
  MOD_RES (352)..(352): Any amino acid
  MOD_RES (363)..(363): Any amino acid
  MOD_RES (365)..(365): Any amino acid
XXSXXXLQXESVFQSGAHAYRIPALLYLPGQQSLLAFAEQRASXXDEHAELIVXRRGDYDAXTHQVQWXAQEVVAQAXLXGHRSMNPCPLYDXQTGTLFLFFIAIPXXVTEXQQLQTRANVTRLXXVTSTDHGRTWSSPRDLTDAAIGPXYREWSTFAVGPGHXLQLHDXXRSLVVPAYAYRKLHPXXXPIPSAFXFLSHDHGRTWARGHFVXQDTXECQVAEVXTGEQRVVTLNARSXXXXRXQAQSXNXGLDFQXXQXVKKLXEPPPXGXQGSVISFPSPRSGPGSPAQXLLYTHPTHXXQRADLGAYLNPRPPAPEAWSEPXLLAKGSXAYSDLQSMGTGPDGSPLFGXLYEANDYEEIXFXMFTLKQAFPAEYLPQ

SEQ ID NO: 120 - protein, 380 aa, Artificial Sequence
  Description of Artificial Sequence Synthetic polypeptide
  MOD_RES (1)..(1): Any amino acid
  MOD_RES (6)..(6): Any amino acid
  MOD_RES (9)..(9): Any amino acid
  MOD_RES (62)..(62): Any amino acid
  MOD_RES (93)..(93): Any amino acid
  MOD_RES (126)..(126): Any amino acid
  MOD_RES (187)..(187): Any amino acid
  MOD_RES (242)..(242): Any amino acid
  MOD_RES (270)..(270): Any amino acid
  MOD_RES (301)..(302): Any amino acid
  MOD_RES (332)..(332): Any amino acid
  MOD_RES (363)..(363): Any amino acid
  MOD_RES (365)..(365): Any amino acid
XASLPXLQXESVFQSGAHAYRIPALLYLPGQQSLLAFAEQRASKKDEHAELIVLRRGDYDAXTHQVQWQAQEVVAQARLDGHRSMNPCPLYDXQTGTLFLFFIAIPGQVTEQQQLQTRANVTRLCXVTSTDHGRTWSSPRDLTDAAIGPAYREWSTFAVGPGHCLQLHDRARSLVVPAYAYRKLHPXQRPIPSAFCFLSHDHGRTWARGHFVAQDTLECQVAEVETGEQRVVTLNARSHLRXRVQAQSTNDGLDFQESQLVKKLVEPPPXGCQGSVISFPSPRSGPGSPAQWLLYTHPTHXXQRADLGAYLNPRPPAPEAWSEPVLLAKGSXAYSDLQSMGTGPDGSPLFGCLYEANDYEEIXFXMFTLKQAFPAEYLPQ

SEQ ID NO: 121 - protein, 5 aa, Artificial Sequence
  Description of Artificial Sequence Synthetic peptide
GGGGS

SEQ ID NO: 122 - protein, 612 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCYVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPTGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 612 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCYVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRFRVQAQSTNDGLDFQESQLVKKLVEPPPTGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - DNA, 1836 bp, Artificial Sequence
  Description of Artificial Sequence Synthetic polynucleotide
gatgcatctctgccttacctgcagaaagaaagcgtgttccagtctggcgcccacgcctacagaattcccgctctgctgtatctgccaggccagcagtctctgctggctttcgctgaacagcgggccagcaagaaggatgagcacgccgaactgatcgtgctgcggagaggcgattacgacgccggcacacatcaggtgcagtggcaggctcaagaggtggtggctcaggctagactggacggccacagatctatgaacccctgtcctctgtacgatgaacagaccggcacactgtttctgttctttatcgctatccccggccaagtgaccgagcagcagcagctgcagacaagagccaacgtgaccagactgtgttacgtgacctccaccgaccacggcagaacctggtctagccctagagatctgaccgacgccgccatcggacctgcctatagagagtggtccaccttcgccgttggacctggacactgtctccagctgcacgacagggctagatctctggtggtgcctgcctacgcctatagaaagctgcaccccaaacagcggcctattcctagcgccttctgctttctgagccacgatcacggcaggacatgggccagaggacatttcgtggcccaggacacactggaatgccaggtggccgaagtggaaaccggcgagcagagagtcgtgaccctgaacgccagatctcacctgagattcagagtgcaggcccagagcacaaacgacggcctggatttccaagagagccagctggtcaagaaactggtggaacctcctccaaccggctgtcagggaagcgtgatcagctttccatctcctagaagcggccctggctctcctgctcagtggctgctgtatacacaccccacacacagctggcagagagccgatctgggcgcctacctgaatcctagacctcctgctcctgaggcttggagcgaacctgttctgctggccaagggcagcgctgcctacagcgatctgcagtctatgggcacaggccctgatggcagccctctgtttggctgtctgtacgaggccaacgactacgaagagatcgtgttcctgatgttcaccctgaagcaggcctttccagccgagtacctgcctcaagagcccaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgtactgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctaagcttgtctccgggtaaa

SEQ ID NO: 125 - protein, 612 aa, Artificial Sequence
  Description of Artificial Sequence Synthetic polypeptide
DASLPYLQKESVFQSGAHAYRIPALLYLPGQQSLLAFAEQRRSKKDEHAELIVLRRGDYDAGTHQVQWQAQEVVAQARLDGHRSMNPCPLYDEQTGTLFLFFIAIPGQVTEQQQLQTRANVTRLCYVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRFRVQAQSTNDGLDFQESQLVKKLVEPPPTGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 380 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCYVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQ

SEQ ID NO: 127 - protein, 612 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCYVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 128 - protein, 617 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCYVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 129 - DNA, 1836 bp, Artificial Sequence
  Description of Artificial Sequence Synthetic polynucleotide
gatgcatctctgccttacctgcagaaagaaagcgtgttccagtctggcgcccacgcctacagaattcccgctctgctgtatctgccaggccagcagtctctgctggctttcgctgaacagcggcggagcaagaaggatgagcacgccgaactgatcgtgctgcggagaggcgattacgacgccggcacacatcaggtgcagtggcaggctcaagaggtggtggctcaggctagactggacggccacagatctatgaacccctgtcctctgtacgatgaacagaccggcacactgtttctgttctttatcgctatccccggccaagtgaccgagcagcagcagctgcagacaagagccaacgtgaccagactgtgttacgtgacctccaccgaccacggcagaacctggtctagccctagagatctgaccgacgccgccatcggacctgcctatagagagtggtccaccttcgccgttggacctggacactgtctccagctgcacgacagggctagatctctggtggtgcctgcctacgcctatagaaagctgcaccccaaacagcggcctattcctagcgccttctgctttctgagccacgatcacggcaggacatgggccagaggacatttcgtggcccaggacacactggaatgccaggtggccgaagtggaaaccggcgagcagagagtcgtgaccctgaacgccagatctcacctgagattcagagtgcaggcccagagcacaaacgacggcctggatttccaagagagccagctggtcaagaaactggtggaacctcctccaaccggctgtcagggaagcgtgatcagctttccatctcctagaagcggccctggctctcctgctcagtggctgctgtatacacaccccacacacagctggcagagagccgatctgggcgcctacctgaatcctagacctcctgctcctgaggcttggagcgaacctgttctgctggccaagggcagcgctgcctacagcgatctgcagtctatgggcacaggccctgatggcagccctctgtttggctgtctgtacgaggccaacgactacgaagagatcgtgttcctgatgttcaccctgaagcaggcctttccagccgagtacctgcctcaagagcccaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgtactgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctaagcttgtctccgggtaaa

SEQ ID NO: 130 - protein, 854 aa, Artificial Sequence
  Description of Artificial Sequence Synthetic polypeptide
DASLPYLQKESVFQSGAHAYRIPALLYLPGQQSLLAFAEQRRSKKDEHAELIVLRRGDYDAGTHQVQWQAQEVVAQARLDGHRSMNPCPLYDEQTGTLFLFFIAIPGQVTEQQQLQTRANVTRLCYVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRFRVQAQSTNDGLDFQESQLVKKLVEPPPTGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTS

SEQ ID NO: 131 - DNA, 2562 bp, Artificial Sequence
  Description of Artificial Sequence Synthetic polynucleotide
gatgcatctctgccttacctgcagaaagaaagcgtgttccagtctggcgcccacgcctacagaattcccgctctgctgtatctgccaggccagcagtctctgctggctttcgctgaacagcggcggagcaagaaggatgagcacgccgaactgatcgtgctgcggagaggcgattacgacgccggcacacatcaggtgcagtggcaggctcaagaggtggtggctcaggctagactggacggccacagatctatgaacccctgtcctctgtacgatgaacagaccggcacactgtttctgttctttatcgctatccccggccaagtgaccgagcagcagcagctgcagacaagagccaacgtgaccagactgtgttacgtgacctccaccgaccacggcagaacctggtctagccctagagatctgaccgacgccgccatcggacctgcctatagagagtggtccaccttcgccgttggacctggacactgtctccagctgcacgacagggctagatctctggtggtgcctgcctacgcctatagaaagctgcaccccaaacagcggcctattcctagcgccttctgctttctgagccacgatcacggcaggacatgggccagaggacatttcgtggcccaggacacactggaatgccaggtggccgaagtggaaaccggcgagcagagagtcgtgaccctgaacgccagatctcacctgagattcagagtgcaggcccagagcacaaacgacggcctggatttccaagagagccagctggtcaagaaactggtggaacctcctccaaccggctgtcagggaagcgtgatcagctttccatctcctagaagcggccctggctctcctgctcagtggctgctgtatacacaccccacacacagctggcagagagccgatctgggcgcctacctgaatcctagacctcctgctcctgaggcttggagcgaacctgttctgctggccaagggcagcgctgcctacagcgatctgcagtctatgggcacaggccctgatggcagccctctgtttggctgtctgtacgaggccaacgactacgaagagatcgtgttcctgatgttcaccctgaagcaggcctttccagccgagtacctgcctcaagagcccaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctaagcttgtctccgggtaaaggaggcggaggatctggcggaggtggaagtggcggaggcggatctcaggttcagctggtgcagtctggcgtggaagtgaagaaacctggggccagcgtgaaggtgtcctgtaaagccagcggctacacctttaccaactactacatgtactgggtccgacaggccccaggccaaggacttgaatggatgggcggcatcaaccccagcaacggcggcaccaacttcaacgagaagttcaagaacagagtgaccctgaccaccgacagcagcaccaccacagcctacatggaactgaagtccctgcagttcgacgacaccgccgtgtactactgcgccagacgggactacagattcgacatgggcttcgactattggggccagggcaccaccgtgacagtctcttctgctagcaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactagc

SEQ ID NO: 132 - protein, 227 aa, Artificial Sequence
  Description of Artificial Sequence Synthetic polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTS

SEQ ID NO: 133 - DNA, 681 bp, Artificial Sequence
  Description of Artificial Sequence Synthetic polynucleotide
caggttcagctggtgcagtctggcgtggaagtgaagaaacctggggccagcgtgaaggtgtcctgtaaagccagcggctacacctttaccaactactacatgtactgggtccgacaggccccaggccaaggacttgaatggatgggcggcatcaaccccagcaacggcggcaccaacttcaacgagaagttcaagaacagagtgaccctgaccaccgacagcagcaccaccacagcctacatggaactgaagtccctgcagttcgacgacaccgccgtgtactactgcgccagacgggactacagattcgacatgggcttcgactattggggccagggcaccaccgtgacagtctcttctgctagcaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactagc

SEQ ID NO: 134 - protein, 845 aa, Artificial Sequence
  Description of Artificial Sequence Synthetic polypeptide
DASLPYLQKESVFQSGAHAYRIPALLYLPGQQSLLAFAEQRRSKKDEHAELIVLRRGDYDAGTHQVQWQAQEVVAQARLDGHRSMNPCPLYDEQTGTLFLFFIAIPGQVTEQQQLQTRANVTRLCYVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRFRVQAQSTNDGLDFQESQLVKKLVEPPPTGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 135 - DNA, 2535 bp, Artificial Sequence
  Description of Artificial Sequence Synthetic polynucleotide
gatgcatctctgccttacctgcagaaagaaagcgtgttccagtctggcgcccacgcctacagaattcccgctctgctgtatctgccaggccagcagtctctgctggctttcgctgaacagcggcggagcaagaaggatgagcacgccgaactgatcgtgctgcggagaggcgattacgacgccggcacacatcaggtgcagtggcaggctcaagaggtggtggctcaggctagactggacggccacagatctatgaacccctgtcctctgtacgatgaacagaccggcacactgtttctgttctttatcgctatccccggccaagtgaccgagcagcagcagctgcagacaagagccaacgtgaccagactgtgttacgtgacctccaccgaccacggcagaacctggtctagccctagagatctgaccgacgccgccatcggacctgcctatagagagtggtccaccttcgccgttggacctggacactgtctccagctgcacgacagggctagatctctggtggtgcctgcctacgcctatagaaagctgcaccccaaacagcggcctattcctagcgccttctgctttctgagccacgatcacggcaggacatgggccagaggacatttcgtggcccaggacacactggaatgccaggtggccgaagtggaaaccggcgagcagagagtcgtgaccctgaacgccagatctcacctgagattcagagtgcaggcccagagcacaaacgacggcctggatttccaagagagccagctggtcaagaaactggtggaacctcctccaaccggctgtcagggaagcgtgatcagctttccatctcctagaagcggccctggctctcctgctcagtggctgctgtatacacaccccacacacagctggcagagagccgatctgggcgcctacctgaatcctagacctcctgctcctgaggcttggagcgaacctgttctgctggccaagggcagcgctgcctacagcgatctgcagtctatgggcacaggccctgatggcagccctctgtttggctgtctgtacgaggccaacgactacgaagagatcgtgttcctgatgttcaccctgaagcaggcctttccagccgagtacctgcctcaagagcccaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctaagcttgtctccgggtaaaggaggcggaggatctggcggaggtggaagtggcggaggcggatctgagatcgtgctgacacagagccctgccacactgtcactgtctccaggcgagagagccacactgagctgtagagcttctaagggcgtgtccaccagcggctactcttacctgcactggtatcagcagaagcccggccaggctcctagactgctgatctacctggccagctacctggaaagcggagtgcctgctagattcagcggctctggctctggcaccgacttcaccctgacaatcagcagcctggaacctgaggacttcgccgtgtactactgccagcacagcagagatctgcccctgacatttggcggaggcaccaaggtggaaatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 136 - protein, 447 aa, Artificial Sequence
  Description of Artificial Sequence Synthetic polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 137 - protein, 120 aa, Artificial Sequence [sequence omitted]

SEQ ID NO: 138 - protein, 111 aa, Artificial Sequence [sequence omitted]

SEQ ID NO: 139 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 142 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 144 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 864 aa, Artificial Sequence
  Description of Artificial Sequence Synthetic polypeptide
MGWSCIILFLVATATGVHSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCYVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRFRVQAQSTNDGLDFQESQLVKKLVEPPPTGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQ

SEQ ID NO: 146 - DNA, 2598 bp, Artificial Sequence
  Description of Artificial Sequence Synthetic polynucleotide
atgggatggagctgtatcatcctcttcttggtagcaacagctacaggtgtacactcccaggttcagctggtgcagtctggcgtggaagtgaagaaacctggggccagcgtgaaggtgtcctgtaaagccagcggctacacctttaccaactactacatgtactgggtccgacaggccccaggccaaggacttgaatggatgggcggcatcaaccccagcaacggcggcaccaacttcaacgagaagttcaagaacagagtgaccctgaccaccgacagcagcaccaccacagcctacatggaactgaagtccctgcagttcgacgacaccgccgtgtactactgcgccagacgggactacagattcgacatgggcttcgactattggggccagggcaccaccgtgacagtctcttctgctagcaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctaagcttgtctccgggtaaaggaggcggaggatctggcggaggtggaagtggcggaggcggatctgatgcatctctgccttacctgcagaaagaaagcgtgttccagtctggcgcccacgcctacagaattcccgctctgctgtatctgccaggccagcagtctctgctggctttcgctgaacagcgggccagcaagaaggatgagcacgccgaactgatcgtgctgcggagaggcgattacgacgccggcacacatcaggtgcagtggcaggctcaagaggtggtggctcaggctagactggacggccacagatctatgaacccctgtcctctgtacgatgaacagaccggcacactgtttctgttctttatcgctatccccggccaagtgaccgagcagcagcagctgcagacaagagccaacgtgaccagactgtgttacgtgacctccaccgaccacggcagaacctggtctagccctagagatctgaccgacgccgccatcggacctgcctatagagagtggtccaccttcgccgttggacctggacactgtctccagctgcacgacagggctagatctctggtggtgcctgcctacgcctatagaaagctgcaccccaaacagcggcctattcctagcgccttctgctttctgagccacgatcacggcaggacatgggccagaggacatttcgtggcccaggacacactggaatgccaggtggccgaagtggaaaccggcgagcagagagtcgtgaccctgaacgccagatctcacctgagattcagagtgcaggcccagagcacaaacgacggcctggatttccaagagagccagctggtcaagaaactggtggaacctcctccaaccggctgtcagggaagcgtgatcagctttccatctcctagaagcggccctggctctcctgctcagtggctgctgtatacacaccccacacacagctggcagagagccgatctgggcgcctacctgaatcctagacctcctgctcctgaggcttggagcgaacctgttctgctggccaagggcagcgctgcctacagcgatctgcagtctatgggcacaggccctgatggcagccctctgtttggctgtctgtacgaggccaacgactacgaagagatcgtgttcctgatgttcaccctgaagcaggcctttccagccgagtacctgcctcaatagtga

SEQ ID NO: 147 - protein, 10 aa, Artificial Sequence
  Description of Artificial Sequence Synthetic 10xHis tag
HHHHHHHHHH

SEQ ID NO: 148 - protein, 15 aa, Artificial Sequence
  Description of Artificial Sequence Synthetic peptide
  SITE (1)..(15): This sequence may encompass 1-5 “Gly Gly Pro” repeating units
GGPGGPGGPGGPGGP

SEQ ID NO: 149 - protein, 25 aa, Artificial Sequence
  Description of Artificial Sequence Synthetic peptide
  SITE (1)..(25): This sequence may encompass 1-5 “Gly Gly Gly Gly Ser” repeating units
GGGGSGGGGSGGGGSGGGGSGGGGS

SEQ ID NO: 150 - protein, 6 aa, Artificial Sequence
  Description of Artificial Sequence Synthetic 6xHis tag
HHHHHH